(19) United States
(12) Patent Application Publication
(10) Pub. No.: US 2022/0195459 A1
(43) Pub. Date: Jun. 23, 2022

(54) REGULATABLE EXPRESSION USING ADENO-ASSOCIATED VIRUS (AAV)

(71) Applicant: Voyager Therapeutics, Inc., Cambridge, MA (US)

(72) Inventor: Robert KOTIN, Boston, MA (US)

(21) Appl. No.: 17/543,310

(22) Filed: Dec. 6, 2021

Related U.S. Application Data
(63) Continuation of application No. 15/772,023, filed on Apr. 27, 2018, now abandoned.
(60) Provisional application No. 62/247,365, filed on Oct. 28, 2015.
(60) Provisional application No. 62/298,640, filed on Feb. 23, 2016.

(51) Int. Cl. C12N 15/86; A61K 35/76; A61K 45/06; C07K 14/72; C12N 9/22; C12N 15/11

(52) U.S. Cl. CPC C12N 15/86; A61K 35/76; A61K 45/06; C07K 14/721; C12N 9/22; C12N 15/11; C12N 2840/002; C12N 2750/14143; Y02A 50/30; C12N 2750/14141; C12N 2310/20; C07K 2319/80; C12N 2830/001

(57) ABSTRACT

The present invention relates to viral particles which exhibit self-regulatory or regulatable features.

### CROSS REFERENCE TO RELATED APPLICATIONS

This application claims the benefit of U.S. Provisional Application No. US 62/247,365 filed Oct. 28, 2015, entitled Regulatable Expression Using Adeno-Associate Virus (AAV), and U.S. Provisional Application No. US 62/298,640 filed Feb. 23, 2016, entitled Regulatable Expression Using Adeno-Associate Virus (AAV), the contents of each are herein incorporated by reference in their entirety.

### REFERENCE TO THE SEQUENCE LISTING

The present application is being filed along with a Sequence Listing in electronic format. The Sequence Listing file, entitled 20571504PCT_SEQLST.txt, was created on Oct. 27, 2016 and is 5,113,878 bytes in size. The information in electronic format of the Sequence Listing is incorporated herein by reference in its entirety.

### FIELD OF THE INVENTION

The present invention relates to compositions, methods and processes for the design, preparation, manufacture and/or formulation of recombinant parvovirus, e.g. adeno-associated virus (AAV), particles having one or more regulatable elements and methods of using the same.

### BACKGROUND OF THE INVENTION

The present invention provides AAV-based compositions and complexes which go beyond those of the art in order to address the need for new technologies for treating genetic disorders caused by abnormalities in the genome whether heritable or acquired, monogenic or multifactorial.

AAV vectors are used for gene therapy for a number of reasons including their reduced immunogenicity and their sustained long-term presence and transgene expression in target tissues. Recombinant AAVs typically remain within target tissues as episomal entities over the lifetime of the animal host. Due to the long term stability of the AAV vector, it is desirable to exert tight control on transgene expression, e.g., the time, place and level of transgene expression must be controlled. For example, if transgene expression is no longer needed or undesirable, for example due to toxicity or side effects, efficient mechanisms are needed to turn expression off. In other cases, it may be desirable to turn reversibly transgene expression on and off very quickly.

A number of switches that function to regulate transgene expression in the context of cell systems and animal models have been described. One such mechanism of regulation relies on a chemical agent, such as a drug, or a physiological stimulus that acts as a switch to turn the expression of a transgene on or off. The most extensively studied regulatory switch mechanism is the Tet ON/OFF system, in which a tet repressor protein can only activate transcription from a promoter with a tet response element in the presence or absence of teracyclin, first described by Bujard and Gossen (Proc Natl Acad Sci U S A. 1992 Jun. 15; 89(12):5547-51, Tight control of gene expression in mammalian cells by tetracycline-responsive promoters; the contents of which are herein incorporated by reference in its entirety). Several ligand and hormone regulatable systems, which employ the dimerization of two separate proteins for activation or repression, have also been described.

Approaches in which a second transgene encodes a regulatory enzyme such as a CRE recombinase, which modulates expression of a target transgene through site specific recombination, is extensively used in transgenic mouse studies, where a tissue restricted or temporally restricted expression pattern is desired.

In addition, transgene expression can also be controlled through regulation of transcript mRNA stability or protein stability, through the inclusion of stabilizing or destabilizing elements.

The present invention provides AAV-based compositions comprising a recombinant adeno-associated virus particles (AAV particles) having at least one regulatable element. These elements when used in association with AAV technology allow, for the first time, the regulatable tuning of payload expression from a viral genome delivered by an AAV particle.

### SUMMARY OF THE INVENTION

The present invention provides regulatable-AAV particles comprising at least one regulatable element to regulate the expression of a transgene or gene. Such regulation can be the inhibition or activation of transgene or gene expression or gene replacement. Such outcomes are achieved by utilizing regulatable elements encoded in the AAV particles (e.g., regulatable-AAV particles) described herein (e.g., the payload or VP2) in such a manner as to tune or control the level or degree of expression of the payload (whether a polynucleotide useful for gene knockdown, activation, or inhibition, or for gene replacement) encoded by the viral genome.

In one embodiment, the present invention is a composition comprising an AAV particle comprising a viral genome encoding at least one payload and the AAV particle may also comprise a viral genome encoding at least one regulatable element. As a non-limiting example, the viral genome encoding at least one regulatable element may be part of the payload. In some embodiments, one or more regulatable elements may include one or more proteins or fusion proteins. In a non-limiting example, the proteins or fusion proteins may be composed of a DNA binding domain, a transactivation domain or a repressor domain, a ligand binding domain and/or a dimerization domain. In some embodiments, the protein or fusion protein may be inducible through a ligand. In another non-limiting example, the proteins or fusion proteins may include a meganuclease, a zinc finger nuclease, a TALEN, a recombinase, an integrase, and/or a CRISPR Cas9. In one embodiment, the regulatable element may comprise a CRISPR Cas9 and may further comprise a single guide RNA (sgRNA).

In some embodiments, the protein or fusion protein may further include a destabilizing domain, which may be stabilized through a ligand and/or may include the estrogen receptor destabilizing domain.

In some embodiments, the regulatable element may comprise a regulatory RNA, such as a siRNA, microRNA (miRNA or miR) or ribozyme.

In some embodiments, the composition may include two or more regulatable elements, wherein the second regulatable element regulates the expression of the first regulatable element. A number of combinations of regulatable elements can be envisioned according to the invention and are described herein. In some embodiments, the payload and the regulatable elements may be located on the same viral genome. In some embodiments, the payload and the regulatable elements may be located on one or more separate viral genome.

In another embodiment, the present invention is a method of synthesizing a regulatable-AAV particle comprising a) introducing into competent bacterial cells i) a payload construct vector comprising a payload and one or more regulatable elements flanked on each side by a parvoviral ITR sequence to produce a payload construct expression vector; and ii) one or more viral construct vector(s) comprising parvoviral rep and/or cap gene sequences under the control of one or more regulatable elements to produce a viral construct expression vector; b) introducing into viral replication cells i) the payload construct expression vector produced in step (a.i) to produce a payload construct particle; and ii) the viral construct expression vector(s) produced in step (a.ii) to produce a viral construct particle; and c) co-infecting a viral replication cell with the payload construct viral particle produced in step (b.i) and the one or more viral construct viral particle(s) of step (b.ii) to produce a regulatable-AAV particle.

In another embodiment, one or more regulatable-AAV particles may be synthesized, wherein the payload and the regulatable element may be on separate payload constructs.

In another embodiment, the present invention may include one or more regulatable-AAV particles comprising a viral genome, the viral genome comprising: (a) at least one payload, and (b) at least one regulatable element.

In another embodiment, the present invention may include a method of treating a CNS disorder in a subject in need thereof, the method comprising administering to the subject a therapeutically effective amount of one or more regulatable-AAV particles comprising one or more viral genome, the viral genome comprising: (a) at least one payload, and (b) at least one regulatable element.

In one embodiment, provided are methods of regulating the expression of a protein of interest using one or more regulatable-AAV particle(s) a viral genome. In one aspect, the viral genome may have at least one payload and at least one regulatable element such as, but not limited to, a DNA binding domain which may be coupled with a transactivation domain. The regulatable element may be located in the VP2 capsid and may increase the expression of a protein of interest in a burst like fashion. The increase may be for at least 2 hours or may be for at least 6 hours.

In another aspect, the viral genome may have at least one payload and at least one CRISPR regulatable element such as, but not limited to, a cas9 endonuclease fused to a destabilizing domain or a Cpf1. The destabilizing domain may be a destabilizing domain from a protein family such as, but not limited to, FK506 Binding Protein (FKBP), E. coli dihyrofolate reductase (DHFR), mouse ornithine decarboxylase (MODC), and estrogen receptors (ER). As a non-limiting example, the destabilizing domain is from an estrogen receptor protein.

The details of one or more embodiments of the invention are set forth in the accompanying description below. Although any materials and methods similar or equivalent to those described herein can be used in the practice or testing of the present invention, the preferred materials and methods are now described. Other features, objects and advantages of the invention will be apparent from the description. In the description, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present description will control.

### DETAILED DESCRIPTION OF THE INVENTION

The present invention relates to compositions, methods and processes for the design, preparation, manufacture and/or formulation of recombinant adeno-associated virus (AAV) particles having one or more regulatable elements and methods of using the same. In one embodiment, the regulatable elements may comprise CRISPR regulatable elements.

### Parvoviridae Virus, Viral Particle and Production of Viral Particles

Viruses of the Parvoviridae family are small non-enveloped icosahedral capsid viruses characterized by a single stranded DNA genome. Parvoviridae family viruses consist of two subfamilies: Parvovirinae, which infect vertebrates, and Densovirinae, which infect invertebrates. The parvoviruses and other members of the Parvoviridae family are generally described in Kenneth I. Berns, “Parvoviridae: The Viruses and Their Replication,” Chapter 69 in FIELDS VIROLOGY (3d Ed. 1996), the contents of which is incorporated by reference in its entirety.

The genome of the viruses of the Parvoviridae family may be modified to contain a minimum of components for the assembly of a functional recombinant virus which is loaded with or engineered to express or deliver a desired nucleic acid construct or payload, e.g., a transgene, polypeptide-encoding polynucleotide or modulatory nucleic acid, which may be delivered to a target cell, tissue or organism. As used herein, a “viral particle” refers to a functional recombinant virus.

The Parvoviridae family may be used as a biological tool due to a relatively simple structure that may be manipulated with standard molecular biology techniques.

The Parvoviridae family comprises the Dependovirus genus which includes adeno-associated viruses (AAVs) which are capable of replication in vertebrate hosts including, but not limited to, human, primate, bovine, canine, equine, and ovine species. The naturally occurring AAV Cap gene expresses VP1, VP2, and VP3 capsid proteins are encoded by a single open reading frame of the Cap gene under control of the p40 promoter. In one embodiment, nucleotide sequences encoding VP1, VP2 and VP3 proteins and/or amino acid sequences of AAV VP capsid proteins may be modified for increased efficiency to target to the central nervous system (e.g., CNS tissue tropism). Any of the VP genes of the serotypes selected from, but not limited to, AAV1, AAV2, AAV3, AAV4, AAV5, AAV6, AAV7, AAV8, AAV9, AAV10, and AAV11, AAV12, AAVrh8, AAVrh10, AAV-DJ, and AAV-DJ/8 capsid serotypes, or variants thereof (e.g., AAV3A and AAV3B) may be modified.

In one embodiment, the present invention provides administration and/or delivery methods for viral particles.

In some embodiments, the present invention provides administration and/or delivery methods for viral particles for the treatment and/or amelioration of diseases or disorders of the CNS. As a non-limiting example, the disease or disorder of the CNS is Alzheimer's Diseases (AD), Amyotrophic lateral sclerosis (ALS), Creutzfeldt-Jakob Disease, Huntingtin's disease (HD), Friedreich's ataxia (FA or FRDA), Parkinson Disease (PD), Multiple System Atrophy (MSA), Spinal Muscular Atrophy (SMA), Multiple Sclerosis (MS), Primary progressive aphasia, Progressive supranuclear palsy, Dementia, Brain Cancer, Degenerative Nerve Diseases, Encephalitis, Epilepsy, Genetic Brain Disorders that cause neurodegeneration, Retinitis pigmentosa (RP), Head and Brain Malformations, Hydrocephalus, Stroke, Prion disease, Infantile neuronal ceroid lipofuscinosis (INCL) (a neurodegenerative disease of children caused by a deficiency in the lysosomal enzyme palmitoyl protein thioesterase-1 (PPT1)).

In one embodiment, provided are particles comprising nucleic acids and cells (in vivo or in culture) comprising the nucleic acids and/or particles of the invention. Suitable particles include without limitation viral particles (e.g., adenovirus, AAV, herpes virus, vaccinia, poxviruses, baculoviruses, and the like), plasmids, phage, YACs, BACs, and the like as are well known in the art. Such nucleic acids, particles and cells can be used, for example, as reagents (e.g., helper packaging constructs or packaging cells) for the production of modified virus capsids or virus particles as described herein.

The particles of the invention which comprise nucleic acids include any genetic element (vector) which may be delivered to a host cell, e.g., naked DNA, plasmid, phage, transposon, cosmid, episome, a protein in a non-viral delivery vehicle (e.g., a lipid-based carrier), virus, etc., which transfers the sequences carried thereon. The methods used to construct any embodiment of this invention are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y.

The polynucleotide (e.g., transgene or payload) can be carried on any suitable vector, e.g., a plasmid, which is delivered to a host cell. The plasmids useful in this invention may be engineered such that they are suitable for replication and, optionally, integration in prokaryotic cells, mammalian cells, or both. These plasmids may contain sequences permitting replication of the transgene in eukaryotes and/or prokaryotes and selection markers for these systems. Selectable markers or reporter genes may include sequences encoding geneticin, hygromicin or purimycin resistance, among others. The plasmids may also contain certain selectable reporters or marker genes that can be used to signal the presence of the vector in bacterial cells, such as ampicillin resistance. Other components of the plasmid may include an origin of replication and an amplicon, such as the amplicon system employing the Epstein Barr virus nuclear antigen. This amplicon system, or other similar amplicon components permit high copy episomal replication in the cells. Preferably, the molecule carrying the transgene or payload is transfected into the cell, where it may exist transiently. Alternatively, the transgene may be stably integrated into the genome of the host cell, either chromosomally or as an episome. In certain embodiments, the transgene may be present in multiple copies, optionally in head-to-head, head-to-tail, or tail-to-tail concatamers. Suitable transfection techniques are known and may readily be utilized to deliver the transgene to the host cell.

In some embodiments, the payload may be delivered in a viral particle derived from an adenoviral vector. In another embodiment, the payload may be delivered in a viral particle derived from a lentiviral vector. In yet another embodiment, the payload may be delivered in a viral particle derived from any other gene delivery vector known in the art.

### AAV Particle

In one embodiment, the present invention provides administration and/or delivery methods for AAV particles. As used herein, “AAV particles” refers to a viral particle where the virus is adeno-associated virus (AAV). An AAV particle comprises a viral genome and a capsid. As used herein, “viral genome” is a polynucleotide encoding at least one inverted terminal repeat (ITR), at least one regulatory sequence, and at least one payload. In one embodiment, the viral genome or any portion thereof may be codon optimized.

The AAV particles described herein may be useful in the fields of human disease, antibodies, viruses, veterinary applications and a variety of in vivo and in vitro settings.

In some embodiments, AAV particles described herein are useful in the field of medicine for the treatment, palliation and/or amelioration of conditions or diseases such as, but not limited to, blood, cardiovascular, CNS, and/or genetic disorders.

In some embodiments, AAV particles in accordance with the present invention may be used for the treatment of disorders, and/or conditions, including but not limited to neurological disorders (e.g., Alzheimer's disease, Huntington's disease, autism, Parkinson's disease, Spinal muscular atrophy, Friedreich's ataxia).

In some embodiments, the present invention provides administration and/or delivery methods for AAV particles for the treatment and/or amelioration of diseases or disorders of the CNS. As a non-limiting example, the disease or disorder of the CNS is Alzheimer's Diseases (AD), Amyotrophic lateral sclerosis (ALS), Creutzfeldt-Jakob Disease, Huntingtin's disease (HD), Friedreich's ataxia (FA or FRDA), Parkinson Disease (PD), Multiple System Atrophy (MSA), Spinal Muscular Atrophy (SMA), Multiple Sclerosis (MS), Primary progressive aphasia, Progressive supranuclear palsy, Dementia, Brain Cancer, Degenerative Nerve Diseases, Encephalitis, Epilepsy, Genetic Brain Disorders that cause neurodegeneration, Retinitis pigmentosa (RP), Head and Brain Malformations, Hydrocephalus, Stroke, Prion disease, Infantile neuronal ceroid lipofuscinosis (INCL) (a neurodegenerative disease of children caused by a deficiency in the lysosomal enzyme palmitoyl protein thioesterase-1 (PPT1)).

In some embodiments, AAV particles produced according to the present invention may target to deliver and/or to transfer a payload of interest to specific population of cells in specific anatomical regions (e.g., dopaminergic (DAergic) neurons in the Substantia Nigra (SN)) in the central nervous system).

In one embodiment, the AAV particles of the invention may be a single-stranded AAV (ssAAV) or a self-complementary AAV (scAAV) described herein or known in the art.

### Payload

AAV particles of the present invention may comprise a nucleic acid sequence encoding at least one “payload.” As used herein, a “payload” refers to one or more polynucleotides or polynucleotide regions encoded by or within a viral genome or an expression product of such polynucleotide or polynucleotide region, e.g., a transgene, a polynucleotide encoding a polypeptide or multi-polypeptide or a modulatory nucleic acid or regulatory nucleic acid.

The payload may comprise any nucleic acid known in the art which is useful for modulating the expression in a target cell transduced or contacted with the AAV particle carrying the payload. In one embodiment, modulation may be by supplementation of the payload in a target cell or tissue. In one embodiment, modulation may be gene replacement of the payload in a target cell or tissue. In one embodiment, modulation may be by inhibition using a modulatory nucleic acid of the payload in a target cell or tissue.

In one embodiment, the payload may comprise a combination of coding and non-coding nucleic acid sequences.

In one embodiment, the payload or any portion thereof may be codon optimized.

In one embodiment, the one or more payloads may comprise one or more regulatable elements. In one embodiment, payload expression may be governed by a regulatable system which comprises one or more regulatable elements.

mRNA

In one embodiment, a messenger RNA (mRNA) may be encoded by a payload. As used herein, the term “messenger RNA” (mRNA) refers to any polynucleotide which encodes a polypeptide of interest and which is capable of being translated to produce the encoded polypeptide of interest in vitro, in vivo, in situ, or ex vivo. The components of an mRNA include, but are not limited to, a coding region, a 5′UTR, a 3′UTR, a 5′ cap and a poly-A tail. In some embodiments, the encoded mRNA or any portion of the mRNA be codon optimized.

Traditionally, the basic components of an mRNA molecule include at least a coding region, a 5′UTR, a 3′UTR, a 5′ cap and a poly-A tail. According to the present invention, payloads encoding mRNA may comprise a coding region only. The payloads may also comprise a coding region and at least one UTR. The payloads may also comprise a coding region, 3′UTR and polyA tail.

In one embodiment, the mRNA may be codon optimized.

In one embodiment, the payload may encode a gene therapy product. A gene therapy product may comprise a polypeptide, RNA molecule, or other gene product that, when expressed in a target cell, provides a desired therapeutic effect. In some embodiments, a gene therapy product may comprise a substitute for a non-functional gene that is absent or mutated.

### Polypeptide

In one embodiment, the payload encodes a polypeptide which may be a peptide or protein. A protein encoded by the payload may comprise a secreted protein, an intracellular protein, an extracellular protein, a membrane protein, and/or fragment or variant thereof.

In one embodiment, the encoded proteins may be structural or functional.

In one embodiment, proteins encoded by the payload construct payload construct include, but are not limited to, mammalian proteins.

In one embodiment the protein encoded by the payload is between 50-5000 amino acids in length. In some embodiments the protein encoded is between 50-2000 amino acids in length. In some embodiments the protein encoded is between 50-1000 amino acids in length. In some embodiments the protein encoded is between 50-1500 amino acids in length. In some embodiments the protein encoded is between 50-1000 amino acids in length. In some embodiments the protein encoded is between 50-800 amino acids in length. In some embodiments the protein encoded is between 50-600 amino acids in length. In some embodiments the protein encoded is between 50-400 amino acids in length. In some embodiments the protein encoded is between 50-200 amino acids in length. In some embodiments the protein encoded is between 50-100 amino acids in length.

In some embodiments the peptide encoded by the payload is between 4-50 amino acids in length. In one embodiment, the shortest length of a region of the payload of the present invention encoding a peptide can be the length that is sufficient to encode for a tetrapeptide, a pentapeptide, a hexapeptide, a heptapeptide, an octapeptide, a nonapeptide, or a decapeptide. In another embodiment, the length may be sufficient to encode a peptide of 2-30 amino acids, e.g. 5-30, 10-30, 2-25, 5-25, 10-25, or 10-20 amino acids. The length may be sufficient to encode for a peptide of at least 11, 12, 13, 14, 15, 17, 20, 25 or 30 amino acids, or a peptide that is no longer than 50 amino acids, e.g. no longer than 35, 30, 25, 20, 17, 15, 14, 13, 12, 11 or 10 amino acids.

Modulatory nucleic acids

In one embodiment, an RNA sequence encoded by the payload may be a tRNA, rRNA, tmRNA, miRNA, RNAi, siRNA, piRNA, shRNA antisense RNA, double stranded RNA, snRNA, snoRNA, and/or long non-coding RNA (lncRNA). These RNA sequences along with siRNA, shRNA, antisense molecules and the like may also be referred to as “modulatory nucleic acids.”

In one embodiment, the RNA encoded by the payload is a lncRNA or RNAi construct designed to target lncRNA. Non-limiting examples of such lncRNA molecules and RNAi constructs designed to target such lncRNA are taught in International Publication, WO2012/018881, the contents of which are incorporated by reference in their entirety.

In one embodiment, the payload encodes a microRNA (miRNA) or engineered precursors thereof, as the payload. MicroRNAs (miRNAs) are 19-25 nucleotide RNAs that bind to nucleic acid molecules and down-regulate gene expression either by reducing nucleic acid molecule stability or by inhibiting translation. As a non-limiting example, the payloads described herein may encode one or more microRNA target sequences, microRNA sequences, or microRNA seeds, or any known precursors thereof such as pre- or pri-microRNAs. Such sequences may correspond to any known microRNA such as those taught in US Publication US2005/0261218 and US Publication US2005/0059005, the contents of which are incorporated herein by reference in their entirety.

A microRNA sequence comprises a “seed” region, i.e., a sequence in the region of positions 2-8 of the mature microRNA, which sequence has perfect Watson-Crick complementarity to the miRNA target sequence. A microRNA seed may comprise positions 2-8 or 2-7 of the mature microRNA. In some embodiments, a microRNA seed may comprise 7 nucleotides (e.g., nucleotides 2-8 of the mature microRNA), wherein the seed-complementary site in the corresponding miRNA target is flanked by an adenine (A) opposed to microRNA position 1. In some embodiments, a microRNA seed may comprise 6 nucleotides (e.g., nucleotides 2-7 of the mature microRNA), wherein the seed-complementary site in the corresponding miRNA target is flanked by an adenine (A) opposed to microRNA position 1. See for example, Grimson A, Farh K K, Johnston W K, Garrett-Engele P, Lim L P, Bartel D P; Mol Cell. 2007 Jul. 6; 27(1):91-105; each of which is herein incorporated by reference in their entirety. The bases of the microRNA seed have complete complementarity with the target sequence.

In one embodiment, tissue specific microRNAs may be used for tissue specific regulation of the payload. For example, to allow for systemic administration targeting liver and brain, Xie et al. (Xie J, Xie Q, Zhang H, Ameres S L, Hung J H, Su Q, et al. MicroRNA-regulated, systemically delivered rAAV9: a step closer to CNS-restricted transgene expression. Molecular Therapy. 2010; 19(3):526-535), the contents of which is herein incorporated by reference in its entirety, used liver-specific, endogenous microRNAs (miRNAs) to repress rAAV expression outside the CNS, by including miRNA-binding sites into the rAAV9 construct.

In one embodiment, one or more microRNA binding sites may be included in the payload construct to de-target, i.e., to reduce or eliminate payload expression in a particular tissue. MicroRNA binding sites may be inserted 5′ or 3′ of the payload or both. In some embodiments, microRNA binding sites may be located within the payload sequence. In one embodiment, the micro RNA binding sites are all specific to one microRNA. In other embodiments, the microRNA binding sites are specific for two or more different microRNAs.

In one embodiment, the payload encodes an RNA sequence that may be processed to produce a siRNA, miRNA or other double stranded (ds) or single stranded (ss) gene modulatory nucleic acids or motifs.

In one embodiment, the siRNA duplexes or dsRNA encoded by the payload can be used to inhibit gene expression in a cell, in particular cells of the CNS. In some aspects, the inhibition of gene expression refers to an inhibition by at least about 20%, 30%, 40%, 50%, 60%, 70%, 80%, 85%, 90%, 95% and 100%. In one aspect, the protein product of the targeted gene may be inhibited by at least about 20%, 30%, 40%, 50%, 60%, 70%, 80%, 85%, 90%, 95% and 100%. The gene can be either a wild type gene or a gene with at least one mutation (mutated gene). The targeted protein may be either a wild type protein or a protein with at least one mutation (mutated protein).

In one embodiment, the present invention provides methods for treating, or ameliorating a disease or condition associated with abnormal gene and/or protein in a subject in need of treatment, the method comprising administering to the subject any effective amount of at least one AAV particle encoding an siRNA duplex targeting the gene, delivering duplex into targeted cells, inhibiting the gene expression and protein production, and ameliorating symptoms of the disease or condition in the subject. Gene replacement or activation

In one embodiment, the payload encodes an RNA sequence to increase the expression of a gene or replace a gene. As a non-limiting example, AAV particles may comprise a viral genome comprising a payload which encodes a normal gene to replace a mutated, defective or nonfunctional copy of that gene in the recipient.

In some aspects, the increase of gene expression refers to an increase by at least about 20%, 30%, 40%, 50%, 60%, 70%, 80%, 85%, 90%, 95% and 100%. In one aspect, the protein product of the targeted gene may be increased by at least about 20%, 30%, 40%, 50%, 60%, 70%, 80%, 85%, 90%, 95% and 100%.

### Functional Payloads

In one embodiment, a payload may encode polypeptides that are or can be a fusion protein.

In one embodiment, a payload may encode polypeptides that are or can be polypeptides having a desired biological activity.

In one embodiment, a payload may encode polypeptides that are or can be gene products that can complement a genetic defect.

In one embodiment, a payload may encode polypeptides that are or can be RNA molecules.

In one embodiment, a payload may encode polypeptides that are or can be transcription factors.

In one embodiment, a payload may encode polypeptides that are or can be other gene products that are of interest in regulation and/or expression.

In one embodiment, a payload may comprise nucleotide sequences that provide a desired effect or regulatory function (e.g., transposons, transcription factors).

In one embodiment, a payload may comprise nucleotide sequences or encode hormone receptors (e.g., mineral corticosteroid, glucocorticoid, and thyroid hormone receptors), intramembrane proteins (e.g., TM-1 and TM-7), intracellular receptors (e.g., orphans, retinoids, vitamin D3 and vitamin A receptors), signaling molecules (e.g., kinases, transcription factors, or molecules such as signal transducers and activators of transcription receptors of the cytokine superfamily (e.g. erythropoietin, growth hormone, interferons, and interleukins, and colony-stimulating factors, G-protein coupled receptors, e.g., hormones, calcitonin, epinephrine, gastrin, and paracrine or autocrine mediators, such as somatostatin or prostaglandins, neurotransmitter receptors (norepinephrine, dopamine, serotonin or acetylcholine), and/or pathogenic antigens which can be of viral, bacterial, allergenic, or cancerous origin, and tyrosine kinase receptors (such as insulin growth factor, and nerve growth factor).

The encoded payload may comprise a gene therapy product. In some embodiments, a gene therapy product may comprise a substitute for a non-functional gene that is absent or mutated.

In one embodiment, a payload may encode polypeptides that are or can be a marker to assess cell transformation and expression.

In one embodiment, a payload may comprise or encode a selectable marker. A selectable marker may comprise a gene sequence or a protein encoded by a gene sequence expressed in a host cell that allows for the identification, selection, and/or purification of the host cell from a population of cells that may or may not express the selectable marker. In one embodiment, the selectable marker provides resistance to survive a selection process that would otherwise kill the host cell, such as treatment with an antibiotic. In another embodiment, an antibiotic selectable marker may comprise one or more antibiotic resistance factors, including but not limited to neomycin resistance (e.g., neo), hygromycin resistance, kanamycin resistance, and/or puromycin resistance.

In some embodiments, a payload may comprise or encode any nucleic acid sequence encoding a polypeptide can be used as a selectable marker comprising recognition by a specific antibody.

In some embodiments, a payload may comprise or encode a cell-surface marker, such as any protein expressed on the surface of the cell including, but not limited to receptors, CD markers, lectins, integrins, or truncated versions thereof. In some embodiments, cells that comprise a cell-surface marker may be selected using an antibody targeted to the cell-surface marker. In some embodiments an antibody targeted to the cell-surface marker may be directly conjugated with a selection agent including, but not limited to a fluorophore, sepharose, or magnetic bead. In some embodiments an antibody targeted to the cell-surface marker may be detected using a secondary labeled antibody or substrate which binds to the antibody targeted to the cell-surface marker. In some embodiments, a selectable marker may comprise negative selection by using an enzyme, including but not limited to Herpes simplex virus thymidine kinase (HSVTK) that converts a pro-toxin (ganciclovir) into a toxin or bacterial Cytosine Deaminase (CD) which converts the pro-toxin 5′-fluorocytosine (5′-FC) into the toxin 5′-fluorouracil (5′-FU). In some embodiments, any nucleic acid sequence encoding a polypeptide can be used as a selectable marker comprising recognition by a specific antibody.

In some embodiments, a payload may comprise or encode a selectable marker including, but not limited to, β-lactamase, luciferase, β-galactosidase, or any other reporter gene as that term is understood in the art, including cell-surface markers, such as CD4 or the truncated nerve growth factor (NGFR) (for GFP, see WO 96/23810; Heim et al., Current Biology 2:178-182 (1996); Heim et al., Proc. Natl. Acad. Sci. USA (1995); or Heim et al., Science 373:663-664 (1995); for β-lactamase, see WO 96/30540; the contents of each of which are herein incorporated by reference in its entirety).

In some embodiments, a payload may comprise or encode a selectable marker comprising a fluorescent protein. A fluorescent protein as herein described may comprise any fluorescent marker including but not limited to green, yellow, and/or red fluorescent protein (GFP, YFP, and/or RFP).

In one embodiment, the AAV particles of the present invention are designed for expression of multiple functional RNAs in a single vector as described in Björklund et al (Expression of multiple functional RNAs or proteins from one viral vector; Methods in Molecular Biology; 2016; 1382:41-56), the contents of which are herein incorporated by reference in their entirety. In one embodiment, the viral genome is a polycistronic vector encoding fusion proteins, or comprising ribosome skipping sequence(s) or internal ribosome entry sites. In one embodiment, the AAV particles of the present invention designed for expression of multiple functional RNAs in a single vector utilize multiple promoters, such as, but not limited to bi-directional promoters (Pol II-based), dual promoters, combined Pol II and Pol III promoters, or dual Pol II promoters.

### Payload Construct

In one embodiment, the AAV particle may comprise a payload construct. As used herein, “payload construct” refers to one or more polynucleotide regions encoding or comprising a payload that is flanked on one or both sides by an inverted terminal repeat (ITR) sequence.

In one embodiment, the payload construct may comprise more than one payload. As a non-limiting example, a target cell transduced with an AAV particle comprising more than one payload may express each of the payloads in a single cell.

In some embodiments, the payload construct may encode a coding or non-coding RNA.

In one embodiment, a payload construct encoding one or more payloads for expression in a target cell may comprise one or more payload or non-payload nucleotide sequences operably linked to at least one target cell-compatible promoter.

In one embodiment, the ITRs in the AAV particle are derived from the same AAV serotype.

In one embodiment, the ITRs in the AAV particle are derived from different AAV serotypes.

In one embodiment, the ITRs in the AAV particle are the same.

In one embodiment, the ITRs in the AAV particle are different. In one aspect, the ITRs may be derived from the same AAV serotype. In another aspect, the ITRs may be derived from different serotypes.

In some embodiments, the payload construct may include a sequence that allows the translation of several proteins from a single construct (i.e., bicistronic or multicistronic construct). In a non-limiting example, such a sequence may include a cleavage site, such as a 2A peptide. In some embodiments, the 2A cleavage site may be a 2A peptide site from foot-and-mouth disease virus (F2A sequence), equine rhinitis A virus (E2A), Thosea asigna virus (T2A), and porcine teschovirus-1 (P2A), as described in US Publication No. 20070116690, the contents of which is herein incorporated by reference in its entirety. In some embodiments, the cleavage site may be a furin cleavage site known in the art. In another non-limiting example, such a regulatory sequence may include an internal ribosomal entry site (IRES), which allows for translation initiation in the middle of the transcript. In some embodiments, an internal stop codon may be positioned 5′ of the IRES.

### Promoters

A payload construct encoding one or more payloads for expression in a target cell may comprise one or more payload or non-payload nucleotide sequences operably linked to at least one target cell-compatible promoter. A person skilled in the art may recognize that a target cell may require a specific promoter including but not limited to a promoter that is species specific, inducible, tissue-specific, or cell cycle-specific (Parr et al., Nat. Med. 3:1145-9 (1997)).

While a constitutive promoter drives ubiquitous and temporally unrestricted expression, tissue specific and/or inducible promoters allow more tight control of desired expression.

In some embodiments, expression of the payload may be desirable only in a particular tissue of interest. In one aspect, it may be desirable to exercise tight temporal control, i.e. turn the payload on or off in a particular tissue of interest. In some embodiments, this means that the promoter is not “leaky”, i.e., the promoter does not drive expression in another unwanted cell type or tissue or does not drive unwanted expression at a time when the promoter is not induced, even if it is at a lower level.

In some embodiments, the payload construct comprises a tissue specific promoter. Non-limiting examples of tissue specific promoters are listed in Table 1 and are for example described in Papadakis et al. 2004; Current Gene Therapy, 4, 98-113 and Kantor et al., 2014 Adv Genet. 2014; 87: 125-197, the contents of each which is herein incorporated by reference in its entirety, and references therein.

TABLE 1
Tissue Specific Promoters

| Promoter | Tissue |
|---|---|
| Apo A-I, ApoE, a1-antitrypsin (hAAT), Apo A-I, Transthyretin, | Liver |
| Liver-enriched activator, Albumin, Phosphoenolpyruvate | |
| carboxykinase (PEPCK), RNAPII promoter | |
| PAI-1 (plasminogen activator inhibitor 1), ICAM-2, Endoglin, | Endothelium |
| ICAM-2 (intercellular adhesion molecule 2), flt-1(fms-like | |
| tyrosine kinase-1), vWF (von-Willebrand factor) | |
| MCK (muscle creatine kinase), SMC a-actin, Myosin heavy- | Muscle |
| chain, Myosin light-chain | |
| Cytokeratin 18, CFTR (cystic fibrosis transmembrane | Epithelium |
| conductance regulator) | |
| GFAP (glial fibrillary acidic protein) | Neuronal (Astrocytes) |
| NSE (neuronal-specific endolase) | Neuronal (Neurons) |
| Synapsin I | Neuronal (Neurons) |
| Preproenkephalin | Neuronal (All of CNS) |
| Dopamine b-hydroxylase (dbH) | Neuronal |
| Prolactin | Neuronal |
| Myelin basic protein | Neuronal |
| | (Oligodendrocytes) |
| F4/80 | Neuronal (Microglia) |
| MeCP2 (MeP229) | Neuronal (Neurons) |
| MCH | Neuronal (MCH neurons) |
| CaMKII | Neuronal |
| Ankyrin, a-spectrin, Globin, HLA-Dra, CD4, Dectin-2 | Erythroid |

In one embodiment, a payload construct encoding one or more payloads for expression in a target cell may comprise one or more payload sequences operably linked to a tissue specific promoter which expresses only in certain tissues or cell types.

In one embodiment, a payload construct may comprise one or more payload sequences operably linked to a constitutive promoter which is continuously, strongly and ubiquitously expressed. Non-limiting examples of constitutive promoters included, but are not limited to, those described in Qin et al., (PLOS One, 2010; DOI: 10.1371/journal.pone.0010611) and those listed in Table 2.

TABLE 2
Constitutive Promoters

| Promoter | Reference |
|---|---|
| Cytomegalovirus (CMV) | Qin et al. and references therein |
| simian virus 40 early promoter (SV40) | Qin et al. and references therein |
| human Ubiquitin C promoter (UBC) | Qin et al. and references therein |
| human elongation factor 1α promoter (EF1A) | Qin et al. and references therein |
| mouse phosphoglycerate kinase 1 promoter | Qin et al. and references therein; Papadakis |
| (PGK); human phosphoglycerate kinase 1 | et al. 2004 and references therein |
| (PGK1) | |
| chicken β-Actin promoter coupled with | Qin et al. and references therein |
| glucose 6-phosphate dehydrogenase | Papadakis et al. 2004 and references therein |

In one embodiment, a payload construct encoding one or more payloads for expression in a target cell may comprise one or more payload sequences operably linked to one or more inducible promoters, such that the payload expression may be regulated in a temporal or spatial manner.

In some embodiments the inducible promoter may be a minimal promoter, which comprises one or more DNA binding elements for a transcription factor DNA binding domain, which is the main or sole driver of transcription from the promoter. In one embodiment, the promoter may be tightly controlled and is not leaky.

In one embodiment, the promoter may be a Pol II promoter, such as the CMV promoter.

In another embodiment, the promoter may be a Pol III promoter, such as the U6 promoter.

In one embodiment, the promoter may be a viral promoter.

In another embodiment, the promoter may be a non-viral promoter.

In one embodiment the promoter may comprise enhancer sequences, such as CMV IE enhancer.

In some embodiments, the payload may be under the control of an inducible promoter, which can be temporally regulated. The regulatable element may comprise a chemical agent (i.e. a natural or artificial ligand, compound or drug) or physiological stimulus.

Promoter inducible elements are regulated by exogenously provided chemical agents or physiological stimuli. In some embodiments, the payload expression occurs in a dose-dependent manner, depending on the dose of the chemical agent or physiological stimulus.

In some embodiments, the promoter may be a minimal promoter containing one or more DNA binding elements to which the DNA binding domain of a particular transcription factor or fusion protein binds, wherein transcription from the promoter can only occur upon binding of the transcription factor. The promoter may contain 1, 2, 3, 4, 5, 6, 7, 8, 9, 10 or more such binding elements.

In some embodiments, the inducible promoter may be tissue specific. In some embodiments, the inducible promoter may be neuron-specific. In some embodiments, the promoter may be the CamKII promoter, which may additionally comprise one or more promoter inducible elements. In some embodiments, the one or more inducible elements may be tetracycline-inducible tetracycline response elements. In one embodiment, the regulatable element may comprise a tetracycline inducible transactivator protein. In one embodiment, the inducible promoter driving payload expression may be the promoter described in US Publication No. US20120004277, the contents of which is herein incorporated by reference in its entirety.

In one embodiment, the promoter may comprise one or more HIV TAT protein binding elements. For example, the promoter may comprise the HIV-1 ITR fused to the Drosophila hsp70 minimal heat shock promoter, as described in U.S. Pat. No. 8,138,327, the contents of which is herein incorporated by reference in its entirety. Without wishing to be bound by theory, this would allow the promoter to be induced upon HIV infection. In this scenario, the promoter may drive the expression of a payload that inhibits HIV gene expression, such as an anti-HIV short hairpin RNA, as described in U.S. Pat. No. 8,138,327.

In one embodiment, the promoter may be the bovine leukemia virus promoter, which is inducible by the viral protein Tax. In another embodiment, Tax may be provided through the regulatable element as described in U.S. Pat. No. 7,297,536, the contents of which is herein incorporated by reference in its entirety.

In some embodiments, the promoter driving the expression of any of the regulatable elements described herein may also be regulated, i.e., may be inducible, repressible, tissue specific, or constitutive.

### Capsids and Capsid Serotypes

In some embodiments, AAV particles of the present invention may be packaged in a capsid structure or may be capsid free. Such capsid free donor and/or acceptor sequences such as AAV, are described in, for example, US Publication 20140107186, the content of which is incorporated by reference in its entirety.

In one embodiment, the present invention, provides nucleic acids encoding the mutated or modified virus capsids and capsid proteins of the invention. In some embodiments the capsids are engineered according to the methods of co-owned and co-pending International Publication No. WO2015191508, the contents of which are herein incorporated by reference in their entirety.

In some embodiments, AAV particles produced according to the present invention may comprise hybrid serotypes with enhanced transduction to specific cell types of interest in the central nervous system, prolonged transgene expression and/or a safety profile. The hybrid serotypes may be generated by transcapsidation, adsorption of bi-specific antibody to capsid surface, mosaic capsid, and chimeric capsid, and/or other capsid protein modifications.

In some embodiments, AAV particles of the present invention may be further modified toward a specific therapeutic application by rational mutagenesis of capsid proteins (see, e.g., Pulicherla et al., Mol Ther, 2011, 19: 1070-1078), incorporation of peptide ligands to the capsid, for example a peptide derived from an NMDA receptor agonist for enhanced retrograde transport (Xu et al., Virology, 2005, 341: 203-214), and directed evolution to produce new AAV variants for increased CNS transduction.

In some embodiments, AAV particles produced according to the present invention may comprise different capsid proteins, either naturally occurring and/or recombinant, including, but not limited to, AAV1, AAV2, AAV3, AAV4, AAVS, AAV6, AAV7, AAV8, AAV9, AAV10, and AAV11, AAV12, AAVrh8, AAVrh10, AAV-DJ, and AAV-DJ/8 capsid serotypes, or variants thereof (e.g., AAV3A and AAV3B). Nucleic acid sequences encoding one or more AAV capsid proteins useful in the present invention are disclosed in the commonly owned International Publication No. WO2015191508, the contents of which are herein incorporated by reference in their entirety.

In some embodiments, AAV particles of the present invention may comprise or be derived from any natural or recombinant AAV serotype. According to the present invention, the AAV particles may utilize or be based on a serotype selected from any of the following AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVCS, AAV-DJ, AAV-DJ8, AAVF3, AAVFS, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T , AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101 , AAV-8h, AAV-8b, AAV-h, AAV-b, AAV SM 10-2 , AAV Shuffle 100-1 , AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8 , AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10 and/or Japanese AAV 10 serotypes, and variants thereof. As a non-limiting example, the capsid of the recombinant AAV virus is AAV2. As a non-limiting example, the capsid of the recombinant AAV virus is AAVrh10. As a non-limiting example, the capsid of the recombinant AAV virus is AAV9(hul4). As a non-limiting example, the capsid of the recombinant AAV virus is AAV-DJ. As a non-limiting example, the capsid of the recombinant AAV virus is AAV9.47. As a non-limiting example, the capsid of the recombinant AAV virus is AAV-DJ8.

In some embodiments, the AAV particles of the present invention may comprise or be derived from an AAV serotype which may be, or have, a sequence as described in United States Publication No. US20030138772, the contents of which are herein incorporated by reference in their entirety, such as, but not limited to, AAV1 (SEQ ID NO: 6 and 64 of US20030138772), AAV2 (SEQ ID NO: 7 and 70 of US20030138772), AAV3 (SEQ ID NO: 8 and 71 of US20030138772), AAV4 (SEQ ID NO: 63 of US20030138772), AAVS (SEQ ID NO: 114 of US20030138772), AAV6 (SEQ ID NO: 65 of US20030138772), AAV7 (SEQ ID NO: 1-3 of US20030138772), AAV8 (SEQ ID NO: 4 and 95 of US20030138772), AAV9 (SEQ ID NO: 5 and 100 of US20030138772), AAV10 (SEQ ID NO: 117 of US20030138772), AAV11 (SEQ ID NO: 118 of US20030138772), AAV12 (SEQ ID NO: 119 of US20030138772), AAVrh10 (amino acids 1 to 738 of SEQ ID NO: 81 of US20030138772), AAV16.3 (US20030138772 SEQ ID NO: 10), AAV29.3/bb.1 (US20030138772 SEQ ID NO: 11), AAV29.4 (US20030138772 SEQ ID NO: 12), AAV29.5/bb.2 (US20030138772 SEQ ID NO: 13), AAV1.3 (US20030138772 SEQ ID NO: 14), AAV13.3 (US20030138772 SEQ ID NO: 15), AAV24.1 (US20030138772 SEQ ID NO: 16), AAV27.3 (US20030138772 SEQ ID NO: 17), AAV7.2 (US20030138772 SEQ ID NO: 18), AAVC1 (US20030138772 SEQ ID NO: 19), AAVC3 (US20030138772 SEQ ID NO: 20), AAVCS (US20030138772 SEQ ID NO: 21), AAVF1 (US20030138772 SEQ ID NO: 22), AAVF3 (US20030138772 SEQ ID NO: 23), AAVFS (US20030138772 SEQ ID NO: 24), AAVH6 (US20030138772 SEQ ID NO: 25), AAVH2 (US20030138772 SEQ ID NO: 26), AAV42-8 (US20030138772 SEQ ID NO: 27), AAV42-15 (US20030138772 SEQ ID NO: 28), AAV42-5b (US20030138772 SEQ ID NO: 29), AAV42-lb (US20030138772 SEQ ID NO: 30), AAV42-13 (US20030138772 SEQ ID NO: 31), AAV42-3a (US20030138772 SEQ ID NO: 32), AAV42-4 (US20030138772 SEQ ID NO: 33), AAV42-5a (US20030138772 SEQ ID NO: 34), AAV42-10 (US20030138772 SEQ ID NO: 35), AAV42-3b (US20030138772 SEQ ID NO: 36), AAV42-11 (US20030138772 SEQ ID NO: 37), AAV42-6b (US20030138772 SEQ ID NO: 38), AAV43-1 (US20030138772 SEQ ID NO: 39), AAV43-5 (US20030138772 SEQ ID NO: 40), AAV43-12 (US20030138772 SEQ ID NO: 41), AAV43-20 (US20030138772 SEQ ID NO: 42), AAV43-21 (US20030138772 SEQ ID NO: 43), AAV43-23 (US20030138772 SEQ ID NO: 44), AAV43-25 (US20030138772 SEQ ID NO: 45), AAV44.1 (US20030138772 SEQ ID NO: 46), AAV44.5 (US20030138772 SEQ ID NO: 47), AAV223.1 (US20030138772 SEQ ID NO: 48), AAV223.2 (US20030138772 SEQ ID NO: 49), AAV223.4 (US20030138772 SEQ ID NO: 50), AAV223.5 (US20030138772 SEQ ID NO: 51), AAV223.6 (US20030138772 SEQ ID NO: 52), AAV223.7 (US20030138772 SEQ ID NO: 53), AAVA3.4 (US20030138772 SEQ ID NO: 54), AAVA3.5 (US20030138772 SEQ ID NO: 55), AAVA3.7 (US20030138772 SEQ ID NO: 56), AAVA3.3 (US20030138772 SEQ ID NO: 57), AAV42.12 (US20030138772 SEQ ID NO: 58), AAV44.2 (US20030138772 SEQ ID NO: 59), AAV42-2 (US20030138772 SEQ ID NO: 9), or variants thereof.

In some embodiments, the AAV particles of the present invention may comprise or be derived from AAV serotype which may be, or have, a sequence as described in United States Publication No. US20150159173, the contents of which are herein incorporated by reference in their entirety, such as, but not limited to, AAV2 (SEQ ID NO: 7 and 23 of US20150159173), rh20 (SEQ ID NO: 1 of US20150159173), rh32/33 (SEQ ID NO: 2 of US20150159173), rh39 (SEQ ID NO: 3, 20 and 36 of US20150159173), rh46 (SEQ ID NO: 4 and 22 of US20150159173), rh73 (SEQ ID NO: 5 of US20150159173), rh74 (SEQ ID NO: 6 of US20150159173), AAV6.1 (SEQ ID NO: 29 of US20150159173), rh.8 (SEQ ID NO: 41 of US20150159173), rh.48.1 (SEQ ID NO: 44 of US20150159173), hu.44 (SEQ ID NO: 45 of US20150159173), hu.29 (SEQ ID NO: 42 of US20150159173), hu.48 (SEQ ID NO: 38 of US20150159173), rh54 (SEQ ID NO: 49 of US20150159173), AAV2 (SEQ ID NO: 7 of US20150159173), cy.5 (SEQ ID NO: 8 and 24 of US20150159173), rh.10 (SEQ ID NO: 9 and 25 of US20150159173), rh.13 (SEQ ID NO: 10 and 26 of US20150159173), AAV1 (SEQ ID NO: 11 and 27 of US20150159173), AAV3 (SEQ ID NO: 12 and 28 of US20150159173), AAV6 (SEQ ID NO: 13 and 29 of US20150159173), AAV7 (SEQ ID NO: 14 and 30 of US20150159173), AAV8 (SEQ ID NO: 15 and 31 of US20150159173), hu.13 (SEQ ID NO: 16 and 32 of US20150159173), hu.26 (SEQ ID NO: 17 and 33 of US20150159173), hu.37 (SEQ ID NO: 18 and 34 of US20150159173), hu.53 (SEQ ID NO: 19 and 35 of US20150159173), rh.43 (SEQ ID NO: 21 and 37 of US20150159173), rh2 (SEQ ID NO: 39 of US20150159173), rh.37 (SEQ ID NO: 40 of US20150159173), rh.64 (SEQ ID NO: 43 of US20150159173), rh.48 (SEQ ID NO: 44 of US20150159173), ch.5 (SEQ ID NO 46 of US20150159173), rh.67 (SEQ ID NO: 47 of US20150159173), rh.58 (SEQ ID NO: 48 of US20150159173), or variants thereof including, but not limited to Cy5R1, Cy5R2, Cy5R3, Cy5R4, rh.13R, rh.37R2, rh.2R, rh.8R, rh.48.1, rh.48.2, rh.48.1.2, hu.44R1, hu.44R2, hu.44R3, hu.29R, ch.5R1, rh64R1, rh64R2, AAV6.2, AAV6.1, AAV6.12, hu.48R1, hu.48R2, and hu.48R3.

In some embodiments, the AAV particles of the present invention may comprise or be derived from AAV serotype which may be, or have, a sequence as described in U.S. Pat. No. 7,198,951, the contents of which are herein incorporated by reference in their entirety, such as, but not limited to, AAV9 (SEQ ID NO: 1-3 of U.S. Pat. No. 7,198,951), AAV2 (SEQ ID NO: 4 of U.S. Pat. No. 7,198,951), AAV1 (SEQ ID NO: 5 of U.S. Pat. No. 7,198,951), AAV3 (SEQ ID NO: 6 of U.S. Pat. No. 7,198,951), and AAV8 (SEQ ID NO: 7 of U.S. Pat. No. 7,198,951).

In some embodiments, the AAV particles of the present invention may comprise or be derived from AAV serotype which may be, or have, a mutation in the AAV9 sequence as described by N Pulicherla et al. (Molecular Therapy 19(6):1070-1078 (2011), herein incorporated by reference in its entirety), such as but not limited to, AAV9.9, AAV9.11, AAV9.13, AAV9.16, AAV9.24, AAV9.45, AAV9.47, AAV9.61, AAV9.68, AAV9.84.

In some embodiments, the AAV particles of the present invention may comprise or be derived from AAV serotype which may be, or have, a sequence as described in U.S. Pat. No. 6,156,303, the contents of which are herein incorporated by reference in their entirety, such as, but not limited to, AAV3B (SEQ ID NO: 1 and 10 of U.S. Pat. No. 6,156,303), AAV6 (SEQ ID NO: 2, 7 and 11 of U.S. Pat. No. 6,156,303), AAV2 (SEQ ID NO: 3 and 8 of U.S. Pat. No. 6,156,303), AAV3A (SEQ ID NO: 4 and 9, of U.S. Pat. No. 6,156,303), or derivatives thereof.

In some embodiments, the AAV particles of the present invention may comprise or be derived from AAV serotype which may be, or have, a sequence as described in United States Publication No. US20140359799, the contents of which are herein incorporated by reference in their entirety, such as, but not limited to, AAV8 (SEQ ID NO: 1 of US20140359799), AAVDJ (SEQ ID NO: 2 and 3 of US20140359799), or variants thereof.

In some embodiments, the AAV particle may comprise a capsid from a serotype such as, but not limited to, AAVDJ or a variant thereof, such as AAVDJ8 (or AAV-DJ8), as described by Grimm et al. (Journal of Virology 82(12): 5887-5911 (2008), herein incorporated by reference in its entirety). The amino acid sequence of AAVDJ8 may comprise two or more mutations in order to remove the heparin binding domain (HBD). As a non-limiting example, the AAV-DJ sequence described as SEQ ID NO: 1 in U.S. Pat. No. 7,588,772, the contents of which are herein incorporated by reference in their entirety, may comprise two mutations: (1) R587Q where arginine (R; Arg) at amino acid 587 is changed to glutamine (Q; Gln) and (2) R590T where arginine (R; Arg) at amino acid 590 is changed to threonine (T; Thr). As another non-limiting example, may comprise three mutations: (1) K406R where lysine (K; Lys) at amino acid 406 is changed to arginine (R; Arg), (2) R587Q where arginine (R; Arg) at amino acid 587 is changed to glutamine (Q; Gln) and (3) R590T where arginine (R; Arg) at amino acid 590 is changed to threonine (T; Thr).

In some embodiments, the AAV particles of the present invention may comprise or be derived from AAV serotype which may be, or have, a sequence of AAV4 as described in International Publication No. WO1998011244, the contents of which are herein incorporated by reference in their entirety, such as, but not limited to AAV4 (SEQ ID NO: 1-20 of WO1998011244).

In some embodiments, the AAV particles of the present invention may comprise or be derived from AAV serotype which may be, or have, a mutation in the AAV2 sequence to generate AAV2G9 as described in International Publication No. WO2014144229 and herein incorporated by reference in its entirety.

In some embodiments, the AAV particles of the present invention may comprise or be derived from AAV serotype which may be, or have, a sequence as described in International Publication No. WO2005033321, the contents of which are herein incorporated by reference in their entirety, such as, but not limited to AAV3-3 (SEQ ID NO: 217 of WO2005033321), AAV1 (SEQ ID NO: 219 and 202 of WO2005033321), AAV106.1/hu.37 (SEQ ID No: 10 of WO2005033321), AAV114.3/hu.40 (SEQ ID No: 11 of WO2005033321), AAV127.2/hu.41 (SEQ ID NO:6 and 8 of WO2005033321), AAV128.3/hu.44 (SEQ ID No: 81 of WO2005033321), AAV130.4/hu.48 (SEQ ID NO: 78 of WO2005033321), AAV145.1/hu.53 (SEQ ID No: 176 and 177 of WO2005033321), AAV145.6/hu.56 (SEQ ID NO: 168 and 192 of WO2005033321), AAV16.12/hu.11 (SEQ ID NO: 153 and 57 of WO2005033321), AAV16.8/hu.10 (SEQ ID NO: 156 and 56 of WO2005033321), AAV161.10/hu.60 (SEQ ID No: 170 of WO2005033321), AAV161.6/hu.61 (SEQ ID No: 174 of WO2005033321), AAV1-7/rh.48 (SEQ ID NO: 32 of WO2005033321), AAV1-8/rh.49 (SEQ ID NOs: 103 and 25 of WO2005033321), AAV2 (SEQ ID NO: 211 and 221 of WO2005033321), AAV2-15/rh.62 (SEQ ID No: 33 and 114 of WO2005033321), AAV2-3/rh.61 (SEQ ID NO: 21 of WO2005033321), AAV2-4/rh.50 (SEQ ID No: 23 and 108 of WO2005033321), AAV2-5/rh.51 (SEQ ID NO: 104 and 22 of WO2005033321), AAV3.1/hu.6 (SEQ ID NO: 5 and 84 of WO2005033321), AAV3.1/hu.9 (SEQ ID NO: 155 and 58 of WO2005033321), AAV3-11/rh.53 (SEQ ID NO: 186 and 176 of WO2005033321), AAV3-3 (SEQ ID NO: 200 of WO2005033321), AAV33.12/hu.17 (SEQ ID NO:4 of WO2005033321), AAV33.4/hu.15 (SEQ ID No: 50 of WO2005033321), AAV33.8/hu.16 (SEQ ID No: 51 of WO2005033321), AAV3-9/rh.52 (SEQ ID NO: 96 and 18 of WO2005033321), AAV4-19/rh.55 (SEQ ID NO: 117 of WO2005033321), AAV4-4 (SEQ ID NO: 201 and 218 of WO2005033321), AAV4-9/rh.54 (SEQ ID NO: 116 of WO2005033321), AAVS (SEQ ID NO: 199 and 216 of WO2005033321), AAV52.1/hu.20 (SEQ ID NO: 63 of WO2005033321), AAV52/hu.19 (SEQ ID NO: 133 of WO2005033321), AAVS-22/rh.58 (SEQ ID No: 27 of WO2005033321), AAVS-3/rh.57 (SEQ ID NO: 105 of WO2005033321), AAVS-3/rh.57 (SEQ ID No: 26 of WO2005033321), AAV58.2/hu.25 (SEQ ID No: 49 of WO2005033321), AAV6 (SEQ ID NO: 203 and 220 of WO2005033321), AAV7 (SEQ ID NO: 222 and 213 of WO2005033321), AAV7.3/hu.7 (SEQ ID No: 55 of WO2005033321), AAV8 (SEQ ID NO: 223 and 214 of WO2005033321), AAVH-1/hu.1 (SEQ ID No: 46 of WO2005033321), AAVH-5/hu.3 (SEQ ID No: 44 of WO2005033321), AAVhu.1 (SEQ ID NO: 144 of WO2005033321), AAVhu.10 (SEQ ID NO: 156 of WO2005033321), AAVhu.11 (SEQ ID NO: 153 of WO2005033321), AAVhu.12 (WO2005033321 SEQ ID NO: 59), AAVhu.13 (SEQ ID NO: 129 of WO2005033321), AAVhu.14/AAV9 (SEQ ID NO: 123 and 3 of WO2005033321), AAVhu.15 (SEQ ID NO: 147 of WO2005033321), AAVhu.16 (SEQ ID NO: 148 of WO2005033321), AAVhu.17 (SEQ ID NO: 83 of WO2005033321), AAVhu.18 (SEQ ID NO: 149 of WO2005033321), AAVhu.19 (SEQ ID NO: 133 of WO2005033321), AAVhu.2 (SEQ ID NO: 143 of WO2005033321), AAVhu.20 (SEQ ID NO: 134 of WO2005033321), AAVhu.21 (SEQ ID NO: 135 of WO2005033321), AAVhu.22 (SEQ ID NO: 138 of WO2005033321), AAVhu.23.2 (SEQ ID NO: 137 of WO2005033321), AAVhu.24 (SEQ ID NO: 136 of WO2005033321), AAVhu.25 (SEQ ID NO: 146 of WO2005033321), AAVhu.27 (SEQ ID NO: 140 of WO2005033321), AAVhu.29 (SEQ ID NO: 132 of WO2005033321), AAVhu.3 (SEQ ID NO: 145 of WO2005033321), AAVhu.31 (SEQ ID NO: 121 of WO2005033321), AAVhu.32 (SEQ ID NO: 122 of WO2005033321), AAVhu.34 (SEQ ID NO: 125 of WO2005033321), AAVhu.35 (SEQ ID NO: 164 of WO2005033321), AAVhu.37 (SEQ ID NO: 88 of WO2005033321), AAVhu.39 (SEQ ID NO: 102 of WO2005033321), AAVhu.4 (SEQ ID NO: 141 of WO2005033321), AAVhu.40 (SEQ ID NO: 87 of WO2005033321), AAVhu.41 (SEQ ID NO: 91 of WO2005033321), AAVhu.42 (SEQ ID NO: 85 of WO2005033321), AAVhu.43 (SEQ ID NO: 160 of WO2005033321), AAVhu.44 (SEQ ID NO: 144 of WO2005033321), AAVhu.45 (SEQ ID NO: 127 of WO2005033321), AAVhu.46 (SEQ ID NO: 159 of WO2005033321), AAVhu.47 (SEQ ID NO: 128 of WO2005033321), AAVhu.48 (SEQ ID NO: 157 of WO2005033321), AAVhu.49 (SEQ ID NO: 189 of WO2005033321), AAVhu.51 (SEQ ID NO: 190 of WO2005033321), AAVhu.52 (SEQ ID NO: 191 of WO2005033321), AAVhu.53 (SEQ ID NO: 186 of WO2005033321), AAVhu.54 (SEQ ID NO: 188 of WO2005033321), AAVhu.55 (SEQ ID NO: 187 of WO2005033321), AAVhu.56 (SEQ ID NO: 192 of WO2005033321), AAVhu.57 (SEQ ID NO: 193 of WO2005033321), AAVhu.58 (SEQ ID NO: 194 of WO2005033321), AAVhu.6 (SEQ ID NO: 84 of WO2005033321), AAVhu.60 (SEQ ID NO: 184 of WO2005033321), AAVhu.61 (SEQ ID NO: 185 of WO2005033321), AAVhu.63 (SEQ ID NO: 195 of WO2005033321), AAVhu.64 (SEQ ID NO: 196 of WO2005033321), AAVhu.66 (SEQ ID NO: 197 of WO2005033321), AAVhu.67 (SEQ ID NO: 198 of WO2005033321), AAVhu.7 (SEQ ID NO: 150 of WO2005033321), AAVhu.8 (WO2005033321 SEQ ID NO: 12), AAVhu.9 (SEQ ID NO: 155 of WO2005033321), AAVLG-10/rh.40 (SEQ ID No: 14 of WO2005033321), AAVLG-4/rh.38 (SEQ ID NO: 86 of WO2005033321), AAVLG-4/rh.38 (SEQ ID No: 7 of WO2005033321), AAVN721-8/rh.43 (SEQ ID NO: 163 of WO2005033321), AAVN721-8/rh.43 (SEQ ID No: 43 of WO2005033321), AAVpi.1 (WO2005033321 SEQ ID NO: 28), AAVpi.2 (WO2005033321 SEQ ID NO: 30), AAVpi.3 (WO2005033321 SEQ ID NO: 29), AAVrh.38 (SEQ ID NO: 86 of WO2005033321), AAVrh.40 (SEQ ID NO: 92 of WO2005033321), AAVrh.43 (SEQ ID NO: 163 of WO2005033321), AAVrh.44 (WO2005033321 SEQ ID NO: 34), AAVrh.45 (WO2005033321 SEQ ID NO: 41), AAVrh.47 (WO2005033321 SEQ ID NO: 38), AAVrh.48 (SEQ ID NO: 115 of WO2005033321), AAVrh.49 (SEQ ID NO: 103 of WO2005033321), AAVrh.50 (SEQ ID NO: 108 of WO2005033321), AAVrh.51 (SEQ ID NO: 104 of WO2005033321), AAVrh.52 (SEQ ID NO: 96 of WO2005033321), AAVrh.53 (SEQ ID NO: 97 of WO2005033321), AAVrh.55 (WO2005033321 SEQ ID NO: 37), AAVrh.56 (SEQ ID NO: 152 of WO2005033321), AAVrh.57 (SEQ ID NO: 105 of WO2005033321), AAVrh.58 (SEQ ID NO: 106 of WO2005033321), AAVrh.59 (WO2005033321 SEQ ID NO: 42), AAVrh.60 (WO2005033321 SEQ ID NO: 31), AAVrh.61 (SEQ ID NO: 107 of WO2005033321), AAVrh.62 (SEQ ID NO: 114 of WO2005033321), AAVrh.64 (SEQ ID NO: 99 of WO2005033321), AAVrh.65 (WO2005033321 SEQ ID NO: 35), AAVrh.68 (WO2005033321 SEQ ID NO: 16), AAVrh.69 (WO2005033321 SEQ ID NO: 39), AAVrh.70 (WO2005033321 SEQ ID NO: 20), AAVrh.72 (WO2005033321 SEQ ID NO: 9), or variants thereof including, but not limited to, AAVcy.2, AAVcy.3, AAVcy.4, AAVcy.5, AAVcy.6, AAVrh.12, AAVrh.17, AAVrh.18, AAVrh.19, AAVrh.21, AAVrh.22, AAVrh.23, AAVrh.24, AAVrh.25, AAVrh.25/42 15, AAVrh.31, AAVrh.32, AAVrh.33, AAVrh.34, AAVrh.35, AAVrh.36, AAVrh.37, AAVrh14. Non limiting examples of variants include SEQ ID NO: 13, 15, 17, 19, 24, 36, 40, 45, 47, 48, 51-54, 60-62, 64-77, 79, 80, 82, 89, 90, 93-95, 98, 100, 101-109-113, 118-120, 124, 126, 131, 139, 142, 151,154, 158, 161, 162, 165-183, 202, 204-212, 215, 219, 224-236, of WO2005033321, the contents of which are herein incorporated by reference in their entirety.

In some embodiments, the AAV particles of the present invention may comprise or be derived from AAV serotype which may be, or have, a sequence as described in International Publication No. WO2015168666, the contents of which are herein incorporated by reference in their entirety, such as, but not limited to, AAVrh8R (SEQ ID NO: 9 of WO2015168666), AAVrh8R A586R mutant (SEQ ID NO: 10 of WO2015168666), AAVrh8R R533A mutant (SEQ ID NO: 11 of WO2015168666), or variants thereof.

In some embodiments, the AAV particles of the present invention may comprise or be derived from AAV serotype which may be, or have, a sequence as described in U.S. Pat. No. 9,233,131, the contents of which are herein incorporated by reference in their entirety, such as, but not limited to, AAVhE1.1 (SEQ ID NO:44 of US9233131), AAVhEr1.5 (SEQ ID NO:45 of U.S. Pat. No. 9,233,131), AAVhER1.14 (SEQ ID NO:46 of U.S. Pat. No. 9,233,131), AAVhEr1.8 (SEQ ID NO:47 of U.S. Pat. No. 9,233,131), AAVhEr1.16 (SEQ ID NO:48 of U.S. Pat. No. 9,233,131), AAVhEr1.18 (SEQ ID NO:49 of U.S. Pat. No. 9,233,131), AAVhEr1.35 (SEQ ID NO:50 of U.S. Pat. No. 9,233,131), AAVhEr1.7 (SEQ ID NO:51 of U.S. Pat. No. 9,233,131), AAVhEr1.36 (SEQ ID NO:52 of U.S. Pat. No. 9,233,131), AAVhEr2.29 (SEQ ID NO:53 of U.S. Pat. No. 9,233,131), AAVhEr2.4 (SEQ ID NO:54 of U.S. Pat. No. 9,233,131), AAVhEr2.16 (SEQ ID NO:55 of U.S. Pat. No. 9,233,131), AAVhEr2.30 (SEQ ID NO:56 of U.S. Pat. No. 9,233,131), AAVhEr2.31 (SEQ ID NO:58 of U.S. Pat. No. 9,233,131), AAVhEr2.36 (SEQ ID NO:57 of U.S. Pat. No. 9,233,131), AAVhER1.23 (SEQ ID NO:53 of U.S. Pat. No. 9,233,131), AAVhEr3.1 (SEQ ID NO:59 of U.S. Pat. No. 9,233,131), AAV2.5T (SEQ ID NO:42 of U.S. Pat. No. 9,233,131), or variants thereof.

In some embodiments, the AAV particles of the present invention may comprise or be derived from AAV serotype which may be, or have, a sequence as described in United States Patent Publication No. US20150376607, the contents of which are herein incorporated by reference in their entirety, such as, but not limited to, AAV-PAEC (SEQ ID NO:1 of US20150376607), AAV-LK01 (SEQ ID NO:2 of US20150376607), AAV-LK02 (SEQ ID NO:3 of US20150376607), AAV-LK03 (SEQ ID NO:4 of US20150376607), AAV-LK04 (SEQ ID NO:5 of US20150376607), AAV-LK05 (SEQ ID NO:6 of US20150376607), AAV-LK06 (SEQ ID NO:7 of US20150376607), AAV-LK07 (SEQ ID NO:8 of US20150376607), AAV-LK08 (SEQ ID NO:9 of US20150376607), AAV-LK09 (SEQ ID NO:10 of US20150376607), AAV-LK10 (SEQ ID NO:11 of US20150376607), AAV-LK11 (SEQ ID NO:12 of US20150376607), AAV-LK12 (SEQ ID NO:13 of US20150376607), AAV-LK13 (SEQ ID NO:14 of US20150376607), AAV-LK14 (SEQ ID NO:15 of US20150376607), AAV-LK15 (SEQ ID NO:16 of US20150376607), AAV-LK16 (SEQ ID NO:17 of US20150376607), AAV-LK17 (SEQ ID NO:18 of US20150376607), AAV-LK18 (SEQ ID NO:19 of US20150376607), AAV-LK19 (SEQ ID NO:20 of US20150376607), AAV-PAEC2 (SEQ ID NO:21 of US20150376607), AAV-PAEC4 (SEQ ID NO:22 of US20150376607), AAV-PAEC6 (SEQ ID NO:23 of US20150376607), AAV-PAEC7 (SEQ ID NO:24 of US20150376607), AAV-PAEC8 (SEQ ID NO:25 of US20150376607), AAV-PAEC11 (SEQ ID NO:26 of US20150376607), AAV-PAEC12 (SEQ ID NO:27, of US20150376607), or variants thereof.

In some embodiments, the AAV particles of the present invention may comprise or be derived from AAV serotype which may be, or have, a sequence as described in U.S. Pat. No. 9,163,261, the contents of which are herein incorporated by reference in their entirety, such as, but not limited to, AAV-2-pre-miRNA-101 (SEQ ID NO: 1 U.S. Pat. No. 9,163,261), or variants thereof.

In some embodiments, the AAV particles of the present invention may comprise or be derived from AAV serotype which may be, or have, a sequence as described in United States Patent Publication No. US20150376240, the contents of which are herein incorporated by reference in their entirety, such as, but not limited to, AAV-8h (SEQ ID NO: 6 of US20150376240), AAV-8b (SEQ ID NO: 5 of US20150376240), AAV-h (SEQ ID NO: 2 of US20150376240), AAV-b (SEQ ID NO: 1 of US20150376240), or variants thereof.

In some embodiments, the AAV particles of the present invention may comprise or be derived from AAV serotype which may be, or have, a sequence as described in United States Patent Publication No. US20160017295, the contents of which are herein incorporated by reference in their entirety, such as, but not limited to, AAV SM 10-2 (SEQ ID NO: 22 of US20160017295), AAV Shuffle 100-1 (SEQ ID NO: 23 of US20160017295), AAV Shuffle 100-3 (SEQ ID NO: 24 of US20160017295), AAV Shuffle 100-7 (SEQ ID NO: 25 of US20160017295), AAV Shuffle 10-2 (SEQ ID NO: 34 of US20160017295), AAV Shuffle 10-6 (SEQ ID NO: 35 of US20160017295), AAV Shuffle 10-8 (SEQ ID NO: 36 of US20160017295), AAV Shuffle 100-2 (SEQ ID NO: 37 of US20160017295), AAV SM 10-1 (SEQ ID NO: 38 of US20160017295), AAV SM 10-8 (SEQ ID NO: 39 of US20160017295), AAV SM 100-3 (SEQ ID NO: 40 of US20160017295), AAV SM 100-10 (SEQ ID NO: 41 of US20160017295), or variants thereof.

In some embodiments, the AAV particles of the present invention may comprise or be derived from AAV serotype which may be, or have, a sequence as described in United States Patent Publication No. US20150238550, the contents of which are herein incorporated by reference in their entirety, such as, but not limited to, BNP61 AAV (SEQ ID NO: 1 of US20150238550), BNP62 AAV (SEQ ID NO: 3 of US20150238550), BNP63 AAV (SEQ ID NO: 4 of US20150238550), or variants thereof

In some embodiments, the AAV particles of the present invention may comprise or be derived from an AAV serotype which may be or may have a sequence as described in United States Patent Publication No. US20150315612, the contents of which are herein incorporated by reference in their entirety, such as, but not limited to, AAVrh.50 (SEQ ID NO: 108 of US20150315612), AAVrh.43 (SEQ ID NO: 163 of US20150315612), AAVrh.62 (SEQ ID NO: 114 of US20150315612), AAVrh.48 (SEQ ID NO: 115 of US20150315612), AAVhu.19 (SEQ ID NO: 133 of US20150315612), AAVhu.11 (SEQ ID NO: 153 of US20150315612), AAVhu.53 (SEQ ID NO: 186 of US20150315612), AAV4-8/rh.64 (SEQ ID No: 15 of US20150315612), AAVLG-9/hu.39 (SEQ ID No: 24 of US20150315612), AAV54.5/hu.23 (SEQ ID No: 60 of US20150315612), AAV54.2/hu.22 (SEQ ID No: 67 of US20150315612), AAV54.7/hu.24 (SEQ ID No: 66 of US20150315612), AAV54.1/hu.21 (SEQ ID No: 65 of US20150315612), AAV54.4R/hu.27 (SEQ ID No: 64 of US20150315612), AAV46.2/hu.28 (SEQ ID No: 68 of US20150315612), AAV46.6/hu.29 (SEQ ID No: 69 of US20150315612), AAV128.1/hu.43 (SEQ ID No: 80 of US20150315612), or variants thereof

In some embodiments, the AAV particles of the present invention may comprise or be derived from AAV serotype which may be, or have, a sequence as described in International Publication No. WO2015121501, the contents of which are herein incorporated by reference in their entirety, such as, but not limited to, true type AAV (ttAAV) (SEQ ID NO: 2 of WO2015121501), “UPenn AAV10” (SEQ ID NO: 8 of WO2015121501), “Japanese AAV10” (SEQ ID NO: 9 of WO2015121501), or variants thereof.

According to the present invention, the AAV particle may comprise an AAV capsid serotype which may be selected from or derived from a variety of species. In one embodiment, the AAV may be an avian AAV (AAAV). The AAAV serotype may be, or have, a sequence as described in U.S. Pat. No. 9,238,800, the contents of which are herein incorporated by reference in their entirety, such as, but not limited to, AAAV (SEQ ID NO: 1, 2, 4, 6, 8, 10, 12, and 14 of U.S. Pat. No. 9,238,800), or variants thereof.

In one embodiment, the AAV particle may comprise an AAV capsid serotype which may be or derived from a bovine AAV (BAAV). The BAAV serotype may be, or have, a sequence as described in U.S. Pat. No. 9,193,769, the contents of which are herein incorporated by reference in their entirety, such as, but not limited to, BAAV (SEQ ID NO: 1 and 6 of U.S. Pat. No. 9,193,769), or variants thereof. The BAAV serotype may be or have a sequence as described in U.S. Pat. No. 7,427,396, the contents of which are herein incorporated by reference in their entirety, such as, but not limited to, BAAV (SEQ ID NO: 5 and 6 of U.S. Pat. No. 7,427,396), or variants thereof.

In one embodiment, the AAV particle may comprise an AAV capsid serotype which may be or derived from a caprine AAV. The caprine AAV serotype may be, or have, a sequence as described in U.S. Pat. No. 7,427,396, the contents of which are herein incorporated by reference in their entirety, such as, but not limited to, caprine AAV (SEQ ID NO: 3 of U.S. Pat. No. 7,427,396), or variants thereof.

In other embodiments, the AAV particle may comprise an AAV capsid serotype which may be engineered as a hybrid AAV from two or more parental serotypes. In one embodiment, the AAV may be AAV2G9 which comprises sequences from AAV2 and AAV9. The AAV2G9 AAV serotype may be, or have, a sequence as described in United States Patent Publication No. US20160017005, the contents of which are herein incorporated by reference in its entirety.

In one embodiment, the AAV particle may comprise an AAV capsid serotype which may be generated by the AAV9 capsid library with mutations in amino acids 390-627 (VP1 numbering) as described by Pulicherla et al. (Molecular Therapy 19(6):1070-1078 (2011), the contents of which are herein incorporated by reference in their entirety. The serotype and corresponding nucleotide and amino acid substitutions may be, but is not limited to, AAV9.1 (G1594C; D532H), AAV6.2 (T1418A and T1436X; V473D and I479K), AAV9.3 (T1238A; F413Y), AAV9.4 (T1250C and A1617T; F417S), AAV9.5 (A1235G, A1314T, A1642G, C1760T; Q412R, T548A, A587V), AAV9.6 (T1231A; F4111), AAV9.9 (G1203A, G1785T; W595C), AAV9.10 (A1500G, T1676C; M559T), AAV9.11 (A1425T, A1702C, A1769T; T568P, Q590L), AAV9.13 (A1369C, A1720T; N457H, T574S), AAV9.14 (T1340A, T1362C, T1560C, G1713A; L447H), AAV9.16 (A1775T; Q592L), AAV9.24 (T1507C, T1521G; W503R), AAV9.26 (A1337G, A1769C; Y446C, Q590P), AAV9.33 (A1667C; D556A), AAV9.34 (A1534G, C1794T; N512D), AAV9.35 (A1289T, T1450A, C1494T, A1515T, C1794A, G1816A; Q430L, Y484N, N98K, V6061), AAV9.40 (A1694T, E565V), AAV9.41 (A1348T, T1362C; T450S), AAV9.44 (A1684C, A1701T, A1737G; N562H, K567N), AAV9.45 (A1492T, C1804T; N498Y, L602F), AAV9.46 (G1441C, T1525C, T1549G; G481R, W509R, L517V), 9.47 (G1241A, G1358A, A1669G, C1745T; S414N, G453D, K557E, T5821), AAV9.48 (C1445T, A1736T; P482L, Q579L), AAV9.50 (A1638T, C1683T, T1805A; Q546H, L602H), AAV9.53 (G1301A, A1405C, C1664T, G1811T; R134Q, 5469R, A555V, G604V), AAV9.54 (C1531A, T1609A; L5111, L537M), AAV9.55 (T1605A; F535L), AAV9.58 (C1475T, C1579A; T4921, H527N), AAV.59 (T1336C; Y446H), AAV9.61 (A1493T; N4981), AAV9.64 (C1531A, A1617T; L5111), AAV9.65 (C1335T, T1530C, C1568A; A523D), AAV9.68 (C1510A; P504T), AAV9.80 (G1441A,;G481R), AAV9.83 (C1402A, A1500T; P468T, E500D), AAV9.87 (T1464C, T1468C; S490P), AAV9.90 (A1196T; Y399F), AAV9.91 (T1316G, A1583T, C1782G, T1806C; L439R, K528I), AAV9.93 (A1273G, A1421G, A1638C, C1712T, G1732A, A1744T, A1832T; S425G, Q474R, Q546H, P571L, G578R, T582S, D611V), AAV9.94 (A1675T; M559L) and AAV9.95 (T1605A; F535L).

In one embodiment, the AAV particle of the present invention may have an AAV9 variant capsid serotype (AAV Clade F) as described in International Publication WO2016049230, the contents of which are herein incorporated by reference in their entirety.

In one embodiment the AAV particle of the present invention may have an AAV2g9 capsid as described in Murlidharan et al (CNS-restricted transduction and CRISPR/Cas9-mediated gene deletion with an engineered AAV vector; Molecular Therapy Nucleic Acids 5, e338; published online July 19, 2016), the contents of which are herein incorporated by reference in their entirety. This capsid variant comprises an exchange of the amino acid residues of the AAV9 capsid required for galactose binding for the corresponding amino acids of the AAV2 capsid (Q464V, A467P, D469N, I470M, R471A, D472V, S474G, Y500F, and S501A). Further, the viral genome of the AAV may comprise one or more gRNAs targeting a microRNA (e.g., MIR137) as described for treatment of any disease or disorder. In one embodiment the disease to be treated is schizophrenia.

In one embodiment, the AAV particle may comprise an AAV capsid serotype which may be a serotype comprising at least one AAV capsid CD8+ T-cell epitope. As a non-limiting example, the serotype may be AAV1, AAV2 or AAV8.

In one embodiment, the AAV particle may comprise an AAV capsid serotype which may be a serotype selected from any of those found in Table 3.

In one embodiment, the AAV particle may comprise an AAV capsid serotype which may comprise a sequence, fragment or variant thereof, of the sequences in Table 3.

In one embodiment, the AAV particle may comprise an AAV capsid serotype which may be encoded by a sequence, fragment or variant as described in Table 3.

TABLE 3.
AAV Serotypes

| | SEQ | |
|---|---|---|
| Serotype | ID NO | Reference Information |
| AAV1 | 1 | US20150159173 SEQ ID NO: 11, US20150315612 SEQ ID NO: 202 |
| AAV1 | 2 | US20160017295 SEQ ID NO: 1, US20030138772 SEQ ID NO: 64, |
| | | US20150159173 SEQ ID NO: 27, US20150315612 SEQ ID NO: 219, |
| | | U.S. Pat. No. 7,198,951 SEQ ID NO: 5 |
| AAV1 | 3 | US20030138772 SEQ ID NO: 6 |
| AAV1.3 | 4 | US20030138772 SEQ ID NO: 14 |
| AAV10 | 5 | US20030138772 SEQ ID NO: 117 |
| AAV10 | 6 | WO2015121501 SEQ ID NO: 9 |
| AAV10 | 7 | WO2015121501 SEQ ID NO: 8 |
| AAV11 | 8 | US20030138772 SEQ ID NO: 118 |
| AAV12 | 9 | US20030138772 SEQ ID NO: 119 |
| AAV2 | 10 | US20150159173 SEQ ID NO: 7, US20150315612 SEQ ID NO: 211 |
| AAV2 | 11 | US20030138772 SEQ ID NO: 70, US20150159173 SEQ ID NO: 23, |
| | | US20150315612 SEQ ID NO: 221, US20160017295 SEQ ID NO: 2, |
| | | U.S. Pat. No. 6,156,303 SEQ ID NO: 4, U.S. Pat. No. 7,198,951 SEQ ID NO: 4, |
| | | WO2015121501 SEQ ID NO: 1 |
| AAV2 | 12 | U.S. Pat. No. 6,156,303 SEQ ID NO: 8 |
| AAV2 | 13 | US20030138772 SEQ ID NO: 7 |
| AAV2 | 14 | U.S. Pat. No. 6,156,303 SEQ ID NO: 3 |
| AAV2.5T | 15 | U.S. Pat. No. 9,233,131 SEQ ID NO: 42 |
| AAV223.10 | 16 | US20030138772 SEQ ID NO: 75 |
| AAV223.2 | 17 | US20030138772 SEQ ID NO: 49 |
| AAV223.2 | 18 | US20030138772 SEQ ID NO: 76 |
| AAV223.4 | 19 | US20030138772 SEQ ID NO: 50 |
| AAV223.4 | 20 | US20030138772 SEQ ID NO: 73 |
| AAV223.5 | 21 | US20030138772 SEQ ID NO: 51 |
| AAV223.5 | 22 | US20030138772 SEQ ID NO: 74 |
| AAV223.6 | 23 | US20030138772 SEQ ID NO: 52 |
| AAV223.6 | 24 | US20030138772 SEQ ID NO: 78 |
| AAV223.7 | 25 | US20030138772 SEQ ID NO: 53 |
| AAV223.7 | 26 | US20030138772 SEQ ID NO: 77 |
| AAV29.3 | 27 | US20030138772 SEQ ID NO: 82 |
| AAV29.4 | 28 | US20030138772 SEQ ID NO: 12 |
| AAV29.5 | 29 | US20030138772 SEQ ID NO: 83 |
| AAV29.5 (AAVbb.2) | 30 | US20030138772 SEQ ID NO: 13 |
| AAV3 | 31 | US20150159173 SEQ ID NO: 12 |
| AAV3 | 32 | US20030138772 SEQ ID NO: 71, US20150159173 SEQ ID NO: 28, |
| | | US20160017295 SEQ ID NO: 3, U.S. Pat. No. 7,198,951 SEQ ID NO: 6 |
| AAV3 | 33 | US20030138772 SEQ ID NO: 8 |
| AAV3.3b | 34 | US20030138772 SEQ ID NO: 72 |
| AAV3-3 | 35 | US20150315612 SEQ ID NO: 200 |
| AAV3-3 | 36 | US20150315612 SEQ ID NO: 217 |
| AAV3a | 37 | U.S. Pat. No. 6,156,303 SEQ ID NO: 5 |
| AAV3a | 38 | U.S. Pat. No. 6,156,303 SEQ ID NO: 9 |
| AAV3b | 39 | U.S. Pat. No. 6,156,303 SEQ ID NO: 6 |
| AAV3b | 40 | U.S. Pat. No. 6,156,303 SEQ ID NO: 10 |
| AAV3b | 41 | U.S. Pat. No. 6,156,303 SEQ ID NO: 1 |
| AAV4 | 42 | US20140348794 SEQ ID NO: 17 |
| AAV4 | 43 | US20140348794 SEQ ID NO: 5 |
| AAV4 | 44 | US20140348794 SEQ ID NO: 3 |
| AAV4 | 45 | US20140348794 SEQ ID NO: 14 |
| AAV4 | 46 | US20140348794 SEQ ID NO: 15 |
| AAV4 | 47 | US20140348794 SEQ ID NO: 19 |
| AAV4 | 48 | US20140348794 SEQ ID NO: 12 |
| AAV4 | 49 | US20140348794 SEQ ID NO: 13 |
| AAV4 | 50 | US20140348794 SEQ ID NO: 7 |
| AAV4 | 51 | US20140348794 SEQ ID NO: 8 |
| AAV4 | 52 | US20140348794 SEQ ID NO: 9 |
| AAV4 | 53 | US20140348794 SEQ ID NO: 2 |
| AAV4 | 54 | US20140348794 SEQ ID NO: 10 |
| AAV4 | 55 | US20140348794 SEQ ID NO: 11 |
| AAV4 | 56 | US20140348794 SEQ ID NO: 18 |
| AAV4 | 57 | US20030138772 SEQ ID NO: 63, US20160017295 SEQ ID NO: 4, |
| | | US20140348794 SEQ ID NO: 4 |
| AAV4 | 58 | US20140348794 SEQ ID NO: 16 |
| AAV4 | 59 | US20140348794 SEQ ID NO: 20 |
| AAV4 | 60 | US20140348794 SEQ ID NO: 6 |
| AAV4 | 61 | US20140348794 SEQ ID NO: 1 |
| AAV42.2 | 62 | US20030138772 SEQ ID NO: 9 |
| AAV42.2 | 63 | US20030138772 SEQ ID NO: 102 |
| AAV42.3b | 64 | US20030138772 SEQ ID NO: 36 |
| AAV42.3B | 65 | US20030138772 SEQ ID NO: 107 |
| AAV42.4 | 66 | US20030138772 SEQ ID NO: 33 |
| AAV42.4 | 67 | US20030138772 SEQ ID NO: 88 |
| AAV42.8 | 68 | US20030138772 SEQ ID NO: 27 |
| AAV42.8 | 69 | US20030138772 SEQ ID NO: 85 |
| AAV43.1 | 70 | US20030138772 SEQ ID NO: 39 |
| AAV43.1 | 71 | US20030138772 SEQ ID NO: 92 |
| AAV43.12 | 72 | US20030138772 SEQ ID NO: 41 |
| AAV43.12 | 73 | US20030138772 SEQ ID NO: 93 |
| AAV43.20 | 74 | US20030138772 SEQ ID NO: 42 |
| AAV43.20 | 75 | US20030138772 SEQ ID NO: 99 |
| AAV43.21 | 76 | US20030138772 SEQ ID NO: 43 |
| AAV43.21 | 77 | US20030138772 SEQ ID NO: 96 |
| AAV43.23 | 78 | US20030138772 SEQ ID NO: 44 |
| AAV43.23 | 79 | US20030138772 SEQ ID NO: 98 |
| AAV43.25 | 80 | US20030138772 SEQ ID NO: 45 |
| AAV43.25 | 81 | US20030138772 SEQ ID NO: 97 |
| AAV43.5 | 82 | US20030138772 SEQ ID NO: 40 |
| AAV43.5 | 83 | US20030138772 SEQ ID NO: 94 |
| AAV4-4 | 84 | US20150315612 SEQ ID NO: 201 |
| AAV4-4 | 85 | US20150315612 SEQ ID NO: 218 |
| AAV44.1 | 86 | US20030138772 SEQ ID NO: 46 |
| AAV44.1 | 87 | US20030138772 SEQ ID NO: 79 |
| AAV44.5 | 88 | US20030138772 SEQ ID NO: 47 |
| AAV44.5 | 89 | US20030138772 SEQ ID NO: 80 |
| AAV4407 | 90 | US20150315612 SEQ ID NO: 90 |
| AAV5 | 91 | U.S. Pat. No. 7,427,396 SEQ ID NO: 1 |
| AAV5 | 92 | US20030138772 SEQ ID NO: 114 |
| AAV5 | 93 | US20160017295 SEQ ID NO: 5, U.S. Pat. No. 7,427,396 SEQ ID NO: 2, |
| | | US20150315612 SEQ ID NO: 216 |
| AAV5 | 94 | US20150315612 SEQ ID NO: 199 |
| AAV6 | 95 | US20150159173 SEQ ID NO: 13 |
| AAV6 | 96 | US20030138772 SEQ ID NO: 65, US20150159173 SEQ ID NO: 29, |
| | | US20160017295 SEQ ID NO: 6, U.S. Pat. No. 6,156,303 SEQ ID NO: 7 |
| AAV6 | 97 | U.S. Pat. No. 6,156,303 SEQ ID NO: 11 |
| AAV6 | 98 | U.S. Pat. No. 6,156,303 SEQ ID NO: 2 |
| AAV6 | 99 | US20150315612 SEQ ID NO: 203 |
| AAV6 | 100 | US20150315612 SEQ ID NO: 220 |
| AAV6.1 | 101 | US20150159173 |
| AAV6.12 | 102 | US20150159173 |
| AAV6.2 | 103 | US20150159173 |
| AAV7 | 104 | US20150159173 SEQ ID NO: 14 |
| AAV7 | 105 | US20150315612 SEQ ID NO: 183 |
| AAV7 | 106 | US20030138772 SEQ ID NO: 2, US20150159173 SEQ ID NO: 30, |
| | | US20150315612 SEQ ID NO: 181, US20160017295 SEQ ID NO: 7 |
| AAV7 | 107 | US20030138772 SEQ ID NO: 3 |
| AAV7 | 108 | US20030138772 SEQ ID NO: 1, US20150315612 SEQ ID NO: 180 |
| AAV7 | 109 | US20150315612 SEQ ID NO: 213 |
| AAV7 | 110 | US20150315612 SEQ ID NO: 222 |
| AAV8 | 111 | US20150159173 SEQ ID NO: 15 |
| AAV8 | 112 | US20150376240 SEQ ID NO: 7 |
| AAV8 | 113 | US20030138772 SEQ ID NO: 4, US20150315612 SEQ ID NO: 182 |
| AAV8 | 114 | US20030138772 SEQ ID NO: 95, US20140359799 SEQ ID NO: 1, |
| | | US20150159173 SEQ ID NO: 31, US20160017295 SEQ ID NO: 8, |
| | | U.S. Pat. No. 7,198,951 SEQ ID NO: 7, US20150315612 SEQ ID NO: 223 |
| AAV8 | 115 | US20150376240 SEQ ID NO: 8 |
| AAV8 | 116 | US20150315612 SEQ ID NO: 214 |
| AAV-8b | 117 | US20150376240 SEQ ID NO: 5 |
| AAV-8b | 118 | US20150376240 SEQ ID NO: 3 |
| AAV-8h | 119 | US20150376240 SEQ ID NO: 6 |
| AAV-8h | 120 | US20150376240 SEQ ID NO: 4 |
| AAV9 | 121 | US20030138772 SEQ ID NO: 5 |
| AAV9 | 122 | U.S. Pat. No. 7,198,951 SEQ ID NO: 1 |
| AAV9 | 123 | US20160017295 SEQ ID NO: 9 |
| AAV9 | 124 | US20030138772 SEQ ID NO: 100, U.S. Pat. No. 7,198,951 SEQ ID NO: 2 |
| AAV9 | 125 | U.S. Pat. No. 7,198,951 SEQ ID NO: 3 |
| AAV9 (AAVhu.14) | 126 | US20150315612 SEQ ID NO: 3 |
| AAV9 (AAVhu.14) | 127 | US20150315612 SEQ ID NO: 123 |
| AAVA3.1 | 128 | US20030138772 SEQ ID NO: 120 |
| AAVA3.3 | 129 | US20030138772 SEQ ID NO: 57 |
| AAVA3.3 | 130 | US20030138772 SEQ ID NO: 66 |
| AAVA3.4 | 131 | US20030138772 SEQ ID NO: 54 |
| AAVA3.4 | 132 | US20030138772 SEQ ID NO: 68 |
| AAVA3.5 | 133 | US20030138772 SEQ ID NO: 55 |
| AAVA3.5 | 134 | US20030138772 SEQ ID NO: 69 |
| AAVA3.7 | 135 | US20030138772 SEQ ID NO: 56 |
| AAVA3.7 | 136 | US20030138772 SEQ ID NO: 67 |
| AAV29.3 (AAVbb.1) | 137 | US20030138772 SEQ ID NO: 11 |
| AAVC2 | 138 | US20030138772 SEQ ID NO: 61 |
| AAVCh.5 | 139 | US20150159173 SEQ ID NO: 46, US20150315612 SEQ ID NO: 234 |
| AAVcy.2 (AAV13.3) | 140 | US20030138772 SEQ ID NO: 15 |
| AAV24.1 | 141 | US20030138772 SEQ ID NO: 101 |
| AAVcy.3 (AAV24.1) | 142 | US20030138772 SEQ ID NO: 16 |
| AAV27.3 | 143 | US20030138772 SEQ ID NO: 104 |
| AAVcy.4 (AAV27.3) | 144 | US20030138772 SEQ ID NO: 17 |
| AAVcy.5 | 145 | US20150315612 SEQ ID NO: 227 |
| AAV7.2 | 146 | US20030138772 SEQ ID NO: 103 |
| AAVcy.5 (AAV7.2) | 147 | US20030138772 SEQ ID NO: 18 |
| AAV16.3 | 148 | US20030138772 SEQ ID NO: 105 |
| AAVcy.6 (AAV16.3) | 149 | US20030138772 SEQ ID NO: 10 |
| AAVcy.5 | 150 | US20150159173 SEQ ID NO: 8 |
| AAVcy.5 | 151 | US20150159173 SEQ ID NO: 24 |
| AAVCy.5R1 | 152 | US20150159173 |
| AAVCy.5R2 | 153 | US20150159173 |
| AAVCy.5R3 | 154 | US20150159173 |
| AAVCy.5R4 | 155 | US20150159173 |
| AAVDJ | 156 | US20140359799 SEQ ID NO: 3, U.S. Pat. No. 7,588,772 SEQ ID NO: 2 |
| AAVDJ | 157 | US20140359799 SEQ ID NO: 2, U.S. Pat. No. 7,588,772 SEQ ID NO: 1 |
| AAVDJ-8 | 158 | U.S. Pat. No. 7,588,772; Grimm et al 2008 |
| AAVDJ-8 | 159 | U.S. Pat. No. 7,588,772; Grimm et al 2008 |
| AAVF5 | 160 | US20030138772 SEQ ID NO: 110 |
| AAVH2 | 161 | US20030138772 SEQ ID NO: 26 |
| AAVH6 | 162 | US20030138772 SEQ ID NO: 25 |
| AAVhE1.1 | 163 | U.S. Pat. No. 9,233,131 SEQ ID NO: 44 |
| AAVhEr1.14 | 164 | U.S. Pat. No. 9,233,131 SEQ ID NO: 46 |
| AAVhEr1.16 | 165 | U.S. Pat. No. 9,233,131 SEQ ID NO: 48 |
| AAVhEr1.18 | 166 | U.S. Pat. No. 9,233,131 SEQ ID NO: 49 |
| AAVhEr1.23 | 167 | U.S. Pat. No. 9,233,131 SEQ ID NO: 53 |
| (AAVhEr2.29) | | |
| AAVhEr1.35 | 168 | U.S. Pat. No. 9,233,131 SEQ ID NO: 50 |
| AAVhEr1.36 | 169 | U.S. Pat. No. 9,233,131 SEQ ID NO: 52 |
| AAVhEr1.5 | 170 | U.S. Pat. No. 9,233,131 SEQ ID NO: 45 |
| AAVhEr1.7 | 171 | U.S. Pat. No. 9,233,131 SEQ ID NO: 51 |
| AAVhEr1.8 | 172 | U.S. Pat. No. 9,233,131 SEQ ID NO: 47 |
| AAVhEr2.16 | 173 | U.S. Pat. No. 9,233,131 SEQ ID NO: 55 |
| AAVhEr2.30 | 174 | U.S. Pat. No. 9,233,131 SEQ ID NO: 56 |
| AAVhEr2.31 | 175 | U.S. Pat. No. 9,233,131 SEQ ID NO: 58 |
| AAVhEr2.36 | 176 | U.S. Pat. No. 9,233,131 SEQ ID NO: 57 |
| AAVhEr2.4 | 177 | U.S. Pat. No. 9,233,131 SEQ ID NO: 54 |
| AAVhEr3.1 | 178 | U.S. Pat. No. 9,233,131 SEQ ID NO: 59 |
| AAVhu.1 | 179 | US20150315612 SEQ ID NO: 46 |
| AAVhu.1 | 180 | US20150315612 SEQ ID NO: 144 |
| AAVhu.10 | 181 | US20150315612 SEQ ID NO: 56 |
| (AAV16.8) | | |
| AAVhu.10 | 182 | US20150315612 SEQ ID NO: 156 |
| (AAV16.8) | | |
| AAVhu.11 | 183 | US20150315612 SEQ ID NO: 57 |
| (AAV16.12) | | |
| AAVhu.11 | 184 | US20150315612 SEQ ID NO: 153 |
| (AAV16.12) | | |
| AAVhu.12 | 185 | US20150315612 SEQ ID NO: 59 |
| AAVhu.12 | 186 | US20150315612 SEQ ID NO: 154 |
| AAVhu.13 | 187 | US20150159173 SEQ ID NO: 16, US20150315612 SEQ ID NO: 71 |
| AAVhu.13 | 188 | US20150159173 SEQ ID NO: 32, US20150315612 SEQ ID NO: 129 |
| AAVhu.136.1 | 189 | US20150315612 SEQ ID NO: 165 |
| AAVhu.140.1 | 190 | US20150315612 SEQ ID NO: 166 |
| AAVhu.140.2 | 191 | US20150315612 SEQ ID NO: 167 |
| AAVhu.145.6 | 192 | US20150315612 SEQ ID No: 178 |
| AAVhu.15 | 193 | US20150315612 SEQ ID NO: 147 |
| AAVhu.15 | 194 | US20150315612 SEQ ID NO: 50 |
| (AAV33.4) | | |
| AAVhu.156.1 | 195 | US20150315612 SEQ ID No: 179 |
| AAVhu.16 | 196 | US20150315612 SEQ ID NO: 148 |
| AAVhu.16 | 197 | US20150315612 SEQ ID NO: 51 |
| (AAV33.8) | | |
| AAVhu.17 | 198 | US20150315612 SEQ ID NO: 83 |
| AAVhu.17 | 199 | US20150315612 SEQ ID NO: 4 |
| (AAV33.12) | | |
| AAVhu.172.1 | 200 | US20150315612 SEQ ID NO: 171 |
| AAVhu.172.2 | 201 | US20150315612 SEQ ID NO: 172 |
| AAVhu.173.4 | 202 | US20150315612 SEQ ID NO: 173 |
| AAVhu.173.8 | 203 | US20150315612 SEQ ID NO: 175 |
| AAVhu.18 | 204 | US20150315612 SEQ ID NO: 52 |
| AAVhu.18 | 205 | US20150315612 SEQ ID NO: 149 |
| AAVhu.19 | 206 | US20150315612 SEQ ID NO: 62 |
| AAVhu.19 | 207 | US20150315612 SEQ ID NO: 133 |
| AAVhu.2 | 208 | US20150315612 SEQ ID NO: 48 |
| AAVhu.2 | 209 | US20150315612 SEQ ID NO: 143 |
| AAVhu.20 | 210 | US20150315612 SEQ ID NO: 63 |
| AAVhu.20 | 211 | US20150315612 SEQ ID NO: 134 |
| AAVhu.21 | 212 | US20150315612 SEQ ID NO: 65 |
| AAVhu.21 | 213 | US20150315612 SEQ ID NO: 135 |
| AAVhu.22 | 214 | US20150315612 SEQ ID NO: 67 |
| AAVhu.22 | 215 | US20150315612 SEQ ID NO: 138 |
| AAVhu.23 | 216 | US20150315612 SEQ ID NO: 60 |
| AAVhu.23.2 | 217 | US20150315612 SEQ ID NO: 137 |
| AAVhu.24 | 218 | US20150315612 SEQ ID NO: 66 |
| AAVhu.24 | 219 | US20150315612 SEQ ID NO: 136 |
| AAVhu.25 | 220 | US20150315612 SEQ ID NO: 49 |
| AAVhu.25 | 221 | US20150315612 SEQ ID NO: 146 |
| AAVhu.26 | 222 | US20150159173 SEQ ID NO: 17, US20150315612 SEQ ID NO: 61 |
| AAVhu.26 | 223 | US20150159173 SEQ ID NO: 33, US20150315612 SEQ ID NO: 139 |
| AAVhu.27 | 224 | US20150315612 SEQ ID NO: 64 |
| AAVhu.27 | 225 | US20150315612 SEQ ID NO: 140 |
| AAVhu.28 | 226 | US20150315612 SEQ ID NO: 68 |
| AAVhu.28 | 227 | US20150315612 SEQ ID NO: 130 |
| AAVhu.29 | 228 | US20150315612 SEQ ID NO: 69 |
| AAVhu.29 | 229 | US20150159173 SEQ ID NO: 42, US20150315612 SEQ ID NO: 132 |
| AAVhu.29 | 230 | US20150315612 SEQ ID NO: 225 |
| AAVhu.29R | 231 | US20150159173 |
| AAVhu.3 | 232 | US20150315612 SEQ ID NO: 44 |
| AAVhu.3 | 233 | US20150315612 SEQ ID NO: 145 |
| AAVhu.30 | 234 | US20150315612 SEQ ID NO: 70 |
| AAVhu.30 | 235 | US20150315612 SEQ ID NO: 131 |
| AAVhu.31 | 236 | US20150315612 SEQ ID NO: 1 |
| AAVhu.31 | 237 | US20150315612 SEQ ID NO: 121 |
| AAVhu.32 | 238 | US20150315612 SEQ ID NO: 2 |
| AAVhu.32 | 239 | US20150315612 SEQ ID NO: 122 |
| AAVhu.33 | 240 | US20150315612 SEQ ID NO: 75 |
| AAVhu.33 | 241 | US20150315612 SEQ ID NO: 124 |
| AAVhu.34 | 242 | US20150315612 SEQ ID NO: 72 |
| AAVhu.34 | 243 | US20150315612 SEQ ID NO: 125 |
| AAVhu.35 | 244 | US20150315612 SEQ ID NO: 73 |
| AAVhu.35 | 245 | US20150315612 SEQ ID NO: 164 |
| AAVhu.36 | 246 | US20150315612 SEQ ID NO: 74 |
| AAVhu.36 | 247 | US20150315612 SEQ ID NO: 126 |
| AAVhu.37 | 248 | US20150159173 SEQ ID NO: 34, US20150315612 SEQ ID NO: 88 |
| AAVhu.37 | 249 | US20150315612 SEQ ID NO: 10, US20150159173 SEQ ID NO: 18 |
| (AAV106.1) | | |
| AAVhu.3 8 | 250 | US20150315612 SEQ ID NO: 161 |
| AAVhu.3 9 | 251 | US20150315612 SEQ ID NO: 102 |
| AAVhu.3 9 | 252 | US20150315612 SEQ ID NO: 24 |
| (AAVLG-9) | | |
| AAVhu.4 | 253 | US20150315612 SEQ ID NO: 47 |
| AAVhu.4 | 254 | US20150315612 SEQ ID NO: 141 |
| AAVhu.40 | 255 | US20150315612 SEQ ID NO: 87 |
| AAVhu.40 | 256 | US20150315612 SEQ ID No: 11 |
| (AAV114.3) | | |
| AAVhu.41 | 257 | US20150315612 SEQ ID NO: 91 |
| AAVhu.41 | 258 | US20150315612 SEQ ID NO: 6 |
| (AAV127.2) | | |
| AAVhu.42 | 259 | US20150315612 SEQ ID NO: 85 |
| AAVhu.42 | 260 | US20150315612 SEQ ID NO: 8 |
| (AAV127.5) | | |
| AAVhu.43 | 261 | US20150315612 SEQ ID NO: 160 |
| AAVhu.43 | 262 | US20150315612 SEQ ID NO: 236 |
| AAVhu.43 | 263 | US20150315612 SEQ ID NO: 80 |
| (AAV128.1) | | |
| AAVhu.44 | 264 | US20150159173 SEQ ID NO: 45, US20150315612 SEQ ID NO: 158 |
| AAVhu.44 | 265 | US20150315612 SEQ ID NO: 81 |
| (AAV128.3) | | |
| AAVhu.44R1 | 266 | US20150159173 |
| AAVhu.44R2 | 267 | US20150159173 |
| AAVhu.44R3 | 268 | US20150159173 |
| AAVhu.45 | 269 | US20150315612 SEQ ID NO: 76 |
| AAVhu.45 | 270 | US20150315612 SEQ ID NO: 127 |
| AAVhu.46 | 271 | US20150315612 SEQ ID NO: 82 |
| AAVhu.46 | 272 | US20150315612 SEQ ID NO: 159 |
| AAVhu.46 | 273 | US20150315612 SEQ ID NO: 224 |
| AAVhu.47 | 274 | US20150315612 SEQ ID NO: 77 |
| AAVhu.47 | 275 | US20150315612 SEQ ID NO: 128 |
| AAVhu.48 | 276 | US20150159173 SEQ ID NO: 38 |
| AAVhu.48 | 277 | US20150315612 SEQ ID NO: 157 |
| AAVhu.48 | 278 | US20150315612 SEQ ID NO: 78 |
| (AAV130.4) | | |
| AAVhu.48R1 | 279 | US20150159173 |
| AAVhu.48R2 | 280 | US20150159173 |
| AAVhu.48R3 | 281 | US20150159173 |
| AAVhu.49 | 282 | US20150315612 SEQ ID NO: 209 |
| AAVhu.49 | 283 | US20150315612 SEQ ID NO: 189 |
| AAVhu.5 | 284 | US20150315612 SEQ ID NO: 45 |
| AAVhu.5 | 285 | US20150315612 SEQ ID NO: 142 |
| AAVhu.51 | 286 | US20150315612 SEQ ID NO: 208 |
| AAVhu.51 | 287 | US20150315612 SEQ ID NO: 190 |
| AAVhu.52 | 288 | US20150315612 SEQ ID NO: 210 |
| AAVhu.52 | 289 | US20150315612 SEQ ID NO: 191 |
| AAVhu.53 | 290 | US20150159173 SEQ ID NO: 19 |
| AAVhu.53 | 291 | US20150159173 SEQ ID NO: 35 |
| AAVhu.53 | 292 | US20150315612 SEQ ID NO: 176 |
| (AAV145.1) | | |
| AAVhu.54 | 293 | US20150315612 SEQ ID NO: 188 |
| AAVhu.54 | 294 | US20150315612 SEQ ID No: 177 |
| (AAV145.5) | | |
| AAVhu.55 | 295 | US20150315612 SEQ ID NO: 187 |
| AAVhu.56 | 296 | US20150315612 SEQ ID NO: 205 |
| AAVhu.56 | 297 | US20150315612 SEQ ID NO: 168 |
| (AAV145.6) | | |
| AAVhu.56 | 298 | US20150315612 SEQ ID NO: 192 |
| (AAV145.6) | | |
| AAVhu.57 | 299 | US20150315612 SEQ ID NO: 206 |
| AAVhu.57 | 300 | US20150315612 SEQ ID NO: 169 |
| AAVhu.57 | 301 | US20150315612 SEQ ID NO: 193 |
| AAVhu.58 | 302 | US20150315612 SEQ ID NO: 207 |
| AAVhu.58 | 303 | US20150315612 SEQ ID NO: 194 |
| AAVhu.6 (AAV3.1) | 304 | US20150315612 SEQ ID NO: 5 |
| AAVhu.6 (AAV3.1) | 305 | US20150315612 SEQ ID NO: 84 |
| AAVhu.60 | 306 | US20150315612 SEQ ID NO: 184 |
| AAVhu.60 | 307 | US20150315612 SEQ ID NO: 170 |
| (AAV161.10) | | |
| AAVhu.61 | 308 | US20150315612 SEQ ID NO: 185 |
| AAVhu.61 | 309 | US20150315612 SEQ ID NO: 174 |
| (AAV161.6) | | |
| AAVhu.63 | 310 | US20150315612 SEQ ID NO: 204 |
| AAVhu.63 | 311 | US20150315612 SEQ ID NO: 195 |
| AAVhu.64 | 312 | US20150315612 SEQ ID NO: 212 |
| AAVhu.64 | 313 | US20150315612 SEQ ID NO: 196 |
| AAVhu.66 | 314 | US20150315612 SEQ ID NO: 197 |
| AAVhu.67 | 315 | US20150315612 SEQ ID NO: 215 |
| AAVhu.67 | 316 | US20150315612 SEQ ID NO: 198 |
| AAVhu.7 | 317 | US20150315612 SEQ ID NO: 226 |
| AAVhu.7 | 318 | US20150315612 SEQ ID NO: 150 |
| AAVhu.7 (AAV7.3) | 319 | US20150315612 SEQ ID NO: 55 |
| AAVhu.71 | 320 | US20150315612 SEQ ID NO: 79 |
| AAVhu.8 | 321 | US20150315612 SEQ ID NO: 53 |
| AAVhu.8 | 322 | US20150315612 SEQ ID NO: 12 |
| AAVhu.8 | 323 | US20150315612 SEQ ID NO: 151 |
| AAVhu.9 (AAV3.1) | 324 | US20150315612 SEQ ID NO: 58 |
| AAVhu.9 (AAV3.1) | 325 | US20150315612 SEQ ID NO: 155 |
| AAV-LK01 | 326 | US20150376607 SEQ ID NO: 2 |
| AAV-LK01 | 327 | US20150376607 SEQ ID NO: 29 |
| AAV-LK02 | 328 | US20150376607 SEQ ID NO: 3 |
| AAV-LK02 | 329 | US20150376607 SEQ ID NO: 30 |
| AAV-LK03 | 330 | US20150376607 SEQ ID NO: 4 |
| AAV-LK03 | 331 | WO2015121501 SEQ ID NO: 12, US20150376607 SEQ ID NO: 31 |
| AAV-LK04 | 332 | US20150376607 SEQ ID NO: 5 |
| AAV-LK04 | 333 | US20150376607 SEQ ID NO: 32 |
| AAV-LK05 | 334 | US20150376607 SEQ ID NO: 6 |
| AAV-LK05 | 335 | US20150376607 SEQ ID NO: 33 |
| AAV-LK06 | 336 | US20150376607 SEQ ID NO: 7 |
| AAV-LK06 | 337 | US20150376607 SEQ ID NO: 34 |
| AAV-LK07 | 338 | US20150376607 SEQ ID NO: 8 |
| AAV-LK07 | 339 | US20150376607 SEQ ID NO: 35 |
| AAV-LK08 | 340 | US20150376607 SEQ ID NO: 9 |
| AAV-LK08 | 341 | US20150376607 SEQ ID NO: 36 |
| AAV-LK09 | 342 | US20150376607 SEQ ID NO: 10 |
| AAV-LK09 | 343 | US20150376607 SEQ ID NO: 37 |
| AAV-LK10 | 344 | US20150376607 SEQ ID NO: 11 |
| AAV-LK10 | 345 | US20150376607 SEQ ID NO: 38 |
| AAV-LK11 | 346 | US20150376607 SEQ ID NO: 12 |
| AAV-LK11 | 347 | US20150376607 SEQ ID NO: 39 |
| AAV-LK12 | 348 | US20150376607 SEQ ID NO: 13 |
| AAV-LK12 | 349 | US20150376607 SEQ ID NO: 40 |
| AAV-LK13 | 350 | US20150376607 SEQ ID NO: 14 |
| AAV-LK13 | 351 | US20150376607 SEQ ID NO: 41 |
| AAV-LK14 | 352 | US20150376607 SEQ ID NO: 15 |
| AAV-LK14 | 353 | US20150376607 SEQ ID NO: 42 |
| AAV-LK15 | 354 | US20150376607 SEQ ID NO: 16 |
| AAV-LK15 | 355 | US20150376607 SEQ ID NO: 43 |
| AAV-LK16 | 356 | US20150376607 SEQ ID NO: 17 |
| AAV-LK16 | 357 | US20150376607 SEQ ID NO: 44 |
| AAV-LK17 | 358 | US20150376607 SEQ ID NO: 18 |
| AAV-LK17 | 359 | US20150376607 SEQ ID NO: 45 |
| AAV-LK18 | 360 | US20150376607 SEQ ID NO: 19 |
| AAV-LK18 | 361 | US20150376607 SEQ ID NO: 46 |
| AAV-LK19 | 362 | US20150376607 SEQ ID NO: 20 |
| AAV-LK19 | 363 | US20150376607 SEQ ID NO: 47 |
| AAV-PAEC | 364 | US20150376607 SEQ ID NO: 1 |
| AAV-PAEC | 365 | US20150376607 SEQ ID NO: 48 |
| AAV-PAEC11 | 366 | US20150376607 SEQ ID NO: 26 |
| AAV-PAEC11 | 367 | US20150376607 SEQ ID NO: 54 |
| AAV-PAEC12 | 368 | US20150376607 SEQ ID NO: 27 |
| AAV-PAEC12 | 369 | US20150376607 SEQ ID NO: 51 |
| AAV-PAEC13 | 370 | US20150376607 SEQ ID NO: 28 |
| AAV-PAEC13 | 371 | US20150376607 SEQ ID NO: 49 |
| AAV-PAEC2 | 372 | US20150376607 SEQ ID NO: 21 |
| AAV-PAEC2 | 373 | US20150376607 SEQ ID NO: 56 |
| AAV-PAEC4 | 374 | US20150376607 SEQ ID NO: 22 |
| AAV-PAEC4 | 375 | US20150376607 SEQ ID NO: 55 |
| AAV-PAEC6 | 376 | US20150376607 SEQ ID NO: 23 |
| AAV-PAEC6 | 377 | US20150376607 SEQ ID NO: 52 |
| AAV-PAEC7 | 378 | US20150376607 SEQ ID NO: 24 |
| AAV-PAEC7 | 379 | US20150376607 SEQ ID NO: 53 |
| AAV-PAEC8 | 380 | US20150376607 SEQ ID NO: 25 |
| AAV-PAEC8 | 381 | US20150376607 SEQ ID NO: 50 |
| AAVpi.1 | 382 | US20150315612 SEQ ID NO: 28 |
| AAVpi.1 | 383 | US20150315612 SEQ ID NO: 93 |
| AAVpi.2 | 384 | US20150315612 SEQ ID NO: 30 |
| AAVpi.2 | 385 | US20150315612 SEQ ID NO: 95 |
| AAVpi.3 | 386 | US20150315612 SEQ ID NO: 29 |
| AAVpi.3 | 387 | US20150315612 SEQ ID NO: 94 |
| AAVrh.10 | 388 | US20150159173 SEQ ID NO: 9 |
| AAVrh.10 | 389 | US20150159173 SEQ ID NO: 25 |
| AAV44.2 | 390 | US20030138772 SEQ ID NO: 59 |
| AAVrh.10 | 391 | US20030138772 SEQ ID NO: 81 |
| (AAV44.2) | | |
| AAV42.1B | 392 | US20030138772 SEQ ID NO: 90 |
| AAVrh.12 | 393 | US20030138772 SEQ ID NO: 30 |
| (AAV42.1b) | | |
| AAVrh.13 | 394 | US20150159173 SEQ ID NO: 10 |
| AAVrh.13 | 395 | US20150159173 SEQ ID NO: 26 |
| AAVrh.13 | 396 | US20150315612 SEQ ID NO: 228 |
| AAVrh.13R | 397 | US20150159173 |
| AAV42.3A | 398 | US20030138772 SEQ ID NO: 87 |
| AAVrh.14 | 399 | US20030138772 SEQ ID NO: 32 |
| (AAV42.3a) | | |
| AAV42.5A | 400 | US20030138772 SEQ ID NO: 89 |
| AAVrh.17 | 401 | US20030138772 SEQ ID NO: 34 |
| (AAV42.5a) | | |
| AAV42.5B | 402 | US20030138772 SEQ ID NO: 91 |
| AAVrh.18 | 403 | US20030138772 SEQ ID NO: 29 |
| (AAV42.5b) | | |
| AAV42.6B | 404 | US20030138772 SEQ ID NO: 112 |
| AAVrh.19 | 405 | US20030138772 SEQ ID NO: 38 |
| (AAV42.6b) | | |
| AAVrh.2 | 406 | US20150159173 SEQ ID NO: 39 |
| AAVrh.2 | 407 | US20150315612 SEQ ID NO: 231 |
| AAVrh.20 | 408 | US20150159173 SEQ ID NO: 1 |
| AAV42.10 | 409 | US20030138772 SEQ ID NO: 106 |
| AAVrh.21 | 410 | US20030138772 SEQ ID NO: 35 |
| (AAV42.10) | | |
| AAV42.11 | 411 | US20030138772 SEQ ID NO: 108 |
| AAVrh.22 | 412 | US20030138772 SEQ ID NO: 37 |
| (AAV42.11) | | |
| AAV42.12 | 413 | US20030138772 SEQ ID NO: 113 |
| AAVrh.23 | 414 | US20030138772 SEQ ID NO: 58 |
| (AAV42.12) | | |
| AAV42.13 | 415 | US20030138772 SEQ ID NO: 86 |
| AAVrh.24 | 416 | US20030138772 SEQ ID NO: 31 |
| (AAV42.13) | | |
| AAV42.15 | 417 | US20030138772 SEQ ID NO: 84 |
| AAVrh.25 | 418 | US20030138772 SEQ ID NO: 28 |
| (AAV42.15) | | |
| AAVrh.2R | 419 | US20150159173 |
| AAVrh.31 | 420 | US20030138772 SEQ ID NO: 48 |
| (AAV223.1) | | |
| AAVC1 | 421 | US20030138772 SEQ ID NO: 60 |
| AAVrh.32 (AAVC1) | 422 | US20030138772 SEQ ID NO: 19 |
| AAVrh.32/33 | 423 | US20150159173 SEQ ID NO: 2 |
| AAVrh.33 (AAVC3) | 424 | US20030138772 SEQ ID NO: 20 |
| AAVC5 | 425 | US20030138772 SEQ ID NO: 62 |
| AAVrh.34 (AAVC5) | 426 | US20030138772 SEQ ID NO: 21 |
| AAVF1 | 427 | US20030138772 SEQ ID NO: 109 |
| AAVrh.35 (AAVF1) | 428 | US20030138772 SEQ ID NO: 22 |
| AAVF3 | 429 | US20030138772 SEQ ID NO: 111 |
| AAVrh.36 (AAVF3) | 430 | US20030138772 SEQ ID NO: 23 |
| AAVrh.37 | 431 | US20030138772 SEQ ID NO: 24 |
| AAVrh.37 | 432 | US20150159173 SEQ ID NO: 40 |
| AAVrh.37 | 433 | US20150315612 SEQ ID NO: 229 |
| AAVrh.37R2 | 434 | US20150159173 |
| AAVrh.38 (AAVLG- | 435 | US20150315612 SEQ ID NO: 7 |
| 4) | | |
| AAVrh.38 (AAVLG- | 436 | US20150315612 SEQ ID NO: 86 |
| 4) | | |
| AAVrh.39 | 437 | US20150159173 SEQ ID NO: 20, US20150315612 SEQ ID NO: 13 |
| AAVrh.39 | 438 | US20150159173 SEQ ID NO: 3, US20150159173 SEQ ID NO: 36, |
| | | US20150315612 SEQ ID NO: 89 |
| AAVrh.40 | 439 | US20150315612 SEQ ID NO: 92 |
| AAVrh.40 (AAVLG- | 440 | US20150315612 SEQ ID No: 14 |
| 10) | | |
| AAVrh.43 | 441 | US20150315612 SEQ ID NO: 43, US20150159173 SEQ ID NO: 21 |
| (AAVN721-8) | | |
| AAVrh.43 | 442 | US20150315612 SEQ ID NO: 163, US20150159173 SEQ ID NO: 37 |
| (AAVN721-8) | | |
| AAVrh.44 | 443 | US20150315612 SEQ ID NO: 34 |
| AAVrh.44 | 444 | US20150315612 SEQ ID NO: 111 |
| AAVrh.45 | 445 | US20150315612 SEQ ID NO: 41 |
| AAVrh.45 | 446 | US20150315612 SEQ ID NO: 109 |
| AAVrh.46 | 447 | US20150159173 SEQ ID NO: 22, US20150315612 SEQ ID NO: 19 |
| AAVrh.46 | 448 | US20150159173 SEQ ID NO: 4, US20150315612 SEQ ID NO: 101 |
| AAVrh.47 | 449 | US20150315612 SEQ ID NO: 38 |
| AAVrh.47 | 450 | US20150315612 SEQ ID NO: 118 |
| AAVrh.48 | 451 | US20150159173 SEQ ID NO: 44, US20150315612 SEQ ID NO: 115 |
| AAVrh.48.1 | 452 | US20150159173 |
| AAVrh.48.1.2 | 453 | US20150159173 |
| AAVrh.48.2 | 454 | US20150159173 |
| AAVrh.48 (AAV1-7) | 455 | US20150315612 SEQ ID NO: 32 |
| AAVrh.49 (AAV1-8) | 456 | US20150315612 SEQ ID NO: 25 |
| AAVrh.49 (AAV1-8) | 457 | US20150315612 SEQ ID NO: 103 |
| AAVrh.50 (AAV2-4) | 458 | US20150315612 SEQ ID NO: 23 |
| AAVrh.50 (AAV2-4) | 459 | US20150315612 SEQ ID NO: 108 |
| AAVrh.51 (AAV2-5) | 460 | US20150315612 SEQ ID No: 22 |
| AAVrh.51 (AAV2-5) | 461 | US20150315612 SEQ ID NO: 104 |
| AAVrh.52 (AAV3-9) | 462 | US20150315612 SEQ ID NO: 18 |
| AAVrh.52 (AAV3-9) | 463 | US20150315612 SEQ ID NO: 96 |
| AAVrh.53 | 464 | US20150315612 SEQ ID NO: 97 |
| AAVrh.53 (AAV3- | 465 | US20150315612 SEQ ID NO: 17 |
| 11) | | |
| AAVrh.53 (AAV3- | 466 | US20150315612 SEQ ID NO: 186 |
| 11) | | |
| AAVrh.54 | 467 | US20150315612 SEQ ID NO: 40 |
| AAVrh.54 | 468 | US20150159173 SEQ ID NO: 49, US20150315612 SEQ ID NO: 116 |
| AAVrh.55 | 469 | US20150315612 SEQ ID NO: 37 |
| AAVrh.55 (AAV4- | 470 | US20150315612 SEQ ID NO: 117 |
| 19) | | |
| AAVrh.56 | 471 | US20150315612 SEQ ID NO: 54 |
| AAVrh.56 | 472 | US20150315612 SEQ ID NO: 152 |
| AAVrh.57 | 473 | US20150315612 SEQ ID NO: 26 |
| AAVrh.57 | 474 | US20150315612 SEQ ID NO: 105 |
| AAVrh.58 | 475 | US20150315612 SEQ ID NO: 27 |
| AAVrh.58 | 476 | US20150159173 SEQ ID NO: 48, US20150315612 SEQ ID NO: 106 |
| AAVrh.58 | 477 | US20150315612 SEQ ID NO: 232 |
| AAVrh.59 | 478 | US20150315612 SEQ ID NO: 42 |
| AAVrh.59 | 479 | US20150315612 SEQ ID NO: 110 |
| AAVrh.60 | 480 | US20150315612 SEQ ID NO: 31 |
| AAVrh.60 | 481 | US20150315612 SEQ ID NO: 120 |
| AAVrh.61 | 482 | US20150315612 SEQ ID NO: 107 |
| AAVrh.61 (AAV2-3) | 483 | US20150315612 SEQ ID NO: 21 |
| AAVrh.62 (AAV2- | 484 | US20150315612 SEQ ID No: 33 |
| 15) | | |
| AAVrh.62 (AAV2- | 485 | US20150315612 SEQ ID NO: 114 |
| 15) | | |
| AAVrh.64 | 486 | US20150315612 SEQ ID No: 15 |
| AAVrh.64 | 487 | US20150159173 SEQ ID NO: 43, US20150315612 SEQ ID NO: 99 |
| AAVrh.64 | 488 | US20150315612 SEQ ID NO: 233 |
| AAVR11.64R1 | 489 | US20150159173 |
| AAVR11.64R2 | 490 | US20150159173 |
| AAVrh.65 | 491 | US20150315612 SEQ ID NO: 35 |
| AAVrh.65 | 492 | US20150315612 SEQ ID NO: 112 |
| AAVrh.67 | 493 | US20150315612 SEQ ID NO: 36 |
| AAVrh.67 | 494 | US20150315612 SEQ ID NO: 230 |
| AAVrh.67 | 495 | US20150159173 SEQ ID NO: 47, US20150315612 SEQ ID NO: 113 |
| AAVrh.68 | 496 | US20150315612 SEQ ID NO: 16 |
| AAVrh.68 | 497 | US20150315612 SEQ ID NO: 100 |
| AAVrh.69 | 498 | US20150315612 SEQ ID NO: 39 |
| AAVrh.69 | 499 | US20150315612 SEQ ID NO: 119 |
| AAVrh.70 | 500 | US20150315612 SEQ ID NO: 20 |
| AAVrh.70 | 501 | US20150315612 SEQ ID NO: 98 |
| AAVrh.71 | 502 | US20150315612 SEQ ID NO: 162 |
| AAVrh.72 | 503 | US20150315612 SEQ ID NO: 9 |
| AAVrh.73 | 504 | US20150159173 SEQ ID NO: 5 |
| AAVrh.74 | 505 | US20150159173 SEQ ID NO: 6 |
| AAVrh.8 | 506 | US20150159173 SEQ ID NO: 41 |
| AAVrh.8 | 507 | US20150315612 SEQ ID NO: 235 |
| AAVrh.8R | 508 | US20150159173, WO2015168666 SEQ ID NO: 9 |
| AAVrh.8R A586R | 509 | WO2015168666 SEQ ID NO: 10 |
| mutant | | |
| AAVrh.8R R533A | 510 | WO2015168666 SEQ ID NO: 11 |
| mutant | | |
| BAAV (bovine | 511 | U.S. Pat. No. 9,193,769 SEQ ID NO: 8 |
| AAV) | | |
| BAAV (bovine | 512 | U.S. Pat. No. 9,193,769 SEQ ID NO: 10 |
| AAV) | | |
| BAAV (bovine | 513 | U.S. Pat. No. 9,193,769 SEQ ID NO: 4 |
| AAV) | | |
| BAAV (bovine | 514 | U.S. Pat. No. 9,193,769 SEQ ID NO: 2 |
| AAV) | | |
| BAAV (bovine | 515 | U.S. Pat. No. 9,193,769 SEQ ID NO: 6 |
| AAV) | | |
| BAAV (bovine | 516 | U.S. Pat. No. 9,193,769 SEQ ID NO: 1 |
| AAV) | | |
| BAAV (bovine | 517 | U.S. Pat. No. 9,193,769 SEQ ID NO: 5 |
| AAV) | | |
| BAAV (bovine | 518 | U.S. Pat. No. 9,193,769 SEQ ID NO: 3 |
| AAV) | | |
| BAAV (bovine | 519 | U.S. Pat. No. 9,193,769 SEQ ID NO: 11 |
| AAV) | | |
| BAAV (bovine | 520 | U.S. Pat. No. 7,427,396 SEQ ID NO: 5 |
| AAV) | | |
| BAAV (bovine | 521 | U.S. Pat. No. 7,427,396 SEQ ID NO: 6 |
| AAV) | | |
| BAAV (bovine | 522 | U.S. Pat. No. 9,193,769 SEQ ID NO: 7 |
| AAV) | | |
| BAAV (bovine | 523 | U.S. Pat. No. 9,193,769 SEQ ID NO: 9 |
| AAV) | | |
| BNP61 AAV | 524 | US20150238550 SEQ ID NO: 1 |
| BNP61 AAV | 525 | US20150238550 SEQ ID NO: 2 |
| BNP62 AAV | 526 | US20150238550 SEQ ID NO: 3 |
| BNP63 AAV | 527 | US20150238550 SEQ ID NO: 4 |
| caprine AAV | 528 | U.S. Pat. No. 7,427,396 SEQ ID NO: 3 |
| caprine AAV | 529 | U.S. Pat. No. 7,427,396 SEQ ID NO: 4 |
| true type AAV | 530 | WO2015121501 SEQ ID NO: 2 |
| (ttAAV) | | |
| AAAV (Avian AAV) | 531 | U.S. Pat. No. 9,238,800 SEQ ID NO: 12 |
| AAAV (Avian AAV) | 532 | U.S. Pat. No. 9,238,800 SEQ ID NO: 2 |
| AAAV (Avian AAV) | 533 | U.S. Pat. No. 9,238,800 SEQ ID NO: 6 |
| AAAV (Avian AAV) | 534 | U.S. Pat. No. 9,238,800 SEQ ID NO: 4 |
| AAAV (Avian AAV) | 535 | U.S. Pat. No. 9,238,800 SEQ ID NO: 8 |
| AAAV (Avian AAV) | 536 | U.S. Pat. No. 9,238,800 SEQ ID NO: 14 |
| AAAV (Avian AAV) | 537 | U.S. Pat. No. 9,238,800 SEQ ID NO: 10 |
| AAAV (Avian AAV) | 538 | U.S. Pat. No. 9,238,800 SEQ ID NO: 15 |
| AAAV (Avian AAV) | 539 | U.S. Pat. No. 9,238,800 SEQ ID NO: 5 |
| AAAV (Avian AAV) | 540 | U.S. Pat. No. 9,238,800 SEQ ID NO: 9 |
| AAAV (Avian AAV) | 541 | U.S. Pat. No. 9,238,800 SEQ ID NO: 3 |
| AAAV (Avian AAV) | 542 | U.S. Pat. No. 9,238,800 SEQ ID NO: 7 |
| AAAV (Avian AAV) | 543 | U.S. Pat. No. 9,238,800 SEQ ID NO: 11 |
| AAAV (Avian AAV) | 544 | U.S. Pat. No. 9,238,800 SEQ ID NO: 13 |
| AAAV (Avian AAV) | 545 | U.S. Pat. No. 9,238,800 SEQ ID NO: 1 |
| AAV Shuffle 100-1 | 546 | US20160017295 SEQ ID NO: 23 |
| AAV Shuffle 100-1 | 547 | US20160017295 SEQ ID NO: 11 |
| AAV Shuffle 100-2 | 548 | US20160017295 SEQ ID NO: 37 |
| AAV Shuffle 100-2 | 549 | US20160017295 SEQ ID NO: 29 |
| AAV Shuffle 100-3 | 550 | US20160017295 SEQ ID NO: 24 |
| AAV Shuffle 100-3 | 551 | US20160017295 SEQ ID NO: 12 |
| AAV Shuffle 100-7 | 552 | US20160017295 SEQ ID NO: 25 |
| AAV Shuffle 100-7 | 553 | US20160017295 SEQ ID NO: 13 |
| AAV Shuffle 10-2 | 554 | US20160017295 SEQ ID NO: 34 |
| AAV Shuffle 10-2 | 555 | US20160017295 SEQ ID NO: 26 |
| AAV Shuffle 10-6 | 556 | US20160017295 SEQ ID NO: 35 |
| AAV Shuffle 10-6 | 557 | US20160017295 SEQ ID NO: 27 |
| AAV Shuffle 10-8 | 558 | US20160017295 SEQ ID NO: 36 |
| AAV Shuffle 10-8 | 559 | US20160017295 SEQ ID NO: 28 |
| AAV SM 100-10 | 560 | US20160017295 SEQ ID NO: 41 |
| AAV SM 100-10 | 561 | US20160017295 SEQ ID NO: 33 |
| AAV SM 100-3 | 562 | US20160017295 SEQ ID NO: 40 |
| AAV SM 100-3 | 563 | US20160017295 SEQ ID NO: 32 |
| AAV SM 10-1 | 564 | US20160017295 SEQ ID NO: 38 |
| AAV SM 10-1 | 565 | US20160017295 SEQ ID NO: 30 |
| AAV SM 10-2 | 566 | US20160017295 SEQ ID NO: 10 |
| AAV SM 10-2 | 567 | US20160017295 SEQ ID NO: 22 |
| AAV SM 10-8 | 568 | US20160017295 SEQ ID NO: 39 |
| AAV SM 10-8 | 569 | US20160017295 SEQ ID NO: 31 |

Each of the patents, applications and/or publications listed in Table 3 are hereby incorporated by reference in their entirety.

In one embodiment, the AAV serotype may be engineered to comprise at least one AAV capsid CD8+ T-cell epitope. Hui et al. (Molecular Therapy—Methods & Clinical Development (2015) 2, 15029 doi:10.1038/mtm.2015.29; the contents of which are herein incorporated by reference in its entirety) identified AAV capsid-specific CD8+ T-cell epitopes for AAV1 and AAV2 (see e.g., Table 2 in the publication). As a non-limiting example, the capsid-specific CD8+ T-cell epitope may be for an AAV2 serotype. As a non-limiting example, the capsid-specific CD8+ T-cell epitope may be for an AAV1 serotype.

In one embodiment, peptides for inclusion in an AAV serotype may be identified using the methods described by Hui et al. (Molecular Therapy—Methods & Clinical Development (2015) 2, 15029 doi:10.1038/mtm.2015.29; the contents of which are herein incorporated by reference in its entirety). As a non-limiting example, the procedure includes isolating human splenocytes, restimulating the splenocytes in vitro using individual peptides spanning the amino acid sequence of the AAV capsid protein, IFN-gamma ELISpot with the individual peptides used for the in vitro restimulation, bioinformatics analysis to determine the HLA restriction of 15-mers identified by IFN-gamma ELISpot, identification of candidate reactive 9-mer epitopes for a given HLA allele, synthesis candidate 9-mers, second IFN-gamma ELISpot screening of splenocytes from subjects carrying the HLA alleles to which identified AAV epitopes are predicted to bind, determine the AAV capsid-reactive CD8+ T cell epitopes and determine the frequency of subjects reacting to a given AAV epitope.

In one embodiment, peptides for inclusion in an AAV serotype may be identified by isolating human splenocytes, restimulating the splenocytes in vitro using individual peptides spanning the amino acid sequence of the AAV capsid protein, IFN-gamma ELISpot with the individual peptides used for the in vitro restimulation, bioinformatics analysis to determine the given allele restriction of 15-mers identified by IFN-gamma ELISpot, identification of candidate reactive 9-mer epitopes for a given allele, synthesis candidate 9-mers, second IFN-gamma ELISpot screening of splenocytes from subjects carrying the specific alleles to which identified AAV epitopes are predicted to bind, determine the AAV capsid-reactive CD8+ T cell epitopes and determine the frequency of subjects reacting to a given AAV epitope.

AAV vectors comprising the nucleic acid sequence for the siRNA molecules may be prepared or derived from various serotypes of AAVs, including, but not limited to, AAV1, AAV2, AAV3, AAV4, AAV5, AAV6, AAV7, AAV8, AAV9, AAV9.47, AAV9(hul4), AAV10, AAV11, AAV12, AAVrh8, AAVrh10, AAV-DJ8 and AAV-DJ. In some cases, different serotypes of AAVs may be mixed together or with other types of viruses to produce chimeric AAV vectors. As a non-limiting example, the AAV vector is derived from the AAV9 serotype.

In one embodiment, AAV particles of the present invention may comprise capsid proteins having sequences of SEQ ID NOs: 1 and 3, which have increased tropism to the brain, of International Publication No. WO2014160092, the content of which is incorporated herein by reference in its entirety.

In one embodiment, AAV particles of the present invention may comprise capsid proteins which may target to oligodendrocytes in the central nervous system. The capsid proteins may comprise AAV capsid coding sequence of SEQ ID NO: 1 or AAV capsid proteins comprising amino acid sequences of SEQ ID NOs: 2 to 4 of International Publication No. WO2014052789, the content of which is herein incorporated by reference in its entirety.

In one embodiment, AAV particles of the present invention may comprise capsid proteins having increased capacity to cross the blood-brain barrier in CNS as disclosed in U.S. Pat. No. 8,927,514, the content of which is incorporated herein by reference in its entirety. The amino acid sequences and nucleic acid sequences of such capsid proteins may include, but are not limited to, SEQ ID NOs: 2-17 and SEQ ID NOs: 25-33, respectively, of U.S. Pat. No. 8,927,514.

In some embodiments, AAV particles of the present invention may comprise AAV2 capsid proteins or variants thereof. AAV particles with AAV2 capsid proteins have been shown to deliver genes to neurons effectively in the brain, retina and spinal cord. In one embodiment, AAV2 capsid proteins may be further modified such as addition of a targeting peptide to the capsid proteins that targets an AAV particle to brain vascular endothelium as disclosed in U.S. Pat. Nos. 6,691,948 and 8, 299,215, the contents of each of which are herein incorporated by reference in their entirety. Such AAV particles may be used to deliver a functional payload of interest to treat a brain disease such as mucopolysaccharide (MPS).

In some embodiments, AAV particles of the present invention may comprise AAVS capsid proteins or variants thereof. AAV particles with AAVS capsid proteins can transduce neurons in various regions of the CNS, including the cortex, the hippocampus (HPC), cerebellum, substantia nigra (SN), striatum, globus pallidus, and spinal cord (Burger C et al., Mol Ther., 2004, 10(2): 302-317; Liu Get al., Mol Ther. 2007, 15(2): 242-247; and Colle M et al., Hum, Mol. Genet. 2010, 19(1): 147-158). In one embodiment, AAV particles having AAVS capsid proteins with increased transduction to cells in CNS may be those particles from U.S. Pat. No. 7,056,502, the content of which is incorporated herein by reference in its entirety.

In some embodiments, AAV particles of the present invention may comprise AAV6 capsid proteins or variants thereof. Recombinant AAV6 serotype can target motor neurons in the spinal cord by Intracerebroventricular (ICV) injection (Dirren E et al., Hum Gene Ther., 2014, 25(2): 109-120). In addition, a study from San Sebastian et al indicated that AAV6 serotype can be retrogradely transported from terminals to neuronal cell bodies in the rat brain (San Sebastian et al, Gen Ther., 2014, 20(12): 1178-1183).

In some embodiments, AAV particles of the present invention may comprise AAV8 capsid proteins or variants thereof. AAV particles with AAV8 capsid proteins can transduce neurons, for example in hippocampus (Klein RL et al., Mol Ther., 2006, 13(3): 517-527). In one embodiment, AAV8 capsid proteins may comprise the amino acid sequence of SEQ ID NO: 2 of U.S. Pat. No. 8,318,480, the content of which is herein incorporated by reference in its entirety.

In some embodiments, AAV particles of the present invention may comprise AAV9 capsid proteins or variants thereof. AAV9 capsid serotype mediated gene delivery has been observed in the brain with efficient and long-term expression of transgene after intraparenchymal injections to the CNS (Klein R L et al., Eur J Neurosci., 2008, 27: 1615-1625). AAV9 serotype can produce robust and wide-scale neuronal transduction throughout the CNS after a peripheral, systemic (e.g., intravenous) administration in neonatal subjects (Foust K D et al., Nat. Biotechnol., 2009, 27: 59-65; and Duque S et al, Mol Ther., 2009, 17: 1187-1196). Intrathecal (intra-cisterna magna routes) administration of AAV9 serotypes can also produce widespread spinal expression. In one embodiment, AAV9 serotype may comprise an AAV capsid protein having the amino acid sequence of SEQ ID NO: 2 of U.S. Pat. No. 7,198,951, the content of which is incorporated herein by reference in its entirety. In another aspect, AAV9 serotype may comprise VP1 capsid proteins of SEQ ID NOs: 2, 4 or 6 in which at least one of surface-exposed tyrosine residues in the amino acid sequence is substituted with another amino acid residue, as disclosed in US patent publication No. US20130224836, the content of which is incorporated herein by reference in its entirety.

In some embodiments, AAV particles of the present invention may comprise AAVrh10 capsid proteins or variants thereof. AAV particles comprising AAVrh10 capsid proteins can target neurons, other cells as well, in the spinal cord after intrathecal (IT) administration. In one embodiment, AAVrh10 capsid proteins may comprise the amino acid sequence of SEQ ID NO: 81 of EP patent NO: 2341068.

In some embodiments, AAV of the present invention may comprise AAVDJ capsid proteins, AAVDJ/8 capsid proteins, or variants thereof. Holehonnur et al showed that AAVDJ/8 serotype can target neurons within the Basal and Lateral Amygdala (BLA) (Holennur Ret al., BMC Neurosci, 2014, Feb. 18:15:28). In one embodiment, AAVDJ capsid proteins and/or AAVDJ/8 capsid proteins may comprise an amino acid sequence comprising a first region that is derived from a first AAV serotype (e.g., AAV2), a second region that is derived from a second AAV serotype (e.g., AAV8), and a third region that is derived from a third AAV serotype (e.g., AAV9), wherein the first, second and third region may include any amino acid sequences that are disclosed in this description.

In some embodiment, AAV particles produced according to the present invention may comprise single stranded DNA viral genomes (ssAAVs) or self-complementary AAV genomes (scAAVs). scAAV genomes contain both DNA strands which anneal together to form double stranded DNA. By skipping second strand synthesis, scAAVs allow for rapid expression in the cell.

In one embodiment, AAV particles of the present invention may comprise capsid proteins that have been shown to or are known to transduce dorsal root ganglions (DRGs).

In one embodiment, AAV particles of the present invention may comprise capsid proteins that have been shown or are known to transduce motor neurons.

In one embodiment, the AAV particles comprise a self-complementary (SC) viral genome.

In one embodiment, the AAV particles comprise a single stranded (SS) genome.

In one embodiment, an AAV particle comprising a self-complementary (sc) viral may be used to yield higher expression than an AAV particle comprising a corresponding single stranded viral genome.

In one embodiment, the serotype of the AAV particles described herein may depend on the desired distribution, transduction efficiency and cellular targeting required. As described by Sorrentino et al. (comprehensive map of CNS transduction by eight adeno-associated virus serotypes upon cerebrospinal fluid administration in pigs, Molecular Therapy accepted article preview online 7 Dec. 2015; doi:10.1038/mt.2015.212; the contents of which are herein incorporated by reference in its entirety), AAV serotypes provided different distributions, transduction efficiencies and cellular targeting. In order to provide the desired efficacy, the AAV serotype needs to be selected that best matches not only the cells to be targeted but also the desired transduction efficiency and distribution.

The invention also provides nucleic acids encoding the mutated or modified virus capsids and capsid proteins of the invention. In some embodiments the capsids are engineered according to the methods of co-owned and co-pending application PCT/US2015/034799 filed Jun. 9, 2015, the contents of which are incorporated herein by reference in their entirety and methods known in the art.

### Regulatable-AAV Particle

Described herein are regulatable-AAV particles comprising regulatable elements, which allow the payload expression to be controlled quickly and tightly in a spatial and temporal manner. This will allow the payload expression to be adjusted to the appropriate levels at the appropriate time.

In one embodiment, the present invention provides administration and/or delivery methods for regulatable-AAV particles. As used herein, the term “regulatable-AAV particle” is an AAV particle which comprises a capsid, a polynucleotide, and one or more regulatable elements and/or a payload which is regulated by one or more regulatable elements. As used herein, the term “regulatable element” (also referred to as regulatory element) refers to one or more components, factors, polynucleotide features or motifs which imparts regulatable or tunable features to regulate the expression of a payload.

In one embodiment, the payload and the regulatable element may be located on the same viral genomes. In one embodiment, the payload and the regulatable element may be located on separate viral genomes.

### Regulatable Elements

In one embodiment, in the development of regulatable-AAV particles several parameters for effective regulation are considered such as, but not limited to, the effect on the endogenous expression of the genes, restricting the expression of the transgene to the intended cell type, allow for an “OFF” state for the payload to provide little to no expression of the payload, allow for the expression of the payload to be turned on and off quickly, induce expression of the payload by stimulus or drug.

In one embodiment, the regulatable-AAV particle comprising at least one regulatable element should have no effects on the endogenous expression of genes and be non-immunogenic, so as not to interfere with the desired outcome of payload expression.

In one embodiment, the regulatable elements may be chosen in a manner that will restrict the expression of the transgene to the intended cell type specific expression.

In one embodiment, the regulatable elements must allow the payload to be in an “OFF” state, which allows very little or no expression of the payload.

In one embodiment, the regulatable elements may allow payload expression be turned on and off quickly, and also provide a means by which the level of payload expression can be regulated over a wide range in a dose dependent manner.

In one embodiment, it may be desirable to provide a regulatable element, which may be induced by a stimulus or drug, which is administered when payload expression is wanted, and removed when the payload expression is no longer needed.

In one embodiment, the viral genome may comprise the regulatable element. In one embodiments, the regulatable element may be the payload. In one embodiment, the viral genome may comprise one or more regulatable elements and the transgene of interest may be located on a separate viral genome. Various arrangements of the regulatable elements are envisioned as part of the invention described herein. Components can be upstream or downstream of each other within the payload construct or viral genome. In some embodiments, they may be located on more than one payload constructs. In some embodiments the payload is a regulatable element. In a non-limiting example, the payload is a CRISPR regulatable element.

In some embodiments, viral genome encoding the gene of interest and payload constructs comprising one or more regulatable elements may be on two or more separate payload constructs, packaged into separate AAV particles. In some embodiments, the optimal ratio of the two or more AAV particles needed to achieve the desired expression and regulation must be determined experimentally. In some embodiments, the two or more AAV particles may be administered in equal amounts. In some embodiments, the two or more AAV particles may be administered in unequal amounts. In a non-limiting example, a AAV particle comprising the payload construct encoding the gene of interest, may be delivered at a lower dose than the one or more viral genomes comprising the one or more regulatable elements. In another non-limiting example, a AAV particle comprising the payload construct encoding the gene of interest, may be delivered at a higher dose than the one or more regulatory-AAV particles comprising the one or more regulatable elements.

In one embodiment, the regulatable element may be positioned within the capsid VP2 domain. The VP2 viral capsid protein may be chosen, since it has been shown to tolerate large insertions. For example, Lux et al. (J Virol. 2005 September; 79(18): 11776-11787; Green Fluorescent Protein-Tagged Adeno-Associated Virus Particles Allow the Study of Cytosolic and Nuclear Trafficking) inserted a 27-kDa GFP protein as a GFP-VP2 fusion protein into an AAV capsid. Incorporation of GFP-VP2 into the AAV capsid did not interfere with viral assembly or viral genome packaging, and the GFP-tagged virions produced in the present study retained infectivity. Since then other studies have used the VP2 capsid as an insertion point, for example for a rapamycin inducible chemical switch, which controls viral infectivity, as described in Hoerner et al., (Chem. Commun., 2014, 50, 10319-10322), the contents of which is herein incorporated by reference in its entirety.

In some embodiments, the regulatable element may be positioned at the N terminus of VP2. As a non-limiting example, the regulatable element may be located within the first 1%, 2%, 3%, 4%, 5%, 6%, 7%, 8%, 9%, 10%, 15%, 20%, 25%, 30%, 35%, 40%, 45%, 46%, 47%, 48,% or 49% of the VP2 capsid. As another non-limiting example, the regulatable element may be located within the last 1%, 2%, 3%, 4%, 5%, 6%, 7%, 8%, 9%, 10%, 15%, 20%, 25%, 30%, 35%, 40%, 45%, 46%, 47%, 48,% or 49% of the VP2 capsid. As a non-limiting example, the regulatable element may be located in the middle of the VP2 capsid. As another non-limiting example, the regulatable element may be located near the beginning of the VP2 capsid (N terminus). As yet another non-limiting example, the promoter and/or transactivation domain may be located near the end of the VP2 capsid (C terminus).

In some embodiments, the regulatable element may be made available upon uncoating of the AAV particle, leading to a burst in the expression of the payload. In some embodiments the regulatable element may be released in the nucleus. In some embodiments, the fusion protein comprised in the regulatable element may be released from the VP2 domain through a 2A peptide sequence.

In one embodiment, the regulatable element may comprise one component. In another embodiment, the regulatable element may comprise two or more components. Without wishing to be bound by theory, a greater number of components may further fine tune the level of control. In some embodiments, the regulatable element components may comprise one or more transcription factors, fusion proteins and/or nucleases or recombinases, CRISPR components, and any combination thereof. In one embodiment, the regulatable element may comprise three or four components. In one embodiment, the regulatable element may comprise at least one protein or fusion protein. In one embodiment, the regulatable element may comprise two or more proteins or fusion proteins. In one embodiment, the one or more regulatable elements may be modulated or regulated by chemical agents (including but not limited to ligands).

In one embodiment, a hierarchy of regulatable elements may exist, in which for example a first regulatable element regulates the expression of a second regulatable element, which in turn regulates the expression of the payload gene of interest. In some embodiments even more tiers of regulation may be provided. Without wishing to be bound by theory, additional components and/or tiers of regulatable elements may help create a tighter more fine-tuned regulation of payload gene expression.

In one embodiment, the regulatable element may act on one or more promoters driving expression of one or more payloads. In one embodiment, the promoter may comprise one or more response elements for a transcription factor, which is specific for a particular tissue, or for a fusion protein, which is inducible by a particular chemical agent or physiological stimulus. In one embodiment, the regulatable element may comprise one or more fusion proteins, which comprise one or more of domains selected from a DNA binding domain, a transactivation domain and optionally a ligand binding domain. In one embodiment, payload expression may be induced by a ligand or stimulus in a dose-dependent manner.

In another embodiment, the regulatable element may regulate the expression of one or more payloads through disruption, e.g., cleavage, of the payload construct(s).

In one embodiment, the regulatable element may include an siRNA or miRNA, which can bind to the payload construct. In one embodiment, the payload construct may comprise a microRNA binding site. In one embodiment, an siRNA binding site may be present in the payload construct. In one embodiment, the regulatable element may comprise a ribozyme.

In one embodiment, the regulatable element may comprise a heterologous domain whose function affects the stability of the payload, e.g. stabilizes or destabilizes the payload.

In one embodiment, the regulatable element may comprise a heterologous domain, whose function may be further regulated or modulated by a ligand that binds to the domain.

In one embodiment, the regulatable element may comprise a heterologous domain which may be stabilized in the presence of the ligand, and destabilized in the absence of the ligand.

In another embodiment, the regulatable element may comprise a heterologous domain which may be destabilized in the presence of the ligand, and stabilized in the absence of the ligand.

In some embodiments, the regulatable element may comprise a heterologous domain which may function to provide burst expression of the payload.

In some embodiments the, the regulatable element may comprise a heterologous domain which may allow low payload expression levels.

In one embodiment, the regulatable element may transiently induce the expression of one or more payloads. In one embodiment, the payload expression may be transiently turned off through the regulatable element. In one embodiment, payload expression may be permanently turned on through the regulatable element. In one embodiment, the payload expression may be irreversibly turned off at a desired time. In one embodiment, the payload expression may be reversibly turned on or off. In one embodiment, the regulatable element may also be temporally and spatially regulated.

In one embodiment, regulatable-AAV particles may comprise at least one regulatable element and/or payloads comprising CRISPR elements, TALEN, or zinc finger nuclease elements.

In one embodiment, the regulatable element may comprise a component which comprises an endonuclease or recombinase. In one embodiment, the endonuclease or recombinase may be a fusion protein with a site specific DNA binding domain and a cleavage domain.

In one embodiment, the regulatable element may comprise a CRISPR (Clustered Regularly Interspersed Short Palindromic Repeats) regulatable element.

In one embodiment, the regulatable-AAV particle comprises a regulatable element where the expression of the protein or fusion protein may be driven by a constitutive promoter.

In another embodiment, the regulatable-AAV particle comprises a regulatable element where the expression of the protein or fusion protein may be driven by an inducible promoter, which may be induced or repressed in the presence of a ligand.

In another embodiment, the regulatable-AAV particle comprises a regulatable element where the expression of the protein or fusion protein may be driven by a tissue-specific promoter such that the regulatable element is only expressed in certain tissues.

In some embodiments, the payload expression occurs in a dose-dependent manner, depending on the dose of the chemical agent or physiological stimulus. Agents and systems must also be tested to ensure that receptors and chemical agents or physiological stimuli have minimal effects on endogenous gene expression and normal cellular responses. A short half-life is also desirable, for fast control of payload expression upon removal of the chemical agent.

In some embodiments, additional levels of regulation may be added, e.g., additional regulatable elements, may provide even tighter tissue-specific and temporal control of the payload. In some embodiments, two or three or more levels of regulatable elements are provided.

Various spatial arrangements of the regulatable elements can be envisioned according to the present invention. Components of the regulatable elements can be upstream or downstream of each other. In some embodiments, they may be located on more than one payload constructs. In some embodiments the regulatable elements may be located within one or more viral genomes.

According to the present invention any of the proteins or fusion proteins described herein, including but not limited to, CRISPR/Cas9, restriction endonucleases, recombinases, integrases, transcriptional activators, transcriptional repressor, dimerization fusion proteins, may be positioned within the VP2 domain. As a non-limiting example, the regulatable element may be a DNA binding domain, and a transactivation domain which may regulate cas9 expression or a DNA binding domain coupled to a transactivating factor, which may regulate cas9 expression. The DNA binding domain may be coupled to the transactivation domain using any methods known in the art or described herein. In one embodiment, the DNA binding domain, which binds to regulatable elements within the cas9 promoter and/or a transactivating factor may be located within the sequence encoding the VP2 capsid. As a non-limiting example, the DNA binding domain and/or transactivation domain may be located within the first 1%, 2%, 3%, 4%, 5%, 6%, 7%, 8%, 9%, 10%, 15%, 20%, 25%, 30%, 35%, 40%, 45%, 46%, 47%, 48,% or 49% of the VP2 capsid. As another non-limiting example, the DNA binding domain and/or transactivation domain may be located within the last 1%, 2%, 3%, 4%, 5%, 6%, 7%, 8%, 9%, 10%, 15%, 20%, 25%, 30%, 35%, 40%, 45%, 46%, 47%, 48,% or 49% of the VP2 capsid. As a non-limiting example, the DNA binding domain and/or transactivation domain may be located in the middle of the VP2 capsid. As another non-limiting example, the DNA binding domain and/or transactivation domain may be located near the beginning of the VP2 capsid (N terminus). As yet another non-limiting example, the promoter and/or transactivation domain may be located near the end of the VP2 capsid (C terminus).

In another embodiment, any of the regulatable element components described herein may be inserted into the VP1 domain. In another non-limiting example, any of the regulatable element components may be inserted into the VP3 domain.

### Regulatable Elements: Fusion Proteins

In one embodiment, the regulatable-AAV particle comprises at least one regulatable element that when expressed may comprise a protein or a fusion protein.

In one embodiment, the regulatable-AAV particle comprises at least one regulatable element that when expressed may comprise one protein or one fusion protein. The protein or fusion protein may be or be part of the Tet ON/OFF system, or the GeneSwitch system or the protein or fusion protein may respond to hormones, physiological stimulus or light.

In one embodiment, the regulatable-AAV particle comprises at least one regulatable element that when expressed may comprise dimerizable fusion proteins. The protein or fusion protein may respond to hormones, physiological stimulus, rapamycin, or light.

In one embodiment, the regulatable-AAV particle comprises at least one regulatable element that when expressed may comprise a fusion proteins which has been modified. A non-limiting example of the modifications may alter their ligand binding domains or the modifications can create novel fusion proteins with new binding specificities.

### Single Fusion Proteins

In one embodiment, the regulatable element, when expressed, may comprise a protein or fusion protein which is capable of driving expression from the promoter of the payload.

In one embodiment, the regulatable element, when expressed, may comprise a fusion protein which may comprise a DNA binding domain, a transactivation domain and optionally a ligand binding domain.

In some embodiments, the regulatable element of the regulatable-AAV particle may encode a protein or fusion protein which may require the presence of a chemical agent, such as a ligand, or a physiological stimulus for transcriptional activation to occur. Without wishing to be bound by theory, the protein or fusion protein may either only be able to bind to the promoter in the presence of a chemical agent or physiological stimulus or may only be able to transactivate transcription in the presence of a chemical agent or physiological stimulus. In another embodiment, the fusion protein may only be able to activate transcription in the absence of the chemical agent or stimulus.

In one embodiment, the regulatable element of the regulatable-AAV particle may encode fusion proteins which may comprise a DNA binding domain. Non-limiting examples of DNA binding domains are helix-turn-helix, zinc finger, leucine zipper, winged helix, winged helix turn helix, helix-loop-helix, HMG-box, Wor3 domain, immunoglobulin fold, B3 domain, TAL effector DNA-binding domains and RNA-guided DNA-binding domains.

Non-limiting examples of transcription factors, from which these DNA binding domains may be derived from are Ga14, CREB, HSF, ZFHD1, Ecdysone Receptor, Nuclear Receptors, such as glucocorticoid receptor, RXR, RAR, Stat proteins, myc, Tal effectors, LexA, and the like. In one embodiment, the DNA binding domain is a ZFHD1 domain. ZFHD1 is DNA binding domain composed of a zinc finger pair and a homeodomain.

In some embodiments, the DNA binding domains may be engineered zinc finger proteins. Zinc finger proteins can be engineered to recognize any suitable target site in a promoter, such as the promoter. Methods are known in the art to design or select a zinc finger protein with high specificity and affinity to its target site and are for example described in U.S. Pat. Nos. 6,933,113, 6,933,113, 6,607,882 and 6,777,185, the contents of each of which is herein incorporated by reference in its entirety.

In one embodiment, the DNA binding domains originate from transcription factors including GAL4, ZFHD1, VP16, VP64 and NFkB (p65).

In one embodiment, the regulatable element of the regulatable-AAV particle may encode fusion proteins which may comprise a transactivation domain. A non-limiting example of a transactivation domains is the nine-amino-acid transactivation domain. Non-limiting examples of transcription factors from which transactivation domains may be derived from are Ga14, Oafl, Leu3, Rtg3, Pho4, Gln3, Gcn4, p53, RTg3, CREB, Gli3, E2A, HSF1, NF-IL6, myc, NFAT, BP64, B42, NF-κB and VP16, and VP64. In one embodiment, the transactivation domains originate from transcription factors including GAL4, ZFHD1, VP16, VP64 and NFkB (p65).

In some embodiments, the regulatable element of the regulatable-AAV particle may encode fusion proteins which may comprise a transcriptional repressor domain. In some embodiments, the transcriptional repressor domain may be a KRAB, ERD, or SID transcriptional repressor domain.

In one embodiment, the regulatable element of the regulatable-AAV particle may encode fusion proteins which may comprise a ligand binding domain. Non-limiting examples of ligand binding domains are those of Ecdysone Receptor, Nuclear Receptors, such as glucocorticoid receptor, RXR, RAR, and modified forms thereof.

### The Tet ON/OFF System

In one embodiment, the regulatable-AAV particle comprises a regulatable element that when expressed may comprise a tetracycline transactivator protein which is part of the Tet-Off or Tet-On system (first described in Bujard and Gossen (Proc Natl Acad Sci U S A. 1992 Jun. 15; 89(12):5547-51, Tight control of gene expression in mammalian cells by tetracycline-responsive promoters, the contents of which is herein incorporated by reference in its entirety)). The Tet-Off system makes use of the tetracycline transactivator (tTA) protein, which is a fusion protein comprising the E. coli tetracycline repressor TetR and the Herpes Simplex Virus VP16 transactivation domain. The tTA protein is able to bind to DNA at specific TetO operator sequences. In most Tet-Off systems, several repeats of such TetO sequences are placed upstream of a minimal promoter, such as the CMV promoter (tetracycline response element (TRE)). In a Tet-Off system, tetracycline binds to the tetracycline transactivator protein and prevents its binding to TRE, thereby repressing expression of TRE-controlled genes. In the Tet-On system, tetracycline bound tetracycline transactivator protein binds to the TRE and activates transcription, i.e. activation of transcription occurs in the presence of tetracycline only. Doxirubicin is a derivative of tetracycline and also can be used.

In one embodiment, the viral genome comprises a promoter which may comprise one or more regulatable elements which imparts regulatable or tunable features to regulate the expression of a payload in the presence of tetracycline. In one embodiment, tetracycline may induce payload expression. In another embodiment, tetracycline may reduce payload expression.

In one embodiment, the regulatable-AAV particle comprises a regulatable element that when expressed may comprise a tetracycline transactivator protein, which binds to the promoter in the presence of tetracycline.

In one embodiment, the regulatable-AAV particle comprises a regulatable element that when expressed may comprise a tetracycline transactivator protein which in the absence of tetracycline, binds to the promoter.

In one embodiment, the regulatable-AAV particle comprises a regulatable element that when expressed may comprise a plurality of transactivator binding domains that are spaced such that when bound by transactivators, the transactivators are substantially rotationally aligned about the DNA helix, as described in US Publication No. US20030221203, the contents of which is herein incorporated by reference in its entirety.

In one embodiment, the viral genome comprises a promoter which may comprise tetracycline resistance operator sequences substantially free of interferon inducible response elements, as described in International Publication No. WO2002070719, the contents of which is herein incorporated by reference in its entirety. In one embodiment, a viral particle (e.g., AAV particle or regulatory-AAV particle) of the present invention may comprise a polynucleotide encoding a payload driven by a promoter and a second polynucleotide encoding a tetracycline-controlled transactivator driven by a regulatable element promoter. The two promoters may drive expression in opposite directions, towards each other and away from the inverted terminal repeats, as described in U.S. Pat. Nos. 7,811,814 and 7,456,015, the contents of each of which is herein incorporated by reference in its entirety. In another embodiment, the two promoters may drive expression in the same direction. As a non-limiting example, the payload may have a therapeutic effect on a nervous system disorder.

### Hormone Responsive Systems

In one embodiment, the regulatable-AAV particles comprise an inducible expression system. Several inducible expression systems utilize steroid response switches using non-human analogs to regulate expression. These systems comprise modular recombinant receptor fusion proteins consisting of mutated ligand-binding domains fused to appropriate transcription factor DNA-binding and activation domains. The receptor fusion proteins can be constitutively expressed and activate transcription of their target genes when a ligand is present and binds to it. For example, a chimeric Drosophila/Bombyx ecdysone receptor (DB-EcR), which is able to bind a modified ecdysone promoter and which achieved transactivation of a reporter gene in the presence of ecdysone agonist GS-E, is described in Hoppe et al. (Mol Ther. 2000 February; 1(2):159-64, the contents of which is herein incorporated by reference in its entirety).

In some embodiments, the viral genome comprises a promoter which may comprise response elements which may be regulated by a steroid response switch using non-human analogs, including but not limited to Ecdysone. In some embodiments, the regulatable element may comprise or encode a chimeric ecdysone receptor.

In some embodiments, the ecdysone receptor binding domain may be selected from, but is not limited to, an invertebrate ecdysone receptor ligand binding domain, an Arthropod ecdysone receptor ligand binding domain, a Lepidopteran ecdysone receptor ligand binding domain, a Dipteran ecdysone receptor ligand binding domain, an Orthopteran ecdysone receptor ligand binding domain, a Homopteran ecdysone receptor ligand binding domain, a Hemipteran ecdysone receptor ligand binding domain, a spruce budworm Choristoneura fumiferana ecdysone receptor ligand binding domain, a beetle Tenebrio molitor ecdysone receptor ligand binding domain, a Manduca sexta ecdysone receptor ligand binding domain, a Heliothies virescens ecdysone receptor ligand binding domain, a midge Chironomus tentans ecdysone receptor ligand binding domain, a silk moth Bombyx mori ecdysone receptor ligand binding domain, a squinting bush brown Bicyclus anynana ecdysone receptor ligand binding domain, a fruit fly Drosophila melanogaster ecdysone receptor ligand binding domain, a mosquito Aedes aegypti ecdysone receptor ligand binding domain, a blowfly Lucilia capitata ecdysone receptor ligand binding domain, a blowfly Lucilia cuprina ecdysone receptor ligand binding domain, a Mediterranean fruit fly Ceratitis capitata ecdysone receptor ligand binding domain, a locust Locusta migratoria ecdysone receptor ligand binding domain, an aphid Myzus persicae ecdysone receptor ligand binding domain, a fiddler crab Celuca pugilator ecdysone receptor ligand binding domain, an ixodid tick Amblyomma americanum ecdysone receptor ligand binding domain, a whitefly Bamecia argentifoli ecdysone receptor ligand binding domain and a leafhopper Nephotetix cincticeps ecdysone receptor ligand binding domain.

In some embodiments, the regulatable-AAV particle comprises a regulatable element that expresses a steroid hormone receptor transactivation fusion protein.

In one embodiment, the steroid hormone receptor transactivator fusion protein may comprise a glucocorticoid receptor. In some embodiments the promoter may comprise one or more glucocorticoid response elements (GRE). In some embodiments, the promoter may be inducible through steroids, such as dexamethasone.

In some embodiments, the regulatable-AAV particle comprises a regulatable element that when expressed may comprise a glucocorticoid receptor as described in US Publication No. US20030031650, the contents of which is herein incorporated by reference in its entirety.

In one embodiment, the regulatable-AAV particle comprises a regulatable element that expresses a tamoxifen dependent expression system.

In one embodiment, payload expression may be regulated by tamoxifen through a tamoxifen dependent expression system.

In some embodiments, the regulatable-AAV particle comprises a regulatable element that when expressed may comprise a tamoxifen regulatable fusion protein, which can bind to regulatable elements in the promoter. In some embodiments, the tamoxifen regulatable fusion protein may comprise a DNA binding domain, a tamoxifen binding domain, and/or a transactivation domain. As a non-limiting example, Roscilli et al., 2002 (Mol Ther. 2002 November; 6(5):653-63. Long-term and tight control of gene expression in mouse skeletal muscle by a new hybrid human transcription factor, the contents of which is herein incorporated by reference in its entirety), describes a hydroxytamoxifen (4-OHT)-dependent fusion protein comprising the DNA binding domain of the human hepatocyte nuclear factor-lalpha (HNF1alpha), which is not expressed in muscle cells, a G(521)R mutant of the ligand binding domain of human estrogen receptor-alpha (ERalpha), and the activation domain derived from human nuclear factor-kappaB p65 subunit (NF-kappaB p65). Efficient expression in muscle cells was achieved from a promoter containing multimeric HNFlalpha binding sites in the presence of ligand.

In one embodiment, the regulatable-AAV particle comprises a regulatable element that expresses an inducible system that utilizes endogenous receptors and/or steroid hormones. In some embodiments, a payload may be regulated by an inducible system that utilizes endogenous receptors and/or steroid hormones. For example, Spiga and Borras (IONS, 2010, Vol. 51, No. 6, Development of a Gene Therapy Virus with a Glucocorticoid-Inducible MMP1 for the Treatment of Steroid Glaucoma, the contents of which is herein incorporated by reference in its entirety), describe a glucocorticoid-inducible vector that expresses transgene in the presence of dexamethasone, which may have utility in the treatment of steroid-induced glaucoma resulting from the use of steroids in the treatment of macular edema.

In one embodiment, the regulatable-AAV particle comprises a regulatable element that expresses a combination of endogenous receptor and endogenous or exogenous ligands. In some embodiments, payload expression may be regulated through the combination of endogenous receptor and endogenous or exogenous ligands.

### The GeneSwitch System

In one embodiment, the regulatable-AAV particle comprises a regulatable element that expresses the GeneSwitch System (Life Technologies). The GeneSwitch System is a mifepristone-inducible mammalian expression system originally described in Wang, Y., B.W. O'Malley, J., Tsai, S. Y., and O'Malley, B. W. (1994), A Regulatory System for Use in Gene Transfer. Proc. Natl. Acad. Sci. USA 91, 8180-8184, the contents of which is herein incorporated by reference in its entirety. The system comprises a hybrid regulatory protein containing a DNA binding domain from the yeast GAL4 protein, a truncated ligand binding domain from the human progesterone receptor, and an activation domain from the human NF-κB protein. This fusion protein binds to the synthetic steroid, mifepristone, and functions as a ligand-dependent transcription factor to induce expression of the gene of interest as well as its own expression. Transgene expression is controlled by a hybrid promoter consisting of Saccharomyces cerevisiae GAL4 upstream activating sequences.

In one embodiment, the regulatable-AAV particle comprises a regulatable element which imparts regulatable or tunable features to regulate the expression of a payload in the presence of mifepristone.

In one embodiment, the regulatable-AAV particle comprises at least one regulatable element that when expressed may comprise a fusion protein as described in Wang et al. (described above, the contents of which is herein incorporated by reference in its entirety).

In one embodiment, payload expression may be regulated simultaneously on the transcriptional and a post-translational level. The regulatable element may be inducible through a first ligand, such as for example mifepristone, thereby driving expression of the payload. The payload, in turn, must dimerize upon administration of a second ligand to be activated. Without wishing to be bound by theory, allowing two levels of regulation may provide for tighter control of payload expression. A non-limiting example of a double inducible system according to the present invention is described in US Publication No. US20090293139, the contents of which is herein incorporated by reference in its entirety.

In one embodiment, the regulatable-AAV particle comprises a regulatable element that when expressed may comprise a fusion protein comprising the bacterial repressor LexA. While not wishing to be bound by theory, LexA does not resemble eukaryotic transcription factors and is thus less likely to bind to endogenous promoters. LexA binding sites can be inserted into the promoter driving the gene of interest.

In one embodiment, the viral genome comprises a promoter may comprise one or more LexA binding sites. In one embodiment, the regulatable element may comprise a fusion protein comprising the DNA binding domain of a bacterial LexA protein, a truncated ligand binding domain of a human progesterone receptor and an activation domain of the p65 subunit of human NF-kappaB. In one embodiment, the payload expression may be mifepristone-inducible.

In one embodiment, the regulatable-AAV particle comprises a regulatable element that when expressed may comprise the fusion protein described in U.S. Pat. No. 8,852,928, the contents of which is herein incorporated by reference in its entirety.

In one embodiment, the regulatable-AAV particle comprises a regulatable element that when expressed may comprise a fusion protein comprising a mutated GAL4 binding domain, which decreases dimerization of the regulator protein occurring in the absence of a anti-progestin ligand, as described in U.S. Pat. No. 7,579,326, the contents of which is herein incorporated by reference in its entirety.

### Physiological Stimulus Inducible Systems

In some embodiments, the expression of the payload may be induced by a physiological or other stimulus.

In one embodiment, the regulatory-AAV particle may comprise one or more regulatable elements which imparts regulatable or tunable features to regulate the expression of a payload in the presence of a physiological or chemical stimulus. These stimuli may include, but are not limited to, light, heat, radiation, glucose levels, hypoxia, or metals.

In some embodiments, the expression of the payload may be regulated by light. As a non-limiting example, Ye H, et al. (Science. 2011 Jun. 24; 332(6037):1565-8, the contents of which is herein incorporated by reference in its entirety) describe a system, in which the expression of a transgene under control of a NFAT-dependent promoter can be driven by illumination and can be regulated over time simply by modulating the patterns of light over periods of hours to days.

In one embodiment, the viral genome comprises a promoter which may comprise one or more NFAT binding elements as described in Ye H., et al. (described above, the contents of which is herein incorporated by reference in its entirety).

In some embodiments, the payload may be induced in the context of radiation therapy, e.g. in the context of a cancer therapy regimen. In one embodiment, the expression of the payload may be dependent on a radiation-inducible promotor. Non-limiting examples of promoters that can be used are Egr-1, VEGF, Rec-A, and WAF-1 promoters (see e.g., Goverdhana et al., 2005, Mol Ther. 2005 August; 12(2): 189-211, the contents which is herein incorporated by reference in its entirety, and references therein). While not wishing to be bound by theory, using these types of promoters provide the potential to restrict expression to the tissue receiving the radiation therapy, while expression in the adjacent, healthy tissue is not induced.

In some embodiments, the expression of the payload may be driven by glucose regulatable elements in the promoter. In a non-limiting example, glucose regulatable elements may be useful to drive an insulin payload.

In some embodiments, the expression of the payload may be regulated by hypoxia regulatable element. While not wishing to be bound by theory, the hypoxia regulatable element binds HIF-1 alpha and beta (hypoxia inducible factor), which permits the selective induction of gene expression in a hypoxic environment. This phenomenon may be exploited in a cancer setting. In one study, a rAAV was generated in which the transgene can be regulated by hypoxia in human brain tumors (Kantor et al., the contents of each which is herein incorporated by reference in its entirety, and references therein).

In one embodiment, the expression of the payload may be regulated by a metal regulatable element. Metal regulatable elements can be found within promoters of metallothionin genes which are recognized by the transcription factor MTF-1 (see e.g., Daniels et al., 2002 (Nucl. Acids Res. 2002 Vol 30, No. 14), the contents of which is herein incorporated by reference in its entirety). In some embodiments, the DNA binding domain of MTF or binding factor of another metal regulatable element may be used in a combination with a metal in an inducible system to regulate payload expression.

In one embodiment, the payload expression may be regulated by a heat shock regulatable element and binding heat shock regulatable element binding protein, either in the context of endogenous expression of proteins as part of the heat shock response, or as part of an artificial system including heat shock response elements and a protein or fusion protein which comprises a heat shock factor DNA binding domain.

In one embodiment, the viral genome comprises a promoter which may comprise elements of a heat shock promoter. Non-limiting examples of the heat shock protein (HSP) promoters include HSP70, HSP90, HSP60, HSP27, HSP72, HSP73, HSP25 and HSP28, or ubiquitin promoter as described in U.S. Pat. No. 7,285,542, the contents of which is herein incorporated by reference in its entirety. A minimal heat shock promoter derived from HSP70 may also be used. The conditions which activate the heat shock promoter are hyperthermic conditions, which may comprise a temperature between about basal temperature and about 42° C. In one embodiment, a viral genome comprises a heat shock promoter such as the promoters described in U.S. Pat. No. 7,595,386, the contents of which is herein incorporated by reference in its entirety.

In one embodiment, a pulsatile signal may be applied with a stimulator to modulate the transcription of a gene of interest in the target tissue. In one embodiment, the tissue is neural tissue. In one aspect, the neural tissue is brain tissue.

In one embodiment, the viral genome comprises a promoter which may comprise one or more regulatable sequences that respond to a pulsatile stimulus. Such pulsatile stimulus responsive regulatable sequences may be identified as described in US20070059290, the contents of which is herein incorporated by reference in its entirety. In some embodiments, payload expression may be activated through the pulsatile stimulus. In some embodiments, payload expression may be repressed through the pulsatile stimulus.

### Light Inducible Single Fusion Proteins

In one embodiment, the regulatory-AAV particle may comprise one or more regulatable elements which imparts regulatable or tunable features to regulate the expression of a payload in the presence of a light stimulus.

In one embodiment, the regulatory-AAV particle may comprise one or more regulatable elements which imparts regulatable or tunable features to regulate the expression of a payload in the presence of a blue light. Expression of only a single fusion protein can be used to directly activate transcription of the payload in response to blue light. The light-responsive DNA-binding protein (LRDP) can be provided containing both a light-oxygen-voltage (LOV) domain and DNA-binding domain, which is fused to a transcriptional activation domain, as described in US Publication No. US20140325692, the contents of which is herein incorporated by reference in its entirety.

In one embodiment, the regulatable element, when expressed, is a light responsive, LOV domain containing DNA binding protein with a transactivation domain, as described in US Publication No. US20140325692, the contents of which is herein incorporated by reference in its entirety.

In one embodiment, the regulatable element, when expressed, comprises a light inducible fusion protein. As a non-limiting example, the light inducible fusion protein is a E. litoralis 222 amino acid protein (EL222)-VP16 chimera.

### Dimerizable Fusion Proteins

In one embodiment, the regulatable-AAV comprises a regulatable element which may comprise two or more fusion proteins which may further fine tune the level of control over the payload expression.

In one embodiment, the regulatable-AAV comprises a regulatable element which when expressed comprises two fusion proteins, each of which comprise a dimerization ligand binding domain. In one embodiment, the first fusion protein may contain a DNA-binding domain of a transcription factor, which binds to the promoter driving expression of the payload, fused to a dimerization ligand binding domain. In one embodiment, the second fusion protein may contain a transactivation domain fused to a dimerization ligand binding domain. In one embodiment, the transactivation domain may be capable of activating the transcription factor, which binds to the promoter in the viral genome.

In one embodiment, the regulatable-AAV comprises a regulatable element which when expressed comprises the dimerization ligand binding domain may require the binding of a chemical agent or physiological stimulus in order for dimerization to occur. In some embodiments, the dimerization ligand binding domains of the two fusion proteins may not be able to bind to each other in the absence of the chemical agent or physiological stimulus. In some embodiments, the chemical agent or physiological stimulus may result in the dimerization of two fusion proteins and subsequent recruitment of the trans activation domain to the promoter in the payload construct. In some embodiments, both fusion proteins may bind simultaneously to the same chemical agent. In other embodiments, both fusion proteins may bind separately to the same or two different chemical agents. In another embodiment, no such chemical agent or physiological stimulus is required for one or both fusion proteins.

In some embodiments, the viral genome comprises a minimal promoter containing DNA binding elements to which the DNA binding domain binds, and transcription from the promoter may only occur in the presence of the chemical agent or stimulus.

In one embodiment, a constitutive promoter drives the expression of the regulatable element encoding the one or more fusion proteins. In another embodiment, at least one component of the regulatable element is driven by an inducible promoter. In another embodiment, the expression of all components of the regulatable element are driven by inducible promoters which may the same or different and be inducible or repressible by the same or by different chemical agents or ligands. In another embodiment, the promoter may drive tissue specific expression, such that the regulatable element is only expressed in certain tissues.

In one embodiment, the regulatable-AAV comprises a regulatable element which when expressed may comprise inducible systems such as, but not limited to, the ecdysone or rapamycin inducible systems.

### Hormone Inducible Systems

In one embodiment, the regulatory-AAV particle may comprise one or more regulatable elements which imparts regulatable or tunable features to regulate the expression of a payload in the presence of a hormone inducible system. Non-limiting examples of hormone inducible systems include steroid small molecules, Ecdysone inducible systems and/or the Rheoswitch system.

In one embodiment, the expression of the payload may be regulated through a non-steroid small molecule including, but not limited to, Ecdysone.

In one embodiment, the regulatable-AAV particle comprises a regulatable element that when expressed may encode an ecdysone inducible system. An Ecdysone inducible system is described in No et al., Proc Natl Acad Sci U S A. 1996 Apr. 16; 93(8):3346-51, the contents of which is herein incorporated by reference in its entirety. The system comprises a system with a modified ecdysone receptor fusion protein with a GAL4 DNA binding domain, VpEcR, and heterodimeric partner fusion protein, RXR with a VP16 transactivation domain. Upon expression of RXR and VpEcR fusion proteins, the two receptors can heterodimerize and transactivate the EcRE-containing promoter, but only in the presence of the insect hormone ecdysone.

In one embodiment, the viral genome comprises a promoter which may comprise one or more regulatable elements suitable for the ecdysone inducible system. In one embodiment, the regulatable element when expressed may comprise a modified ecdysone receptor fusion protein with a GAL4 DNA binding domain, and RXR with a VP16 transactivation domain as a heterodimeric partner fusion protein. In one embodiment, the regulatable element when expressed may comprise one or more fusion proteins which are part of the multiple inducible gene regulation system described in U.S. Pat. No. 8,105,825, assigned to Intrexon, the contents of which is herein incorporated by reference in its entirety. In one embodiment, the ligand which induces the regulatable element may be described in U.S. Pat. No. 8,105,825 assigned to Intrexon.

In one embodiment, the regulatable-AAV particle comprises a regulatable element that when expressed may encode an Ecdysone inducible systems such as, but not limited to, the Rheoswitch system (Intrexon). In the Rheoswitch system controlled expression of the gene of interest is activated by the RheoSwitch® receptor (a heterodimer of an engineered ecdysone receptor ligand binding domain fused to a GAL4 DNA binding domain (GAL4:EcR) and a retinoid X receptor (RXR) protein fused to a VP16 activation domain (VP16:RXR)) only in the presence of an activator drug, as described in Karzenowski et al., 2006, Molecular Therapy (2006) 13, S194-S194, the contents of which is herein incorporated by reference in its entirety. While not wishing to be bound by theory, the small molecule ligand or activator drug, such as Intrexon's synthetic diacylhydrazine molecule veledimex or analogs thereof, triggers the conformational changes needed to activate transcription. Additionally, the ecdysone may demonstrate safety in mammals because the chemical agents or ligands are designed to activate the insect ecdysone receptor rather than mammalian receptors thus avoiding potential off target effects.

In one embodiment, the regulatable element when expressed may comprise a Rheoswitch system. In one embodiment, the regulatable element when expressed may comprise an ecdysone receptor fusion protein and a heterodimeric partner fusion protein. In some embodiments, the fusion proteins are those described in No et al. or derivatives thereof. In some embodiments, the fusion proteins are those described in Karzenowsiki et al. In one embodiment, the fusion proteins are ecdysone regulatable. In one embodiment, the fusion proteins are regulatable by a diacylhydrazine molecule or an analog thereof. In one embodiment, the chemical agent or ligand may be administered orally.

In one embodiment, the regulatable element when expressed may comprise a first fusion protein comprising a DNA-binding domain and an ecdysone receptor ligand binding domain; and a second fusion protein a transactivation domain and a chimeric RXR ligand binding domain comprising a vertebrate RXR amino acid sequence and an invertebrate RXR amino acid sequence, as described in U.S. Pat. No. 8,598,409, the contents of which is herein incorporated by reference in its entirety.

### Rapamycin Inducible Systems

In one embodiment, the regulatory-AAV particle may comprise one or more regulatable elements which imparts regulatable or tunable features to regulate the expression of a payload in the presence of a rapamycin inducible system. The rapamycin inducible system takes advantage of the dimerizing function of the antibiotic rapamycin, which links FK506-binding protein (FKBP) and FKBP12-rapamycin-associated protein (FRAP). FKBP contains the DNA-binding domain, such as ZFHD, while the activation domain of NF-κB p65 is fused to FRAP. The promoter containing the target DNA sequence is only induced when the two are dimerized by the action of rapamycin or alternative analogues (e.g. Kantor et al., and references therein). In one embodiment, the promoter may have a target sequence suitable for regulation through this system.

In one embodiment, the regulatable element when expressed may comprise a rapamycin regulatable element. In one embodiment, the regulatable element when expressed may comprise a rapamycin or rapamycin analog regulatable DNA binding domain fusion protein and a transactivation domain fusion protein, each of which comprise a rapamycin or analog binding domain. As a non-limiting example, the rapamycin or analog binding domain comprised in both the DNA binding domain fusion protein and transactivation domain fusion proteins may be FKBP (FK506-binding protein). FKBP is an abundant 12 kDa cytoplasmic protein that acts as the intracellular receptor for the immunosuppressive drugs FK506 and rapamycin. As described in US Publication No. US20130023033, the contents of which is herein incorporated by reference in its entirety, and references therein, one or more copies of FKBP may be fused to a DNA binding domain and a transactivation domain of a transcription factor. While not wishing to be bound by theory, they will dimerize upon addition of FK1012 (a homodimer of FK506).

As another non-limiting example, the rapamycin or analog binding domain in the DNA binding domain fusion protein and the activation domain fusion protein may be derived from two different proteins, allowing FK506 and rapamycin to promote heterodimerization. Without wishing to be bound by theory, heterodimerization more closely follows their natural mechanism of action.

Examples of dimerization domains/ligand binding domains useful in the rapamycin system include but are not limited to FKBP, calcineurin A, minimal calcineurin domain termed a CAB, and FRAP (mTOR, e.g., amino acids 2021-2113) as described in US Publication No. US20130023033, the contents of each of which is herein incorporated by reference in its entirety, and references therein. In some embodiments, the FRAP sequence may incorporate the single point-mutation Thr2098Leu (FRAP L) to allow use of certain nonimmunosuppressive rapamycin analogs (rapalogs).

In some embodiments, the dimerization domains/ligand binding domain may be N-terminal, C-terminal, or interspersed with respect to the DNA binding domain and activation domain. In some embodiments, the fusion proteins may comprise multiple copies of a dimerization domains/ligand binding domain, e.g. 2, 3 or 4 copies. The various domains of the fusion proteins may be connected to each other by linkers.

In some embodiments FRAP may be fused to a transactivator portion of human NF-KB p65 (190 amino acids) and FKBP may be fused to a ZFHD DNA binding domain as described in US Publication No. US20130023033, the contents of which is herein incorporated by reference in its entirety. In some embodiments the FKBP-ZFHD fusion protein may contain one, two, three, four or more copies of FKBP.

Suitable compounds for use in this system are disclosed in US Patent Publication No. US20130023033, incorporated herein by reference in its entirety, and references therein. Non-limiting examples of compounds include rapamycin, FK506, FK1012 (a homodimer of FK506), rapamycin analogs (“rapalogs”) which modified to add a “bump” that reduces or eliminates affinity for endogenous FKBP and/or FRAP, including, but not limited to, AP26113 (Ariad), AP1510, AP22660, AP22594, AP21370, AP22594, AP23054, AP1855, AP1856, AP1701, AP1861, AP1692 and AP1889.

In one embodiment, the regulatable element when expressed may comprise a chemically induced system as described in US Patent Publication No. US20130023033, the contents of which is herein incorporated by reference in its entirety. In one embodiment, the domains used in the present invention may be the domains of or derived from those described in US Publication No. US20130023033. In some embodiments the domains may be those or derived from the Ariad ARGENT® system as described in US Publication No. US20130023033, and references therein, including in U.S. Pat. Nos. 5,834,266 and 7,109,317, US Publication No. 20020173474, U.S. Publication No. 200910100535, U.S. Pat. Nos. 5,834,266, 7,109,317, 7,485,441, 5,830,462, 5,869,337, 5,871,753, 6,011,018, 6,043,082, 6,046,047, 6,063,625, 6,140,120, 6,165,787, 6,972,193, 6,326,166, 7,008,780, 6,133,456, 6,150,527, 6,506,379, 6,258,823, 6,693,189, 6,127,521, 6,150,137, 6,464,974, 6,509,152, 6,015,709, 6,117,680, 6,479,653, 6,187,757, 6,649,595, 6,984,635, 7,067,526, 7,196,192, 6,476,200, 6,492,106, WO199418347, WO199620951, WO199606097, WO199731898, WO199641865, WO199802441, WO199533052, WO1999110508, WO1999110510, WO199936553, WO199941258, WO2001114387, ARGENT™ Regulated Transcription Retrovirus Kit, Version 2.0 (9109102), and ARGENT™ Regulated Transcription Plasmid Kit, Version 2.0 (91 0902), each of which is incorporated herein by reference in its entirety.

In some embodiments, ligands for use in these inducible systems may be or be any of those described in U.S. Pat. Nos. 5,834,266 and 7,109,317, US Publication No. 20020173474, US Publication No. 200910100535, U.S. Pat. Nos. 5,834,266, 7,109,317, 7,485,441, 5,830,462, 5,869,337, 5,871,753, 6,011,018, 6,043,082, 6,046,047, 6,063,625, 6,140,120, 6,165,787, 6,972,193, 6,326,166, 7,008,780, 6,133,456, 6,150,527, 6,506,379, 6,258,823, 6,693,189, 6,127,521, 6,150,137, 6,464,974, 6,509,152, 6,015,709, 6,117,680, 6,479,653, 6,187,757, 6,649,595, 6,984,635, 7,067,526, 7,196,192, 6,476,200, 6,492,106, International Publication No. WO199418347, International Publication No. WO199620951, International Publication No. WO199606097, International Publication No. WO199731898, International Publication No. WO199641865, International Publication No. WO199802441, International Publication No. WO199533052, International Publication No. WO1999110508, International Publication No. WO1999110510, International Publication No. WO199936553, International Publication No. WO199941258, International Publication No. WO2001114387, the contents of each of which is incorporated herein by reference in its entirety.

In one embodiment, the regulatable element when expressed may comprise a first fusion protein and a second fusion protein, which both contain a dimerization domain and can be induced to associate with each other through binding of a ligand. In one embodiment, the first and second fusion protein may be any of those described in U.S. Pat. Nos. 6,165,787, 6,011,018, 5,869,337, US Publication No. US20090060888, U.S. Pat. No. 6,046,047, European Publication No. EP1978095, European Publication No. EP0804561, U.S. Pat. Nos. 6,140,120 and 6,063,625, the contents of each of which is herein incorporated by reference in its entirety. In one embodiment, the ligand may be any of the ligands described in U.S. Pat. Nos. 6,165,787, 6,011,018, 5,869,337, US Publication No. US20090060888, U.S. Pat. No. 6,046,047, European Publication No. EP1978095, European Publication No. EP0804561, U.S. Pat. No. 6,140,120 and U.S. Pat. No. 6,063,625, the contents of each of which are herein incorporated by reference in its entirety.

In one embodiment, the regulatable element when expressed may comprise a rapamycin analog AP21967 regulatable transcription factor as described in International Publication No. WO2006063247, the contents of which is herein incorporated by reference in its entirety. In one embodiment, the payload may comprise a therapy for a neurological disorder.

In one embodiment, the viral genome comprises a promoter which may be inducible through a rapamycin inducible system, wherein the two rapamycin inducible fusion proteins of the regulatable element are under control of a tissue specific promoter. Such a system is for example described in Chen et al., 2013 (Hum Gene Ther Methods. 2013 August; 24(4): 270-278. Enhancing the Utility of Adeno-Associated Virus Gene Transfer through Inducible Tissue-Specific Expression), in which the transcription factor domains under the control of either a heart-specific promoter (cardiac troponin T, cTnT) or a liver-specific promoter (thyroxine-binding globulin, TBG).

### Light Inducible Systems

In one embodiment, the regulatory-AAV particle may comprise one or more regulatable elements which imparts regulatable or tunable features to regulate the expression of a payload encoding dimerizable fusion proteins in the presence of a light stimulus.

In one embodiment, the regulatable element when expressed may comprise a DNA binding fusion protein and a transactivating fusion protein, both of which comprise a light inducible dimerization domain. Any of the light inducible domains described in Jinek et al., A programmable dual-RNA-guided DNA endonuclease in adaptive bacterial immunity. 2012 Aug. 17; 337(6096):816-21, the contents of which is herein incorporated by reference in its entirety, may be used.

### Modified Fusion Proteins

In one embodiment, the regulatory-AAV particle may comprise one or more regulatable elements which imparts regulatable or tunable features to regulate the expression of a payload encoding modified fusion proteins. Fusion proteins can be modified, e.g. in their ligand binding domains or novel fusion proteins can be developed with new binding specificities, according to methods well known in the art. Dimerization domains and ligands or compounds that bind to them may be readily identified using methods and compound characteristics known in the art, for example, described in US Publication No. US20130023033, the contents of which is herein incorporated by reference in its entirety. The design of fusion proteins for modulation of gene expression, has been described, for example in Salis and Kaznessis; Phys Biol. 2006 Dec. 22; 3(4):295-310. “Computer-aided design of modular protein devices: Boolean AND gene activation.” Techniques for the creation of novel fusion proteins are well known in the art, for example as described in Zhu et al., BioTechniques, Vol. 43, No. 3, September 2007, pp. 354-359, the contents of which is herein incorporated by reference in its entirety.

Chemical libraries of ligands can be panned against these modified or new fusion receptors to identify new ligands with the desired binding affinities. These ligands can then be tested for their physical and pharmacological properties (e.g., affinity, biodistribution, toxicology, half-life, etc.).

In addition, fusion proteins should be extensively tested to ensure their binding specificity and to avoid off-target effects. The DNA binding domain, and the corresponding fusion protein should bind to DNA sequence element with specificity and affinity as compared to other sequences, such as host genomic sequences. This means that the DNA binding domain binds much more strongly to the response element than to any other sequence presented in in vitro binding studies.

### Regulatable Elements: Endonucleases, Recombinases and Restriction Enzymes

In one embodiment, the regulatable-AAV particle comprises at least one regulatable element that when expressed may comprise an enzyme.

In some embodiments, the regulatable element when expressed comprises an enzyme such as an endonuclease, recombinase, restriction enzyme or related enzyme which can function to turn off payload expression. For example, the enzyme may comprise a meganuclease, a zinc finger nuclease, a recombinase, an integrase, a TALEN, CRISPR Cas9 enzyme or a restriction enzyme specific to a sequence that rarely occurs in the human genome.

In one embodiment, the viral genome may comprise one or more recognition sites specific to the endonuclease or recombinase encoded by the regulatable element, such that expression of the payload can be turned off upon expression of the regulatable element. Such one or more recognition sites may be located in or may be flanking one or more regions of the payload construct. As used herein, “payload construct regions” include the 5′ITR, the region downstream of the 5′ITR and upstream of the promoter, the promoter, the 5′UTR (which is located 3′ of the promoter and 5′ of the coding sequence), the coding sequence, the payload 3′UTR (which is located 3′ of the coding sequence and 5′ of the ITR and which optionally comprises a polyadenylation site), the region downstream of the 3′UTR and upstream of the 3′ITR, and the 3′ITR.

In one embodiment, the one or more sites may be located in the promoter region. In another embodiment, the one or more recognition sites may be located within the coding region. In another embodiment, the one or more recognition sites may be located in the 5′UTR region. In another embodiment, the one or more recognition sites may be located within the 3′UTR region. In another embodiment, the one or more recognition sites may be located in a region 3′ of the 5′ITR and 5′ of the promoter. In another embodiment, the one or more recognition sites may be located in a region 3′ of the 3′ UTR and 5′ of the 3′ ITR. The locations of the one or more recognition sites are not limited to any particular position within each of these regions and can be at any position within the regions, i.e., at the 5′ end, and the 3′ end or in the middle of a region.

In one embodiment, two or more different types of recognition sites may be present within or flanking any of the regions of the payload construct. In a non-limiting example, these sites may be recognized by different enzymes or fusion proteins. In a non-limiting example, these sites may be recognized by two or more different types of recombinases and may be in the same or in a different orientation. In another non-limiting example, these sites may be recognized by two or more different single guide RNAs (sgRNAs).

In some embodiments, the regulatable-AAV particle comprises a regulatable element that when expressed may comprise at least one enzyme which may be a chimeric enzyme or fusion protein, wherein the nuclease has at least two domains comprising a sequence specific DNA binding domain and a catalytic domain. In one embodiment, these are expressed independently of each other and are on separate polypeptide chains. In one embodiment, heterodimerization is inducible, e.g. through ligand binding or a physiological stimulus. In another embodiment, the at least two domains are on one polypeptide chain. The polypeptide chains can be arranged in various ways such that the domains may be N terminal or C terminal with respect to each other, and may be encoded upstream or downstream of each other.

The enzymes must be extensively tested to ensure their binding specificity and avoid off-target effects, according to methods know in the art. The DNA binding domain should bind to the appropriate DNA sequence element, e.g., within the payload construct, with specificity and affinity as compared to other sequences, such as host genomic sequences. This means that the DNA binding domain binds much more strongly to the response element than to any other sequence presented in in vitro binding studies.

In one embodiment, an inducible promoter drives the expression of the regulatable element encoding one or more enzymes or fusion proteins. As a non-limiting example, any of the regulatable elements described herein may be useful in regulating the enzyme. In another embodiment, the promoter of the regulatable element encoding the one or more enzymes drives tissue specific expression, such that the regulatable element is only expressed in certain tissues.

In one embodiment, a constitutive promoter drives the expression of the regulatable element encoding the endonuclease.

### CRE/LoxP Recombinase and CRE/LOXP System

In one embodiment, the regulatable-AAV particle comprises at least one regulatable element that when expressed may comprise a CRE/Lox recombinase. In one embodiment, the viral genome may comprise one or more CRE/Lox regulatable elements, i.e., Lox sites. In one embodiment, the viral genome may comprise two Lox sites. In some embodiment, the viral genome may comprise 2, 3, 4, 5, 6 or more Lox sites.

In some embodiments, the recombinase recognition sites may be LoxP sites. In some embodiments, variant LoxP sequences, for example Lox2272 and LoxN may also be used. Lox variants are known in the art and are for example described in Missirlis et al. (A high-throughput screen identifying sequence and promiscuity characteristics of the LoxP spacer region in Cre-mediated recombination, BMC Genomics. 2006; 7: 73), the contents of which is herein incorporated by reference in its entirety.

In some embodiments herein, there may be at least one pair of identical Lox sequences in the viral genome. While not wishing to be bound by theory, Cre recombinase typically cannot induce recombination between a pair of non-identical Lox sites, so that a pair of identical Lox sites (e.g. two LoxP sites, or two Lox2272 sites) must be present for recombination to occur.

The Cre recombinase recognizes 34 bp LoxP sites, whose orientation and location relative to each other determine the way in which the genetic material is rearranged. If the LoxP sites are in opposite orientation to each other on the same DNA strand, recombination results in the inversion of the DNA in between the two sites. If the sites are in the same orientation, the recombination event will result in a deletion; this orientation results in the excision of the sequence as a circular DNA. If the sites are not on the same DNA strand, the recombination will result in a translocation at the LoxP sites.

In one embodiment, the LoxP sites may be in opposite orientation relative to each other on the same DNA strand. In one embodiment, the LoxP sites may be in the same orientation relative to each other on the same DNA strand. Without wishing to be bound by theory, expression of the CRE recombinase will result in inversion or in excision of the region or part of a region which is flanked by the recognition sites. This region may be, but is not limited to, the coding region or any other region of the viral genome.

In one embodiment, the payload may be irreversibly turned off. In another embodiment, the recombination event may be reversible.

In one embodiment of the present invention, the payload construct may comprise two or more LoxP sites located in or flanking one or more payload construct regions. These regions include the 5′ITR region, the region downstream of the 5′ITR and upstream of the promoter, the promoter region, the 5′UTR region, the coding region, the 3′UTR region, the region downstream of the 3′UTR and upstream of the 3′ITR, and the 3′ITR region.

In one embodiment, the two or more LoxP sites may flank two or more regions. In one embodiment, the two or more LoxP site may be comprised within one region. In one embodiment, the two or more LoxP sites may be positioned within more than one region. The two or more LoxP sites may be located at any position within any of the regions, i.e., at the 5′ end, and the 3′ end or in the middle of a region. In some aspects, the two or more LoxP sites may flank certain regulatable elements located within a certain region or across one or more regions. In some aspects, the two or more LoxP sites may flank the coding sequence or a part thereof.

As a non-limiting example, the payload construct may comprise two sites, each of which is located in a different region. In another non-limiting example, the payload construct may comprise two sites and the two LoxP sites may flank one or more regions. In another non liming example, the payload construct may comprise two sites, which are both located in one region. In one embodiment, the payload construct comprises only one recognition site.

Non-limiting examples of constructs with recombinase recognition sites are described in US20140127162, the contents of which is herein incorporated by reference in its entirety.

In one embodiment, the payload construct may comprise a synthetic intron flanked on the 5′ end by a splice donor site and on the 3′ end by a splice acceptor site. In one embodiment, the synthetic intron may be located in the promoter. In one embodiment, the synthetic intron within the promoter may comprise an enhancer element. In a non-limiting example, the enhancer element may be a UBC enhancer. In one embodiment, the splice donor sites and the splice acceptor sites may each be flanked on both sides by a recombinase recognition site, including but not limited to LoxP. In one embodiment, all four recombinase recognition sites are in the same orientation. In one embodiment the promoter may be a modified CAS1 promoter, comprising, from 5′ to 3′, a CMV enhancer fragment, beta-actin promoter fragment, a splice donor, a UBC enhancer fragment, a splice acceptor, in which a first pair of LoxP sites flank the splice donor, and a second pair of LoxP site flank the splice acceptor, and in which all four of the LoxP sites are in the same orientation, as described in US Publication No. US20140127162, the contents of which is herein incorporated by reference in its entirety.

In one embodiment, a recombinase recognition site may be located in the 3′UTR region. The 3′UTR region may comprise one or more posttranscriptional regulatable elements, such as woodchuck hepatitis virus posttranscriptional regulatable elements (WPRE), hepatitis B virus posttranscriptional regulatable elements HBV/PRE, or RNA transport elements (RTE) or variants thereof. The posttranscriptional regulatable element may be flanked on each side by a recombinase recognition site, as described in US Publication No. US20140127162, the contents of which is herein incorporated by reference in its entirety. Alternatively, the posttranscriptional regulatable element, such as a WPRE, may comprise one or more recombinase recognition sites. The 3′UTR may further comprise a polyadenylation site, including but not limited to the SV40 polyadenylation site. The polyadenylation site may be flanked by two recombinase recognition sites or may comprise one or more recognition sites, as described in US Publication No. US20140127162. In one embodiment, the payload construct may comprise a first recombinase recognition site downstream (or 3′) of the first ITR, and upstream (or 5′) of the 3′ end of promoter and a second recombinase recognition site positioned downstream (3′) of the 5′ end of the promoter and a third recombinase recognition site positioned downstream (or 3′) of the payload and upstream (or 5′) of a second ITR. All three of the recombinase sites may be oriented in the same direction. In another embodiment, the payload construct may also include a WPRE in the 3′ UTR and a fourth recombinase recognition site positioned upstream (or 5′) of a 3′ end of the WPRE. In another embodiment, the payload construct may include a fifth recombinase recognition site positioned downstream (or 3′) of a 5′ end of the WPRE. In another embodiment, the payload construct may also include a sixth recombinase recognition site positioned downstream (or 3′) of the WPRE and upstream (or 5′) of the second ITR. In one aspect, all six recombinase target sites may all be oriented in the same direction. In some embodiments the WPRE is a short WPRE as described in US Publication No. US20140127162.

In some embodiments, the payload construct may comprise recombinase recognition sites in different orientations. In a non-limiting example, two or more of a first recombinase target site are oriented in a first orientation, and two or more of a different, second recombinase recognition site are oriented in a second orientation, such that recombination events cannot be induced between the first and second sites. In some embodiments, two or more of a first recombinase recognition site (e.g., a LoxP site) are oriented in one direction flanking a first sequence, and two or more of a second, different, recombinase recognition site (e.g., an FRT site, the target recognition site for the FLP recombinase) are oriented in the other direction, flanking a second sequence. Without wishing to be bound by theory, CRE would then induce recombination between the LoxP sites to excise the first sequence, while the addition of FLP would induce recombination between the FRT sites to excise the second sequence.

In one embodiment, the payload construct may comprise any of the regulatory sequences comprised in the vectors described in US Publication No. US20140127162, the contents of which is herein incorporated by reference in its entirety.

In one embodiment, the payload expression may be induced by a regulatable element comprising a recombinase. In one embodiment, the payload construct may comprise a stop cassette element. The stop cassette element may be located within or in between one or more of the payload construct regions. In one embodiment, the stop cassette element may be constructed of the elements described in US Publication No. US20150020223, the contents of which is herein incorporated by reference in its entirety. In one embodiment, the stop cassette element may be located between the promoter and the coding sequence. In one embodiment, the stop cassette element may be LoxP-SV40 polyA x3-LoxP. Without being bound by theory, the CRE recombinase may excise the stop cassette element and induce transcription of the payload. In one embodiment, a viral genome comprises a promoter which may comprise a stop cassette element. In a non-limiting example, the stop cassette element located in the promoter may be the one described in US Publication No. US20150020223, the contents of which is herein incorporated by reference in its entirety. In one embodiment, the viral genome comprises a promoter which comprises a stop cassette element may drive the expression of a Cas9 and/or single guide RNA (sgRNA).

In one embodiment, the expression of CRE recombinase may be under control of a constitutive promoter. In one embodiment, the expression of CRE recombinase may be under control of an inducible promoter. Non-limiting examples of inducible systems that may govern the expression of CRE recombinase are described herein. In a non-limiting example, the promoter driving expression of the CRE recombinase may be inducible by a rapamycin inducible system as described herein. In one embodiment, the CRE regulatable element of the invention may comprise a pharmacologically induced transgene ablation system as described in US Publication No. US20130023033, the contents of which is herein incorporated by reference in its entirety. In another embodiment, the CRE recombinase may be under control of a tissue specific promoter.

In one embodiment, the CRE recombinase may be an inducible fusion protein also containing a ligand binding domain. Non-limiting examples are described in Jaisser at al. (Jaisser, F. Inducible gene expression and gene modification in transgenic mice. J. Am. Soc. Nephrol. 11 (suppl. 1), S95-S100(2000)), the contents of which is herein incorporated by reference in its entirety. In some embodiments, the ligand binding domain may be mutated so as not to be induced by the endogenous ligand. In a non-limiting example, the CRE recombinase may be a CRE recombinase fusion with the estrogen ligand-binding domain, which may be active only upon induction by tamoxifen and not endogenous circulating estrogens, as described in Metzger D, Clifford J, Chiba H, Chambon P: Conditional site-specific recombination in mammalian cells using a ligand-dependent chimeric Cre recombinase. Proc Natl Acad Sci USA92: 6991-6995, 1995, the contents of which is herein incorporated by reference in its entirety.

### FLP/FRT System

In one embodiment, the regulatable-AAV particle comprises at least one regulatable element that when expressed may comprise a FLP recombinase. The FLP/FRT system is analogous to the CRE/Lox recombination system. The targets of the FLP recombinase are the two FLP recognition target sites (FRT) and the recombination events that can occur are the same as those described above for CRE/Lox.

In one embodiment of the present invention, the payload construct may comprise two or more FRT sites located within a payload construct region. These regions include, but are not limited to, the 5′ITR region, the region downstream of the 5′ITR and upstream of the promoter, the promoter region, the 5′UTR region, the coding region, the 3′UTR region, the region downstream of the 3′UTR and upstream of the 3′ITR, and the 3′ITR region.

In one embodiment, the FRT sites may be in opposite orientation to each other on the same DNA strand. In one embodiment, the FRT sites may be in the same orientation on the same DNA strand. Without wishing to be bound by theory, expression of the FLP recombinase may result in inversion or in excision of the region flanked by the FLP sites from the payload construct. In one embodiment, the payload expression may be irreversibly turned off. In another embodiment, the recombination event may be reversible.

In one embodiment, the two or more FRT sites may flank two or more regions. In one embodiment, the two or more FRT site may be comprised within one region. In one embodiment, the two or more FRT sites may be positioned within more than one region. The two or more FRT sites may be located at any position within any of the regions, i.e., at the 5′ end, and the 3′ end or in the middle of a region. In some aspects, the two or more FRT sites may flank certain regulatable elements located within a certain region or across one or more regions. In some aspects, the two or more FRT sites may flank the payload coding sequence or a part thereof.

Non-limiting examples of constructs with recombinase recognition sites which can be used with the FLP/FRT system are described above for the CRE/LOXP system. Exemplary constructs are also described in US20140127162 and US20130023033, the contents of each of which are herein incorporated by reference in their entirety.

In one embodiment, the expression of FLP recombinase may be under control of a constitutive promoter.

In one embodiment, the expression of FLP recombinase may be under control of an inducible promoter. Non-limiting examples of inducible systems that may govern the expression of FLP recombinase are described herein. In one embodiment, the FLP regulatable element of the invention may comprise a pharmacologically induced transgene ablation system as described in US Publication No. US20130023033, the contents of which is herein incorporated by reference in its entirety.

In another embodiment, the FLP recombinase may be under control of a tissue specific promoter.

In one embodiment, the payload expression may be induced by a regulatable element comprising a FLP recombinase. In one embodiment, the payload construct may comprise a stop cassette element positioned within or in between one or more of the payload construct regions. In one embodiment the stop cassette element is located between the promoter and the coding sequence. Similar as described supra for CRE recombinase, FLT recombinase may excise the stop cassette element, including but not limited to the stop cassette element described in US Publication No. US20150020223, the contents of which is herein incorporated by reference in its entirety, and induce transcription. In one embodiment, a viral genome comprises a promoter which may comprise a stop cassette element.

In one embodiment, the FLP recombinase may comprise a ligand binding domain and may be inducible through a ligand.

### Serine Integrases

In one embodiment, the regulatable-AAV particle comprises at least one regulatable element that when expressed may comprise a serine integrase, such as, but not limited to, ΦC31. The phiC31 integrase is a sequence-specific recombinase encoded within the genome of the bacteriophage phiC31. The phiC31 integrase mediates recombination between two 34 base pair sequences, one found in the phage and the other in the bacterial host and can function in many cell types including mammalian cells.

In one embodiment, the payload construct may comprise two or more ΦC31 recognition sequences within one or more payload construct regions. These regions include the 5′ITR region, the region downstream of the 5′ITR and upstream of the promoter, the promoter region, the 5′UTR region, the coding region, the 3′UTR region, the region downstream of the 3′UTR and upstream of the 3′ITR, and the 3′ITR region.

In one embodiment, the two or more ΦC31 sites may flank two or more regions. In one embodiment, the two or more ΦC31 sites may be comprised within one region. In one embodiment, the two or more ΦC31 sites may be positioned within more than one region. The two or more FRT sites may be located at any position within any of the regions, i.e., at the 5′ end, and the 3′ end or in the middle of a region. In some aspects, the two or more ΦC31 sites may flank certain regulatable elements located within a certain region or across one or more regions. In some aspects, the two or more ΦC31 sites may flank the coding sequence or a part thereof.

Non-limiting examples of constructs with recombinase recognition sites which can be used with an integrase system such as the ΦC31 system are described above for the CRE/LOXP system. Exemplary constructs are also described in US20140127162 and US20130023033, the contents of each of which is herein incorporated by reference in its entirety.

In one embodiment, the payload expression will be irreversibly turned off. In another embodiment, the recombination event is reversible.

In one embodiment, the expression of ΦC31 integrase is under control of a constitutive promoter.

In one embodiment, the expression of ΦC31 integrase is under control of an inducible promoter. Non-limiting examples of inducible systems that may govern the expression of ΦC31 integrase are described herein. In one embodiment, the integrase regulatable element of the invention may comprise a pharmacologically induced transgene ablation system as described in US Publication No. US20130023033, the contents of which is herein incorporated by reference in its entirety.

In another embodiment, the integrase may be under control of a tissue specific promoter.

### Zinc Finger Nucleases, TALENs, and Meganucleases

In one embodiment, the regulatable-AAV particle comprises at least one regulatable element that when expressed may comprise a protein or fusion protein such as, but not limited to, zinc finger nucleases, TALENS, and meganucleases. Zinc finger nucleases are artificial restriction enzymes generated by fusing a zinc finger DNA-binding domain to a DNA-cleavage domain. Zinc finger domains can be engineered to target specific desired DNA sequences and this enables zinc-finger nucleases to target unique sequences.

Transcription activator-like effector nucleases (TALENs) are artificial restriction enzymes generated by fusing a TAL effector DNA-binding domain to a DNA cleavage domain (FokI cleavage domain). In their natural context, TAL effector proteins are secreted by bacteria and bind promoter sequences in the host plant and activate the expression of plant genes that aid bacterial infection. TAL effector proteins DNA binding domains contain variable numbers of amino acid sequence repeats. There is a simple relationship between the identity of two hypervariable amino acid residues in the DNA and sequential DNA bases in the TAL effector's target site, a circumstance, which has been extensively used to design artificial custom TAL effectors domains capable of recognizing new DNA sequences in other hosts. Novel restriction enzyme and novel transcription factor fusion proteins have been created using the TAL effector DNA binding domain.

Meganucleases are endodeoxyribonucleases characterized by a large recognition site (double-stranded DNA sequences of 12 to 40 base pairs); for example, I-Scel recognizes a specific asymmetric 18 bp element (TAGGGATAACAGGGTAAT (SEQ ID NO: 570) and creates double strand breaks, as described in US Publication No. US20130023033, the contents of which is herein incorporated by reference in its entirety, and references therein.

TALENs, zinc finger nucleases and meganucleases are contemplated in the instant invention as regulators of payload expression. In a non-limiting example, the payload construct may comprise at least one recognition sequence for a TALEN, Zinc finger nuclease or meganuclease. Regions in which the recognition sequences may be located include the 5′ITR region, the region downstream of the 5′ITR and upstream of the promoter, the promoter region, the 5′UTR region, the coding region, the 3′UTR region, the region downstream of the 3′UTR and upstream of the 3′ITR, and the 3′ITR region.

Methods of designing the enzymes and their recognition sites so that they do not recognize other genomic sequences in the cell are well known in the art.

In one embodiment, the payload construct comprises at least one recognition sequence that may be recognized and cleaved by a zinc finger nuclease, TALEN or meganuclease. In one embodiment, this recognition sequence is specific. In one embodiment, the payload expression will be irreversibly turned off.

In one embodiment, the expression of zinc finger nuclease, TALEN or meganuclease is under control of a constitutive promoter.

In one embodiment, the expression of zinc finger nuclease, a TALEN or meganuclease is under control of an inducible promoter. Non-limiting examples of inducible systems that may govern the expression of zinc finger nuclease, TALEN or meganuclease are described supra herein. In one embodiment, the endonuclease regulatable element of the invention may comprise a pharmacologically induced transgene ablation system as described in US Publication No. US20130023033, the contents of which is herein incorporated by reference in its entirety.

In another embodiment, the TALEN or meganuclease may be under control of a tissue specific promoter.

### CRISPR

The CRISPR (Clustered Regularly Interspersed Short Palindromic Repeats) system functions as an adaptive immune response defense in the genomes of several bacteria and Archaea.

In bacteria, CRISPR, along with CRISPR-associated or cas genes, function in association with non-coding RNAs to recognize and destroy foreign DNA and to ensure survival against subsequent invasions by a similar pathogen, whether a virus or plasmid. Three types of CRISPR systems have been identified in bacteria with the Type II system being the most widely explored.

The Type II natural RNA-guided DNA nuclease system includes the Cas9 nuclease (also known as Csn1 and formerly known as Cas5) and two small RNAs known as “crRNA” or CRISPR RNA and “tracrRNA” or trans-activating CRISPR RNA. Both are processed from the clustered repeats encoded in the bacterial host genome. The type II CRISPR system requires both the crRNA and the tracrRNA to be functional. In this system, the crRNA associates with the cas9 endonuclease and acts as a hybridization strand providing localization to the complementary target DNA while the tracrRNA associates with the crRNA through partial hybridization and has been shown to be necessary for Cas9 complex binding to the target dsDNA. Once associated with the target dsDNA site, the cas9 enzyme cleaves both strands of the dsDNA thereby destroying the invading organism.

The Type II system is exemplified by the systems found in Streptococcus pyogens and Streptococcus thermophilus. Here the effector complex involves a single Cas9 protein. Early studies of CRISPR-dependent immunity in Streptococcus thermophilus were performed by Barrangou and colleagues (Barrangou et al., Science; 2007; 315:1709-12; Horvath and Barrangou, Science, 2010; 327:167-170). In the S. thermophilus strain, the tracrRNA of approximately 65 nucleotides co-purifies with the Cas9 protein and a 42 nucleotide crRNA (Karvelis et al., RNA Biology, 2013; 10:5: 841-851). Sapranauskas et al. demonstrated the transfer of the S. thermophilus CRISPR3/Cas system into E. coli and that this transfer could provide protection against plasmid transformation. It was also shown that the protection was sequence specific (NAR, 2011; 39(21): 9275-9282).

Studies of the maturation process of the crRNA and tracrRNA in S. pyogens illustrated the necessity of the tracrRNA for maturation of the functional complex and the involvement of an RNase III (Deltcheva, et al., Nature, 2011; 471:602-607). The work of Deltcheva et al. led to the work of Jinek, et al. (Science, 2012; 337:816-821) showing that the crRNA and tracrRNA could be combined into one chimeric RNA molecule to produce a functional ribonucleoprotein complex which could cleave a plasmid or oligonucleotide duplex bearing the required protospacer and PAM. Jinek et al. showed that each Cas9 domain cleaved only one strand of the dsDNA duplex and that point mutations in conserved catalytic amino acids of the two domains (D10A and H840A) resulted in the determination that the HNH domain (mutation H840A) cleaves the strand complementary to the crRNA (or template strand) while the RuvC-like domain (mutation D10A) cleaves the non-complementary or displaced strand (or non-template strand) which comprises the protospacer.

It has since been discovered that Type II CRISPR nuclease-guided cleavage of dsDNA can be reprogrammed to work in higher organism by providing a Cas9 enzyme and altering the features of the two small RNAs associated with the Cas enzyme. In higher organisms such as in mammalian cells, targeting and cleavage of the endogenous or genomic dsDNA triggers the cell's natural repair mechanisms through either non-homologous end joining (NHEJ) or homology directed repair (HDR) pathways, thereby editing the target genomic site.

This observation inspired a series of studies exploring the requirements of tracrRNA size, DNA binding and hybridization, mutant Cas9 enzymes, delivery of the enzyme and RNA molecules to mammalian cells, localization to the nucleus, off-target effects, the specificity of genome editing and multiplexing target sites (Karvelis et al., RNA Biology, 2013; 10:5; 841-851; Gasiunas et al., PNAS, USA 2012; 109:E2579-E2586; Cong et al., Science, 2013; 339: 819-823; Mali, et al., Science; 2013;339: 823-826; Hwang et al., Nat. Biotechnol., 2013; doi 10.1038/nbt.2501; Cho et al., Nat. Biotechnol., 2013; doi 10.1038/nbt.2507; Jiang et al., Nat. Biotechnol., 2013; 31; 233-241; Fu et al., Nat. Biotechnol. 2013; doi: 10.1038/nbt2623; and Chylinski et al., RNA Biology; 2013; 10:5; 726-737).

The use of the CRISPR/Cas9 system in mice has been studied by Shen and Wang et al., (Cell, 2013; 153:910-918). Shen et al. targeted a GFP transgene in the mouse genome by administering a Cas9 mRNA and pre-annealed crRNA-tracrRNA chimera to mouse embryos. They showed site specific cleavage in a chromosomal locus. Wang et al. explored triggering homologous recombination in the mouse and utilized a Cas9 mRNA and chimeric crRNA-tracrRNA. In these studies, conversion of an EcoRV site to an EcoRI restriction sites was successful upon a two base pair insertion. Still neither group demonstrated insertion of a larger polynucleotide.

Cleavage deficient Cas9 enzymes of Streptococcus pyogens and S. thermophilus have been explored further by Sapranauskas et al., (NAR, 2011; 39(21); 9275-9282), Qi et al., (Cell, 2013; 152:1173-1183) and Bikard et al., (NAR, 2013; 1-9) where the effects on transcription modulation including upregulation and silencing were investigated.

Studies in organisms other than bacteria include those in yeast (DiCarlo et al., NAR; 2013; 41:4336-4343), Drosophila (Gratz; Yu et al., Genetics, 2013; doi10.1534/genetics.113.153825) and Zebrafish (Hwang et al., Nat. Biotechnol. Doi 10.1038/nbt.2501).

CRISPR/Cas9 is an RNA-guided DNA endonuclease enzyme and target specificity stems from the guide RNA. Certain CRISPR based methods can be used to control the copies and residence time of a gene product delivered in host human cells.

In embodiments, the regulatable-AAV particles may comprise one or more CRISPR regulatable elements. Such regulatable-AAV particles may be termed “CRISPR-AAV particles.” As used herein, a “CRISPR regulatable element” includes any component of a CRISPR system including but not limited to a Cas9, Cas9 related nucleases, or Cas9-fusion proteins, one or more sgRNA (small guide RNAs), one or more tracrRNAs and/or other polynucleotide feature or motif which imparts regulatable or tunable features to a viral genome encoding them.

CRISPR-AAV particles may be designed for gene knockdown or for gene replacement (resulting in e.g., activation, initiation, and increased expression).

In an embodiment contemplated by the current invention, a CRISPR regulatable element may selectively disrupt fragments of a regulatable-AAV particle. Upon delivery, a CRISPR regulatable element comprising Cas9 and a custom RNA sequence for guidance of Cas9 may cleave the payload construct or viral genome at a strategically placed target site, thereby inactivating the payload.

In some embodiments, the cas9 protein or nuclease is the CRISPR regulatable element and may be selected from any of the known or putative Streptococcus cas9 enzymes in the Uniprot cluster and listed in Table 4. Such proteins, if they are to be delivered as a nucleic acid such as an encoded mRNA and expressed in an organism other than Streptococcus, may be codon optimized for expression in the recipient cell or organism.

TABLE 4
Cas 9 proteins of Streptococcus sp.

| | | | SEQ |
|---|---|---|---|
| Uniprot | | | ID |
| Entry | Protein name | Organism | NO |
| Q03JI6 | CRISPR-associated endonuclease Cas9 | Streptococcus thermophilus (strain | 571 |
| | 2 (EC 3.1.—.—) | ATCC BAA-491/LMD-9) | |
| Q99ZW2 | CRISPR-associated endonuclease | Streptococcus pyogenes serotype M1 | 572 |
| | Cas9/Csnl (EC 3.1.—.—) | | |
| G3ECR1 | CRISPR-associated endonuclease Cas9 | Streptococcus thermophilus | 573 |
| | (EC 3.1.—.—) | | |
| J3JPT0 | CRISPR-associated protein csn1 | Streptococcus ratti FA-1 = DSM 20564 | 574 |
| | (Uncharacterized protein) | | |
| I5BLK7 | Uncharacterized protein | Streptococcus agalactiae ZQ0910 | 575 |
| K4Q9P5 | Uncharacterized protein | Streptococcus dysgalactiae subsp. | 576 |
| | | equisimilis AC-2713 | |
| Q3D2H4 | Reticulocyte binding protein | Streptococcus agalactiae H36B | 577 |
| M2GS30 | Uncharacterized protein | Streptococcus mutans A19 | 578 |
| Q3DG33 | Reticulocyte binding protein | Streptococcus agalactiae CJB111 | 579 |
| M1YDU0 | Uncharacterized protein | Streptococcus agalactiae LADL-90-503 | 580 |
| M7DS80 | Uncharacterized protein | Streptococcus mutans ATCC 25175 | 581 |
| M7DAQ6 | Uncharacterized protein | Streptococcus mutans KK23 | 582 |
| M2KYB4 | Uncharacterized protein | Streptococcus mutans S1B | 583 |
| I0Q2W2 | CRISPR-associated protein Cas9/Csn1, | Streptococcus oralis SK610 | 584 |
| | subtype II/NMEMI | | |
| Q3DN68 | Reticulocyte binding protein | Streptococcus agalactiae 515 | 585 |
| E9FPR9 | CRISPR-associated protein, Csn1 | Streptococcus sp. M334 | 586 |
| | family | | |
| F5U0T2 | CRISPR-associated protein, Csn1 | Streptococcus anginosus SK52 = DSM | 587 |
| | family | 20563 | |
| H8HE09 | CRISPR-associated protein Csn1 | Streptococcus pyogenes MGAS1882 | 588 |
| M2E8A3 | Uncharacterized protein | Streptococcus mutans 8ID3 | 589 |
| Q3DAP7 | Reticulocyte binding protein | Streptococcus agalactiae COH1 | 590 |
| J7TMY5 | Putative cytosolic protein | Streptococcus salivarius K12 | 591 |
| F2C4I5 | Csn1 family CRISPR-associated protein | Streptococcus sanguinis SK330 | 592 |
| F9HIG7 | CRISPR-associated protein Cas9/Csn1, | Streptococcus sp. oral taxon 056 str. | 593 |
| | subtype II/NMEMI | F0418 | |
| E0PL18 | Csn1 family CRISPR-associated protein | Streptococcus gallolyticus subsp. | 594 |
| | | gallolyticus TX20005 | |
| M4YX12 | CRISPR-associated protein | Streptococcus dysgalactiae subsp. | 595 |
| | | equisimilis RE378 | |
| M1XVB4 | Uncharacterized protein | Streptococcus agalactiae SS1219 | 596 |
| M7E3Z6 | CRISPR-associated protein csn1 | Streptococcus mutans NCTC 11060 | 597 |
| M2ECS5 | Uncharacterized protein | Streptococcus mutans 4SM1 | 598 |
| F0I6Z8 | Csn1 family CRISPR-associated protein | Streptococcus sanguinis SK115 | 599 |
| M2FSD0 | Uncharacterized protein | Streptococcus mutans 2VS1 | 600 |
| J7M7J1 | Uncharacterized protein | Streptococcus pyogenes M1 476 | 601 |
| M2LXP5 | Uncharacterized protein | Streptococcus mutans U2B | 602 |
| M2IJW5 | Uncharacterized protein | Streptococcus mutans M2A | 603 |
| M2KKV5 | Uncharacterized protein | Streptococcus mutans 66-2A | 604 |
| M2IIP5 | Uncharacterized protein | Streptococcus mutans NLML9 | 605 |
| M2DYK8 | Uncharacterized protein | Streptococcus mutans 4VF1 | 606 |
| M2HBR4 | Uncharacterized protein | Streptococcus mutans N66 | 607 |
| G5KAN2 | CRISPR-associated protein Cas9/Csn1, | Streptococcus pseudoporcinus LQ 940- | 608 |
| | subtype II/NMEMI | 04 | |
| M2F746 | Uncharacterized protein | Streptococcus mutans 11VS1 | 609 |
| M2KCP8 | Uncharacterized protein | Streptococcus mutans SA38 | 610 |
| K4N5K1 | CRISPR-associated protein, Csn1 | Streptococcus pyogenes A20 | 611 |
| | family | | |
| M2G9R5 | Uncharacterized protein | Streptococcus mutans A9 | 612 |
| M2KJE3 | Uncharacterized protein | Streptococcus mutans SM4 | 613 |
| M7CZ76 | Uncharacterized protein | Streptococcus mutans KK21 | 614 |
| M2FYT7 | Uncharacterized protein | Streptococcus mutans M21 | 615 |
| F5U4D7 | CRISPR-associated protein, Csn1 | Streptococcus dysgalactiae subsp. | 616 |
| | family (Fragment) | equisimilis SK1249 | |
| F5U5Q4 | Putative uncharacterized protein | Streptococcus dysgalactiae subsp. | 617 |
| | (Fragment) | equisimilis SK1249 | |
| M2H646 | Uncharacterized protein | Streptococcus mutans U138 | 618 |
| M2KHB4 | Uncharacterized protein | Streptococcus mutans SM1 | 619 |
| M2J4V9 | Uncharacterized protein | Streptococcus mutans ST6 | 620 |
| M2F2U6 | Uncharacterized protein | Streptococcus mutans 11SSST2 | 621 |
| G7SP82 | CRISPR-system-like protein | Streptococcus suis ST1 | 622 |
| M2FXA5 | Uncharacterized protein | Streptococcus mutans G123 | 623 |
| M2IJB5 | Uncharacterized protein | Streptococcus mutans NV1996 | 624 |
| M7E6C3 | Uncharacterized protein | Streptococcus mutans AC4446 | 625 |
| K1LK43 | Csn1 family CRISPR-associated protein | Streptococcus iniae 9117 | 626 |
| I0SF74 | CRISPR-associated protein Cas9/Csn1, | Streptococcus constellatus subsp. | 627 |
| | subtype II/NMEMI | constellatus SK53 | |
| M1YIE1 | Uncharacterized protein | Streptococcus agalactiae CF01173 | 628 |
| M2J1X3 | Uncharacterized protein | Streptococcus mutans W6 | 629 |
| I6SW88 | CRISPR-associated protein csn1 | Streptococcus mutans GS-5 | 630 |
| I6Q294 | Csn1 | Streptococcus thermophilus MN-ZLW- | 631 |
| | | 002 | |
| F0FD37 | Csn1 family CRISPR-associated protein | Streptococcus sanguinis SK353 | 632 |
| F9NIK9 | CRISPR-associated protein Cas9/Csn1, | Streptococcus dysgalactiae subsp. | 633 |
| | subtype II/NMEMI | equisimilis SK1250 | |
| F9NIK8 | Putative uncharacterized protein | Streptococcus dysgalactiae subsp. | 634 |
| | | equisimilis SK1250 | |
| M2JCP4 | Uncharacterized protein | Streptococcus mutans B | 635 |
| K4PPI8 | CRISPR-associated protein | Streptococcus agalactiae SA20-06 | 636 |
| E1LI65 | HNH endonuclease family protein | Streptococcus mitis SK321 | 637 |
| E9FJ16 | CRISPR-associated protein, Csn1 | Streptococcus sp. C300 | 638 |
| | family | | |
| M2KGB0 | Uncharacterized protein | Streptococcus mutans 14D | 639 |
| M7DIF2 | CRISPR-associated protein csn1 | Streptococcus mutans 5DC8 | 640 |
| M2INU6 | Uncharacterized protein | Streptococcus mutans SF1 | 641 |
| M2LHR5 | Uncharacterized protein | Streptococcus mutans 24 | 642 |
| J4TM44 | CRISPR-associated protein Cas9/Csn1, | Streptococcus anginosus SK1138 | 643 |
| | subtype II/NMEMI | | |
| M2JLG8 | Uncharacterized protein | Streptococcus mutans SM6 | 644 |
| I7QXF2 | Putative cytoplasmic protein | Streptococcus canis FSL Z3-227 | 645 |
| I3I1V4 | Putative cytoplasmic protein | Streptococcus pyogenes HKU | 646 |
| | | QMH11M0907901 | |
| J8T4Q2 | Uncharacterized protein | Streptococcus agalactiae GB00112 | 647 |
| E7S4M3 | Csn1 family CRISPR-associated protein | Streptococcus agalactiae ATCC 13813 | 648 |
| M2HZK2 | Uncharacterized protein | Streptococcus mutans NLML4 | 649 |
| M2DAT4 | Uncharacterized protein | Streptococcus mutans 1SM1 | 650 |
| M2GPV5 | CRISPR-associated protein | Streptococcus mutans NMT4863 | 651 |
| M5PJI2 | CRISPR-associated protein | Streptococcus parauberis KRS-02109 | 652 |
| F8Y040 | Putative uncharacterized protein | Streptococcus agalactiae FSL S3-026 | 653 |
| M2FXC0 | Uncharacterized protein | Streptococcus mutans 5SM3 | 654 |
| M2ENP9 | Uncharacterized protein | Streptococcus mutans NFSM2 | 655 |
| M1XWD6 | Uncharacterized protein | Streptococcus agalactiae SS1014 | 656 |
| K0U976 | Uncharacterized protein | Streptococcus agalactiae STIR-CD-17 | 657 |
| E0PEL3 | Csn1 family CRISPR-associated protein | Streptococcus bovisATCC 700338 | 658 |
| M2EUD0 | Uncharacterized protein | Streptococcus mutans 3SN1 | 659 |
| E4L3R1 | CRISPR-associated protein, Csn1 | Streptococcus pseudoporcinus SPIN | 660 |
| | family | 20026 | |
| M2LYU7 | Uncharacterized protein | Streptococcus mutans R221 | 661 |
| M2IAS5 | Uncharacterized protein | Streptococcus mutans N3209 | 662 |
| M2KAP8 | Uncharacterized protein | Streptococcus mutans NLML1 | 663 |
| M2F4E1 | Uncharacterized protein | Streptococcus mutans N29 | 664 |
| E8JP81 | Csn1 family CRISPR-associated protein | Streptococcus equinus ATCC 9812 | 665 |
| M2G1L7 | Uncharacterized protein | Streptococcus mutans NVAB | 666 |
| J4K985 | CRISPR-associated protein Cas9/Csn1, | Streptococcus oralis SK304 | 667 |
| | subtype II/NMEMI | | |
| M2E7C1 | CRISPR-associated protein | Streptococcus mutans 15VF2 | 668 |
| Q3DQT5 | CRISPR-associated protein, SAG0894 | Streptococcus agalactiae 18RS21 | 669 |
| | family (Fragment) | | |
| M2IP01 | Uncharacterized protein | Streptococcus mutans SF14 | 670 |
| E6J3R0 | CRISPR-associated protein, Csn1 | Streptococcus anginosus F0211 | 671 |
| | family | | |
| G5JVJ9 | CRISPR-associated protein Cas9/Csn1, | Streptococcus macacae NCTC 11558 | 672 |
| | subtype II/NMEMI | | |
| K8MQ90 | CRISPR-associated protein cas9/csn1, | Streptococcus sp. F0441 | 673 |
| | subtype II/nmemi | | |
| M2KYT3 | Uncharacterized protein | Streptococcus mutans M230 | 674 |
| H8HAK7 | CRISPR-associated protein Csn1 | Streptococcus pyogenes MGAS15252 | 675 |
| M2HZJ5 | Uncharacterized protein | Streptococcus mutans NFSM1 | 676 |
| E4SQY2 | CRISPR-associated endonuclease, Csn1 | Streptococcus thermophilus (strain | 677 |
| | family | ND03) | |
| Q1JC13 | Hypothetical cytosolic protein | Streptococcus pyogenes serotype M12 | 678 |
| | | (strain MGAS2096) | |
| C6SPS8 | Uncharacterized protein | Streptococcus mutans serotype c (strain | 679 |
| | | NN2025) | |
| C5WH61 | CRISPR-associated protein Csn1 | Streptococcus dysgalactiae subsp. | 680 |
| | | equisimilis (strain GGS_124) | |
| Q8E042 | Putative uncharacterized protein | Streptococcus agalactiae serotype V | 681 |
| | | (strain ATCC BAA-611/2603 VZR) | |
| F5WVJ4 | CRISPR-associated protein | Streptococcus gallolyticus (strain ATCC | 682 |
| | | 43143/F-1867) | |
| D3HEH4 | CRISPR-associated protein | Streptococcus gallolyticus (strain | 683 |
| | | UCN34) | |
| J9YP56 | Uncharacterized protein | Streptococcus agalactiae serotype Ia | 684 |
| | | (strain GD201008-001) | |
| Q8E5R9 | Putative uncharacterized protein | Streptococcus agalactiae serotype III | 685 |
| | gbs0911 | (strain NEM316) | |
| F7IUC8 | Putative uncharacterized protein | Streptococcus pyogenes serotype M3 | 686 |
| | SPS1176 | (strain SSI-1) | |
| Q8DTE3 | Putative uncharacterized protein | Streptococcus mutans serotype c (strain | 687 |
| | | ATCC 700610/UA159) | |
| F0VS85 | CRISPR-associated protein | Streptococcus gallolyticus (strain ATCC | 688 |
| | | BAA-2069) | |
| B5XLC1 | Putative uncharacterized protein | Streptococcus pyogenes serotype M49 | 689 |
| | | (strain NZ131) | |
| Q3K1G4 | CRISPR-associated protein, SAG0894 | Streptococcus agalactiae serotype Ia | 690 |
| | family | (strain ATCC 27591/A909/CDC | |
| | | SS700) | |
| E8QAX4 | Hypothetical cytosolic protein | Streptococcus dysgalactiae subsp. | 691 |
| | | equisimilis (strain ATCC 12394/ | |
| | | D166B) | |
| H6PBR9 | CRISPR-associated protein, SAG0894 | Streptococcus infantarius (strain CJ18) | 692 |
| | family | | |
| Q48Z31 | Hypothetical cytosolic protein | Streptococcus pyogenes serotype M1 | 693 |
| Q1J6W2 | Hypothetical cytosolic protein | Streptococcus pyogenes serotype M4 | 694 |
| | | (strain MGAS10750) | |
| Q8K7R2 | Putative uncharacterized protein | Streptococcus pyogenes serotype M3 | 695 |
| | | (strain ATCC BAA-595/MGAS315) | |
| Q1JLZ6 | Hypothetical cytosolic protein | Streptococcus pyogenes serotype M12 | 696 |
| | | (strain MGAS9429) | |
| Q48TU5 | Hypothetical cytosolic protein | Streptococcus pyogenes serotype M28 | 697 |
| | | (strain MGAS6180) | |
| Q1JH43 | Hypothetical cytosolic protein | Streptococcus pyogenes serotype M2 | 698 |
| | | (strain MGAS10270) | |

Chylinski et al. (RNA Biology, 2013, 10:5, 726-737, the contents of which are herein incorporated by reference in their entirety including supplemental materials), has identified several Cas9 orthologs from Type II CRISPR-Cas loci. Any of these may be used as the CRISPR regulatable element.

According to the present invention, these Cas9 enzymes may also serve as genome editing enzymes, e.g., CRISPR regulatable elements, of the invention and are given in Table 5. Given in the table are the gi accession numbers from NCBI and the name of the bacterial strain. It will be understood that such enzymes, when expressed in any organism other than the wild type strain may be codon optimized for that organism at the nucleic acid level.

TABLE 5
Cas9 Orthologs

| | | SEQ ID |
|---|---|---|
| gi Number | Strain | NO |
| 491523080 | Veillonella atypica ACS-134-V-Col7a | 699 |
| 492568239 | Fusobacterium nucleatum subsp. vincentii | 700 |
| | ATCC 49256 | |
| 291166249 | Filifactor alocis ATCC 35896 | 701 |
| 320130861 | Solobacterium moorei F0204 | 702 |
| 291520705 | Coprococcus catus GD-7 | 703 |
| 42525843 | Treponema denticola ATCC 35405 | 704 |
| 496176552 | Peptoniphilus duerdenii ATCC BAA-1640 | 705 |
| 493553119 | Catenibacterium mitsuokai DSM 15897 | 706 |
| 24379809 | Streptococcus mutans UA159 | 707 |
| 15675041 | Streptococcus pyogenes SF370 | 708 |
| 499300419 | Listeria innocua Clip11262 | 709 |
| 500000752 | Streptococcus thermophilus LMD-9 | 710 |
| 323463801 | Staphylococcus pseudintermedius | 711 |
| 352684361 | Acidaminococcus intestini RyC-MR95 | 712 |
| 503017123 | Olsenella uli DSM 7084 | 713 |
| 366983953 | Oenococcus kitaharae DSM 17330 | 714 |
| 503128334 | Bifidobacterium bifidum S17 | 715 |
| 504382875 | Lactobacillus rhamnosus GG | 716 |
| 489744644 | Lactobacillus gasseri JV-V03 | 717 |
| 501247123 | Finegoldia magna ATCC 29328 | 718 |
| 47458196 | Mycoplasma mobile 163K | 719 |
| 284931710 | Mycoplasma gallisepticum str. F | 720 |
| 498006766 | Mycoplasma ovipneumoniae SC01 | 721 |
| 384393286 | Mycoplasma canis PG 14 | 722 |
| 144575181 | Mycoplasma synoviae 53 | 723 |
| 238875750 | Eubacterium rectale ATCC 33656 | 724 |
| 500000239 | Streptococcus thermophilus LMD-9 | 725 |
| 315149830 | Enterococcus faecalis TX0012 | 726 |
| 488391463 | Staphylococcus lugdunensis M23590 | 727 |
| 158432258 | Eubacterium dolichum DSM 3991 | 728 |
| 497700222 | Lactobacillus coiyniformis subsp. torquens | 729 |
| | KCTC 3535 | |
| 503154365 | Ilyobacter polytropus DSM 2926 | 730 |
| 488935851 | Ruminococcus albus 8 | 731 |
| 187426541 | Akkermansia muciniphila ATCC BAA-835 | 732 |
| 117649621 | Acidothermus cellulolyticus 11B | 733 |
| 189429199 | Bifidobacterium longum DJO10A | 734 |
| 502666262 | Bifidobacterium dentium Bd1 | 735 |
| 499236428 | Corynebacterium diphtheriae NCTC 13129 | 736 |
| 501382854 | Elusimicrobium minutum Pei191 | 737 |
| 319419610 | Nitratifractor salsuginis DSM 16511 | 738 |
| 324027241 | Sphaerochaeta globus str. Buddy | 739 |
| 502574305 | Fibrobacter succinogenes subsp. succinogenes | 740 |
| | S85 | |
| 496648031 | Bacteroides fragilis NCTC 9343 | 741 |
| 506262077 | Capnocytophaga ochracea DSM 7271 | 742 |
| 499794158 | Rhodopseudomonas palustris BisB18 | 743 |
| 494010777 | Prevotella micans F0438 | 744 |
| 294472455 | Prevotella ruminicola 23 | 745 |
| 503930464 | Flavobacterium columnare ATCC 49512 | 746 |
| 310782306 | Aminomonas paucivorans DSM 12260 | 747 |
| 83591793 | Rhodospirillum rubrum ATCC 11170 | 748 |
| 502812437 | Candidatus Puniceispirillum marinum | 749 |
| | IMCC1322 | |
| 500133006 | Verminephrobacter eiseniae EF01-2 | 750 |
| 344171927 | Ralstonia syzygii R24 | 751 |
| 159042956 | Dinoroseobacter shibae DEL 12 | 752 |
| 288910049 | Azospirillum sp- B510 | 753 |
| 91802344 | Nitrobacter hamburgensis X14 | 754 |
| 146407516 | Brady rhizobium sp- BTAi1 | 755 |
| 499451825 | Wolinella succinogenes DSM 1740 | 756 |
| 218563121 | Campylobacter jejuni subsp. jejuni NCTC 11168 | 757 |
| 502787413 | Helicobacter mustelae 12198 | 758 |
| 447027826 | Bacillus cereus Rock1-15 | 759 |
| 501844634 | Acidovorax ebreus TPSY | 760 |
| 189485225 | uncultured Termite group 1 bacterium phylotype | 761 |
| | RsD17 | |
| 489569047 | Clostridium perfringens D str. JGS1721 | 762 |
| 506406750 | Clostridium cellulolyticum H10 | 763 |
| 154154505 | Parvibaculum lavamentivorans DS-1 | 764 |
| 493910016 | Roseburia intestinalis L1-82 | 765 |
| 488163954 | Neisseria meningitidis Z2491 | 766 |
| 499209493 | Pasteurella multocida subsp. | 767 |
| | multocida str. Pm70 | |
| 491573077 | Sutterella wadsworthensis 3 1 45B | 768 |
| 495559660 | gamma proteobacterium HTCC5015 | 769 |
| 499526152 | Legionella pneumophila str. Paris | 770 |
| 496140336 | Parasutterella excrementiho minis YIT 11859 | 771 |
| 499451967 | Wolinella succinogenes DSM 1740 | 772 |
| 489129153 | Francisella novicida U112 | 773 |

Any of the enzymes or proteins of Tables 4 or 5 may be a CRISPR regulatable element. Several protospacer adjacent motifs (PAMs) have been identified in the art (Westra et al., Annu Rev. Genet. 2012, 46: 311-39; the contents of which are incorporated herein by reference in their entirety). Such PAMs may be used to inform the selection of and/or design of the nucleic acid compositions, e.g., CRISPR-AAV particles, of the present invention. It is also contemplated that utilization of certain Type III enzymes such as those from Staphylococcus epidermidis, Pyrococcus furiosus or S. solfatarcicus will not require the presence of a PAM sequence. PAMs useful in the present invention are given in Table 6. In the table the PAM either follows or precedes the protospacer (that region of the DNA found immediately upstream or downstream of the PAM and on the opposite DNA strand that hybridizes with the sgRNA).

TABLE 6
PAM sequences

| PAM | SEQ ID NO |
|---|---|
| Protospacer-NGG | 774-777 |
| Protospacer-GAA | 778-779 |
| Protospacer-CTT | 780-781 |
| Protospacer-CAT | 782 |
| Protospacer-CCT | 783 |
| Protospacer-CTC | 784 |
| Protospacer-GG | 785-786 |
| WTTCTNN-Protospacer | 787 |
| TTTYRNNN-Protospacer | 788 |
| CNCCN-Protospacer | 789 |
| CCN-Protospacer | 790-791 |

Recently several improvements have been made to the CRISPR system, which address the more limited AAV packaging size. Recent work with minimal promoter and polyadenylation sequences has produced functional SpCas9 constructs that can be effectively packaged in AAVs, as described in Swiech et al., (In vivo interrogation of gene function in the mammalian brain using CRISPR-Cas9; Nat Biotechnol. 2015 January; 33(1):102-6) and Senis et al. (CRISPR/Cas9-mediated genome engineering: an adeno-associated viral (AAV) vector toolbox; Biotechnol J. 2014 November; 9(11):1402-12), the contents of each of which is herein incorporated by reference in its entirety. In addition, Ran et al., characterized six smaller Cas9 orthologues, including Cas9 from Staphylococcus aureus, which they packed with a guide RNA into a single AAV vector to target PCSK9 in the liver, as described in Ran et al., In vivo genome editing using Staphylococcus aureus Cas9, Nature. 2015 Apr. 9; 520(7546):186-91, the contents of which is herein incorporated by reference in its entirety. Other smaller Cas enzymes have also been described by Cong L et al. (Multiplex genome engineering using CRISPR/Cas systems; Science 339, 819-823, 2013) and Nishimasu et al. (Crystal structure of Cas9 in complex with guide RNA and target DNA; Cell 156, 935-949, 2014), the contents of each of which is herein incorporated by reference in its entirety.

Fine et al., describe a split-intein Cas9, which can be separated into two AAV cassettes, each less than 4 kb, providing room for regulatory sequences and additional gRNAs in each cassette (Sci Rep. 2015; 5: 10777; Trans-spliced Cas9 allows cleavage of HBB and CCRS genes in human cells using compact expression cassettes), the contents of which is herein incorporated by reference in its entirety. Fusion of effector domains which can be added to the cassette without space constraints include dCas9, FokI, VP64, and KRAB.

In one embodiment, the regulatable-AAV particle of the present invention may be an AAV-split-Cas9 system as described in Chew et al (A multifunctional AAV-CRISPR-Cas9 and its host response; Nature Methods; published online Sep. 5, 2016), the contents of which are herein incorporated by reference in their entirety. In this split-Cas9 system, SpCas9 is split at its disordered linker (V713-D718), which allows reconstitution of full length Cas9 in vivo by split intein protein trans-splicing. The N-terminal lob of Cas9 is fused to the Rhodothermus marinus N-split intein, while the C-terminal lobe is fused with C-split intein, together shortening the coding sequences below those of other known Cas9 orthologs. In some embodiments the split-Cas9 system may incorporate transcription-activator fusion domains, allowing for targeted upregulation of gene expression. Further, nuclease-active Cas9 combined with truncated gRNAs can bind genomic loci and generate gene activation rather than DNA breaks, thereby allowing a single Cas-9 activator fusion protein to function in gene editing or gene activation processes, depending on the gRNAs and spacer lengths.

Yang et al., describe administering two AAVs, one expressing Cas9 and the other one expressing a guide RNA and the donor DNA (Nature Biotechnology, 2016, A dual AAV system enables the Cas9-mediated correction of a metabolic liver disease in newborn mice; the contents of which are herein incorporated by reference in its entirety). The Cas9 may be a Cas9 enzyme from Staphylococcus aureus (SaCas9) and the AAV may comprise a TBG promoter. The second AAV may comprise a U6 promoter, a single guide RNA (sgRNA) sequence and a donor DNA.

Yin et al., describe combining lipid nanoparticle-mediated delivery of Cas9 mRNA with AAV encoding a sgRNA and a repair template to induce repair of a disease gene in animals (Nature Biotechnology, 2016, Therapeutic genome editing by combined viral and non-viral delivery of CRISPR system components in vivo; the contents of which are herein incorporated by reference in its entirety). Additionally the Cas9 may be the smaller form version of Cas9 (Staphylococcus aureus Cas9).

It is contemplated that of the constructs and enzymes described in Swiech et al, Senis et al., Ran et al., Cong et al., Nishimasu et al., or Fine et al. or any other CRISPR systems optimized for smaller packaging size, may be used as part of the instant invention as CRISPR regulatable elements and/or CRISPR-AAV particles.

In one embodiment, the expression of the payload may be regulated by a CRISPR Cas9 enzyme and a guide RNA that targets the enzyme to a site within the payload. In some embodiments, the regulatable element may comprise two or more guide RNAs. In some embodiments, the two or more guide RNAs may be specific for two or more different sequences within one or more viral genomes.

In one embodiment, the CRISPR regulatable element may be a “self-destructing message,” i.e. as Cas9 mRNA is transcribed and then translated, the protein Cas9 together with the sgRNA may bind and create a double strand break in the same delivery vehicle, effectively disrupting its function and destroying the delivery vehicle. An exemplary “self-destructing message is described in Moore et al., Nucl. Acids Res., 2014, the contents of which is herein incorporated in its entirety.

In one embodiment, the regulatable element of the present invention, when expressed, may be a CRISPR effector protein as described in United States Publication No. US 20160208243, the contents of which are herein incorporated by reference in their entirety. In one embodiment, the CRISPR effector protein is a Cpf1 effector protein, comprising a C-terminal RuvC domain, an N-terminal alpha-helical region and a mixed alpha and beta region located between the RuvC and alpha-helical domains. In these cases, the CRISPR arrays are processed into mature crRNAs without the need of a tracrRNA, wherein the crRNAs comprise a spacer sequence and a direct repeat sequence. A Cpf1p-crRNA complex is alone sufficient to cleave target DNA. In one embodiment, the CRISPR effector protein is a C2c1 loci effector protein. In some embodiments the CRISPR effector protein may have mutations or modifications therein. Further the Cpf1 CRISPR-Cas system may be a split-Cpf1, an inducible system, a self-inactivating system, or a multiplex-tandem targeting approach system. The Cpf1 CRISPR effector proteins described herein may be delivered to plants, animals, stem cells or the like and may be used to treat diseases or disorders.

In one embodiment, the payload expression may be regulated through the stability of the guide RNA. In one embodiment, the guide RNA may be stabilized through stabilizing elements known in the art. For example, extending the 5′ end of the gRNA may increase the half-life of the gRNA (Mali P, et al. CAS9 transcriptional activators for target specificity screening and paired nickases for cooperative genome engineering. Nat Biotechnol. 2013; 31:833-838). In another embodiment, the guide RNA may be destabilized through destabilizing sequences including those described herein.

In one embodiment, the expression of Cas9 and the guide RNA may be under control of one promoter. In another embodiment, the expression of Cas9 and the guide RNA may be under control of separate promoters. In one embodiment, Cas9 and the guide RNA expression may both be driven by the same or two different constitutive promoter(s). In one embodiment, the Cas9 and the guide RNA expression may both be driven by the same or two different inducible promoter(s). In another embodiment, the expression of one of the components may be driven by a constitutive promoter, and the other by an inducible promoter. In another embodiment, the viral genome comprises a promoter which can drive tissue specific expression, such that the CRISPR regulatable element is only expressed in certain tissues.

In one embodiment, the VP2 capsid may comprise a DNA binding domain for the cas9 promoter and/or a transactivating factor for cas9. The transactivating factor may be pre-engineered to induce cas9 expression.

In one embodiment, the expression of cas9 with a VP2 capsid comprising a DNA binding domain for the cas9 promoter and/or a transactivating factor for cas9 may be consistent over a period of time. The expression may be consistent for minutes (e.g., 1, 2, 3, 4, 5, 6, 7, 8, 9, 10, 15, 20, 25, 30, 35, 40, 45, 50, 55, or more than 55 minutes), hours (e.g., 1, 2, 3, 4, 5, 6, 7, 8, 9, 10, 11, 12, 13, 14, 15, 16, 17, 18, 19, 20, 21, 22, 23 or more than 24 hours), days (e.g., 1, 2, 3, 4, 5, 6, 7, 8, 9, 10, 11, 12, 13, 14, 15, 16, 17, 18, 19, 20 or more than 20 days), weeks (e.g., 1, 2, 3, 4, 5, 6, 7, 8, 9, 10 or more than 10 weeks), or months (e.g., 1, 2, 3, 4, 5, 6, 7, 8, 9, 10, 11, or more than 11 months). As another non-limiting example, expression of cas9 may be a burst of expression for a predetermined period of time (e.g., burst of expression for one hours, two hours, three hours, four hours, five hours, six hours or greater than six hours after administration).

In one embodiment, the promoter and/or transactivating domain for cas9 may be located within the first 1%, 2%, 3%, 4%, 5%, 6%, 7%, 8%, 9%, 10%, 15%, 20%, 25%, 30%, 35%, 40%, 45%, 46%, 47%, 48,% or 49% of the VP2 capsid. In another embodiment, the promoter and/or transactivating domain for cas9 may be located within the last 1%, 2%, 3%, 4%, 5%, 6%, 7%, 8%, 9%, 10%, 15%, 20%, 25%, 30%, 35%, 40%, 45%, 46%, 47%, 48,% or 49% of the VP2 capsid. In another embodiment, the promoter and/or transactivating factor for cas9 may be located in the middle of the VP2 capsid. In another embodiment, the promoter and/or transactivating domain for cas9 may be located near the beginning of the VP2 capsid. In another embodiment, the promoter and/or transactivating domain for cas9 may be located near the end of the VP2 capsid.

In some embodiments, the CRISPR regulatable element may encode Cpf1. Cpf1, a class II CRISPR endonuclease, is a single RNA-guided endonuclease lacking tracrRNA, which utilizes a T-rich protospacer-adjacent motif. Cpf1 cleaves DNA via a staggered DNA double-stranded break. Cpf1 is smaller than the standard Cas9, facilitating delivery to desired tissues, as described in Zetsche et al. (Cpf1 Is a Single RNA-Guided Endonuclease of a Class 2 CRISPR-Cas System, Cell, published online Sep. 25, 2015), the contents of which is herein incorporated by reference in its entirety.

In one embodiment, the expression of Cpf1 and the guide RNA may be under control of one promoter. In another embodiment, the expression of Cpf1 and the guide RNA may be under control of separate promoters. In one embodiment, Cpf1 and the guide RNA expression may both be driven by the same or two different constitutive promoter(s). In one embodiment, the Cpf1 and the guide RNA expression may both be driven by the same or two different inducible promoter(s). In another embodiment, the expression of one of the components may be driven by a constitutive promoter, and the other by an inducible promoter. In another embodiment, the viral genome comprises a promoter which can drive tissue specific expression, such that the CRISPR regulatable element is only expressed in certain tissues.

In one embodiment, the VP2 capsid may comprise a DNA binding domain for the Cpf1 promoter and/or a transactivating factor for Cpf1. The transactivating factor may be pre-engineered to induce Cpf1 expression.

In one embodiment, the expression of Cpf1 with a VP2 capsid comprising a DNA binding domain for the Cpf1 promoter and/or a transactivating factor for Cpf1 may be consistent over a period of time. The expression may be consistent for minutes (e.g., 1, 2, 3, 4, 5, 6, 7, 8, 9, 10, 15, 20, 25, 30, 35, 40, 45, 50, 55, or more than 55 minutes), hours (e.g., 1, 2, 3, 4, 5, 6, 7, 8, 9, 10, 11, 12, 13, 14, 15, 16, 17, 18, 19, 20, 21, 22, 23 or more than 24 hours), days (e.g., 1, 2, 3, 4, 5, 6, 7, 8, 9, 10, 11, 12, 13, 14, 15, 16, 17, 18, 19, 20 or more than 20 days), weeks (e.g., 1, 2, 3, 4, 5, 6, 7, 8, 9, 10 or more than 10 weeks), or months (e.g., 1, 2, 3, 4, 5, 6, 7, 8, 9, 10, 11, or more than 11 months). As another non-limiting example, expression of Cpf1 may be a burst of expression for a predetermined period of time (e.g., burst of expression for one hours, two hours, three hours, four hours, five hours, six hours or greater than six hours after administration).

In one embodiment, the promoter and/or transactivating domain for Cpf1 may be located within the first 1%, 2%, 3%, 4%, 5%, 6%, 7%, 8%, 9%, 10%, 15%, 20%, 25%, 30%, 35%, 40%, 45%, 46%, 47%, 48,% or 49% of the VP2 capsid. In another embodiment, the promoter and/or transactivating domain for Cpf1 may be located within the last 1%, 2%, 3%, 4%, 5%, 6%, 7%, 8%, 9%, 10%, 15%, 20%, 25%, 30%, 35%, 40%, 45%, 46%, 47%, 48,% or 49% of the VP2 capsid. In another embodiment, the promoter and/or transactivating factor for Cpf1 may be located in the middle of the VP2 capsid. In another embodiment, the promoter and/or transactivating domain for Cpf1 may be located near the beginning of the VP2 capsid. In another embodiment, the promoter and/or transactivating domain for Cpf1 may be located near the end of the VP2 capsid.

In one embodiment, the payload comprises one or more CRISPR recognition sequences. As used herein, the term “CRISPR recognition sequence” refers to a sequence in a construct that the CRISPR system can recognize and can target for regulation of that construct. In one embodiment, the one or more CRISPR recognition sequence is within the coding region. In one embodiment, the CRISPR recognition sequence is outside of the coding region. These regions in which the CRISPR recognition sequence may be located include, but are not limited to, the 5′ITR region, the region downstream of the 5′ITR and upstream of the promoter, the promoter region, the 5′UTR region, the coding region, the 3′UTR region, the region downstream of the 3′UTR and upstream of the 3′ITR, and the 3′ITR region.

In one embodiment, the guide RNAs, which may be encoded within or used in combination with the regulatable-AAV particles of the present invention, target specific nucleotide sequences of the DMD gene, and can be used for the treatment of Duchenne muscular dystrophy or Becker muscular dystrophy as described in International Publication No. WO2016161380, the contents of which are herein incorporated by reference in their entirety. This genome editing system comprises a first and second gRNA each targeting a domain of 19-24 nucleotides in length of the DMD gene, and at least one Cas9 molecule that recognizes a PAM of either NNGRRT or NNGRRV. This genome editing system results in a first and second double strand break in the first and second intron flanking exon 51 of the DMD gene, thereby causing its deletion. In one embodiment, the gRNA may have a targeting domain that comprises a nucleotide sequence as set forth by any of SEQ ID NOs: 206-826366 as described in WO2016161380.

In one embodiment, the CRISPR regulatable elements may be located on the same viral genome as the payload. In another embodiment, the CRISPR regulatable elements and payload are on a separate viral genomes and packaged in separate AAV particles.

### Other Cas9 Fusion Proteins

In one embodiment, the CRISPR/Cas9 system can be reengineered for transcriptional regulation and used with or encapsulated by the viral particles described herein (e.g., AAV particles or CRISPR-AAV particles). Cas9 catalyzes DNA double-stranded breaks via RuvC and HNH endonuclease domains, each of which cleaves one strand of the target DNA. Both of these enzymatic domains can be inactivated by a single amino acid substitution (D10A and H840A), generating a Cas9 protein that has no endonuclease activity but maintains its RNA-guided DNA-binding capacity, as described in Kabadi and Gersbach, Engineering Synthetic TALE and CRISPR/Cas9 Transcription Factors for Regulating Gene Expression, Methods. 2014 Sep; 69(2): 188-197 and references therein, the contents of which is herein incorporated by reference in its entirety. This deactivated Cas9 (dCas9), in conjunction with the gRNA, can function as a modular DNA-binding scaffold. Both activators and repressors have been generated using dCas9, in which the dCas9 is fused to transactivation domains or repressor domains known in the art. Non-limiting examples of these activators and repressors are described in WIPO Patent Publication No. WO2014197748, the contents of which is herein incorporated by reference in its entirety.

In one embodiment, the CRISPR regulatable element may comprise dCas9 fusion protein further comprising a transactivation or repression domain. In one embodiment, the CRISPR regulatable element may further comprise a guide RNA. In one embodiment, the regulatable element may comprise a dCas9 fusion protein as described in International Publication No. WO2014197748, the contents of which is herein incorporated by reference in its entirety. In some embodiments, the CRISPR regulatable element may comprise a dCas9 fusion protein which is fused to the Krtippel-associated box (KRAB) repressor. In some embodiments, the CRISPR regulatable element may comprise a dCas9 fusion protein which is fused to a transactivation domain selected from VP16, VP64, and NFkappaB p65 transactivation domains or the omega subunit of RNA polymerase.

In one embodiment, the CRISPR regulatable element may comprise a Cas9 fusion protein described in WIPO Patent Publication WO2014089290, the contents of which is herein incorporated by reference in its entirety. In one embodiment, the CRISPR regulatable element may comprise a Cas9 fusion protein described in International Publication No. WO2015070083, the contents of which is herein incorporated in its entirety. In one embodiment, the Cas9 protein may be enzymatically active, or enzymatically inactive, and is operably linked or fused to the payload, as described in WIPO Patent Publication WO2015070083.

In one embodiment, the regulatable-AAV particles of the present invention comprise a system of a Cas9 heterodimer, and a Cas9 guide RNA and/or a dimerizing agent, as described in International Publication WO2016114972, the contents of which are herein incorporated by reference in their entirety. In one embodiment, the Cas9 heterodimer comprises a first and a second fusion polypeptide. The first polypeptide comprises a RuvCI polypeptide, a RuvCII polypeptide, an HNH polypeptide, a RuvCIII polypeptide, a PAM-interacting polypeptide and a first fusion partner that is the first member of a dimerization pair. The second polypeptide of the Cas9 heterodimer comprises an alpha-helical recognition region and a second fusion partner that is the second member of the dimerization pair. In some embodiments, the first or second fusion protein may also comprise a nuclear localization signal (NLS). The Cas9 heterodimer may have a sequence having at least 75% sequence identity to SEQ ID NOs: 1-259; 795-1346; or 1545 of International Publication WO2016114972, the contents of which are herein incorporated by reference in their entirety. In some embodiments, the Cas9 heterodimer is used with a guide RNA that comprises stem loop 1, but does not include stem loop 2 and/or stem loop 3.

In one embodiment, the CRISPR/Cas9 system used with or encapsulated by the viral particles described herein (e.g., AAV particles or CRISPR-AAV particles) is a light inducible CRISPR system. Light inducible CRISPR systems have also been engineered, by fusing the light-inducible heterodimerizing proteins CRY2 and CIB1 from Arabidopsis thaliana to the VP64 transactivation domain at either the N- or C- terminus of dCas9, respectively, as described in Polstein and Gersbach, A light-inducible CRISPR/Cas9 system for control of endogenous gene activation, the contents of which is herein incorporated by reference in its entirety. In one embodiment, the regulatable element of the current invention comprises a dCas9 fusion protein and a transactivating fusion protein, both comprising a light inducible dimerization domain. Any of the light inducible domains described in Jinek et al., A programmable dual-RNA-guided DNA endonuclease in adaptive bacterial immunity, 2012 Aug. 17; 337(6096):816-21, or supra herein, may be used.

In one embodiment, any Cas9 or Cas9 orthologs may be fused to a destabilizing domain and used with or encapsulated by the viral particles described herein (e.g., AAV particles or CRISPR-AAV particles). Non-limiting examples of destabilizing domains include FK506 Binding Protein (FKBP), E. coli dihydrofolate reductase (DHFR), mouse ornithine decarboxylase (MODC), and estrogen receptors (ER).

In one embodiment, the regulatable-AAV particle of the present invention may comprise a CRISPR/Cas9 system that is inducible with doxycycline, as described by de Solis et al (The development of a viral mediated CRISPR/Cas9 system with doxycycline dependent gRNA expression for inducible in vitro and in vivo genome editing; Frontiers Molecular Neuroscience; August 2016; 9:70), the contents of which are herein incorporated by reference in their entirety. In this two-vector system, the viral genome of the first AAV particle comprises an H1/TO or U6/TO promoter for expression of the gRNA as well as a Tet repressor element to regulate the expression of the gRNA in a doxycycline dependent manner and the second AAV particle delivers the Cas9. In another embodiment, it is the Cas9 expression that is regulatable by doxycycline, through the use of a truncated second generation tetracycline response element promoter. In the presence of doxycycline, either the gRNAs or the Cas9 are expressed and gene editing or regulatable expression may occur.

### Suicide Regulatable Elements

In one embodiment, the regulatable element comprises a suicide mechanism.

Several inducible suicide mechanisms are known in the art. One of the best characterized suicide systems is the herpes virus thymidine kinase/ganciclovir system. HSV/Tk phosphorylates the prodrug ganciclovir, which is then further converted by endogenous kinases into its triphosphate form. During replication, DNA polymerases then incorporate ganciclovir-triphosphate into DNA, causing polymerase inhibition and induction of apoptosis, and cell death.

Other inducible suicide systems involve inducible Caspase 9, and enzyme which functions as an executioner in the apoptotic pathway. For example, an inducible Caspase 9 system, iCasp9, was described by Di Stasi, et al. (2011) - Inducible apoptosis as a safety switch for adoptive cell therapy. N Engl J Med 365: 1673-1683. iCasp9 is composed of two inactive parts, fused to the FK506-binding protein FKBP12. Dimerization of the parts is induced by addition of the dimerizing ligand AP1903. Other examples of suicide genes include caspase-8 or cytosine deaminase.

In one embodiment, payload expression is turned off through a suicide mechanism. In one embodiment, the suicide mechanism comprises a regulatable element comprising herpes virus thymidine kinase. In one embodiment, the regulatable element when expressed comprises iCasp9.

In some embodiments, Caspase-9 may be activated using a specific chemical inducer of dimerization (CID) AP20187. In some embodiments, the regulatable element comprises the inducible Caspase-9 described in US Publication No. US20130071414, the contents of which is herein incorporated by reference in its entirety.

### Regulatable Elements: Protein Stability

In some embodiments, the payload expression may be regulated by fusion of a stabilizing or a destabilizing domain. Stabilizing and destabilizing domains which can be used are well known in the art. Non-limiting examples of destabilizing domains include FK506 Binding Protein (FKBP), E. coli dihydrofolate reductase (DHFR), mouse ornithine decarboxylase (MODC), or estrogen receptors (ER). In one embodiment, the destabilizing domain may be from an estrogen receptor.

In some embodiments the destabilizing domain may be inducible. In some embodiments the destabilizing domain may be a “single ligand-single domain,” which allows control of protein stability through a small molecule ligand. In some embodiments the destabilizing domain may be FK506-and rapamycin-binding protein (FKBP12) destabilizing domain, which can be regulated by rapamycin and its analogs, and is unstable in the absence of its ligand. In one embodiment, a point mutant (L106P) of the 107-amino acid protein FKBP confers instability to fusion partners, and this instability is reversed by a synthetic ligand named Shield-1, as described in Banaszynski, L., Chen, L., Maynard-Smith, L. A., Ooi, G. L. and Wandless, T. J. A rapid, reversible, and tunable method to regulate protein function in living cells using synthetic small molecules. Cell 126, 995-1004 (2006), the contents of which is herein incorporated by reference in its entirety.

In another embodiment, the destabilizing domain may be derived from E. coli dihydrofolate reductase. In some embodiments the small molecule trimethoprim (TMP) can bind to the domain and act as a stabilizer, for example, as described in Iwamoto et al. (Chem Biol. 2010 Sep. 24; 17(9):981-8. A general chemical method to regulate protein stability in the mammalian central nervous system) the contents of which is herein incorporated by reference in its entirety. This system has been shown to be applied to regulation of glia cell derived neurotrophic factor (GDNF), as described in Tai et al. (DOI: 10.1371/journal.pone.0046269, Destabilizing Domains Mediate Reversible Transgene Expression in the Brain), the contents of which is herein incorporated by reference in its entirety.

In another embodiment, the destabilizing domain may be a light sensitive degradation domain. In a non-limiting example, the light sensitive degradation domain may be one of the domains described in U.S. Pat. No. 9,115,184, the contents of which is herein incorporated by reference in its entirety. In one embodiment, the domain comprises LOV24.

It is contemplated as part of the invention that any of the destabilizing domains may be combined with any of the enzymes, proteins and fusion proteins described herein.

In one embodiment, a CRISPR/Cas9 or ortholog may be fused to a destabilizing domain. In one embodiment, a CRISPR/Cas9 or ortholog may be fused to a destabilizing domain which can be further regulated by a ligand.

In one embodiment, CRE recombinase may be fused to a destabilizing domain. In one embodiment, the CRE recombinase may be fused to a destabilizing domain which can be further regulated by a ligand.

In one embodiment, FLP recombinase may be fused to a destabilizing domain. In one embodiment, the FLP recombinase may be fused to a destabilizing domain which can be further regulated by a ligand.

In one embodiment, a meganuclease may be fused to a destabilizing domain. In one embodiment, the meganuclease may be fused to a destabilizing domain which can be further regulated by a ligand.

In one embodiment, a serine integrase may be fused to a destabilizing domain. In one embodiment, the serine integrase may fused to a destabilizing domain which can be further regulated by a ligand.

In one embodiment, a zinc finger nuclease may be fused to a destabilizing domain. In one embodiment, the zinc finger nuclease may be fused to a destabilizing domain which can be further regulated by a ligand.

In one embodiment a fusion protein comprising a TAL effector domain may be fused to a destabilizing domain. In one embodiment, the fusion protein comprising a TAL effector domain may be fused to a destabilizing domain which can be further regulated by a ligand.

In one embodiment, one or more fusion proteins described herein comprising DNA binding domains and/or transactivation domains may further comprise a destabilizing domain. In one embodiment, tetracycline transactivator protein may further comprise a destabilizing domain. In another embodiment, a hormone responsive protein or fusion protein may further comprise a destabilizing domain. In one embodiment, an ecdysone inducible fusion protein further comprises a destabilizing domain. In one embodiment, one or more fusion proteins within a rapamycin inducible system may further comprise a destabilizing domain.

### Regulatable Element: RNA Transcripts

Various polynucleotide based methods are also contemplated as useful for regulation of payload expression in certain aspects of the instant invention.

In one embodiment, the regulatable element comprises a polynucleotide which may bind to the transcript encoded by the payload construct.

In one embodiment, the regulatable element comprises one or more interfering RNA sequences. In one embodiment, the regulatable element comprising the one or more interfering RNA sequences functions to temporarily turn off or reduce expression of the payload. In one embodiment, the regulatable element comprising the one or more interfering RNA sequences functions to permanently reduce or turn off expression of the payload. In one embodiment, the regulatable element comprises one or more siRNA sequences. In one embodiment, the regulatable element comprises one or more shRNA sequences. In one embodiment, the regulatable element comprises one or more microRNA sequences. In one embodiment, the payload construct may comprise a microRNA binding site or an siRNA binding site. MicroRNA binding sites may be inserted in the 5′ UTR or the 3′UTR of the payload, or within the coding sequence of the payload. siRNA or shRNA binding sites may be inserted in the 5′UTR or the 3′UTR of the payload or within the payload coding sequence. The payload construct may comprise at least one or more binding sites for a miRNA, siRNA or shRNA. These one or more binding sites may be target sites for the same or for various different microRNAs, siRNAs or shRNAs.

In one embodiment, the payload expression may be regulated by an antisense oligonucleotide. In one embodiment, the regulatable element comprises an antisense oligonucleotide.

In one embodiment, the regulatable element may comprise a ribozyme. In one embodiment, the ribozyme may be a trans-acting hammerhead ribozyme. A ribozyme site may be inserted in the payload construct within the 5′UTR or the 3′UTR of the payload or within the coding sequence.

In one embodiment, the regulatable element may be a riboswitch. In a non-limiting example, the riboswitch may be the inducible guanine-responsive GuaM8HDV aptazyme, which attenuates transgene expression upon a single addition of guanine, as described in Strobel et al., “Riboswitch-mediated Attenuation of Transgene Cytotoxicity Increases Adeno-associated Virus Vector Yields in HEK-293 Cells.” Mol Ther. 2015 Jul. 3, the contents of which is herein incorporated by reference in its entirety.

In one embodiment, the regulatable element comprising at least one polynucleotide, e.g. the siRNA, miRNA, antisense polynucleotide, ribozyme or riboswitch, is driven by a constitutive promoter.

In one embodiment, the regulatable element comprising at least one polynucleotide, e.g. the siRNA, miRNA, antisense polynucleotide, ribozyme or riboswitch, is driven by an inducible promoter.

In one embodiment, the regulatable element comprising at least one polynucleotide, e.g. the siRNA, miRNA, antisense polynucleotide, ribozyme or riboswitch, is driven by a tissue specific promoter.

In one embodiment, the regulatable system may further include additional regulatable elements, which may comprise any of the fusion proteins, enzymes and/or chemical compounds described herein.

As mentioned herein, tissue specific regulation of payload expression may also be mediated through tissue specific microRNAs. In one embodiment, one or more microRNA binding sites may be included in the payload construct to reduce or eliminate payload expression in a particular tissue.

### Regulatable Element: Destabilizing RNA Sequences

Heterologous UTRs or regulatable elements from heterologous UTRs can be incorporated into the UTRs of the payload or the regulatable element. Regulatable elements or sequences within the 5′ and 3′ UTRs will contribute to stabilizing or destabilizing the payload or regulatable element mRNA. For example, the 5′UTRs and 3′UTRs may include translational enhancer elements, which are well known in the art.

In one embodiment, the payload transcript may comprise a destabilizing sequence such as, but not limited to, a 3′UTR with AU-rich elements or AUUUA motifs. Destabilizing sequences are well known in the art and can for example be chosen from those in cytokines, proto-oncogenes, interferon mRNAs or human estrogen receptor alpha.

In one embodiment, the regulatable element may comprise a RNA binding protein, which can stabilize or destabilize the payload transcript.

As another a non-limiting example, the mRNA sequence for any regulatable element, including the inducible fusion proteins, and nucleases, such as cas9 or cas9 orthologs may comprise a destabilizing sequence, such as, but not limited to, a 3′UTR with AU-rich elements or AUUUA motifs. In some embodiments the UTRs employed are heterologous relative to the payload.

### Production of Viral Particles

The present disclosure provides a method for the generation of viral particles, by viral genome replication in a viral replication cell comprising contacting the viral replication cell with a payload construct vector and a viral construct vector.

The present disclosure provides a method for producing a viral particle having enhanced (increased, improved) transduction efficiency comprising the steps of: 1) co-transfecting competent bacterial cells with a bacmid vector and either a viral construct vector and/or payload construct vector, 2) isolating the resultant viral construct vector and payload construct vector and separately transfecting viral replication cells, 3) isolating and purifying resultant payload and viral construct particles comprising viral construct vector or payload construct vector, 4) co-infecting a viral replication cell with both the payload construct vector and viral construct vector, 5) harvesting and purifying the viral particle comprising a parvoviral genome. Production methods are further disclosed in commonly owned and co-pending International Publication No. WO2015191508, the contents of which are herein incorporated by reference in their entirety.

Vectors used in the production of viral particles include those encoding the payload, e.g. payload construct vectors, and those encoding accessory proteins necessary for production, e.g. viral construct vectors.

### Cells

Viral production of the invention disclosed herein describes processes and methods for producing viral particles (e.g., AAV particles and regulatable-AAV particles) that contact a target cell to deliver a payload construct, e.g. a recombinant viral construct, which comprises a nucleotide encoding a payload molecule.

In one embodiment, the viral particles (e.g., AAV particles and regulatable-AAV particles) of the invention may be produced in a viral replication cell that comprises an insect cell.

Growing conditions for insect cells in culture, and production of heterologous products in insect cells in culture are well-known in the art, see U.S. Pat. No. 6,204,059, the contents of which are herein incorporated by reference in their entirety.

Any insect cell which allows for replication of parvovirus and which can be maintained in culture can be used in accordance with the present invention. Cell lines may be used from Spodoptera frugiperda, including, but not limited to the Sf9 or Sf21 cell lines, drosophila cell lines, or mosquito cell lines, such as Aedes albopictus derived cell lines. Use of insect cells for expression of heterologous proteins is well documented, as are methods of introducing nucleic acids, such as vectors, e.g., insect-cell compatible vectors, into such cells and methods of maintaining such cells in culture. See, for example, METHODS IN MOLECULAR BIOLOGY, ed. Richard, Humana Press, N.J. (1995); O'Reilly et al., BACULOVIRUS EXPRESSION VECTORS, A LABORATORY MANUAL, Oxford Univ. Press (1994); Samulski et al., J. Vir. 63:3822-8 (1989); Kajigaya et al., Proc. Nat'l. Acad. Sci. USA 88: 4646-50 (1991); Ruffing et al., J. Vir. 66:6922-30 (1992); Kimbauer et al., Vir. 219:37-44 (1996); Zhao et al., Vir.272:382-93 (2000); and Samulski et al., U.S. Pat. No. 6,204,059, the contents of each of which are herein incorporated by reference in their entirety.

The viral replication cell may be selected from any biological organism, including prokaryotic (e.g., bacterial) cells, and eukaryotic cells, including, insect cells, yeast cells and mammalian cells. Viral replication cells may comprise mammalian cells such as HEK293, A549, WEH1, 3T3, 10T1/2, BHK, MDCK, COS 1, COS 7, BSC 1, BSC 40, BMT 10, VERO. W138, HeLa, HEK293, Saos, C2C12, L cells, HT1080, HepG2 and primary fibroblast, hepatocyte and myoblast cells derived from mammals. Viral replication cells of the invention comprise cells derived from mammalian species including, but not limited to, human, monkey, mouse, rat, rabbit, and hamster or cell type, including but not limited to fibroblast, hepatocyte, tumor cell, cell line transformed cell, etc.

### Small Scale Production

Viral production of the invention disclosed herein describes processes and methods for producing viral particles (e.g., AAV particles and regulatable-AAV particles) that contact a target cell to deliver a payload, e.g. a recombinant viral construct, which comprises a nucleotide encoding a payload.

In one embodiment, the viral particles (e.g., AAV particles and regulatable-AAV particles) of the invention may be produced in a viral replication cell that comprises a mammalian cell.

Viral replication cells commonly used for production of recombinant viral particles (e.g., AAV particles and regulatable-AAV particles) include, but is not limited to HEK293 cells, COS cells, HeLa cells, KB cells, and other mammalian cell lines as described in U.S. Pat. Nos. 6,156,303, 5,387,484, 5,741,683, 5,691,176, and 5,688,676; U.S. Patent Publication 2002/0081721, and International Patent Publications WO 00/47757, WO 00/24916, and WO 96/17947, the contents of each of which are herein incorporated by reference in their entireties.

In one embodiment, viral particles (e.g., AAV particles and regulatable-AAV particles) are produced in mammalian-cells wherein all three VP proteins are expressed at a stoichiometry approaching 1:1:10 (VP1:VP2:VP3). The regulatory mechanisms that allow this controlled level of expression include the production of two mRNAs, one for VP1, and the other for VP2 and VP3, produced by differential splicing.

In another embodiment, viral particles (e.g., AAV particles and regulatable-AAV particles) are produced in mammalian cells using a triple transfection method. As a non-limiting example, the payload construct, parvoviral Rep, and parvoviral Cap are comprised within three different constructs. The triple transfection method of the three components of AAV particle production may be utilized to produce small lots of virus for assays including transduction efficiency, target tissue (tropism) evaluation, and stability. As a non-limiting example, the triple transfection method includes, a payload construct, a rep/cap and a helper.

### Baculovirus

Provided herein are processes and methods for producing viral particles (e.g., AAV particles and regulatable-AAV particles) that contact a target cell to deliver a payload construct which comprises a nucleotide encoding a payload.

Briefly, the viral construct vector and the payload construct vector of the invention are each incorporated by a transposon donor/acceptor system into a bacmid, also known as a baculovirus plasmid, by standard molecular biology techniques known and performed by a person skilled in the art. Transfection of separate viral replication cell populations produces two baculoviruses, one that comprises the viral construct expression vector, and another that comprises the payload construct expression vector. The two baculoviruses may be used to infect a single viral replication cell population for production of particles.

Baculovirus expression vectors for producing viral particles in insect cells, including but not limited to Spodoptera frugiperda (Sf9) cells, provide high titers of viral particle product. Recombinant baculovirus encoding the viral construct expression vector and payload construct expression vector initiates a productive infection of viral replicating cells. Infectious baculovirus particles released from the primary infection secondarily infect additional cells in the culture, exponentially infecting the entire cell culture population in a number of infection cycles that is a function of the initial multiplicity of infection, see Urabe, M. et al. J Virol. 2006 February; 80 (4):1874-85, the contents of which are herein incorporated by reference in their entirety.

Production of particles with baculovirus in an insect cell system may address known baculovirus genetic and physical instability. In one embodiment, the production system of the invention addresses baculovirus instability over multiple passages by utilizing a titerless infected-cells preservation and scale-up system. Small scale seed cultures of viral producing cells are transfected with viral expression constructs encoding the structural, non-structural, components of the viral particle. Baculovirus-infected viral producing cells are harvested into aliquots that may be cryopreserved in liquid nitrogen; the aliquots retain viability and infectivity for infection of large scale viral producing cell culture Wasilko D J et al. Protein Expr Purif. 2009 June; 65(2):122-32, the contents of which are herein incorporated by reference in their entirety.

A genetically stable baculovirus may be used to produce the source of one or more of the components for producing particles in invertebrate cells. In one embodiment, defective baculovirus expression vectors may be maintained episomally in insect cells. In such an embodiment, the bacmid vector is engineered with replication control elements, including but not limited to promoters, enhancers, and/or cell-cycle regulated replication elements.

In one embodiment, baculoviruses may be engineered with a (non-) selectable marker for recombination into the chitinase/cathepsin locus. The chia/v-cath locus is non-essential for propagating baculovirus in tissue culture, and the V-cath (EC 3.4.22.50) is a cysteine endoprotease that is most active on Arg-Arg dipeptide containing substrates. The Arg-Arg dipeptide is present in densovirus and parvovirus capsid structural proteins but infrequently occurs in dependovirus VP1.

In one embodiment, stable viral replication cells permissive for baculovirus infection are engineered with at least one stable integrated copy of any of the elements necessary for AAV replication and viral particle production including, but not limited to, the entire AAV genome, Rep and Cap genes, Rep genes, Cap genes, each Rep protein as a separate transcription cassette, each VP protein as a separate transcription cassette, the AAP (assembly activation protein), or at least one of the baculovirus helper genes with native or non-native promoters.

### Large-Scale Production

In some embodiments, viral particles (e.g., AAV particles and regulatable-AAV particles) production may be modified to increase the scale of production. Large scale viral production methods according to the present invention may include any of those taught in U.S. Pat. Nos. 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference by reference in their entirety. Methods of increasing viral particle production scale typically comprise increasing the number of viral replication cells. In some embodiments, viral replication cells comprise adherent cells. To increase the scale of viral particle production by adherent viral replication cells, larger cell culture surfaces are required. In some cases, large-scale production methods comprise the use of roller bottles to increase cell culture surfaces. Other cell culture substrates with increased surface areas are known in the art. Examples of additional adherent cell culture products with increased surface areas include, but are not limited to CELLSTACK®, CELLCUBE® (Corning Corp., Corning, N.Y.) and NUNC CELL FACTORY™ (Thermo Scientific, Waltham, Mass.) In some cases, large-scale adherent cell surfaces may comprise from about 1,000 cm² to about 100,000 cm². In some cases, large-scale adherent cell cultures may comprise from about 10⁷ to about 10⁹ cells, from about 10⁸ to about 10¹⁰ cells, from about 10⁹ to about 10¹² cells or at least 10¹² cells. In some cases, large-scale adherent cultures may produce from about 10⁹ to about 10¹², from about 10¹⁰ to about 10¹³, from about 10¹¹ to about 10¹⁴, from about 10¹² to about 10¹⁵ or at least 10¹⁵ viral particles.

In some embodiments, large-scale viral production methods of the present invention may comprise the use of suspension cell cultures. Suspension cell culture allows for significantly increased numbers of cells. Typically, the number of adherent cells that can be grown on about 10-50 cm² of surface area can be grown in about 1 cm³ volume in suspension.

Transfection of replication cells in large-scale culture formats may be carried out according to any methods known in the art. For large-scale adherent cell cultures, transfection methods may include, but are not limited to the use of inorganic compounds (e.g. calcium phosphate,) organic compounds (e.g. polyethylenimine (PEI)) or the use of non-chemical methods (e.g. electroporation). With cells grown in suspension, transfection methods may include, but are not limited to the use of calcium phosphate and the use of PEI. In some cases, transfection of large scale suspension cultures may be carried out according to the section entitled “Transfection Procedure” described in Feng, L. et al., 2008. Biotechnol Appl. Biochem. 50:121-32, the contents of which are herein incorporated by reference in their entirety. According to such embodiments, PEI-DNA complexes may be formed for introduction of plasmids to be transfected. In some cases, cells being transfected with PEI-DNA complexes may be ‘shocked’ prior to transfection. This comprises lowering cell culture temperatures to 4° C. for a period of about 1 hour. In some cases, cell cultures may be shocked for a period of from about 10 minutes to about 5 hours. In some cases, cell cultures may be shocked at a temperature of from about 0° C. to about 20° C.

In some cases, transfections may include one or more vectors for expression of an RNA effector molecule to reduce expression of nucleic acids expression from one or more viral genomes. Such methods may enhance the production of viral particles by reducing cellular resources wasted on expressing payload constructs. In some cases, such methods may be carried according to those taught in US Publication No. US2014/0099666, the contents of which are herein incorporated by reference in their entirety.

### Bioreactors

In some embodiments, cell culture bioreactors may be used for large scale viral production. In some cases, bioreactors comprise stirred tank reactors. Such reactors generally comprise a vessel, typically cylindrical in shape, with a stirrer (e.g. impeller.) In some embodiments, such bioreactor vessels may be placed within a water jacket to control vessel temperature and/or to minimize effects from ambient temperature changes. Bioreactor vessel volume may range in size from about 500 ml to about 2 L, from about 1 L to about 5 L, from about 2.5 L to about 20 L, from about 10 L to about 50 L, from about 25 L to about 100 L, from about 75 L to about 500 L, from about 250 L to about 2,000 L, from about 1,000 L to about 10,000 L, from about 5,000 L to about 50,000 L or at least 50,000 L. Vessel bottoms may be rounded or flat. In some cases, animal cell cultures may be maintained in bioreactors with rounded vessel bottoms.

In some cases, bioreactor vessels may be warmed through the use of a thermocirculator. Thermocirculators pump heated water around water jackets. In some cases, heated water may be pumped through pipes (e.g. coiled pipes) that are present within bioreactor vessels. In some cases, warm air may be circulated around bioreactors, including, but not limited to air space directly above culture medium. Additionally, pH and CO₂ levels may be maintained to optimize cell viability.

In some cases, bioreactors may comprise hollow-fiber reactors. Hollow-fiber bioreactors may support the culture of both anchorage dependent and anchorage independent cells. Further bioreactors may include, but are not limited to packed-bed or fixed-bed bioreactors. Such bioreactors may comprise vessels with glass beads for adherent cell attachment. Further packed-bed reactors may comprise ceramic beads.

In some cases, viral particles are produced through the use of a disposable bioreactor. In some embodiments, such bioreactors may include WAVE disposable bioreactors.

In some embodiments, viral particle (e.g., AAV particles and regulatable-AAV particles) production in animal cell bioreactor cultures may be carried out according to the methods taught in U.S. Pat. Nos. 5,064764, 6,194,191, 6,566,118, 8,137,948 or US Patent Publication No. US2011/0229971, the contents of each of which are herein incorporated by reference in their entirety.

### Cell Lysis

Cells of the invention, including, but not limited to viral production cells, may be subjected to cell lysis according to any methods known in the art. Cell lysis may be carried out to obtain one or more agents (e.g. viral particles) present within any cells of the invention. In some embodiments, cell lysis may be carried out according to any of the methods listed in U.S. Pat. Nos. 7,326,555, 7,579,181, 7,048,920, 6,410,300, 6,436,394, 7,732,129, 7,510,875, 7,445,930, 6,726,907, 6,194,191, 7,125,706, 6,995,006, 6,676,935, 7,968,333, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference in their entirety. Cell lysis methods may be chemical or mechanical. Chemical cell lysis typically comprises contacting one or more cells with one or more lysis agent. Mechanical lysis typically comprises subjecting one or more cells to one or more lysis conditions and/or one or more lysis forces.

In some embodiments, chemical lysis may be used to lyse cells. As used herein, the term “lysis agent” refers to any agent that may aid in the disruption of a cell. In some cases, lysis agents are introduced in solutions, termed lysis solutions or lysis buffers. As used herein, the term “lysis solution” refers to a solution (typically aqueous) comprising one or more lysis agents. In addition to lysis agents, lysis solutions may include one or more buffering agents, solubilizing agents, surfactants, preservatives, cryoprotectants, enzymes, enzyme inhibitors and/or chelators. Lysis buffers are lysis solutions comprising one or more buffering agent. Additional components of lysis solutions may include one or more solubilizing agents. As used herein, the term “solubilizing agent” refers to a compound that enhances the solubility of one or more components of a solution and/or the solubility of one or more entities to which solutions are applied. In some cases, solubilizing agents enhance protein solubility. In some cases, solubilizing agents are selected based on their ability to enhance protein solubility while maintaining protein conformation and/or activity.

Exemplary lysis agents may include any of those described in U.S. Pat. Nos. 8,685,734, 7,901,921, 7,732,129, 7,223,585, 7,125,706, 8,236,495, 8,110,351, 7,419,956, 7,300,797, 6,699,706 and 6,143,567, the contents of each of which are herein incorporated by reference in their entirety. In some cases, lysis agents may be selected from lysis salts, amphoteric agents, cationic agents, ionic detergents and non-ionic detergents. Lysis salts may include, but are not limited to sodium chloride (NaCl) and potassium chloride (KCl) Further lysis salts may include any of those described in U.S. Pat. Nos. 8,614,101, 7,326,555, 7,579,181, 7,048,920, 6,410,300, 6,436,394, 7,732,129, 7,510,875, 7,445,930, 6,726,907, 6,194,191, 7,125,706, 6,995,006, 6,676,935 and 7,968,333, the contents of each of which are herein incorporated by reference in their entirety. Concentrations of salts may be increased or decreased to obtain an effective concentration for rupture of cell membranes. Amphoteric agents, as referred to herein, are compounds capable of reacting as an acid or a base. Amphoteric agents may include, but are not limited to lysophosphatidylcholine, 3-((3-Cholamidopropyl) dimethylammonium)-1-propanesulfonate (CHAPS,) ZWITTERGENT® and the like. Cationic agents may include, but are not limited to cetyltrimethylammonium bromide (C (16) TAB) and Benzalkonium chloride. Lysis agents comprising detergents may include ionic detergents or non-ionic detergents. Detergents may function to break apart or dissolve cell structures including, but not limited to cell membranes, cell walls, lipids, carbohydrates, lipoproteins and glycoproteins. Exemplary ionic detergents include any of those taught in U.S. Pat. Nos. 7,625,570 and 6,593,123 or US Publication No. US2014/0087361, the contents of each of which are herein incorporated by reference in their entirety. Some ionic detergents may include, but are not limited to sodium dodecyl sulfate (SDS), cholate and deoxycholate. In some cases, ionic detergents may be included in lysis solutions as a solubilizing agent. Non-ionic detergents may include, but are not limited to octylglucoside, digitonin, lubrol, C12E8, TWEEN®-20, TWEEN®-80, Triton X-100 and Noniodet P-40. Non-ionic detergents are typically weaker lysis agents, but may be included as solubilizing agents for solubilizing cellular and/or viral proteins. Further lysis agents may include enzymes and urea. In some cases, one or more lysis agents may be combined in a lysis solution in order to enhance one or more of cell lysis and protein solubility. In some cases, enzyme inhibitors may be included in lysis solutions in order to prevent proteolysis that may be triggered by cell membrane disruption.

In some embodiments, mechanical cell lysis is carried out. Mechanical cell lysis methods may include the use of one or more lysis condition and/or one or more lysis force. As used herein, the term “lysis condition” refers to a state or circumstance that promotes cellular disruption. Lysis conditions may comprise certain temperatures, pressures, osmotic purity, salinity and the like. In some cases, lysis conditions comprise increased or decreased temperatures. According to some embodiments, lysis conditions comprise changes in temperature to promote cellular disruption. Cell lysis carried out according to such embodiments may include freeze-thaw lysis. As used herein, the term “freeze-thaw lysis” refers to cellular lysis in which a cell solution is subjected to one or more freeze-thaw cycle. According to freeze-thaw lysis methods, cells in solution are frozen to induce a mechanical disruption of cellular membranes caused by the formation and expansion of ice crystals. Cell solutions used according to freeze-thaw lysis methods, may further comprise one or more lysis agents, solubilizing agents, buffering agents, cryoprotectants, surfactants, preservatives, enzymes, enzyme inhibitors and/or chelators. Once cell solutions subjected to freezing are thawed, such components may enhance the recovery of desired cellular products. In some cases, one or more cyroprotectants are included in cell solutions undergoing freeze-thaw lysis. As used herein, the term “cryoprotectant” refers to an agent used to protect one or more substance from damage due to freezing. Cryoprotectants of the invention may include any of those taught in US Publication No. US2013/0323302 or U.S. Pat. Nos. 6,503,888, 6,180,613, 7,888,096, 7,091,030, the contents of each of which are herein incorporated by reference in their entirety. In some cases, cryoprotectants may include, but are not limited to dimethyl sulfoxide, 1,2-propanediol, 2,3-butanediol, formamide, glycerol, ethylene glycol, 1,3-propanediol and n-dimethyl formamide, polyvinylpyrrolidone, hydroxyethyl starch, agarose, dextrans, inositol, glucose, hydroxyethylstarch, lactose, sorbitol, methyl glucose, sucrose and urea. In some embodiments, freeze-thaw lysis may be carried out according to any of the methods described in U.S. Pat. No. 7,704,721, the contents of which are herein incorporated by reference in their entirety.

As used herein, the term “lysis force” refers to a physical activity used to disrupt a cell. Lysis forces may include, but are not limited to mechanical forces, sonic forces, gravitational forces, optical forces, electrical forces and the like. Cell lysis carried out by mechanical force is referred to herein as “mechanical lysis.” Mechanical forces that may be used according to mechanical lysis may include high shear fluid forces. According to such methods of mechanical lysis, a microfluidizer may be used. Microfluidizers typically comprise an inlet reservoir where cell solutions may be applied. Cell solutions may then be pumped into an interaction chamber via a pump (e.g. high-pressure pump) at high speed and/or pressure to produce shear fluid forces. Resulting lysates may then be collected in one or more output reservoirs. Pump speed and/or pressure may be adjusted to modulate cell lysis and enhance recovery of products (e.g. viral particles.) Other mechanical lysis methods may include physical disruption of cells by scraping.

Cell lysis methods may be selected based on the cell culture format of cells to be lysed. For example, with adherent cell cultures, some chemical and mechanical lysis methods may be used. Such mechanical lysis methods may include freeze-thaw lysis or scraping. In another example, chemical lysis of adherent cell cultures may be carried out through incubation with lysis solutions comprising surfactant, such as Triton-X-100. In some cases, cell lysates generated from adherent cell cultures may be treated with one more nucleases to lower the viscosity of the lysates caused by liberated DNA.

In one embodiment, a method for harvesting viral particles (e.g., AAV particles and regulatable-AAV particles) without lysis may be used for efficient and scalable viral particle production. In a non-limiting example, viral particles (e.g., AAV particles and regulatable-AAV particles) may be produced by culturing a viral particle lacking a heparin binding site, thereby allowing the viral particle to pass into the supernatant, in a cell culture, collecting supernatant from the culture; and isolating the viral particle from the supernatant, as described in US Patent Publication No. 20090275107, the contents of which is incorporated herein by reference in its entirety.

### Clarification

Cell lysates comprising viral particles (e.g., AAV particles and regulatable-AAV particles) may be subjected to clarification. Clarification refers to initial steps taken in purification of viral particles from cell lysates. Clarification serves to prepare lysates for further purification by removing larger, insoluble debris. Clarification steps may include, but are not limited to centrifugation and filtration. During clarification, centrifugation may be carried out at low speeds to remove larger debris, only. Similarly, filtration may be carried out using filters with larger pore sizes so that only larger debris is removed. In some cases, tangential flow filtration may be used during clarification. Objectives of viral clarification include high throughput processing of cell lysates and to optimize ultimate viral recovery. Advantages of including a clarification step include scalability for processing of larger volumes of lysate. In some embodiments, clarification may be carried out according to any of the methods presented in U.S. Pat. Nos. 8,524,446, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498, 7,491,508, US Publication Nos. US2013/0045186, US2011/0263027, US2011/0151434, US2003/0138772, and International Publication Nos. WO2002012455, WO1996039530, WO1998010088, WO1999014354, WO1999015685, WO1999047691, WO2000055342, WO2000075353 and WO2001023597, the contents of each of which are herein incorporated by reference in their entirety.

Methods of cell lysate clarification by filtration are well understood in the art and may be carried out according to a variety of available methods including, but not limited to passive filtration and flow filtration. Filters used may comprise a variety of materials and pore sizes. For example, cell lysate filters may comprise pore sizes of from about 1 μM to about 5 μM, from about 0.5 μM to about 2 μM, from about 0.1 μM to about 1 μM, from about 0.05 μM to about 0.05 μM and from about 0.001 μM to about 0.1 μM. Exemplary pore sizes for cell lysate filters may include, but are not limited to, 2.0, 1.9, 1.8, 1.7, 1.6, 1.5, 1.4, 1.3, 1.2, 1.1, 1, 0.9, 0.8, 0.7, 0.6, 0.5, 0.4, 0.3, 0.2, 0.1, 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, 0.05, 0.22, 0.21, 0.20, 0.19, 0.18, 0.17, 0.16, 0.15, 0.14, 0.13, 0.12, 0.11, 0.1, 0.09, 0.08, 0.07, 0.06, 0.05, 0.04, 0.03, 0.02, 0.01, 0.02, 0.019, 0.018, 0.017, 0.016, 0.015, 0.014, 0.013, 0.012, 0.011, 0.01, 0.009, 0.008, 0.007, 0.006, 0.005, 0.004, 0.003, 0.002, 0.001 and 0.001 μM. In one embodiment, clarification may comprise filtration through a filter with 2.0 μM pore size to remove large debris, followed by passage through a filter with 0.45 μM pore size to remove intact cells.

Filter materials may be composed of a variety of materials. Such materials may include, but are not limited to polymeric materials and metal materials (e.g. sintered metal and pored aluminum.) Exemplary materials may include, but are not limited to nylon, cellulose materials (e.g. cellulose acetate), polyvinylidene fluoride (PVDF), polyethersulfone, polyamide, polysulfone, polypropylene, and polyethylene terephthalate. In some cases, filters useful for clarification of cell lysates may include, but are not limited to ULTIPLEAT PROFILE™ filters (Pall Corporation, Port Washington, N.Y.), SUPOR™ membrane filters (Pall Corporation, Port Washington, N.Y.)

In some cases, flow filtration may be carried out to increase filtration speed and/or effectiveness. In some cases, flow filtration may comprise vacuum filtration. According to such methods, a vacuum is created on the side of the filter opposite that of cell lysate to be filtered. In some cases, cell lysates may be passed through filters by centrifugal forces. In some cases, a pump is used to force cell lysate through clarification filters. Flow rate of cell lysate through one or more filters may be modulated by adjusting channel size and/or fluid pressure.

According to some embodiments, cell lysates may be clarified by centrifugation. Centrifugation may be used to pellet insoluble particles in the lysate. During clarification, centrifugation strength [expressed in terms of gravitational units (g), which represents multiples of standard gravitational force] may be lower than in subsequent purification steps. In some cases, centrifugation may be carried out on cell lysates at from about 200 g to about 800 g, from about 500 g to about 1500 g, from about 1000 g to about 5000 g, from about 1200 g to about 10000 g or from about 8000 g to about 15000 g. In some embodiments, cell lysate centrifugation is carried out at 8000 g for 15 minutes. In some cases, density gradient centrifugation may be carried out in order to partition particulates in the cell lysate by sedimentation rate. Gradients used according to methods of the present invention may include, but are not limited to cesium chloride gradients and iodixanol step gradients.

### Purification: Chromatography

In some embodiments, viral particles (e.g., AAV particles and regulatable-AAV particles) may be purified from clarified cell lysates by one or more methods of chromatography. Chromatography refers to any number of methods known in the art for separating out one or more elements from a mixture. Such methods may include, but are not limited to ion exchange chromatography (e.g. cation exchange chromatography and anion exchange chromatography), immunoaffinity chromatography and size-exclusion chromatography. In some embodiments, methods of viral chromatography may include any of those taught in U.S. Pat. Nos. 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference by reference in their entirety.

In some embodiments, ion exchange chromatography may be used to isolate viral particles. Ion exchange chromatography is used to bind viral particles based on charge-charge interactions between capsid proteins and charged sites present on a stationary phase, typically a column through which viral preparations (e.g. clarified lysates) are passed. After application of viral preparations, bound viral particles may then be eluted by applying an elution solution to disrupt the charge-charge interactions. Elution solutions may be optimized by adjusting salt concentration and/or pH to enhance recovery of bound viral particles. Depending on the charge of viral capsids being isolated, cation or anion exchange chromatography methods may be selected. Methods of ion exchange chromatography may include, but are not limited to any of those taught in U.S. Pat. Nos. 7,419,817, 6,143,548, 7,094,604, 6,593,123, 7,015,026 and 8,137,948, the contents of each of which are herein incorporated by reference in their entirety.

In some embodiments, immunoaffinity chromatography may be used. Immunoaffinity chromatography is a form of chromatography that utilizes one or more immune compounds (e.g. antibodies or antibody-related structures) to retain viral particles. Immune compounds may bind specifically to one or more structures on viral particle surfaces, including, but not limited to one or more viral coat proteins. In some cases, immune compounds may be specific for a particular viral variant. In some cases, immune compounds may bind to multiple viral variants. In some embodiments, immune compounds may include recombinant single-chain antibodies. Such recombinant single chain antibodies may include those described in Smith, R. H. et al., 2009. Mol. Ther. 17(11):1888-96, the contents of which are herein incorporated by reference in their entirety. Such immune compounds are capable of binding to several AAV capsid variants, including, but not limited to AAV1, AAV2, AAV6 and AAV8.

In some embodiments, size-exclusion chromatography (SEC) may be used. SEC may comprise the use of a gel to separate particles according to size. In viral particle purification, SEC filtration is sometimes referred to as “polishing.” In some cases, SEC may be carried out to generate a final product that is near-homogenous. Such final products may in some cases be used in pre-clinical studies and/or clinical studies (Kotin, R. M. 2011. Human Molecular Genetics. 20(1):R2-R6, the contents of which are herein incorporated by reference in their entirety.) In some cases, SEC may be carried out according to any of the methods taught in U.S. Pat. Nos. 6,143,548, 7,015,026, 8,476,418, 6,410,300, 8,476,418, 7,419,817, 7,094,604, 6,593,123, and 8,137,948, the contents of each of which are herein incorporated by reference in their entirety.

In one embodiment, the compositions comprising at least one viral particle (e.g., AAV particle and regulatable-AAV particle) may be isolated or purified using the methods described in U.S. Pat. No. 6,146,874, the contents of which are herein incorporated by reference in its entirety.

In one embodiment, the compositions comprising at least one re viral particle (e.g., AAV particle and regulatable-AAV particle) may be isolated or purified using the methods described in U.S. Pat. No. 6,660,514, the contents of which are herein incorporated by reference in its entirety.

In one embodiment, the compositions comprising at least one viral particle (e.g., AAV particle and regulatable-AAV particle) may be isolated or purified using the methods described in U.S. Pat. No. 8,283,151, the contents of which are herein incorporated by reference in its entirety.

In one embodiment, the compositions comprising at least one viral particle (e.g., AAV particle and regulatable-AAV particle) may be isolated or purified using the methods described in U.S. Pat. No. 8,524,446, the contents of which are herein incorporated by reference in its entirety.

### Formulation and Delivery

According to the present invention the viral particles (e.g., AAV particles or regulatable-AAV particles) may be prepared as pharmaceutical compositions. It will be understood that such compositions necessarily comprise one or more active ingredients and, most often, a pharmaceutically acceptable excipient.

Relative amounts of the active ingredient (e.g. AAV particle or regulatable-AAV particle), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

In some embodiments, the viral particles (e.g., AAV particle or regulatable-AAV particle) pharmaceutical compositions described herein may comprise at least one payload. As a non-limiting example, the pharmaceutical compositions may contain a viral particle with 1, 2, 3, 4 or 5 payloads.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, rats, birds, including commercially relevant birds such as poultry, chickens, ducks, geese, and/or turkeys.

In some embodiments, compositions are administered to humans, human patients or subjects.

In some embodiments, the chemical agent (e.g., drug, compound or ligand) may be formulated for delivery. The delivery compositions may comprise a pharmaceutically acceptable carrier or excipient and optionally a suitable adjuvant and may be administered via routes described in the art including but not limited to inhalation, insufflation, oral, buccal, parenteral, rectal, or transdermal.

In one embodiment, the present invention also provides pharmaceutical compositions comprising at least one viral particle (e.g., AAV particle or regulatable-AAV particle) having a regulatable element and a pharmaceutically acceptable excipient. In some aspects, one or more regulatable elements are contained in a viral particle (e.g., AAV particle or regulatable-AAV particle).

### Formulations

The viral particles (e.g., AAV particles or regulatable-AAV particles) of the invention can be formulated using one or more excipients to: (1) increase stability; (2) increase cell transfection or transduction; (3) permit the sustained or delayed expression of the payload; (4) alter the biodistribution (e.g., target the viral particle to specific tissues or cell types); (5) increase the translation of encoded protein; (6) alter the release profile of encoded protein and/or (7) allow for regulatable expression of the payload.

Formulations of the present invention can include, without limitation, saline, liposomes, lipid nanoparticles, polymers, peptides, proteins, cells transfected with viral genomes (e.g., for transfer or transplantation into a subject) and combinations thereof

Formulations of the pharmaceutical compositions described herein may be prepared by any method known or hereafter developed in the art of pharmacology. As used herein the term “pharmaceutical composition” refers to compositions comprising at least one active ingredient and optionally one or more pharmaceutically acceptable excipients.

In general, such preparatory methods include the step of associating the active ingredient with an excipient and/or one or more other accessory ingredients. As used herein, the phrase “active ingredient” generally refers either to a viral particle (e.g., AAV particle or regulatable-AAV particle) carrying a payload region encoding the polypeptides of the invention.

Formulations of the viral particles (e.g., AAV particles or regulatable-AAV particles) and pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

In one embodiment, the viral particle (e.g., AAV particle or regulatable-AAV particle) of the invention may be formulated in PBS with 0.001% of pluronic acid (F-68) at a pH of about 7.0.

In some embodiments, the formulations described herein may contain sufficient viral particles (e.g., AAV particles or regulatable-AAV particles) for expression of at least one expressed payload. As a non-limiting example, the viral particles (e.g., AAV particles or regulatable-AAV particles) may contain viral genomes encoding 1, 2, 3, 4 or 5 payloads. In one embodiment, the formulation may contain a payload encoding proteins selected from categories such as, but not limited to, human proteins, veterinary proteins, bacterial proteins, biological proteins, antibodies, immunogenic proteins, therapeutic peptides and proteins, secreted proteins, plasma membrane proteins, cytoplasmic and cytoskeletal proteins, intracellular membrane bound proteins, nuclear proteins, proteins associated with human disease and/or proteins associated with non-human diseases.

According to the present invention, viral particles (e.g., AAV particles or regulatable-AAV particles) may be formulated for CNS delivery. Agents that cross the brain blood barrier may be used. For example, some cell penetrating peptides that can target molecules to the brain blood barrier endothelium may be used for formulation (e.g., Mathupala, Expert Opin Ther Pat., 2009, 19, 137-140; the content of which is incorporated herein by reference in its entirety).

In one embodiment, the viral particle may also encode a cell penetrating peptide. Delivery of a fusion protein comprising a recombinase and a cell penetrating peptide, the use of which is contemplated in one aspect of the current invention, is described in US Publication No. US20140127162, the contents of which is herein incorporated by reference in its entirety. Cell penetrating peptides are well known in the art and are for example described in Lundberg et al. (2002) A brief introduction to cell-penetrating peptides, J. Mol. Recognit. 16: 227-233, the contents of which is herein incorporated by reference in its entirety.

### Excipients and Diluents

The viral particles (e.g., AAV particles or regulatable-AAV particles) of the invention can be formulated using one or more excipients or diluents to (1) increase stability; (2) increase cell transfection or transduction; (3) permit the sustained or delayed release; (4) alter the biodistribution (e.g., target the viral particle to specific tissues or cell types); (5) increase the translation of encoded protein in vivo; (6) alter the release profile of encoded protein in vivo and/or (7) allow for regulatable expression of the polypeptides of the invention.

In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

### Inactive Ingredients

In some embodiments, viral particle (e.g., AAV particle or regulatable-AAV particle) formulations may comprise at least one inactive ingredient. As used herein, the term “inactive ingredient” refers to one or more agents that do not contribute to the activity of the active ingredient of the pharmaceutical composition included in formulations. In some embodiments, all, none or some of the inactive ingredients which may be used in the formulations of the present invention may be approved by the US Food and Drug Administration (FDA).

In one embodiment, the viral particle pharmaceutical compositions comprise at least one inactive ingredient such as, but not limited to, 1,2,6-Hexanetriol; 1,2-Dimyristoyl-Sn-Glycero-3-(Phospho-S-(1-Glycerol)); 1,2-Dimyristoyl-Sn-Glycero-3-Phosphocholine; 1,2-Dioleoyl-Sn-Glycero-3-Phosphocholine; 1,2-Dipalmitoyl-Sn-Glycero-3-(Phospho-Rac-(1-Glycerol)); 1,2-Distearoyl-Sn-Glycero-3-(Phospho-Rac-(1-Glycerol)); 1,2-Distearoyl-Sn-Glycero-3-Phosphocholine; 1-0-Tolylbiguanide; 2-Ethyl-1,6-Hexanediol; Acetic Acid; Acetic Acid, Glacial; Acetic Anhydride; Acetone; Acetone Sodium Bisulfite; Acetylated Lanolin Alcohols; Acetylated Monoglycerides; Acetylcysteine; Acetyltryptophan, DL-; Acrylates Copolymer; Acrylic Acid-Isooctyl Acrylate Copolymer; Acrylic Adhesive 788; Activated Charcoal; Adcote 72A103; Adhesive Tape; Adipic Acid; Aerotex Resin 3730; Alanine; Albumin Aggregated; Albumin Colloidal; Albumin Human; Alcohol; Alcohol, Dehydrated; Alcohol, Denatured; Alcohol, Diluted; Alfadex; Alginic Acid; Alkyl Ammonium Sulfonic Acid Betaine; Alkyl Aryl Sodium Sulfonate; Allantoin; Allyl .Alpha.-Ionone; Almond Oil; Alpha-Terpineol; Alpha-Tocopherol; Alpha-Tocopherol Acetate, Dl-; Alpha-Tocopherol, Dl-; Aluminum Acetate; Aluminum Chlorhydroxy Allantoinate; Aluminum Hydroxide; Aluminum Hydroxide - Sucrose, Hydrated; Aluminum Hydroxide Gel; Aluminum Hydroxide Gel F 500; Aluminum Hydroxide Gel F 5000; Aluminum Monostearate; Aluminum Oxide; Aluminum Polyester; Aluminum Silicate; Aluminum Starch Octenylsuccinate; Aluminum Stearate; Aluminum Subacetate; Aluminum Sulfate Anhydrous; Amerchol C; Amerchol-Cab; Aminomethylpropanol; Ammonia; Ammonia Solution; Ammonia Solution, Strong; Ammonium Acetate; Ammonium Hydroxide; Ammonium Lauryl Sulfate; Ammonium Nonoxynol-4 Sulfate; Ammonium Salt Of C-12-C-15 Linear Primary Alcohol Ethoxylate; Ammonium Sulfate; Ammonyx; Amphoteric-2; Amphoteric-9; Anethole; Anhydrous Citric Acid; Anhydrous Dextrose; Anhydrous Lactose; Anhydrous Trisodium Citrate; Aniseed Oil; Anoxid Sbn; Antifoam; Antipyrine; Apaflurane; Apricot Kernel Oil Peg-6 Esters; Aquaphor; Arginine; Arlacel; Ascorbic Acid; Ascorbyl Palmitate; Aspartic Acid; Balsam Peru; Barium Sulfate; Beeswax; Beeswax, Synthetic; Beheneth-10; Bentonite; Benzalkonium Chloride; Benzenesulfonic Acid; Benzethonium Chloride; Benzododecinium Bromide; Benzoic Acid; Benzyl Alcohol; Benzyl Benzoate; Benzyl Chloride; Betadex; Bibapcitide; Bismuth Subgallate; Boric Acid; Brocrinat; Butane; Butyl Alcohol; Butyl Ester Of Vinyl Methyl Ether/Maleic Anhydride Copolymer (125000 Mw); Butyl Stearate; Butylated Hydroxyanisole; Butylated Hydroxytoluene; Butylene Glycol; Butylparaben; Butyric Acid; C20-40 Pareth-24; Caffeine; Calcium; Calcium Carbonate; Calcium Chloride; Calcium Gluceptate; Calcium Hydroxide; Calcium Lactate; Calcobutrol; Caldiamide Sodium; Caloxetate Trisodium; Calteridol Calcium; Canada Balsam; Caprylic/Capric Triglyceride; Caprylic/Capric/Stearic Triglyceride; Captan; Captisol; Caramel; Carbomer 1342; Carbomer 1382; Carbomer 934; Carbomer 934p; Carbomer 940; Carbomer 941; Carbomer 980; Carbomer 981; Carbomer Homopolymer Type B (Allyl Pentaerythritol Crosslinked); Carbomer Homopolymer Type C (Allyl Pentaerythritol Crosslinked); Carbon Dioxide; Carboxy Vinyl Copolymer; Carboxymethylcellulose; Carboxymethylcellulose Sodium; Carboxypolymethylene; Carrageenan; Carrageenan Salt; Castor Oil; Cedar Leaf Oil; Cellulose; Cellulose, Microcrystalline; Cerasynt-Se; Ceresin; Ceteareth-12; Ceteareth-15; Ceteareth-30; Cetearyl Alcohol/Ceteareth-20; Cetearyl Ethylhexanoate; Ceteth-10; Ceteth-2; Ceteth-20; Ceteth-23; Cetostearyl Alcohol; Cetrimonium Chloride; Cetyl Alcohol; Cetyl Esters Wax; Cetyl Palmitate; Cetylpyridinium Chloride; Chlorobutanol; Chlorobutanol Hemihydrate; Chlorobutanol, Anhydrous; Chlorocresol; Chloroxylenol; Cholesterol; Choleth; Choleth-24; Citrate; Citric Acid; Citric Acid Monohydrate; Citric Acid, Hydrous; Cocamide Ether Sulfate; Cocamine Oxide; Coco Betaine; Coco Diethanolamide; Coco Monoethanolamide; Cocoa Butter; Coco-Glycerides; Coconut Oil; Coconut Oil, Hydrogenated; Coconut Oil/Palm Kernel Oil Glycerides, Hydrogenated; Cocoyl Caprylocaprate; Cola Nitida Seed Extract; Collagen; Coloring Suspension; Corn Oil; Cottonseed Oil; Cream Base; Creatine; Creatinine; Cresol; Croscarmellose Sodium; Crospovidone; Cupric Sulfate; Cupric Sulfate Anhydrous; Cyclomethicone; Cyclomethicone/Dimethicone Copolyol; Cysteine; Cysteine Hydrochloride; Cysteine Hydrochloride Anhydrous; Cysteine, Dl-; D&C Red No. 28; D&C Red No. 33; D&C Red No. 36; D&C Red No. 39; D&C Yellow No. 10; Dalfampridine; Daubert 1-5 Pestr (Matte) 164z; Decyl Methyl Sulfoxide; Dehydag Wax Sx; Dehydroacetic Acid; Dehymuls E; Denatonium Benzoate; Deoxycholic Acid; Dextran; Dextran 40; Dextrin; Dextrose; Dextrose Monohydrate; Dextrose Solution; Diatrizoic Acid; Diazolidinyl Urea; Dichlorobenzyl Alcohol; Dichlorodifluoromethane; Dichlorotetrafluoroethane; Diethanolamine; Diethyl Pyrocarbonate; Diethyl Sebacate; Diethylene Glycol Monoethyl Ether; Diethylhexyl Phthalate; Dihydroxyaluminum Aminoacetate; Diisopropanolamine; Diisopropyl Adipate; Diisopropyl Dilinoleate; Dimethicone 350; Dimethicone Copolyol; Dimethicone Mdx4-4210; Dimethicone Medical Fluid 360; Dimethyl Isosorbide; Dimethyl Sulfoxide; Dimethylaminoethyl Methacrylate-Butyl Methacrylate-Methyl Methacrylate Copolymer; Dimethyldioctadecylammonium Bentonite; Dimethylsiloxane/Methylvinylsiloxane Copolymer; Dinoseb Ammonium Salt; Dipalmitoylphosphatidylglycerol, Dl-; Dipropylene Glycol; Disodium Cocoamphodiacetate; Disodium Laureth Sulfosuccinate; Disodium Lauryl Sulfosuccinate; Disodium Sulfosalicylate; Disofenin; Divinylbenzene Styrene Copolymer; Dmdm Hydantoin; Docosanol; Docusate Sodium; Duro-Tak 280-2516; Duro-Tak 387-2516; Duro-Tak 80-1196; Duro-Tak 87-2070; Duro-Tak 87-2194; Duro-Tak 87-2287; Duro-Tak 87-2296; Duro-Tak 87-2888; Duro-Tak 87-2979; Edetate Calcium Disodium; Edetate Disodium; Edetate Disodium Anhydrous; Edetate Sodium; Edetic Acid; Egg Phospholipids; Entsufon; Entsufon Sodium; Epilactose; Epitetracycline Hydrochloride; Essence Bouquet 9200; Ethanolamine Hydrochloride; Ethyl Acetate; Ethyl Oleate; Ethylcelluloses; Ethylene Glycol; Ethylene Vinyl Acetate Copolymer; Ethylenediamine; Ethylenediamine Dihydrochloride; Ethylene-Propylene Copolymer; Ethylene-Vinyl Acetate Copolymer (28% Vinyl Acetate); Ethylene-Vinyl Acetate Copolymer (9% Vinylacetate); Ethylhexyl Hydroxystearate; Ethylparaben; Eucalyptol; Exametazime; Fat, Edible; Fat, Hard; Fatty Acid Esters; Fatty Acid Pentaerythriol Ester; Fatty Acids; Fatty Alcohol Citrate; Fatty Alcohols; Fd&C Blue No. 1; Fd&C Green No. 3; Fd&C Red No. 4; Fd&C Red No. 40; Fd&C Yellow No. 10 (Delisted); Fd&C Yellow No. 5; Fd&C Yellow No. 6; Ferric Chloride; Ferric Oxide; Flavor 89-186; Flavor 89-259; Flavor Df-119; Flavor Df-1530; Flavor Enhancer; Flavor Fig 827118; Flavor Raspberry Pfc-8407; Flavor Rhodia Pharmaceutical No. Rf 451; Fluorochlorohydrocarbons; Formaldehyde; Formaldehyde Solution; Fractionated Coconut Oil; Fragrance 3949-5; Fragrance 520a; Fragrance 6.007; Fragrance 91-122; Fragrance 9128-Y; Fragrance 93498g; Fragrance Balsam Pine No. 5124; Fragrance Bouquet 10328; Fragrance Chemoderm 6401-B; Fragrance Chemoderm 6411; Fragrance Cream No. 73457; Fragrance Cs-28197; Fragrance Felton 066m; Fragrance Firmenich 47373; Fragrance Givaudan Ess 9090/lc; Fragrance H-6540; Fragrance Herbal 10396; Fragrance Nj-1085; Fragrance P O Fl-147; Fragrance Pa 52805; Fragrance Pera Derm D; Fragrance Rbd-9819; Fragrance Shaw Mudge U-7776; Fragrance Tf 044078; Fragrance Ungerer Honeysuckle K 2771; Fragrance Ungerer N5195; Fructose; Gadolinium Oxide; Galactose; Gamma Cyclodextrin; Gelatin; Gelatin, Crosslinked; Gelfoam Sponge; Gellan Gum (Low Acyl); Gelva 737; Gentisic Acid; Gentisic Acid Ethanolamide; Gluceptate Sodium; Gluceptate Sodium Dihydrate; Gluconolactone; Glucuronic Acid; Glutamic Acid, Dl-; Glutathione; Glycerin; Glycerol Ester Of Hydrogenated Rosin; Glyceryl Citrate; Glyceryl Isostearate; Glyceryl Laurate; Glyceryl Monostearate; Glyceryl Oleate; Glyceryl Oleate/Propylene Glycol; Glyceryl Palmitate; Glyceryl Ricinoleate; Glyceryl Stearate; Glyceryl Stearate-Laureth-23; Glyceryl Stearate/Peg Stearate; Glyceryl Stearate/Peg-100 Stearate; Glyceryl Stearate/Peg-40 Stearate; Glyceryl Stearate-Stearamidoethyl Diethylamine; Glyceryl Trioleate; Glycine; Glycine Hydrochloride; Glycol Distearate; Glycol Stearate; Guanidine Hydrochloride; Guar Gum; Hair Conditioner (18n195-1m); Heptane; Hetastarch; Hexylene Glycol; High Density Polyethylene; Histidine; Human Albumin Microspheres; Hyaluronate Sodium; Hydrocarbon; Hydrocarbon Gel, Plasticized; Hydrochloric Acid; Hydrochloric Acid, Diluted; Hydrocortisone; Hydrogel Polymer; Hydrogen Peroxide; Hydrogenated Castor Oil; Hydrogenated Palm Oil; Hydrogenated Palm/Palm Kernel Oil Peg-6 Esters; Hydrogenated Polybutene 635-690; Hydroxide Ion; Hydroxyethyl Cellulose; Hydroxyethylpiperazine Ethane Sulfonic Acid; Hydroxymethyl Cellulose; Hydroxyoctacosanyl Hydroxystearate; Hydroxypropyl Cellulose; Hydroxypropyl Methylcellulose 2906; Hydroxypropyl-Beta-cyclodextrin; Hypromellose 2208 (15000 Mpa.S); Hypromellose 2910 (15000 Mpa.S); Hypromelloses; Imidurea; Iodine; lodoxamic Acid; lofetamine Hydrochloride; Irish Moss Extract; Isobutane; Isoceteth-20; Isoleucine; Isooctyl Acrylate; Isopropyl Alcohol; Isopropyl Isostearate; Isopropyl Myristate; Isopropyl Myristate-Myristyl Alcohol; Isopropyl Palmitate; Isopropyl Stearate; Isostearic Acid; Isostearyl Alcohol; Isotonic Sodium Chloride Solution; Jelene; Kaolin; Kathon Cg; Kathon Cg II; Lactate; Lactic Acid; Lactic Acid, Dl-; Lactic Acid, L-; Lactobionic Acid; Lactose; Lactose Monohydrate; Lactose, Hydrous; Laneth; Lanolin; Lanolin Alcohol-Mineral Oil; Lanolin Alcohols; Lanolin Anhydrous; Lanolin Cholesterols; Lanolin Nonionic Derivatives; Lanolin, Ethoxylated; Lanolin, Hydrogenated; Lauralkonium Chloride; Lauramine Oxide; Laurdimonium Hydrolyzed Animal Collagen; Laureth Sulfate; Laureth-2; Laureth-23; Laureth-4; Lauric Diethanolamide; Lauric Myristic Diethanolamide; Lauroyl Sarcosine; Lauryl Lactate; Lauryl Sulfate; Lavandula Angustifolia Flowering Top; Lecithin; Lecithin Unbleached; Lecithin, Egg; Lecithin, Hydrogenated; Lecithin, Hydrogenated Soy; Lecithin, Soybean; Lemon Oil; Leucine; Levulinic Acid; Lidofenin; Light Mineral Oil; Light Mineral Oil (85 Ssu); Limonene, (+/−)-; Lipocol Sc-15; Lysine; Lysine Acetate; Lysine Monohydrate; Magnesium Aluminum Silicate; Magnesium Aluminum Silicate Hydrate; Magnesium Chloride; Magnesium Nitrate; Magnesium Stearate; Maleic Acid; Mannitol; Maprofix; Mebrofenin; Medical Adhesive Modified 5-15; Medical Antiform A-F Emulsion; Medronate Disodium; Medronic Acid; Meglumine; Menthol; Metacresol; Metaphosphoric Acid; Methanesulfonic Acid; Methionine; Methyl Alcohol; Methyl Gluceth-10; Methyl Gluceth-20; Methyl Gluceth-20 Sesquistearate; Methyl Glucose Sesquistearate; Methyl Laurate; Methyl Pyrrolidone; Methyl Salicylate; Methyl Stearate; Methylboronic Acid; Methylcellulose (4000 Mpa. S); Methylcelluloses; Methylchloroisothiazolinone; Methylene Blue; Methylisothiazolinone; Methylparaben; Microcrystalline Wax; Mineral Oil; Mono And Diglyceride; Monostearyl Citrate; Monothioglycerol; Multisterol Extract; Myristyl Alcohol; Myristyl Lactate; Myristyl-.Gamma.-Picolinium Chloride; N-(Carbamoyl-Methoxy Peg-40)-1,2-Distearoyl-Cephalin Sodium; N,N-Dimethylacetamide; Niacinamide; Nioxime; Nitric Acid; Nitrogen; Nonoxynol Iodine; Nonoxynol-15; Nonoxynol-9; Norflurane; Oatmeal; Octadecene-1/Maleic Acid Copolymer; Octanoic Acid; Octisalate; Octoxynol-1; Octoxynol-40; Octoxynol-9; Octyldodecanol; Octylphenol Polymethylene; Oleic Acid; Oleth-10/01eth-5; Oleth-2; Oleth-20; Oleyl Alcohol; Oleyl Oleate; Olive Oil; Oxidronate Disodium; Oxyquinoline; Palm Kernel Oil; Palmitamine Oxide; Parabens; Paraffin; Paraffin, White Soft; Parfum Creme 45/3; Peanut Oil; Peanut Oil, Refined; Pectin; Peg 6-32 Stearate/Glycol Stearate; Peg Vegetable Oil; Peg-100 Stearate; Peg-12 Glyceryl Laurate; Peg-120 Glyceryl Stearate; Peg-120 Methyl Glucose Dioleate; Peg-15 Cocamine; Peg-150 Distearate; Peg-2 Stearate; Peg-20 Sorbitan Isostearate; Peg-22 Methyl Ether/Dodecyl Glycol Copolymer; Peg-25 Propylene Glycol Stearate; Peg-4 Dilaurate; Peg-4 Laurate; Peg-40 Castor Oil; Peg-40 Sorbitan Diisostearate; Peg-45/Dodecyl Glycol Copolymer; Peg-5 Oleate; Peg-50 Stearate; Peg-54 Hydrogenated Castor Oil; Peg-6 Isostearate; Peg-60 Castor Oil; Peg-60 Hydrogenated Castor Oil; Peg-7 Methyl Ether; Peg-75 Lanolin; Peg-8 Laurate; Peg-8 Stearate; Pegoxol 7 Stearate; Pentadecalactone; Pentaerythritol Cocoate; Pentasodium Pentetate; Pentetate Calcium Trisodium; Pentetic Acid; Peppermint Oil; Perflutren; Perfume 25677; Perfume Bouquet; Perfume E-1991; Perfume Gd 5604; Perfume Tana 90/42 Scba; Perfume W-1952-1; Petrolatum; Petrolatum, White; Petroleum Distillates; Phenol; Phenol, Liquefied; Phenonip; Phenoxyethanol; Phenylalanine; Phenylethyl Alcohol; Phenylmercuric Acetate; Phenylmercuric Nitrate; Phosphatidyl Glycerol, Egg; Phospholipid; Phospholipid, Egg; Phospholipon 90g; Phosphoric Acid; Pine Needle Oil (Pinus Sylvestris); Piperazine Hexahydrate; Plastibase-50w; Polacrilin; Polidronium Chloride; Poloxamer 124; Poloxamer 181; Poloxamer 182; Poloxamer 188; Poloxamer 237; Poloxamer 407; Poly(Bis(P-Carboxyphenoxy)Propane Anhydride): Sebacic Acid; Poly(Dimethylsiloxane/Methylvinylsiloxane/Methylhydrogensiloxane) Dimethylvinyl Or Dimethylhydroxy Or Trimethyl Endblocked; Poly(D1-Lactic-Co-Glycolic Acid), (50:50; Poly(Dl-Lactic-Co-Glycolic Acid), Ethyl Ester Terminated, (50:50; Polyacrylic Acid (250000 Mw); Polybutene (1400 Mw); Polycarbophil; Polyester; Polyester Polyamine Copolymer; Polyester Rayon; Polyethylene Glycol 1000; Polyethylene Glycol 1450; Polyethylene Glycol 1500; Polyethylene Glycol 1540; Polyethylene Glycol 200; Polyethylene Glycol 300; Polyethylene Glycol 300-1600; Polyethylene Glycol 3350; Polyethylene Glycol 400; Polyethylene Glycol 4000; Polyethylene Glycol 540; Polyethylene Glycol 600; Polyethylene Glycol 6000; Polyethylene Glycol 8000; Polyethylene Glycol 900; Polyethylene High Density Containing Ferric Oxide Black (<1%); Polyethylene Low Density Containing Barium Sulfate (20-24%); Polyethylene T; Polyethylene Terephthalates; Polyglactin; Polyglyceryl-3 Oleate; Polyglyceryl-4 Oleate; Polyhydroxyethyl Methacrylate; Polyisobutylene; Polyisobutylene (1100000 Mw); Polyisobutylene (35000 Mw); Polyisobutylene 178-236; Polyisobutylene 241-294; Polyisobutylene 35-39; Polyisobutylene Low Molecular Weight; Polyisobutylene Medium Molecular Weight; Polyisobutylene/Polybutene Adhesive; Polylactide; Polyols; Polyoxyethylene-Polyoxypropylene 1800; Polyoxyethylene Alcohols; Polyoxyethylene Fatty Acid Esters; Polyoxyethylene Propylene; Polyoxyl 20 Cetostearyl Ether; Polyoxyl 35 Castor Oil; Polyoxyl 40 Hydrogenated Castor Oil; Polyoxyl 40 Stearate; Polyoxyl 400 Stearate; Polyoxyl 6 And Polyoxyl 32 Palmitostearate; Polyoxyl Distearate; Polyoxyl Glyceryl Stearate; Polyoxyl Lanolin; Polyoxyl Palmitate; Polyoxyl Stearate; Polypropylene; Polypropylene Glycol; Polyquaternium-10; Polyquaternium-7 (70/30 Acrylamide/Dadmac; Polysiloxane; Polysorbate 20; Polysorbate 40; Polysorbate 60; Polysorbate 65; Polysorbate 80; Polyurethane; Polyvinyl Acetate; Polyvinyl Alcohol; Polyvinyl Chloride; Polyvinyl Chloride-Polyvinyl Acetate Copolymer; Polyvinylpyridine; Poppy Seed Oil; Potash; Potassium Acetate; Potassium Alum; Potassium Bicarbonate; Potassium Bisulfite; Potassium Chloride; Potassium Citrate; Potassium Hydroxide; Potassium Metabisulfite; Potassium Phosphate, Dibasic; Potassium Phosphate, Monobasic; Potassium Soap; Potassium Sorbate; Povidone Acrylate Copolymer; Povidone Hydrogel; Povidone K17; Povidone K25; Povidone K29/32; Povidone K30; Povidone K90; Povidone K90f; Povidone/Eicosene Copolymer; Povidones; Ppg-12/Smdi Copolymer; Ppg-15 Stearyl Ether; Ppg-20 Methyl Glucose Ether Distearate; Ppg-26 Oleate; Product Wat; Proline; Promulgen D; Promulgen G; Propane; Propellant A-46; Propyl Gallate; Propylene Carbonate; Propylene Glycol; Propylene Glycol Diacetate; Propylene Glycol Dicaprylate; Propylene Glycol Monolaurate; Propylene Glycol Monopalmitostearate; Propylene Glycol Palmitostearate; Propylene Glycol Ricinoleate; Propylene Glycol/Diazolidinyl Urea/Methylparaben/Propylparben; Propylparaben; Protamine Sulfate; Protein Hydrolysate; Pvm/Ma Copolymer; Quaternium-15; Quaternium-15 Cis-Form; Quaternium-52; Ra-2397; Ra-3011; Saccharin; Saccharin Sodium; Saccharin Sodium Anhydrous; Safflower Oil; Sd Alcohol 3a; Sd Alcohol 40; Sd Alcohol 40-2; Sd Alcohol 40b; Sepineo P 600; Serine; Sesame Oil; Shea Butter; Silastic Brand Medical Grade Tubing; Silastic Medical Adhesive,Silicone Type A; Silica, Dental; Silicon; Silicon Dioxide; Silicon Dioxide, Colloidal; Silicone; Silicone Adhesive 4102; Silicone Adhesive 4502; Silicone Adhesive Bio-Psa Q7-4201; Silicone Adhesive Bio-Psa Q7-4301; Silicone Emulsion; Silicone/Polyester Film Strip; Simethicone; Simethicone Emulsion; Sipon Ls 20np; Soda Ash; Sodium Acetate; Sodium Acetate Anhydrous; Sodium Alkyl Sulfate; Sodium Ascorbate; Sodium Benzoate; Sodium Bicarbonate; Sodium Bisulfate; Sodium Bisulfite; Sodium Borate; Sodium Borate Decahydrate; Sodium Carbonate; Sodium Carbonate Decahydrate; Sodium Carbonate Monohydrate; Sodium Cetostearyl Sulfate; Sodium Chlorate; Sodium Chloride; Sodium Chloride Injection; Sodium Chloride Injection, Bacteriostatic; Sodium Cholesteryl Sulfate; Sodium Citrate; Sodium Cocoyl Sarcosinate; Sodium Desoxycholate; Sodium Dithionite; Sodium Dodecylbenzenesulfonate; Sodium Formaldehyde Sulfoxylate; Sodium Gluconate; Sodium Hydroxide; Sodium Hypochlorite; Sodium Iodide; Sodium Lactate; Sodium Lactate, L-; Sodium Laureth-2 Sulfate; Sodium Laureth-3 Sulfate; Sodium Laureth-5 Sulfate; Sodium Lauroyl Sarcosinate; Sodium Lauryl Sulfate; Sodium Lauryl Sulfoacetate; Sodium Metabisulfite; Sodium Nitrate; Sodium Phosphate; Sodium Phosphate Dihydrate; Sodium Phosphate, Dibasic; Sodium Phosphate, Dibasic, Anhydrous; Sodium Phosphate, Dibasic, Dihydrate; Sodium Phosphate, Dibasic, Dodecahydrate; Sodium Phosphate, Dibasic, Heptahydrate; Sodium Phosphate, Monobasic; Sodium Phosphate, Monobasic, Anhydrous; Sodium Phosphate, Monobasic, Dihydrate; Sodium Phosphate, Monobasic, Monohydrate; Sodium Polyacrylate (2500000 Mw); Sodium Pyrophosphate; Sodium Pyrrolidone Carboxylate; Sodium Starch Glycolate; Sodium Succinate Hexahydrate; Sodium Sulfate; Sodium Sulfate Anhydrous; Sodium Sulfate Decahydrate; Sodium Sulfite; Sodium Sulfosuccinated Undecyclenic Monoalkylolamide; Sodium Tartrate; Sodium Thioglycolate; Sodium Thiomalate; Sodium Thiosulfate; Sodium Thiosulfate Anhydrous; Sodium Trimetaphosphate; Sodium Xylenesulfonate; Somay 44; Sorbic Acid; Sorbitan; Sorbitan Isostearate; Sorbitan Monolaurate; Sorbitan Monooleate; Sorbitan Monopalmitate; Sorbitan Monostearate; Sorbitan Sesquioleate; Sorbitan Trioleate; Sorbitan Tristearate; Sorbitol; Sorbitol Solution; Soybean Flour; Soybean Oil; Spearmint Oil; Spermaceti; Squalane; Stabilized Oxychloro Complex; Stannous 2-Ethylhexanoate; Stannous Chloride; Stannous Chloride Anhydrous; Stannous Fluoride; Stannous Tartrate; Starch; Starch 1500, Pregelatinized; Starch, Corn; Stearalkonium Chloride; Stearalkonium Hectorite/Propylene Carbonate; Stearamidoethyl Diethylamine; Steareth-10; Steareth-100; Steareth-2; Steareth-20; Steareth-21; Steareth-40; Stearic Acid; Stearic Diethanolamide; Stearoxytrimethylsilane; Steartrimonium Hydrolyzed Animal Collagen; Stearyl Alcohol; Sterile Water For Inhalation; Styrene/Isoprene/Styrene Block Copolymer; Succimer; Succinic Acid; Sucralose; Sucrose; Sucrose Distearate; Sucrose Polyesters; Sulfacetamide Sodium; Sulfobutylether.Beta.-Cyclodextrin; Sulfur Dioxide; Sulfuric Acid; Sulfurous Acid; Surfactol Qs; Tagatose, D-; Talc; Tall Oil; Tallow Glycerides; Tartaric Acid; Tartaric Acid, Dl-; Tenox; Tenox-2; Tert-Butyl Alcohol; Tert-Butyl Hydroperoxide; Tert-Butylhydroquinone; Tetrakis(2-Methoxyisobutylisocyanide)Copper(I) Tetrafluoroborate; Tetrapropyl Orthosilicate; Tetrofosmin; Theophylline; Thimerosal; Threonine; Thymol; Tin; Titanium Dioxide; Tocopherol; Tocophersolan; Total parenteral nutrition, lipid emulsion; Triacetin; Tricaprylin; Trichloromonofluoromethane; Trideceth-10; Triethanolamine Lauryl Sulfate; Trifluoroacetic Acid; Triglycerides, Medium Chain; Trihydroxystearin; Trilaneth-4 Phosphate; Trilaureth-4 Phosphate; Trisodium Citrate Dihydrate; Trisodium Hedta; Triton 720; Triton X-200; Trolamine; Tromantadine; Tromethamine (TRIS); Tryptophan; Tyloxapol; Tyrosine; Undecylenic Acid; Union 76 Amsco-Res 6038; Urea; Valine; Vegetable Oil; Vegetable Oil Glyceride, Hydrogenated; Vegetable Oil, Hydrogenated; Versetamide; Viscarin; Viscose/Cotton; Vitamin E; Wax, Emulsifying; Wecobee Fs; White Ceresin Wax; White Wax; Xanthan Gum; Zinc; Zinc Acetate; Zinc Carbonate; Zinc Chloride; and Zinc Oxide.

Pharmaceutical composition formulations of viral particles (e.g., AAV particles or regulatable-AAV particles) disclosed herein may include cations or anions. In one embodiment, the formulations include metal cations such as, but not limited to, Zn2+, Ca2+, Cu2+, Mn2+, Mg+ and combinations thereof. As a non-limiting example, formulations may include polymers and complexes with a metal cation (See e.g., U.S. Pat. Nos. 6,265,389 and 6,555,525, each of which is herein incorporated by reference in its entirety).

Formulations of the invention may also include one or more pharmaceutically acceptable salts. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids.

Solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

### Administration

In one embodiment, the viral particles (e.g., AAV particles and regulatable-AAV particles) may be administered by any route which results in a therapeutically effective outcome. These include, but are not limited to enteral (into the intestine), gastroenteral, epidural (into the dura mater), oral (by way of the mouth), transdermal, peridural, intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis or spinal. In specific embodiments, compositions may be administered in a way which allows them cross the blood-brain barrier, vascular barrier, or other epithelial barrier.

In one embodiment, a formulation for a route of administration may include at least one inactive ingredient.

In some embodiments, compositions may be administered in a way which allows them to cross the blood-brain barrier, vascular barrier, or other epithelial barrier. The viral particles (e.g., AAV particles or regulatable-AAV particles) of the present invention may be administered in any suitable form, either as a liquid solution or suspension, as a solid form suitable for liquid solution or suspension in a liquid solution. The viral particles (e.g., AAV particles or regulatable-AAV particles) may be formulated with any appropriate and pharmaceutically acceptable excipient.

In one embodiment, the viral particles (e.g., AAV particles or regulatable-AAV particles) of the present invention may be delivered to a subject via a single route administration.

In one embodiment, the viral particles (e.g., AAV particles or regulatable-AAV particles) of the present invention may be delivered to a subject via a multi-site route of administration. A subject may be administered at 2, 3, 4, 5 or more than 5 sites.

In one embodiment, a subject may be administered the viral particles (e.g., AAV particles or regulatable-AAV particles) of the present invention using a bolus infusion.

In one embodiment, a subject may be administered the viral particles (e.g., AAV particles or regulatable-AAV particles) of the present invention using sustained delivery over a period of minutes, hours or days. The infusion rate may be changed depending on the subject, distribution, formulation or another delivery parameter.

In one embodiment, the viral particles (e.g., AAV particles or regulatable-AAV particles) of the present invention may be delivered by intramuscular delivery route. (See, e.g., U.S. Pat. No. 6,506,379; the content of which is incorporated herein by reference in its entirety). Non-limiting examples of intramuscular administration include an intravenous injection or a subcutaneous injection.

In one embodiment, the viral particles (e.g., AAV particles or regulatable-AAV particles) of the present invention may be delivered by oral administration. Non-limiting examples of oral administration include a digestive tract administration and a buccal administration.

In one embodiment, the viral particles (e.g., AAV particles or regulatable-AAV particles) of the present invention may be delivered by intraocular delivery route. A non-limiting example of intraocular administration include an intravitreal injection.

In one embodiment, the viral particles (e.g., AAV particles or regulatable-AAV particles) of the present invention may be delivered by intranasal delivery route. Non-limiting examples of intranasal delivery include administration of nasal drops or nasal sprays.

In some embodiments, the viral particles (e.g., AAV particles or regulatable-AAV particles) that may be administered to a subject by peripheral injections. Non-limiting examples of peripheral injections include intraperitoneal, intramuscular, intravenous, conjunctival or joint injection. It was disclosed in the art that the peripheral administration of AAV vectors can be transported to the central nervous system, for example, to the motor neurons (e.g., U. S. Patent Publication Nos. 20100240739; and 20100130594; the content of each of which is incorporated herein by reference in their entirety).

In one embodiment, the viral particles (e.g., AAV particles or regulatable-AAV particles) may be delivered by injection into the CSF pathway. Non-limiting examples of delivery to the CSF pathway include intrathecal and intracerebroventricular administration.

In one embodiment, the viral particles (e.g., AAV particles or regulatable-AAV particles) may be delivered by systemic delivery. As a non-limiting example, the systemic delivery may be by intravascular administration.

In one embodiment, the viral particles (e.g., AAV particles or regulatable-AAV particles) of the present invention may be administered to a subject by intracranial delivery (See, e.g., U.S. Pat. No. 8,119,611; the content of which is incorporated herein by reference in its entirety).

In one embodiment, the viral particles (e.g., AAV particles or regulatable-AAV particles) of the present invention may be administered to a subject by intraparenchymal administration.

In one embodiment, the viral particles (e.g., AAV particles or regulatable-AAV particles) of the present invention may be administered to a subject by intramuscular administration.

In one embodiment, the viral particles (e.g., AAV particles or regulatable-AAV particles) of the present invention are administered to a subject and transduce muscle of a subject. As a non-limiting example, the viral particles (e.g., AAV particles or regulatable-AAV particles) are administered by intramuscular administration.

In one embodiment, the viral particles (e.g., AAV particles or regulatable-AAV particles) of the present invention may be administered to a subject by intravenous administration.

In one embodiment, the viral particles (e.g., AAV particles or regulatable-AAV particles) of the present invention may be administered to a subject by subcutaneous administration.

In one embodiment, the viral particles (e.g., AAV particles or regulatable-AAV particles) of the present invention may be administered to a subject by topical administration.

In one embodiment, the viral particles (e.g., AAV particles or regulatable-AAV particles) may be delivered by direct injection into the brain. As a non-limiting example, the brain delivery may be by intrastriatal administration.

In one embodiment, the viral particles (e.g., AAV particles or regulatable-AAV particles) may be delivered by more than one route of administration. As non-limiting examples of combination administrations, viral particles (e.g., AAV particles or regulatable-AAV particles) may be delivered by intrathecal and intracerebroventricular, or by intravenous and intraparenchymal administration.

In one embodiment, the viral particles (e.g., AAV particles or regulatable-AAV particles) comprising the various payload constructs may be administered simultaneously. In another embodiment, the viral particles (e.g., AAV particles or regulatable-AAV particles) may be administered sequentially. In some embodiments, the regulatable-AAV particles comprising the regulatable element may be delivered, 1, 2, 3, 4, 5, 6, or 7 days before or after the viral particles (e.g., AAV particles) comprising the payload encoding the gene of interest. In some embodiments, the regulatable-AAV particles comprising the regulatable element may be delivered, 1 to 4 weeks before or after the viral particles (e.g., AAV particles) comprising the payload encoding the gene of interest. In some embodiments, the regulatable-AAV particles comprising the regulatable element may be delivered, 1 month to 1 year before or after the viral particles (e.g., AAV particles) comprising the payload encoding the gene of interest. In some embodiments, the regulatable-AAV particles comprising the regulatable element may be delivered, 1 year to up to 5, 10, 20, 30, 40, 50, 60, or 70 years before or after the viral particles (e.g., AAV particles) comprising the payload encoding the gene of interest.

In some embodiments, multiple types of delivery methods may be used in combination to deliver different payload components. In one embodiment, a viral particle (e.g., AAV particle or regulatable-AAV particle) comprising the payload comprising the gene of interest is delivered in combination (simultaneously or sequentially) with one or more regulatable elements, which are delivered via a non-AAV vehicle or method. In another embodiment, the payload may be delivered using a non-AAV delivery vehicle or method and the one or more regulatable elements may be delivered as payloads contained in one or more regulatable-AAV particles. In one embodiment, a regulatable element may be delivered by a viral particle (e.g., AAV vector or regulatable-AAV vector). In one embodiment, the regulatable element may be delivered as an mRNA or formulated mRNA. In another embodiment, the payload may be delivered as an mRNA or formulated mRNA. In one embodiment, the regulatable element may be delivered as a protein or a formulated protein. In another embodiment, the payload may be delivered as a protein or a formulated protein.

### Combinations

In one embodiment, the viral particles may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of pharmaceutical, prophylactic, diagnostic, or imaging compositions in combination with agents that may improve their bioavailability, reduce and/or modify their metabolism, inhibit their excretion, and/or modify their distribution within the body.

### Delivery to Cells

The present disclosure provides a method of delivering to a cell or tissue any of the viral particles, comprising contacting the cell or tissue with the viral particles or contacting the cell or tissue with a particle comprising the viral particles, or contacting the cell or tissue with any of the described compositions, including pharmaceutical compositions. The method of delivering the viral particles to a cell or tissue can be accomplished in vitro, ex vivo, or in vivo.

In one embodiment, the viral particles of the present invention may be administered to stem cells. The present disclosure provides a method of delivering to a stem cell any of the viral particles, comprising contacting the stem cell with the viral particles or contacting the stem cell with a particle comprising the viral particles, or contacting the stem cell with any of the described compositions, including pharmaceutical compositions.

In one embodiment, the viral particle may be an AAV particle which is delivered to a stem cell and may comprise an AAV Clade F capsid.

In one embodiment, the viral particle administered to cells comprises a correction genome as described in International Publication WO2016049230, the contents of which are herein incorporated by reference in their entirety. The correction genome comprises three elements, an internucleotide bond or nucleotide sequence for integration into a target locus of a mammalian chromosome, which serves as the editing element, and a 5′ and a 3′ homologous arm sequence at the 5′ and 3′ ends of the editing element respectively, which have homology to the 5′ region and the 3′ region of the mammalian chromosome relative to the target locus. Further, the correction genome may be characterized by the absence of a promoter operatively linked to the editing element nucleotide sequence. In some embodiments, the viral particle comprising a correction genome may be introduced to a stem cell (e.g., CD34+). In some embodiments, the cell may be transduced without exogenous nucleases. Genome editing by the correction genome (insertions, deletions, point mutations, alterations or any combination thereof) may be conducted ex vivo or in vivo in a subject in need thereof and may be used to treat a disease or disorder.

### Delivery to Subjects

The present disclosure additionally provides a method of delivering to a subject, including a mammalian subject, any of the above-described viral particles comprising administering to the subject the viral particles, or administering to the subject a viral particles, or administering to the subject any of the described compositions, including pharmaceutical compositions.

### Dosing

The present invention provides methods of administering viral particles (e.g., AAV particles or regulatable-AAV particles) and their payload or complexes in accordance with the invention to a subject in need thereof. Viral particle pharmaceutical, imaging, diagnostic, or prophylactic compositions thereof, may be administered to a subject using any amount and any route of administration effective for preventing, treating, diagnosing, or imaging a disease, disorder, and/or condition (e.g., a disease, disorder, and/or condition relating to working memory deficits). The exact amount required will vary from subject to subject, depending on the species, age, and general condition of the subject, the severity of the disease, the particular composition, its mode of administration, its mode of activity, and the like. Compositions in accordance with the invention are typically formulated in unit dosage form for ease of administration and uniformity of dosage. It will be understood, however, that the total daily usage of the compositions of the present invention may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific payload employed; the specific composition employed; the age, body weight, general health, sex and diet of the patient; the time of administration, route of administration, and rate of excretion of the specific payload employed; the duration of the treatment; drugs used in combination or coincidental with the specific payload employed; and like factors well known in the medical arts.

In certain embodiments, viral particle pharmaceutical compositions in accordance with the present invention may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, prophylactic, or imaging effect. It will be understood that the above dosing concentrations may be converted to vg or viral genomes per kg or into total viral genomes administered by one of skill in the art.

In certain embodiments, viral particle pharmaceutical compositions in accordance with the present disclosure may be administered at about 10 to about 600 μl/site, 50 to about 500 μl/site, 100 to about 400 μl/site, 120 to about 300 μl/site, 140 to about 200 μl/site, about 160 μl/site. As non-limiting examples, viral particles may be administered at 50 μl/site and/or 150 μl/site.

The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. As used herein, a “split dose” is the division of single unit dose or total daily dose into two or more doses, e.g., two or more administrations of the single unit dose. As used herein, a “single unit dose” is a dose of any therapeutic administered in one dose/at one time/single route/single point of contact, i.e., single administration event. As used herein, a “total daily dose” is an amount given or prescribed in 24 hour period. It may be administered as a single unit dose. In one embodiment, the viral particles of the present invention are administered to a subject in split doses. The viral particles may be formulated in buffer only or in a formulation described herein.

A pharmaceutical composition described herein can be formulated into a dosage form described herein, such as a topical, intranasal, pulmonary, intratracheal, or injectable (e.g., intravenous, intraocular, intravitreal, intramuscular, intracardiac, intraperitoneal, and/or subcutaneous).

In one embodiment, delivery of the viral particles of the present invention to a subject provides neutralizing activity to a subject. The neutralizing activity can be for at least 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 20 months, 21 months, 22 months, 23 months, 2 years, 3 years, 4 years, 5 years, 6 years, 7 years, 8 years, 9 years, 10 years or more than 10 years.

In one embodiment, delivery of the viral particles of the present invention results in minimal serious adverse events (SAEs) as a result of the delivery of the viral particles.

In one embodiment, delivery of viral particles to cells of the central nervous system (e.g., parenchyma) may comprise a total dose between about 1×10⁶ VG and about 1×10¹⁶ VG. In some embodiments, delivery may comprise a total dose of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 1.9×10¹⁰, 2×10¹⁰, 3×10¹⁰, 3.73×10¹⁰, 4×10¹⁰, 5×10¹⁰, 6×10¹⁰, 7×10¹⁰, 8×10¹⁰, 9×10¹⁰, 1×10¹¹, 2×10¹¹, 2.5×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG. As a non-limiting example, the total dose is 1×10¹³ VG. As another non-limiting example, the total dose is 2.1×10¹² VG.

In one embodiment, delivery of viral particles to cells of the central nervous system may comprise a composition concentration between about 1×10⁶ VG/mL and about 1×10¹⁶ VG/mL. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/mL. In one embodiment, the delivery comprises a composition concentration of 1×10¹³ VG/mL. In one embodiment, the delivery comprises a composition concentration of 2.1×10¹² VG/mL.

### Measurement of Expression

Expression of payloads from viral genomes may be determined using various methods known in the art such as, but not limited to immunochemistry (e.g., IHC), in situ hybridization (ISH), enzyme-linked immunosorbent assay (ELISA), affinity ELISA, ELISPOT, flow cytometry, immunocytology, surface plasmon resonance analysis, kinetic exclusion assay, liquid chromatography-mass spectrometry (LCMS), high-performance liquid chromatography (HPLC), BCA assay, immunoelectrophoresis, Western blot, SDS-PAGE, protein immunoprecipitation, and/or PCR.

### Bioavailability

The viral particles, when formulated into a composition with a delivery agent as described herein, can exhibit an increase in bioavailability as compared to a composition lacking a delivery agent as described herein. As used herein, the term “bioavailability” refers to the systemic availability of a given amount of viral particle or expressed payload administered to a mammal. Bioavailability can be assessed by measuring the area under the curve (AUC) or the maximum serum or plasma concentration (Cₘₐₓ) of the composition following. AUC is a determination of the area under the curve plotting the serum or plasma concentration of a compound (e.g., viral particles or expressed payloads) along the ordinate (Y-axis) against time along the abscissa (X-axis). Generally, the AUC for a particular compound can be calculated using methods known to those of ordinary skill in the art and as described in G. S. Banker, Modern Pharmaceutics, Drugs and the Pharmaceutical Sciences, v. 72, Marcel Dekker, New York, Inc., 1996, the contents of which are herein incorporated by reference in its entirety.

The Cₘₐₓ value is the maximum concentration of the viral particle or expressed payload achieved in the serum or plasma of a mammal following administration of the viral particle to the mammal. The Cₘₐₓ value of can be measured using methods known to those of ordinary skill in the art. The phrases “increasing bioavailability” or “improving the pharmacokinetics,” as used herein mean that the systemic availability of a first viral particle or expressed payload, measured as AUC, Cₘₐₓ, or Cₘᵢₙ in a mammal is greater, when co-administered with a delivery agent as described herein, than when such co-administration does not take place. In some embodiments, the bioavailability can increase by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%.

### Therapeutic Window

As used herein “therapeutic window” refers to the range of plasma concentrations, or the range of levels of therapeutically active substance at the site of action, with a high probability of eliciting a therapeutic effect. In some embodiments, the therapeutic window of the viral particle as described herein can increase by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%.

### Volume of Distribution

As used herein, the term “volume of distribution” refers to the fluid volume that would be required to contain the total amount of the drug in the body at the same concentration as in the blood or plasma: Vdist equals the amount of drug in the body/concentration of drug in blood or plasma. For example, for a 10 mg dose and a plasma concentration of 10 mg/L, the volume of distribution would be 1 liter. The volume of distribution reflects the extent to which the drug is present in the extravascular tissue. A large volume of distribution reflects the tendency of a compound to bind to the tissue components compared with plasma protein binding. In a clinical setting, V_{d}ist can be used to determine a loading dose to achieve a steady state concentration. In some embodiments, the volume of distribution of the viral particles as described herein can decrease at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%.

### Biological Effect

In one embodiment, the biological effect of the viral particles delivered to the animals may be categorized by analyzing the payload expression in the animals. The payload expression may be determined from analyzing a biological sample collected from a mammal administered the viral particles of the present invention. For example, a protein expression of 50-200 pg/ml for the protein encoded by the viral particles delivered to the mammal may be seen as a therapeutically effective amount of protein in the mammal.

### Methods of Use

The present disclosure additionally provides a method for treating a disease, disorder and/or condition in a mammalian subject, including a human subject, comprising administering to the subject any of viral genomes (“VG”) or administering to the subject a viral particle comprising a viral genome, or administering to the subject any of the described compositions, including pharmaceutical compositions.

In some embodiments, the present invention provides methods for inhibiting, silencing or inducing, activating, and/or initiating expression of a gene in a cell.

In one embodiment, the disease, disorder and/or condition is a neurological disease, disorder and/or condition.

In one embodiment, the neurological disease, disorder and/or condition is Parkinson's disease.

In one embodiment, the neurological disease, disorder and/or condition is Friedreich's Ataxia.

In one embodiment, the neurological disease, disorder and/or condition is Amyotrophic lateral sclerosis (ALS).

In one embodiment, the neurological disease, disorder and/or condition is Huntington's disease.

In one embodiment, the neurological disease, disorder and/or condition is spinal muscular atrophy (SMA).

In one embodiment, the neurological disease, disorder and/or condition is a muscular or cardiac disease, disorder and/or condition.

In one embodiment, the neurological disease, disorder and/or condition is an immune system disease, disorder and/or condition.

In one embodiment, the neurological disease, disorder and/or condition is a disease, disorder and/or condition of the CNS.

In one embodiment, neurological disease, disorder and/or condition is Alzheimer's Disease.

The polynucleotides encoding polypeptides (e.g., mRNA) of the invention may be useful in the fields of human disease, antibodies, viruses, veterinary applications and a variety of in vivo and in vitro settings.

In some embodiments, the viral particles are useful in the field of medicine for the treatment, palliation or amelioration of conditions or diseases such as, but not limited to, blood, cardiovascular, CNS, dermatology, endocrinology, genetic, genitourinary, gastrointestinal, musculoskeletal, oncology, and immunology, respiratory, sensory and anti-infective.

In one embodiment, the viral particles are useful in the treatment of Duchenne muscular dystrophy or Becker muscular dystrophy.

In some embodiments, viral particles in accordance with the present invention may be used for the treatment of disorders, and/or conditions, including but not limited to one or more of the following: autoimmune disorders (e.g. diabetes, lupus, multiple sclerosis, psoriasis, rheumatoid arthritis); inflammatory disorders (e.g. arthritis, pelvic inflammatory disease); neurological disorders (e.g. Alzheimer's disease, Huntington's disease; autism; Parkinson's disease, amyotrophic lateral sclerosis, Friedrich's Ataxia, spinal muscular atrophy, schizophrenia); cardiovascular disorders (e.g. atherosclerosis, hypercholesterolemia, thrombosis, clotting disorders, angiogenic disorders such as macular degeneration; proliferative disorders (e.g. cancer, benign neoplasms); respiratory disorders (e.g. chronic obstructive pulmonary disease); digestive disorders (e.g. inflammatory bowel disease, ulcers); musculoskeletal disorders (e.g. fibromyalgia, arthritis, Duchenne muscular dystrophy, Becker muscular dystrophy); endocrine, metabolic, and nutritional disorders (e.g. diabetes, osteoporosis); urological disorders (e.g. renal disease); psychological disorders (e.g. depression, schizophrenia); skin disorders (e.g. wounds, eczema); blood and lymphatic disorders (e.g. anemia, hemophilia).

### Definitions

At various places in the present specification, substituents of compounds of the present disclosure are disclosed in groups or in ranges. It is specifically intended that the present disclosure include each and every individual sub-combination of the members of such groups and ranges. The following is a non-limiting list of term definitions.

Adeno-associated virus: The term “adeno-associated virus” or “AAV” as used herein refers to members of the dependovirus genus comprising any particle, sequence, gene, protein, or component derived therefrom.

Adeno-associated virus particle or AAV particle: As used herein, the term “adeno-associated virus particle” or “AAV particle” refers to a viral particle where the virus is adeno-associated virus (AAV). The AAV particle may be derived from any serotype, described herein or known in the art, including combinations of serotypes (i.e., “pseudotyped” AAV) or from various genomes (e.g., single stranded or self-complementary). In addition, the AAV particle may be replication defective and/or targeted.

Activity: As used herein, the term “activity” refers to the condition in which things are happening or being done. Compositions of the invention may have activity and this activity may involve one or more biological events.

Administered in combination: As used herein, the term “administered in combination” or “combined administration” refers to simultaneous exposure to two or more agents administered at the same time or within an interval such that the subject is at some point in time simultaneously exposed to both and/or such that there may be an overlap in the effect of each agent on the patient. In some embodiments, at least one dose of one or more agents is administered within about 24 hours, 12 hours, 6 hours, 3 hours, 1 hour, 30 minutes, 15 minutes, 10 minutes, 5 minutes, or 1 minute of at least one dose of one or more other agents. In some embodiments, administration occurs in overlapping dosage regimens. As used herein, the term “dosage regimen” refers to a plurality of doses spaced apart in time. Such doses may occur at regular intervals or may include one or more hiatus in administration. In some embodiments, the administration of individual doses of one or more compounds and/or compositions of the present invention, as described herein, are spaced sufficiently closely together such that a combinatorial (e.g., a synergistic) effect is achieved.

Amelioration: As used herein, the term “amelioration” or “ameliorating” refers to a lessening of severity of at least one indicator of a condition or disease. For example, in the context of neurodegeneration disorder, amelioration includes the reduction of neuron loss.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Antisense strand: As used herein, the term “the antisense strand” or “the first strand” or “the guide strand” of a siRNA molecule refers to a strand that is substantially complementary to a section of about 10-50 nucleotides, e.g., about 15-30, 16-25, 18-23 or 19-22 nucleotides of the mRNA of the gene targeted for silencing. The antisense strand or first strand has sequence sufficiently complementary to the desired target mRNA sequence to direct target-specific silencing, e.g., complementarity sufficient to trigger the destruction of the desired target mRNA by the RNAi machinery or process.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, mean that the moieties are physically associated or connected with one another, either directly or via one or more additional moieties that serve as linking agents, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization based connectivity sufficiently stable such that the “associated” entities remain physically associated.

Biomolecule: As used herein, the term “biomolecule” is any natural molecule which is amino acid-based, nucleic acid-based, carbohydrate-based or lipid-based, and the like.

Biologically active: As used herein, the phrase “biologically active” refers to a characteristic of any substance that has activity in or on a biological system and/or organism. For instance, a substance that, when administered to an organism, has a biological effect on that organism, is considered to be biologically active. In particular embodiments, a compounds and/or compositions of the present invention may be considered biologically active if even a portion of is biologically active or mimics an activity considered to biologically relevant.

Biological system: As used herein, the term “biological system” refers to a group of organs, tissues, cells, intracellular components, proteins, nucleic acids, molecules (including, but not limited to biomolecules) that function together to perform a certain biological task within cellular membranes, cellular compartments, cells, tissues, organs, organ systems, multicellular organisms, or any biological entity. In some embodiments, biological systems are cell signaling pathways comprising intracellular and/or extracellular cell signaling biomolecules. In some embodiments, biological systems comprise growth factor signaling events within the extracellular/cellular matrix and/or cellular niches.

Complementary and substantially complementary: As used herein, the term “complementary” refers to the ability of polynucleotides to form base pairs with one another. Base pairs are typically formed by hydrogen bonds between nucleotide units in antiparallel polynucleotide strands. Complementary polynucleotide strands can form base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present invention, the ability to substitute a T is implied, unless otherwise stated. Perfect complementarity or 100% complementarity refers to the situation in which each nucleotide unit of one polynucleotide strand can form hydrogen bond with a nucleotide unit of a second polynucleotide strand. Less than perfect complementarity refers to the situation in which some, but not all, nucleotide units of two strands can form hydrogen bond with each other. For example, for two 20-mers, if only two base pairs on each strand can form hydrogen bond with each other, the polynucleotide strands exhibit 10% complementarity. In the same example, if 18 base pairs on each strand can form hydrogen bonds with each other, the polynucleotide strands exhibit 90% complementarity. As used herein, the term “substantially complementary” means that the siRNA has a sequence (e.g., in the antisense strand) which is sufficient to bind the desired target mRNA, and to trigger the RNA silencing of the target mRNA.

Compound: As used herein, the term “compound” refers to a distinct chemical entity. In some embodiments, a particular compound may exist in one or more isomeric or isotopic forms (including, but not limited to stereoisomers, geometric isomers and isotopes). In some embodiments, a compound is provided or utilized in only a single such form. In some embodiments, a compound is provided or utilized as a mixture of two or more such forms (including, but not limited to a racemic mixture of stereoisomers). Those of skill in the art appreciate that some compounds exist in different such forms, show different properties and/or activities (including, but not limited to biological activities). In such cases it is within the ordinary skill of those in the art to select or avoid particular forms of the compound for use in accordance with the present invention. For example, compounds that contain asymmetrically substituted carbon atoms can be isolated in optically active or racemic forms. Methods on how to prepare optically active forms from optically active starting materials are known in the art, such as by resolution of racemic mixtures or by stereoselective synthesis.

Conserved: As used herein, the term “conserved” refers to nucleotides or amino acid residues of polynucleotide or polypeptide sequences, respectively, that are those that occur unaltered in the same position of two or more sequences being compared. Nucleotides or amino acids that are relatively conserved are those that are conserved among more related sequences than nucleotides or amino acids appearing elsewhere in the sequences.

In some embodiments, two or more sequences are said to be “completely conserved” if they are 100% identical to one another. In some embodiments, two or more sequences are said to be “highly conserved” if they are at least 70% identical, at least 80% identical, at least 90% identical, or at least 95% identical to one another. In some embodiments, two or more sequences are said to be “highly conserved” if they are about 70% identical, about 80% identical, about 90% identical, about 95%, about 98%, or about 99% identical to one another. In some embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In some embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of an oligonucleotide or polypeptide or may apply to a portion, region or feature thereof.

In one embodiment, conserved sequences are not contiguous. Those skilled in the art are able to appreciate how to achieve alignment when gaps in contiguous alignment are present between sequences, and to align corresponding residues not withstanding insertions or deletions present.

Delivery: As used herein, “delivery” refers to the act or manner of delivering compounds, substances, entities, moieties, cargoes or payloads to a target. Such target may be a cell, tissue, organ, organism, or system (whether biological or production).

Delivery Agent: As used herein, “delivery agent” refers to any agent which facilitates, at least in part, the delivery of one or more substances (including, but not limited to a compounds and/or compositions of the present invention, e.g., viral particles or expression vectors) to targeted cells.

Destabilized: As used herein, the term “destable,” “destabilize,” or “destabilizing region” means a region or molecule that is less stable than a starting, reference, wild-type or native form of the same region or molecule.

Detectable label: As used herein, “detectable label” refers to one or more markers, signals, or moieties which are attached, incorporated or associated with another entity, which markers, signals or moieties are readily detected by methods known in the art including radiography, fluorescence, chemiluminescence, enzymatic activity, absorbance, immunological detection and the like. Detectable labels may include radioisotopes, fluorophores, chromophores, enzymes, dyes, metal ions, ligands, biotin, avidin, streptavidin and haptens, quantum dots, polyhistidine tags, myc tags, flag tags, human influenza hemagglutinin (HA) tags and the like. Detectable labels may be located at any position in the entity with which they are attached, incorporated or associated. For example, when attached, incorporated in or associated with a peptide or protein, they may be within the amino acids, the peptides, or proteins, or located at the N- or C- termini.

Effective amount: As used herein, the term “effective amount” of an agent is that amount sufficient to effect beneficial or desired results, for example, upon single or multiple dose administration to a subject cell, in curing, alleviating, relieving or improving one or more symptoms of a disorder and, as such, an “effective amount” depends upon the context in which it is being applied. For example, in the context of administering an agent that treats ALS, an effective amount of an agent is, for example, an amount sufficient to achieve treatment, as defined herein, of ALS, as compared to the response obtained without administration of the agent.

Engineered: As used herein, embodiments of the invention are “engineered” when they are designed to have a feature or property, whether structural or chemical, that varies from a starting point, wild-type or native molecule. Thus, engineered agents or entities are those whose design and/or production include an act of the hand of man.

Epitope: As used herein, an “epitope” refers to a surface or region on a molecule that is capable of interacting with a biomolecule. For example a protein may contain one or more amino acids, e.g., an epitope, which interacts with an antibody, e.g., a biomolecule. In some embodiments, when referring to a protein or protein module, an epitope may comprise a linear stretch of amino acids or a three dimensional structure formed by folded amino acid chains.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; (4) folding of a polypeptide or protein; and (5) post-translational modification of a polypeptide or protein.

Feature: As used herein, a “feature” refers to a characteristic, a property, or a distinctive element.

Formulation: As used herein, a “formulation” includes at least a compound and/or composition of the present invention and a delivery agent.

Fragment: A “fragment,” as used herein, refers to a contiguous portion of a whole. For example, fragments of proteins may comprise polypeptides obtained by digesting full-length protein isolated from cultured cells. In some embodiments, a fragment of a protein includes at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250 or more amino acids. In some embodiments, fragments of an antibody include portions of an antibody subjected to enzymatic digestion or synthesized as such.

Functional: As used herein, a “functional” biological molecule is a biological entity with a structure and in a form in which it exhibits a property and/or activity by which it is characterized.

Fusion protein: As used herein, a “fusion protein” or “chimeric protein” is a protein created through the combination of two or more genes encoding separate proteins. Translation of this fusion gene results in one or more polypeptides with functional properties derived from each of the original proteins. In some embodiments, the fusion protein or chimeric protein can be engineered to include the full sequence of both original proteins. In other embodiments, the fusion protein or chimeric protein includes a part, such as a functional part or domain, from each of the proteins. Various spatial arrangements of the domains may be envisioned according to the present invention. It is contemplated as part of the current invention that the domains may be N-terminal, C-terminal, or interspersed with respect to each other in various orientations. In some embodiments, the fusion proteins may comprise multiple copies of a particular domain. The various domains of the fusion proteins may be connected to each other by linkers. In some embodiments, the domains may be encoded on separate polynucleotides. In some embodiments, they may be encoded on the same polynucleotide. In some embodiments, the various domains may be on the same polypeptide. In other embodiments, the domains may be on separate polypeptides. It is also understood that the fusion proteins or proteins of the present invention may be codon optimized for expression in the system of interest according to methods known in the art.

Gene expression: The term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides. Methods of measuring the amount or levels of RNA, mRNA, polypeptides and peptides are well known in the art.

Homology: As used herein, the term “homology” refers to the overall relatedness between polymeric molecules, e.g. between nucleic acid molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the invention, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is typically determined by the ability to encode a stretch of at least 4-5 uniquely specified amino acids. In accordance with the invention, two protein sequences are considered to be homologous if the proteins are at least about 50%, 60%, 70%, 80%, or 90% identical for at least one stretch of at least about 20 amino acids. In many embodiments, homologous protein may show a large overall degree of homology and a high degree of homology over at least one short stretch of at least 3, 4, 5, 6, 7, 8, 9, 10, 11, 12, 13, 14, 15, 16, 17, 18, 19, 20, 25, 30, 35, 40, 45, 50 or more amino acids. In many embodiments, homologous proteins share one or more characteristic sequence elements. As used herein, the term “characteristic sequence element” refers to a motif present in related proteins. In some embodiments, the presence of such motifs correlates with a particular activity (such as biological activity).

Identity: As used herein, the term “identity” refers to the overall relatedness between polymeric molecules, e.g., between oligonucleotide molecules (e.g. DNA molecules and/or RNA molecules) and/or between polypeptide molecules. Calculation of the percent identity of two polynucleotide sequences, for example, may be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined, for example using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

Inhibit expression of a gene: As used herein, the phrase “inhibit expression of a gene” means to cause a reduction in the amount of an expression product of the gene. The expression product may be RNA transcribed from the gene (e.g. mRNA) or a polypeptide translated from mRNA transcribed from the gene. Typically a reduction in the level of mRNA results in a reduction in the level of a polypeptide translated therefrom. The level of expression may be determined using standard techniques for measuring mRNA or protein.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Isolated: As used herein, the term “isolated” is synonymous with “separated”, but carries with it the inference separation was carried out by the hand of man. In one embodiment, an isolated substance or entity is one that has been separated from at least some of the components with which it was previously associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Substantially isolated: By “substantially isolated” is meant that the compound is substantially separated from the environment in which it was formed or detected. Partial separation can include, for example, a composition enriched in the compound of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art. In some embodiments, isolation of a substance or entity includes disruption of chemical associations and/or bonds. In some embodiments, isolation includes only the separation from components with which the isolated substance or entity was previously combined and does not include such disruption.

Ligand: As used herein, a “ligand” or “chemical agent” is a substance that forms a complex with a protein or fusion protein. Typically, binding is reversible. Ligand binding to a protein often leads to a conformational change in the protein which typically alters the functional state of the protein. In a non-limiting example, ligands may be activators or inhibitors. In other non-limiting examples, a ligand or chemical agent may be a small molecule, ion or protein.

Modified: As used herein, the term “modified” refers to a changed state or structure of a molecule or entity as compared with a parent or reference molecule or entity. Molecules may be modified in many ways including chemically, structurally, and functionally. In some embodiments, compounds and/or compositions of the present invention are modified by the introduction of non-natural amino acids, or non-natural nucleotides.

Mutation: As used herein, the term “mutation” refers to a change and/or alteration. In some embodiments, mutations may be changes and/or alterations to proteins (including peptides and polypeptides) and/or nucleic acids (including polynucleic acids). In some embodiments, mutations comprise changes and/or alterations to a protein and/or nucleic acid sequence. Such changes and/or alterations may comprise the addition, substitution and or deletion of one or more amino acids (in the case of proteins and/or peptides) and/or nucleotides (in the case of nucleic acids and or polynucleic acids). In embodiments wherein mutations comprise the addition and/or substitution of amino acids and/or nucleotides, such additions and/or substitutions may comprise one or more amino acid and/or nucleotide residues and may include modified amino acids and/or nucleotides.

Naturally occurring: As used herein, “naturally occurring” means existing in nature without artificial aid, or involvement of the hand of man.

Non-human vertebrate: As used herein, a “non-human vertebrate” includes all vertebrates except Homo sapiens, including wild and domesticated species. Examples of non-human vertebrates include, but are not limited to, mammals, such as alpaca, banteng, bison, camel, cat, cattle, deer, dog, donkey, gayal, goat, guinea pig, horse, llama, mule, pig, rabbit, reindeer, sheep water buffalo, and yak.

Nucleic acid: As used herein, the term “nucleic acid”, “polynucleotide” and “oligonucleotide” refer to any nucleic acid polymers composed of either polydeoxyribonucleotides (containing 2-deoxy-D-ribose), or polyribonucleotides (containing D-ribose), or any other type of polynucleotide which is an N glycoside of a purine or pyrimidine base, or modified purine or pyrimidine bases. There is no intended distinction in length between the term “nucleic acid”, “polynucleotide” and “oligonucleotide”, and these terms will be used interchangeably. These terms refer only to the primary structure of the molecule. Thus, these terms include double- and single-stranded DNA, as well as double- and single stranded RNA.

Off-target: As used herein, “off target” refers to any unintended effect on any one or more target, gene and/or cellular transcript.

Operably linked: As used herein, the phrase “operably linked” refers to a functional connection between two or more molecules, constructs, transcripts, entities, moieties or the like.

Particle: As used herein, a “particle” is a virus comprised of at least two components, a protein capsid and a polynucleotide sequence enclosed within the capsid.

Patient: As used herein, “patient” refers to a subject who may seek or be in need of treatment, requires treatment, is receiving treatment, will receive treatment, or a subject who is under care by a trained (e.g., licensed) professional for a particular disease or condition.

Payload: As used herein, “payload” refers to one or more polynucleotides or polynucleotide regions encoded by or within a viral genome or an expression product of such polynucleotide or polynucleotide region, e.g., a transgene, a polynucleotide encoding a polypeptide or multi-polypeptide or a modulatory nucleic acid or regulatory nucleic acid. Where regulatable elements are encoded or the payload is regulated by regulatable elements, the payload may also be referred to as a “regulatable-AAV payload.” Where CRISPR regulatable elements are encoded or the payload is regulated by a CRISPR regulatable element, the payload may also be referred to as a “CRISPR-AAV payload.”

Payload construct: As used herein, “payload construct” is one or more polynucleotide regions encoding or comprising a payload that is flanked on one or both sides by an inverted terminal repeat (ITR) sequence. The payload construct is a template that is replicated in a viral production cell to produce a viral genome. Where regulatable elements are encoded or the payload is regulated by a regulatable element, the payload construct may also be referred to as a “regulatable-AAV payload construct.” Where CRISPR regulatable elements are encoded or the payload is regulated by a CRISPR regulatable element, the payload construct may also be referred to as a “CRISPR-AAV payload construct.” In addition to the sequence encoding the payload, the payload construct may also comprise 5′ and 3′ untranslated regions (UTRs) and may also include the promoter. Payload construct vector: As used herein, “payload construct vector” is a vector encoding or comprising a payload construct, and regulatory regions for replication and expression in bacterial cells.

Payload construct expression vector: As used herein, a “payload construct expression vector” is a vector encoding or comprising a payload construct and which further comprises one or more polynucleotide regions encoding or comprising components for viral expression in a viral replication cell.

Peptide: As used herein, the term “peptide” refers to a chain of amino acids that is less than or equal to about 50 amino acids long, e.g., about 5, 10, 15, 20, 25, 30, 35, 40, 45, or 50 amino acids long.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: As used herein, the term “pharmaceutically acceptable excipient,” as used herein, refers to any ingredient other than active agents (e.g., as described herein) present in pharmaceutical compositions and having the properties of being substantially nontoxic and non-inflammatory in subjects. In some embodiments, pharmaceutically acceptable excipients are vehicles capable of suspending and/or dissolving active agents. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Excipients include, but are not limited to: butylated hydroxytoluene (BHT), calcium carbonate, calcium phosphate (dibasic), calcium stearate, croscarmellose, cross-linked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: Pharmaceutically acceptable salts of the compounds described herein are forms of the disclosed compounds wherein the acid or base moiety is in its salt form (e.g., as generated by reacting a free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Pharmaceutically acceptable salts include the conventional non-toxic salts, for example, from non-toxic inorganic or organic acids. In some embodiments a pharmaceutically acceptable salt is prepared from a parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety. Pharmaceutically acceptable solvate: The term “pharmaceutically acceptable solvate,” as used herein, refers to a crystalline form of a compound wherein molecules of a suitable solvent are incorporated in the crystal lattice. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.” In some embodiments, the solvent incorporated into a solvate is of a type or at a level that is physiologically tolerable to an organism to which the solvate is administered (e.g., in a unit dosage form of a pharmaceutical composition).

Pharmacokinetic: As used herein, “pharmacokinetic” refers to any one or more properties of a molecule or compound as it relates to the determination of the fate of substances administered to living organisms. Pharmacokinetics are divided into several areas including the extent and rate of absorption, distribution, metabolism and excretion. This is commonly referred to as ADME where: (A) Absorption is the process of a substance entering the blood circulation; (D) Distribution is the dispersion or dissemination of substances throughout the fluids and tissues of the body; (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue.

Physicochemical: As used herein, “physicochemical” means of or relating to a physical and/or chemical property.

Preventing: As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Proliferate: As used herein, the term “proliferate” means to grow, expand, replicate or increase or cause to grow, expand, replicate or increase. “Proliferative” means having the ability to proliferate. “Anti-proliferative” means having properties counter to or in opposition to proliferative properties.

Promoter: As used herein, the term “promoter” is a nucleotide sequence that permits binding of RNA polymerase and directs the transcription of a gene. A promoter is typically proximal to the transcriptional start site of the gene. A promoter can be inducible, repressible, constitutive, ubiquitous, tissue specific and/or synthetic (may be composed of sequences derived from different sources). The promoter may be located 5′ of the transcription start site of the payload, contained within the payload construct and may drives expression of the payload. The promoter may be located 5′ of the transcription start site of the regulatable element, and may drive the expression of the regulatable element. The size of the promoter can vary. Due to the limited packaging capacity of AAV, it is desirable to have a promoter as short as possible, while still able to provide the desired expression levels and regulation.

A synthetic promoter may be composed of sequences, such as various transcriptional elements, which are derived from different sources. A synthetic promoter may include regulatable elements, which are binding sites for transactivator or repressor fusion proteins. These fusion proteins may be components of regulatable elements. A synthetic promoter may contain transcriptional enhancer sequences. As used herein, the term “enhancer” refers to a regulatory region or element, which functions to enhance transcription. Non-limiting examples of enhancers include the CMV enhancer or portions thereof, or the UBC enhancer or portions thereof.

Protein of interest: As used herein, the terms “proteins of interest” or “desired proteins” include those provided herein and fragments, mutants, variants, and alterations thereof

Purified: As used herein, the term “purify” means to make substantially pure or clear from unwanted components, material defilement, admixture or imperfection. “Purified” refers to the state of being pure. “Purification” refers to the process of making pure.

Region: As used herein, the term “region” refers to a zone or general area. In some embodiments, when referring to a protein or protein module, a region may comprise a linear sequence of amino acids along the protein or protein module or may comprise a three dimensional area, an epitope and/or a cluster of epitopes. In some embodiments, regions comprise terminal regions. As used herein, the term “terminal region” refers to regions located at the ends or termini of a given agent. When referring to proteins, terminal regions may comprise N- and/or C-termini. N-termini refer to the end of a protein comprising an amino acid with a free amino group. C-termini refer to the end of a protein comprising an amino acid with a free carboxyl group. N- and/or C-terminal regions may there for comprise the N- and/or C-termini as well as surrounding amino acids. In some embodiments, N- and/or C-terminal regions comprise from about 3 amino acid to about 30 amino acids, from about 5 amino acids to about 40 amino acids, from about 10 amino acids to about 50 amino acids, from about 20 amino acids to about 100 amino acids and/or at least 100 amino acids. In some embodiments, N-terminal regions may comprise any length of amino acids that includes the N-terminus, but does not include the C-terminus. In some embodiments, C-terminal regions may comprise any length of amino acids, which include the C-terminus, but do not comprise the N-terminus.

In some embodiments, when referring to a polynucleotide, a region may comprise a linear sequence of nucleic acids along the polynucleotide or may comprise a three dimensional area, secondary structure, or tertiary structure. In some embodiments, regions comprise terminal regions. As used herein, the term “terminal region” refers to regions located at the ends or termini of a given agent. When referring to polynucleotides, terminal regions may comprise 5′ and 3′ termini. 5′ termini refer to the end of a polynucleotide comprising a nucleic acid with a free phosphate group. 3′ termini refer to the end of a polynucleotide comprising a nucleic acid with a free hydroxyl group. 5′ and 3′ regions may there for comprise the 5′ and 3′ termini as well as surrounding nucleic acids. In some embodiments, 5′ and 3′ terminal regions comprise from about 9 nucleic acids to about 90 nucleic acids, from about 15 nucleic acids to about 120 nucleic acids, from about 30 nucleic acids to about 150 nucleic acids, from about 60 nucleic acids to about 300 nucleic acids and/or at least 300 nucleic acids. In some embodiments, 5′ regions may comprise any length of nucleic acids that includes the 5′ terminus, but does not include the 3′ terminus. In some embodiments, 3′ regions may comprise any length of nucleic acids, which include the 3′ terminus, but does not comprise the 5′ terminus.

Regulatable AAV Particle or Regulatable AAV Particle: As used herein, the term “regulatable-AAV particle” is an AAV particle which comprises a capsid, a polynucleotide, and one or more regulatable elements and/or a payload which is regulated by one or more regulatable elements. Where CRISPR regulatable elements are present, the AAV particle may be referred to as a “Regulatable CRISPR-AAV particle”.

Regulatable Elements: As used herein, the term “regulatable element” refers to one or more components, factors, polynucleotide features or motifs which imparts regulatable or tunable features to regulate the expression of a payload. The expression of the regulatable elements may also be further regulated.

RNA or RNA molecule: As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single-stranded (i.e., ssRNA or ssDNA, respectively) or multi-stranded (e.g., double stranded, i.e., dsRNA and dsDNA, respectively). The term “mRNA” or “messenger RNA”, as used herein, refers to a single stranded RNA that encodes the amino acid sequence of one or more polypeptide chains.

RNA interference: As used herein, the term “RNA interference” or “RNAi” refers to a sequence specific regulatory mechanism mediated by RNA molecules which results in the inhibition or interference or “silencing” of the expression of a corresponding protein-coding gene.

Sample: As used herein, the term “sample” refers to an aliquot or portion taken from a source and/or provided for analysis or processing. In some embodiments, a sample is from a biological source such as a tissue, cell or component part (e.g. a body fluid, including but not limited to blood, mucus, lymphatic fluid, synovial fluid, cerebrospinal fluid, saliva, amniotic fluid, amniotic cord blood, urine, vaginal fluid and semen). In some embodiments, a sample may be or comprise a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. In some embodiments, a sample is or comprises a medium, such as a nutrient broth or gel, which may contain cellular components, such as proteins or nucleic acid molecule. In some embodiments, a “primary” sample is an aliquot of the source. In some embodiments, a primary sample is subjected to one or more processing (e.g., separation, purification, etc.) steps to prepare a sample for analysis or other use.

Self-complementary viral particle: As used herein, a “self-complementary viral particle” is a particle comprised of at least two components, a protein capsid and a polynucleotide sequence encoding a self-complementary genome enclosed within the capsid.

Sense strand: As used herein, the term “the sense strand” or “the second strand” or “the passenger strand” of a siRNA molecule refers to a strand that is complementary to the antisense strand or first strand. The antisense and sense strands of a siRNA molecule are hybridized to form a duplex structure. As used herein, a “siRNA duplex” includes a siRNA strand having sufficient complementarity to a section of about 10-50 nucleotides of the mRNA of the gene targeted for silencing and a siRNA strand having sufficient complementarity to form a duplex with the siRNA strand.

Signal Sequences: As used herein, the phrase “signal sequences” refers to a sequence which can direct the transport or localization.

Single unit dose: As used herein, a “single unit dose” is a dose of any therapeutic administered in one dose/at one time/single route/single point of contact, i.e., single administration event. In some embodiments, a single unit dose is provided as a discrete dosage form (e.g., a tablet, capsule, patch, loaded syringe, vial, etc.).

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Small/short interfering RNA: As used herein, the term “small/short interfering RNA” or “siRNA” refers to an RNA molecule (or RNA analog) comprising between about 5-60 nucleotides (or nucleotide analogs) which is capable of directing or mediating RNAi. Preferably, a siRNA molecule comprises between about 15-30 nucleotides or nucleotide analogs, more preferably between about 16-25 nucleotides (or nucleotide analogs), even more preferably between about 18-23 nucleotides (or nucleotide analogs), and even more preferably between about 19-22 nucleotides (or nucleotide analogs) (e.g., 19, 20, 21 or 22 nucleotides or nucleotide analogs). The term “short” siRNA refers to a siRNA comprising 5-23 nucleotides, preferably 21 nucleotides (or nucleotide analogs), for example, 19, 20, 21 or 22 nucleotides. The term “long” siRNA refers to a siRNA comprising 24-60 nucleotides, preferably about 24-25 nucleotides, for example, 23, 24, 25 or 26 nucleotides. Short siRNAs may, in some instances, include fewer than 19 nucleotides, e.g., 16, 17 or 18 nucleotides, or as few as 5 nucleotides, provided that the shorter siRNA retains the ability to mediate RNAi. Likewise, long siRNAs may, in some instances, include more than 26 nucleotides, e.g., 27, 28, 29, 30, 35, 40, 45, 50, 55, or even 60 nucleotides, provided that the longer siRNA retains the ability to mediate RNAi or translational repression absent further processing, e.g., enzymatic processing, to a short siRNA. siRNAs can be single stranded RNA molecules (ss-siRNAs) or double stranded RNA molecules (ds-siRNAs) comprising a sense strand and an antisense strand which hybridized to form a duplex structure called siRNA duplex.

Split dose: As used herein, a “split dose” is the division of single unit dose or total daily dose into two or more doses.

Stable: As used herein “stable” refers to a compound or entity that is sufficiently robust to survive isolation to a useful degree of purity from a reaction mixture, and preferably capable of formulation into an efficacious therapeutic agent.

Stabilized: As used herein, the term “stabilize”, “stabilized,” “stabilized region” means to make or become stable. In some embodiments, stability is measured relative to an absolute value. In some embodiments, stability is measured relative to a reference compound or entity.

Subject: As used herein, the term “subject” or “patient” refers to any organism to which a composition in accordance with the invention may be administered, e.g., for experimental, diagnostic, prophylactic, and/or therapeutic purposes. Typical subjects include animals (e.g., mammals such as mice, rats, rabbits, non-human primates, and humans) and/or plants.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially equal: As used herein as it relates to time differences between doses, the term means plus/minus 2%.

Substantially simultaneously: As used herein and as it relates to plurality of doses, the term typically means within about 2 seconds.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Synthetic: The term “synthetic” means produced, prepared, and/or manufactured by the hand of man. Synthesis of polynucleotides or polypeptides or other molecules of the present invention may be chemical or enzymatic.

Targeting: As used herein, “targeting” means the process of design and selection of nucleic acid sequence that will hybridize to a target nucleic acid and induce a desired effect.

Targeted Cells: As used herein, “targeted cells” refers to any one or more cells of interest. The cells may be found in vitro, in vivo, in situ or in the tissue or organ of an organism. The organism may be an animal, preferably a mammal, more preferably a human and most preferably a patient.

Therapeutic Agent: The term “therapeutic agent” refers to any agent that, when administered to a subject has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In some embodiments, a therapeutically effective amount is provided in a single dose. In some embodiments, a therapeutically effective amount is administered in a dosage regimen comprising a plurality of doses. Those skilled in the art will appreciate that in some embodiments, a unit dosage form may be considered to comprise a therapeutically effective amount of a particular agent or entity if it comprises an amount that is effective when administered as part of such a dosage regimen.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Total daily dose: As used herein, a “total daily dose” is an amount given or prescribed in a 24 hour period. It may be administered as a single unit dose.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Unmodified: As used herein, “unmodified” refers to any substance, compound or molecule prior to being changed in any way. Unmodified may, but does not always, refer to the wild-type or native form of a biomolecule or entity. Molecules or entities may undergo a series of modifications whereby each modified product may serve as the “unmodified” starting molecule or entity for a subsequent modification.

Untranslated regions: As used herein, “Untranslated regions” or UTRs refers to two polynucleotide sequences located on each side of a coding sequence. The polynucleotide sequence located on the 5′ side of the coding sequence is referred to as a 5′UTR and the polynucleotide sequence located on the 3′ side of the coding sequence is referred to as the 3′ UTR. As used herein, “payload 5′ UTR” and “payload 3′ UTR” refers to the polynucleotide sequences framing the payload on the 3′ and 5′ sides. Payload 5′ and 3′ UTRs are located between the payload coding sequence and the ITRs on each side of the payload.

The 3′ UTR can contain various post-transcriptional regulatable elements which can be used to affect the stability of the message. The 3′UTR may comprise a polyadenylation sequence, such as SV40 late polyadenylation site and others known in the art. The polyadenylation sequence is typically 3′ of other regulatable elements within the 3′UTR. Post-transcriptional regulatable elements which can be used to stabilize the payload mRNA include woodchuck hepatitis virus posttranscriptional regulatable element (WPRE), hepatitis B virus posttranscriptional regulatable element (HBVPRE) RNA transport element (RTE), and any variants thereof. For example, US Publication No. US20140127162, which is herein incorporated by reference in its entirety, describes a shorter WPRE sequence, which functions comparably to the full length WPRE sequence. AU rich elements (AREs) known in the art may also be used to destabilize or to stabilize an mRNA. The 3′ end of the payload construct 3′UTR may also contain a string of Adenosines.

Vector: As used herein, a “vector” is any molecule or moiety which transports, transduces or otherwise acts as a carrier of a heterologous molecule.

Vectors of the present invention may be produced recombinantly and may be based on and/or may comprise adeno-associated virus (AAV) parent or reference sequence. Such parent or reference AAV sequences may serve as an original, second, third or subsequent sequence for engineering vectors. In non-limiting examples, such parent or reference AAV sequences may comprise any one or more of the following sequences: a polynucleotide sequence encoding a polypeptide or multi-polypeptide, which sequence may be wild-type or modified from wild-type and which sequence may encode full-length or partial sequence of a protein, protein domain, or one or more subunits of a protein; a polynucleotide comprising a modulatory or regulatory nucleic acid which sequence may be wild-type or modified from wild-type; and a transgene that may or may not be modified from wild-type sequence . These AAV sequences may serve as either the “donor” sequence of one or more codons (at the nucleic acid level) or amino acids (at the polypeptide level) or “acceptor” sequences of one or more codons (at the nucleic acid level) or amino acids (at the polypeptide level).

Viral construct vector: As used herein, a “viral construct vector” is a vector which comprises one or more polynucleotide regions encoding or comprising Rep and or Cap protein.

Viral construct expression vector: As used herein, a “viral construct expression vector” is a vector which comprises one or more polynucleotide regions encoding or comprising Rep and or Cap that further comprises one or more polynucleotide regions encoding or comprising components for viral expression in a viral replication cell.

Viral genome: As used herein, a “viral genome” is a polynucleotide encoding at least one inverted terminal repeat (ITR), at least one regulatory sequence, and at least one payload. The viral genome is derived by replication of a payload construct from the payload construct expression vector. A viral genome encodes at least one copy of the payload construct. Where regulatable elements are encoded, a viral genome encodes at least one copy of the payload construct.

Viral particle: As used herein, “viral particle” refers to a functional recombinant virus.

### Equivalents and Scope

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment, of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment, of the compositions of the invention (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the invention in its broader aspects.

### EXAMPLES

### Example 1

### Gene Expression

The level of transgene expression by regulatable-AAV particles produced and purified by the methods described herein is determined by real-time quantitative polymerase chain reaction (qPCR). A culture of 293 cells engineered to produce helper components required for AAV production is infected by regulatable-AAV particles produced as described herein.

The target 293 cells are harvested at a series of time points, lysed and the mRNA is purified. The level of transgene expressed is determined by reverse transcription (qPCR) on a thermal cycler equipped with an excitation source filters, and detector for quantification of the reaction such as, but not limited to, the 7500 FAST Real-Time PCR system (Applied Biosystems, Foster City Calif.).

Regulatable-AAV particles produced and purified by the methods described herein are treated with proteinase K, serially diluted, and PCR-amplified using a fluor such as, but not limited to, SYBR green (Applied Biosystems, Foster City, Calif.) with primers specific to the transgene sequence. A reference transgene oligonucleotide is used as a copy number standard. The cycling conditions are: 95° C. for 3 min, followed by 35 cycles of 95° C. for 30 sec, 60° C. for 30 sec, and 72° C. for 30 sec.

### Example 2

### Recombinant Regulatable-AAV Production in Invertebrate Cells

The viral construct vector encodes the three structural cap proteins, VP1, VP2, and VP3, in a single open reading frame regulated by utilization of both alternative splice acceptor and non-canonical translational initiation codon(s). In-frame and out-of-frame ATG triplets preventing translation initiation at a position between the VP1 and VP2 start codons are eliminated. Both Rep78 and Rep52 are translated from a single transcript: Rep78 translation initiates at a non-AUG codon and Rep52 translation initiates at the first AUG in the transcript.

The nucleotides that encode the structural VP1, VP2, and VP3 capsid proteins and non-structural Rep78 and Rep52 proteins are contained on one viral expression construct under control of the baculovirus major late promoter.

The payload construct vector encodes two ITR sequences flanking a transgene polynucleotide encoding a polypeptide or modulatory nucleic acid and/or one or more regulatable elements. The ITR sequences allow for replication of a polynucleotide encoding the transgene and ITR sequences alone that will be packaged within the capsid of the viral construct vector. The replicated polynucleotide encodes ITR sequences on the 5′ and 3′ ends of the molecule.

The payload construct vector and viral construct vector each comprise a Tn7 transposon element that transposes the ITR and payload sequences or the Rep and Cap sequences respectively to a bacmid that comprises the attTn7 attachment site. Competent bacterial DH10 cells are transfected with either the payload construct vector or viral construct vector. The resultant viral construct expression vector and payload construct expression vector produced in the competent cell are then purified by detergent lysis and purification on DNA columns.

Separate seed cultures of Sf9 cells in serum free suspension culture are transfected with the viral construct expression vector or payload construct expression vector. The cultures are maintained for 48 hours while baculovirus is produced and released into the medium. The baculovirus released into the media continue to infect Sf9 cells in an exponential manner until all of the Sf9 cells in the culture are infected at least once. The baculoviral infected insect cells (BIIC) and media of the seed culture is harvested and divided into aliquots before being frozen in liquid nitrogen.

A naïve population of un-transfected Sf9 cells is expanded in serum free suspension cell culture conditions. Once the culture growth has reached peak log phase in 1 L of media as measured by optical density the culture is added to a large volume 20 L bioreactor. The bioreactor culture is co-inoculated with a frozen viral construct expression vector and payload construct expression vector BIIC aliquot. The conditions of the Sf9 cell suspension culture is monitored by instruments that measure and/or control external variables that support the growth and activity of viral replication cells such as mass, temperature, CO2, O2, pH, and/or optical density (OD). The Sf9 culture is maintained at optimal conditions until cell population growth has reached peak log phase and before cell growth has plateaued, as measured by optical density.

In each viral replication cell that has been infected with both baculoviruses the payload flanked on one end with an ITR sequence is replicated pathway producing a viral genome and packaged in a capsid assembled from the proteins VP1, VP2, and VP3.

The viral replication cells are lysed using the Microfluidizer™ (Microfluidics International Corp., Newton, Mass.), high shear force fluid processor. The resultant cell lysate is clarified by low speed centrifugation followed by tangential flow filtration. The resultant clarified lysate is filtered by a size exclusion column to remove any remaining baculoviral particles from solution. The final steps utilize ultracentrifugation and sterile filtration to produce viral particles suitable for use as described herein.

The titer of regulatable-AAV particles produced and purified by the methods described herein is determined by real-time quantitative polymerase chain reaction (qPCR) on a thermal cycler equipped with an excitation source filters, and detector for quantification of the reaction such as, but not limited to, the 7500 FAST Real-Time PCR system (Applied Biosystems, Foster City Calif.). Regulatable-AAV particles produced and purified by the methods described herein is treated with proteinase K, serially diluted, and PCR-amplified using a fluor such as, but not limited to, SYBR green (Applied Biosystems, Foster City, Calif.) with primers specific to the AAV genome ITR sequences. A linearized viral genome is used as a copy number standard. The cycling conditions are: 95° C. for 3 min, followed by 35 cycles of 95° C. for 30 sec, 60° C. for 30 sec, and 72° C. for 30 sec.

### Example 3

### Incorporation of Destabilizing Sequences or Domains

To minimize long-term dsDNA cleavage, regulatable-AAV particles with destabilizing domains and destabilizing RNA sequences are prepared, produced and tested.

### A. Destabilizing Domains—Cas9 Fusion Protein

### Overview

While not wishing to be bound by theory, destabilizing domains are known to confer instability and decrease transgene expression. The presence of the destabilizing domain can trigger the cell's proteasomal degradation systems, which then can lead to cas9 destruction. Destabilizing domains may comprise peptide sequences which are rich in a particular subset of amino acids which are thought to signal degradation, such as, but not limited to, proline, glutamic acid, serine and threonine (known as PEST sequences).

In some embodiments, destabilizing domains, which can be used in the cas9-fusion protein encoded by the payload construct expression vector include, but are not limited to, destabilizing domains from FK506 Binding Protein (FKBP), E. coli dihyrofolate reductase (DHFR), mouse ornithine decarboxylase (MODC), and estrogen receptors (ER). As a non-limiting example, the destabilizing domain may be from an estrogen receptor.

### AAV Construct Preparation and Analysis

In order to study methods to minimize unwanted long-term cleavage of dsDNA, a regulatable particle of Example 2 with a cas9-fusion protein (e.g., a cas9 endonuclease fused to a destabilizing domain) is prepared, produced and tested by methods known in the art and described herein.

The decreased protein half-life of cas9 endonuclease with a destabilizing domain is tested by transfecting HEK293 cells with the payload construct expression vectors comprising cas9 with or without destabilizing domain. Cells are lysed over a predetermined time course (e.g., 24 hours) post infection and protein extracts prepared for ELISA and Western blot analysis. Further, in separate plates, fluorescently tagged payload construct expression vectors are infected into the same cell system and fluorescence intensity is monitored over time. Additionally, cycloheximide blocking and pulse chase experiments are performed in the same cell system.

### B. Destabilizing RNA Sequences

To prepare a destabilized RNA sequence, the AAV particle of Example 2 includes a 3′UTR destabilizing sequence. This destabilized RNA sequence may be used to shorten the half-life of cas9.

One type of 3′UTR destabilizing sequence is AU-rich elements (AREs) and AUUUA motifs. While not wishing to be bound by theory these elements and motifs may be the primary means for mediating mRNA destabilization. Examples of this type of motif are evident in the 3′UTR of human estrogen receptor alpha (hERa) as well as in cytokines, proto-oncogenes and interferon mRNAs.

To determine the effectiveness of the destabilizing RNA sequence, an AAV particle which encodes cas9 and contains a destabilizing sequence, is produced and purified as in example 2. An AAV particle without the destabilizing sequence is used as a control and produced in parallel. The half-life of cas9 mRNA and/or the corresponding protein levels are analyzed over a predetermined time course in HEK293 cells. Cells are lysed over a time course post infection and RNA and protein extracts are prepared. Half-life of mRNA in both samples is measured by methods known in the art and described herein such as monitoring deadenylation via transcriptional pulsing techniques.

### Example 4

### Punctuated Expression of cas9

In order to study methods to induce transient, burst expression of cas9, an AAV particle of Example 2 which encodes cas9, a DNA binding domain and a transactivating factor is prepared, produced and tested by methods known in the art and described herein. The cas9 sequence is located in the open reading frame of the vector and a DNA binding domain (DBD) and transactivating factor are located in VP2. The transactivating factor may be coupled to the DBD.

The DBD may be a pre-engineered DBD targeted specifically to the promoter used for cas9. While not wishing to be bound by theory, upon expression of VP2 in a biological system, the DBD locates and binds to the cas9 promoter. If the transactivating factor is for cas9 and the transactivating factor is coupled to the DBD, upon DBD binding to the cas9 promoter, the transactivating factor drives expression of cas9.

The nature of the interaction between the promoter and the DBD coupled to a transactivating factor generates a transient, burst expression of cas9. This punctuated expression may be beneficial as it may limit the possibility of side-effects of extended elevated expression of cas9.

To study the burst expression of cas9, an AAV particle with the DBD and the transactivating factor is purified and produced as described in Example 2. As a control, an AAV particle lacking the DBD and transactivating domain is produced in parallel.

Expression of cas9 is measured by methods described herein and known in the art, such as in HEK293 cells for the AAV particle with or without the DBD and transactivating factor. Cells are lysed at different time points and protein extracts are prepared for ELISA and Western blot analysis.

The specificity of cas9 cleavage is confirmed by deep sequencing of samples collected and the extent of dsDNA cleavage by cas9 or cas9-destabilizing domain fusion protein is measured by ligation-mediated purification or genome modification assays such as SURVEYOR. Indel percentage is calculated from the integrated intensities of the undigested PCR product and each of the cleavage products.

### Example 5

### Regulation Through a Rapamycin Inducible System

The ability of a regulatable element composed of two rapamycin inducible fusion proteins to regulate the expression of a luciferase payload is tested in vitro and in vivo.

### In Vitro Testing

The following regulatable-AAV constructs are prepared, produced and tested: AAV construct 1 contains a luciferase payload which is driven by a promoter, which is a minimal promoter into which one or more ZHFD1 binding sites are inserted. AAV construct 2 contains the elements of construct 1 and further encodes two dimerizable fusion proteins, FKBP containing the DNA-binding domain of ZHFD1 and FRAP fused to the NF-kappaB p65 transactivation domain. The dimerizable fusion proteins are expressed from one constitutive promotor, and linked together through a 2A peptide sequence. The transcription factor and the transactivation domain fusion proteins both contain a nuclear localization sequence. AAV construct 3 encodes luciferase driven by a CMV promoter for strong constitutive expression (positive control). AAV construct 4 encodes luciferase driven by a CMV promoter for strong constitutive expression and the dimerization fusion proteins driven by a constitutive promoter (positive control).

Two AAV vectors can be used for transduction, one expressing the regulatable elements (dimerizable transcription factors) and the other expressing the luciferase payload. These are be delivered at a ratio determined to be optimal. Alternatively, one vector is used for transduction, expressing the regulatable elements and the luciferase payload.

HeLa cells are transduced with the AAV vector(s) constructs 1 through 4, according to methods known in the art in triplicate for each assay. The transduced HeLa cells are incubated in medium in the presence or absence of rapamycin for a set time. Subsequently, cells are harvested according to a time course which includes 24, 48, and 72 hours. The cells are lysed and luciferase activity is measured and compared between untreated and rapamycin treated samples 1 and 2 and the controls (samples 3 and 4).

### In Vivo Testing

The AAV constructs 1, 2 and 3 are prepared, produced and tested. In addition, a fifth construct derived from construct 2 is prepared, in which the constitutive promoter driving the expression of the dimerizable fusion proteins is replaced by a tissue-specific promoter (liver specific).

AAV particles containing constructs 1-3 and 5 are each injected into two mice of 3 to 5 months of age according to methods known in the art. At a set time post injection, a further injection of rapamycin is administered to half of the mice. At a predetermined time, mice are injected with luciferin. Animals are then anesthetized and images are acquired with an imaging system. Bioluminescence is measured as total flux (photons/second) of the entire mouse.

### Example 6

### Regulation Through a CRISPR Regulatable Element

The ability of a CRISPR regulatable elements to regulate the expression of a luciferase payload composed of one or more CRISPR recognition sequences is tested in vitro and in vivo. Cleavage of the payload construct at the CRISPR recognition sequence ablates the payload expression. Using Cas9 with a destabilizing domain shortens the half-life of Cas9.

### In Vitro Testing

The following CRISPR-AAV constructs are prepared, produced and tested.

AAV construct 1 encodes a luciferase payload, which is driven by a constitutive promoter and which contains a CRISPR recognition sequence. The recognition sequence is chosen using methods known in the art and described herein to ensure that the sequence is unique to the viral genome and does not occur in the host genome. The AAV construct 2, which contains all of the components of construct 1, and additionally encodes a guide RNA specific to the CRISPR recognition sequence in the luciferase payload and a codon optimized Cas9 with a destabilizing domain. The guide RNA and Cas9 are both expressed from a constitutive promotor. Cas9 also contains a nuclear localization sequence. AAV construct 3 encodes luciferase driven by a CMV promoter without the CRISPR recognition sequence (positive control). AAV construct 4 contains all of the elements of construct 3, and further encodes a guide RNA specific to the CRISPR recognition sequence located in the luciferase payload and a Cas9 with a destabilizing domain. The guide RNA and Cas9 are both expressed from a constitutive promotor. Cas9 also contains a nuclear localization sequence.

In one example, two AAV vectors are used for transduction, one expressing the regulatable elements (Cas9 and guide RNA) and the other expressing the luciferase payload. These two vectors are delivered at a ratio determined to be optimal. Alternatively, one vector is used for transduction, expressing the regulatable elements and the luciferase payload.

HeLa cells are transduced with the AAV vector(s) constructs 1 through 4, according to methods known in the art in triplicate for each assay. The transduced HeLa cells are incubated in medium. Subsequently, cells are harvested according to a time course which includes 24, 48, and 72 hours. The cells are lysed and luciferase activity is measured and compared between the samples.

If the regulatable elements and the luciferase payload are on two separate vectors, the effective dose of the CRISPR regulatable element is determined. HeLa cells are transduced with construct 1 and different doses of the AAV vector encoding Cas9 and the guide RNA, each in triplicate. Cells are harvested, lysed and luciferase activity is measured for each dose of Cas9 and guide RNA employed.

### In Vivo Testing

The AAV constructs 1 and 2 are prepared, produced and tested. AAV particles 1 and 2 are each injected into mice of 3 to 5 months of age according to methods known in the art. At a set time post injection, such as for example 2 weeks, mice are injected with luciferin. Animals are then anesthetized and images are acquired with an imaging system. Bioluminescence is measured as total flux (photons/second) of the entire mouse and compared between the samples.

### Example 7

### Regulation Through an Inducible Element and a CRISPR Regulatable Element

The ability of an inducible system to regulate a CRISPR regulatable element, which in turn can regulate the payload in the context of AAV transduction is tested in vitro and in vivo. The ability of a regulatable element composed of two rapamycin inducible fusion proteins to regulate the expression of a CRISPR regulatable element is tested in vitro.

### In Vitro Testing

The following CRISPR-AAV constructs are prepared, produced and tested. AAV construct 1 encodes a luciferase payload, which contains a CRISPR recognition sequence, driven by a constitutive promoter. The recognition sequence is chosen using methods known in the art and described herein to ensure that the sequence is unique to the viral genome and does not occur in the host genome. AAV construct 2 contains the components of construct 1 and additionally encodes a guide RNA specific to the CRISPR recognition sequence located in the luciferase payload and a codon optimized Cas9 with a destabilizing domain. The guide RNA and Cas9 are both expressed from a constitutive promotor. Cas9 also contains a nuclear localization sequence. AAV construct 3 contains all of the components of construct 1, and additionally encodes a guide RNA specific to the CRISPR recognition sequence located in the luciferase payload and a codon optimized Cas9 with a destabilizing domain. The guide RNA and Cas9 are both expressed from a minimal promotor into which one or more ZHFD1 binding sites are inserted. Cas9 also contains a nuclear localization sequence. AAV construct 4 contains all of the components of construct 3, and additionally encodes two dimerizable fusion proteins, FKBP containing the DNA-binding domain of ZHFD1 and FRAP fused to the NF-kappaB p65 transactivation domain. The dimerizable fusion proteins are expressed from one constitutive promotor, and linked together through a 2A peptide sequence. The transcription factor and the transactivation domain fusion proteins both contain a nuclear localization sequence.

Two or more AAV vectors can be used for transduction, one or more expressing the regulatable elements (Cas9 and guide RNA, and dimerizable fusion proteins) and an additional vector expressing the luciferase payload. Alternatively, three AAV vectors are used, one encoding the CRISPR regulatable elements, one encoding the dimerizable fusion proteins, and one encoding the luciferase payload. These vectors are delivered at a ratio determined to be optimal. In another embodiment, one vector is used for transduction, expressing the regulatable elements and the luciferase payload. In one embodiment, an open reading frame for the DNA binding domain fusion protein and/or transactivation domain fusion protein is located in VP2.

HeLa cells are transduced with the AAV vector constructs 1 through 4, according to methods known in the art in triplicate for each assay. The transduced HeLa cells are incubated in medium in the presence or absence of rapamycin for a set time. Subsequently, cells are harvested according to a time course which includes 24, 48, and 72 hours. The cells are lysed and luciferase activity is measured. Throughout the time course, luciferase activity is compared between untreated and rapamycin treated samples for each AAV vector construct and also between samples transduced with the vector constructs 1 through 4.

### In Vivo Testing

The AAV constructs 1-4 are prepared, produced and tested. In addition, a 5^{th} construct may be added, which is the same as 4, except that the fusion proteins are driven by a tissue specific promoter, such as a liver specific promoter. AAV particles 1-4 are each injected into two mice of 3 to 5 months of age according to methods known in the art. At approximately 2 weeks post injection, a further injection of rapamycin is administered to half of the mice. At a predetermined time, mice are injected with luciferin. Animals are then anesthetized and images are acquired with an imaging system. Bioluminescence is measured as total flux (photons/second) of the entire mouse. Luciferase activity is compared between untreated and rapamycin treated animals and also between animals injected with the AAV particles 1-4.

### Example 8

### Regulation Through an Inducible Element and a TALEN Regulatable Element

The ability of an inducible system to regulate a TALEN regulatable element, which in turn can regulate the payload in the context of AAV transduction is tested in vitro and in vivo. The ability of a tetracycline regulatable element to regulate the expression of a TALEN regulatable element is tested in vitro.

### In Vitro Testing

The following regulatable-AAV constructs are prepared, produced and tested. AAV construct 1 encodes a luciferase payload which contains a TALEN recognition sequence driven by a constitutive promoter. The recognition sequence is chosen using methods known in the art and described herein to ensure that the sequence is unique to the viral genome and does not occur in the host genome. AAV construct 2 contains all of the components of construct 1 and further encodes a TALEN, expressed from a constitutive promoter. The TALEN also contains a nuclear localization sequence. AAV construct 3 contains all of the components of construct 1 and additionally encodes a TALEN, whose expression is driven from a minimal promotor into which one or more tetracycline response elements (TRE) are inserted. The TALEN also contains a nuclear localization sequence. AAV construct 4 contains all of the components of construct 3, and additionally encodes a tetracycline transactivator protein (fusion protein of tetracycline repressor TetR and VP16 transactivation domain), driven by a constitutive promoter. The tetracycline transactivator protein contains a nuclear localization sequence.

In one example, two AAV vectors are used for transduction, one expressing the tetracycline regulatable element and the other expressing the luciferase payload. These vectors are delivered at a ratio determined to be optimal. Alternatively, one vector is used for transduction, expressing the tetracycline regulatable element and the luciferase payload. In one exemplary setup, an open reading frame for the tetracycline regulatable element fusion protein is located in VP2.

HeLa cells are transduced with the AAV vector constructs 1 through 4, according to methods known in the art in triplicate for each assay. The transduced HeLa cells are incubated in medium in the presence or absence of tetracycline for a set time. Subsequently, cells are harvested according to a time course which includes 24, 48, and 72 hours. The cells are lysed and luciferase activity is measured. Throughout the time course, luciferase activity is compared between untreated and tetracycline treated samples for each AAV vector construct and also between samples transduced with the vector constructs 1 through 4.

### Example 9

### Regulation of a CRISPR Regulatable Element Through a Rapamycin-Inducible Destabilizing Domain

The ability of a CRISPR regulatable element proteins to regulate the expression of a luciferase payload containing one or more CRISPR recognition sequences is tested. Cleavage of the payload construct at the CRISPR recognition sequence ablates the payload expression. Using Cas9 with a destabilizing domain, which is stabilized by an inducer, allows the Cas9 to be permanently turned off in the absence of inducer. FK506- and rapamycin-binding protein domain is chosen, which can be regulated by rapamycin and its analogs, and is unstable in the absence of its ligand.

### In Vitro Testing

The following CRISPR-AAV constructs are prepared, produced and tested. AAV construct 1 encodes a luciferase payload, which contains a CRISPR recognition sequence, driven by a constitutive promoter. The recognition sequence is chosen using methods known in the art and described herein to ensure that the sequence is unique to the viral genome and does not occur in the host genome. AAV construct 2 contains the components of construct 1 and additionally encodes a guide RNA specific to the CRISPR recognition sequence located in the luciferase payload and a Cas9 without a destabilizing domain. The guide RNA and Cas9 are both expressed from a constitutive promotor. Cas9 also contains a nuclear localization sequence. AAV construct 3 includes all of the components of construct 1, and additionally encodes a guide RNA specific to the CRISPR recognition sequence found in the luciferase payload and a codon optimized Cas9 with FKBP12 destabilizing domain. The guide RNA and Cas9 are both expressed from a constitutive promotor. Cas9 also contains a nuclear localization sequence.

In one experimental setup, two or more AAV vectors are used for transduction, one or more vectors expressing the regulatable elements (Cas9 and guide RNA) and the other expressing the luciferase payload. These two vectors are delivered at a ratio determined to be optimal. Alternatively, one vector is used for transduction, expressing the regulatable elements and the luciferase payload.

HeLa cells are transduced with the AAV vector constructs 1 through 4, according to methods known in the art in triplicate for each assay. The transduced HeLa cells are incubated in medium in the presence or absence of rapamycin for a set time. Subsequently, cells are harvested according to a time course which includes 24, 48, and 72 hours. The cells are lysed and luciferase activity is measured. Throughout the time course, luciferase activity is compared between untreated and rapamycin treated samples for each AAV vector construct and also between samples transduced with the vector constructs 1 through 4.

Reversibility of the treatment is also tested. For samples transduced with AAV vector 3 and treated with rapamycin for 72 hours, rapamycin is removed from the growth medium for about 24 hours, and the cells are lysed and luciferase activity is measured.

### Example 10

### Regulation Through an siRNA Regulatable Element

The ability of a siRNA regulatable element proteins to regulate the expression of a luciferase payload containing an siRNA binding sequence is tested in vitro and in vivo. Upon binding of the siRNA to the payload construct mRNA, the mRNA is targeted for degradation.

### In Vitro Testing

The following regulatable-AAV constructs are prepared, produced and tested. AAV construct 1 contains a luciferase payload, which contains a siRNA binding sequence, driven by a constitutive promoter. The binding sequence is chosen using methods known in the art to ensure that the sequence is unique to the viral mRNA and does not occur in the host exome. AAV construct 2 contains all of the components of construct 1 and additionally encodes a siRNA specific to the siRNA binding sequence located in the luciferase payload construct. The siRNA is expressed from a constitutive promotor. AAV construct 3 encodes luciferase driven by a CMV promoter without the siRNA binding sequence (positive control). The AAV construct 4 encodes luciferase driven by a CMV promoter without the siRNA binding sequence and additionally encodes the siRNA, expressed from a constitutive promoter.

Two AAV vectors can be used for transduction, one expressing the siRNA and the other expressing the luciferase payload. These two vectors are delivered at a ratio determined to be optimal. Alternatively, one vector may be used for transduction, expressing the siRNA and the luciferase payload.

HeLa cells are transduced with the AAV vector(s) constructs 1 through 4, according to methods known in the art in triplicate for each assay. The transduced HeLa cells are incubated in medium. Subsequently, cells are harvested according to a time course which includes 24, 48, and 72 hours. The cells are lysed and luciferase activity is measured and compared between the samples.

If the regulatable elements and the luciferase payload are on 2 separate vectors, the effective dose of the siRNA is determined. HeLa cells are transduced with Construct 1 and different doses of the AAV vector comprising the siRNA, each in triplicate. Cells are harvested, lysed and luciferase activity is measured for each dose of Cas9 and guide RNA employed.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

1-48. (canceled)

49. A regulatable-AAV particle comprising a viral genome, the viral genome comprising:
(a) a sequence encoding at least one payload;
(b) a sequence encoding at least one regulatable element,
wherein the regulatable element comprises a DNA binding domain, a transactivation domain, a repressor domain, a ligand binding domain, and/or an enzyme.

50. The regulatable-AAV particle of claim 49, wherein the genome comprises a sequence encoding a first regulatable element, and the AAV particle comprises an AAV capsid which comprises a sequence encoding a second regulatable element.

51. The regulatable-AAV particle of claim 50, wherein the first regulatable element regulates the expression of the second regulatable element.

52. The regulatable-AAV particle of claim 49, wherein the regulatable element comprises a heterologous domain which:
(a) stabilizes the payload;
(b) destabilizes the payload;
(c) is stabilized in the presence of a ligand and destabilized in the absence of the ligand; or
(d) is destabilized in the presence of a ligand and stabilized in the absence of the ligand.

53. The regulatable-AAV particle of claim 49, wherein the regulatable element comprises a DNA binding domain, and wherein the DNA binding domain is selected from the group consisting of Gal4, CREB, HSF, ZFHD1, Ecdysone Receptor, glucocorticoid receptor, RXR, RAR, Stat proteins, myc, zinc finger nuclease, TAL effectors, and RNA guided DNA binding domains.

54. The regulatable-AAV particle of claim 49, wherein the regulatable element comprises a transactivation domain, and wherein the transactivation domain is selected from the group consisting of Gal4, Oaf1, Leu3, Rtg3, Pho4, Gln3, Gcn4, p53, RTg3, CREB, Gli3, E2A, HSF1, NF-IL6, myc, NFAT, NF-κB, and VP16.

55. The regulatable-AAV particle of claim 49, wherein the regulatable element comprises a repressor domain, and wherein the repressor domain is selected from the group consisting of KRAB, ERD, and SID.

56. The regulatable-AAV particle of claim 49, wherein the regulatable element comprises a ligand binding domain, and wherein the ligand binding domain is selected from the group consisting of Ecdysone Receptor, glucocorticoid receptor, RXR, RAR, tet repressor, rapamycin, and rapamycin analog binding domains.

57. The regulatable-AAV particle of claim 49, wherein the regulatable element comprises an enzyme to cleave the payload.

58. The regulatable-AAV particle of claim 57, wherein the enzyme is selected from the group consisting of meganuclease, zinc finger nuclease, TALEN, recombinase, integrase, and Cas9.

59. The regulatable-AAV particle of claim 58, wherein the enzyme is Cas9 and the regulatable element further comprises a single guide RNA (sgRNA).

60. The regulatable-AAV particle of claim 49, wherein the nucleic acid encoding the payload comprises one or more CRISPR recognition sequences.

61. The regulatable-AAV particle of claim 49, wherein the payload is a polypeptide of interest or a nucleic acid of interest.

62. The regulatable-AAV particle of claim 61, wherein the polypeptide of interest is a therapeutic protein.

63. The regulatable-AAV particle of claim 61, wherein the nucleic acid of interest is a modulatory nucleic acid selected from the group consisting of tRNA, rRNA, tmRNA, miRNA, RNAi, siRNA, piRNA, shRNA antisense RNA, double stranded RNA, snRNA, snoRNA, and long non-coding RNA.

64. A method of synthesizing a regulatable-AAV particle comprising:
a) providing viral replication cells comprising:
i) a payload construct expression vector comprising a payload and one or more regulatable elements flanked on each side by a parvoviral ITR sequence to produce a payload construct expression vector to produce a payload construct particle; and
ii) a viral construct expression vector(s) comprising parvoviral rep and/or cap gene sequences under the control of one or more regulatable elements to produce a viral construct expression vector to produce a viral construct particle; and
b) co-infecting a viral replication cell with the payload construct particle the viral construct particle to produce a regulatable-AAV particle.

65. A method of synthesizing a regulatable-AAV particle comprising co-infecting a viral replication cell with (a) a payload construct viral particle which comprises a payload and one or more regulatable elements, and (b) a viral construct viral particle comprising parvoviral rep and/or cap gene sequences under the control of the one or more regulatable elements.

66. A method of regulating the expression of a protein of interest or a nucleic acid of interest, the method comprising contacting a subject with the regulatable-AAV particle of claim 49.

67. A method of treating a CNS disorder in a subject in need thereof, the method comprising administering to the subject a therapeutically effective amount of one or more regulatable-AAV particles of claim 49.

68. The method of claim 67, wherein the CNS disorder is selected from the group consisting of: Alzheimer's Disease, Amyotrophic lateral sclerosis, Creutzfeldt-Jakob Disease, Huntingtin's Disease, Friedreich's ataxia, Parkinson's Disease, Multiple System Atrophy, Spinal Muscular Atrophy, Multiple Sclerosis, Primary progressive aphasia, Progressive supranuclear palsy, Dementia, Brain Cancer, Degenerative Nerve Diseases, Encephalitis, Epilepsy, Genetic Brain Disorders that cause neurodegeneration, Retinitis pigmentosa, Head and Brain Malformations, Hydrocephalus, Stroke, Prion disease, and Infantile neuronal ceroid lipofuscinosis.
